(19) United States
(12) Patent Application Publication
(10) Pub. No.: US 2014/0234364 A1
(43) Pub. Date: Aug. 21, 2014

(54) TOTAL SYNTHESIS AND IMMUNOLOGICAL EVALUATION OF SACCHARIDE MOIETIES OF THE LIPOPOLYSACCHARIDE FROM NEISSERIA MENINGITIDIS

(71) Applicant: MAX-PLANCK-GESELLSCHAFT ZUR FÖRDERUNG DER WISSENSCHAFTEN E.V., Munchen (DE)

(72) Inventors: Peter H. Seeberger, Kleinmachnow (DE); You Yang, Berlin (DE); Chakkumkal Anish, Berlin (DE); Anika Reinhardt, Berlin (DE)

(73) Assignee: Max-Planck-Gesellschaft zur Förderung der Wissenschaften e.V., München (DE)

(21) Appl. No.: 14/346,858

(22) PCT Filed: Sep. 24, 2012
(86) PCT No.: PCT/EP2012/068811

§ 371 Date: Mar. 24, 2014

(30) Foreign Application Priority Data
Sep. 23, 2011 (EP) 11075215.1

(51) Int. Cl. A61K 39/095; G01N 33/569; A61K 39/385; C07H 15/04; C07K 16/12

(52) U.S. Cl. CPC A61K 39/095; C07H 15/04; C07K 16/1217; A61K 39/385; G01N 33/56911
USPC 424/190.1; 536/16.8; 536/17.9; 530/395; 530/387.5

(57) ABSTRACT

The present invention relates to the total chemical synthesis of the monosaccharide 35^{#} (R′═H), the disaccharide 36^{#} (R′≠H; R″═H), the trisaccharide 37^{#} (R′≠H; R″≠H; R′″≠H) and the tetrasaccharide 1^{#} (R′≠H; R″≠H; R′″≠H) of the following general formula wherein R represents —Y—NH₂Y represents a linker R′ is H or R″ is H or R′″ is H or of the lipopolysaccharide from Neisseria meningitidis, as well as to the trisaccharide 37^{#} and the tetrasaccharide 1^{#}, to vaccines containing at least one of the saccharides 1^{#}, 35^{#}, 36^{#}, and 37^{#} and to the use of such vaccine for immunization against diseases caused by infection with bacteria containing the tetrasaccharide α-GlcNAc-(1→2)-α-Hep-(1→3)-α-Hep-(1→5)-α-Kdo or the trisaccharide α-Hep-(1→3)-α-Hep-(1→5)-α-Kdo or α-GlcNAc-(1→2)-α-Hep-(1→3)-α-Hep, especially for immunization against meningitis, septicaemia, pneumonia and nasopharyngitis caused by Neisseria meningitidis.

### FIELD OF THE INVENTION

The present invention relates to the total chemical synthesis of the monosaccharide 35^{#}, the disaccharide 36^{#}, the trisaccharide 37^{#} and the tetrasaccharide 1^{#} of the lipopolysaccharide from Neisseria meningitidis, as well as to the trisaccharide 37^{#} and the tetrasaccharide 1^{#}, to vaccines containing at least one of the saccharides 1^{#}, 35^{#}, 36^{#}, and 37^{#} and to the use of such vaccine for immunization against diseases caused by infection with bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep, especially for immunization against meningitis, septicaemia, pneumonia and nasopharyngitis caused by Neisseria meningitidis.

### BACKGROUND OF THE INVENTION

Bacterial meningitis is a severe infection of the meninges, and accounts for about 1,200,000 cases of the disease worldwide annually, affecting mostly infants, children, and young adults who do not have specific protective antibodies. Neisseria meningitidis often referred to as meningococcus is a leading cause of bacterial meningitis and the only Gram-negative encapsulated bacterium responsible for large epidemics of the disease. Rapid development of meningococcal infection often leads to death within a few hours; even with appropriate care at least 10% of patients die, while up to 20% of survivors are left with permanent aftereffects such as deafness, epilepsy, mental retardation, or other neurological disorders.

Two types of polysaccharides, capsular polysaccharide (CPS) and lipopolysaccharide (LPS), are the major virulence factors in N. meningitidis infections. Meningococcal CPS consists of a slimy polysaccharide capsule surrounding the bacteria, and is a linear chain of mono or disaccharide repeating units. Based on these CPS structures, N. meningitidis can be divided into 13 serogroups. Five of these serogroups (A, B, C, Y, W135) are associated with the vast majority of meningococcal disease in humans, and their distribution is strictly limited geographically. For example, in Europe and North America, most cases of meningitis are caused by bacteria from serogroups B and C, while in the African meningitis belt, where the infection rate ranges from 100 to 800 per 100,000 population during epidemics, group A bacteria are the predominant cause. CPS-based meningococcal vaccines were first introduced in the 1970s and are now commercially available. These vaccines are comprised of CPS isolated from natural sources, assembled as bivalent polysaccharides containing combinations of glycans from A+C, tetravalent polysaccharides combinations of A+C+Y+W135, as well as the group A glycocojugate, the group C glycoconjugate and the tetravalent groups A+C+Y+W135 glycoconjugate made by chemical conjugation of each polysaccharide to a carrier protein (typically tetanus toxoid or CRM₁₉₇). Although CPS-based vaccines can provide large-scale protection of public health against most CPS serogroups, there is currently no vaccine to prevent group B infection. This is because CPS of serogroups B consists of α-2→8-linked polysialic acid, which is an autoantigen due to its presence in neural tissue of infants and therefore does not qualify as a suitable vaccine candidate due to the risk of triggering an autoimmune response. Beside that polysaccharide vaccines are generally poorly immunogenic in young children. So developing a vaccine based on N. meningitidis B capsular polysaccharide is difficult. In this context, subcapsular antigens would be an ideal candidate for developing vaccines against N. meningitis B infections.

Lipopolysaccharide (LPS) is an important virulence factor present in the outer membrane of N. meningitidis (FIG. 1). So far 13 distinct meningococcal lipooligosaccharide (LOS) immunotypes have been observed and all of them have common structural elements like the core structure, the O-antigen (or O-polysaccharide), and the lipid A moiety. However, the LPS of N. meningitidis do not possess a polymeric O-antigen, thus, in this case, the core structure is the most exposed and hence immunodominant part of LPS. All clinically relevant meningococcal strains share a common LPS inner core structure (see FIG. 1), therefore an LPS-based vaccine may generate broadly protective antibodies against the major disease-causing serotypes of N. meningitidis, including group B. Over the past two decades, efforts have been focused on developing LPS-based vaccines that would provide populationwide, comprehensive protection against meningococcal disease across serogroups A, C and most importantly group B.

However, relying on bacterial isolates limits the availability of LPS to scarce amounts, and the isolated glycans frequently show high degrees of microheterogenicity in the outer core, which complicates structural and biological analysis. Consequently, antibody responses to different batches of isolated lipopolysaccharides can vary. Another complication is that isolated LPS must be detoxified to remove the lipid A which is as endotoxin responsible for much of the toxicity of N. meningitidis before it can be considered as a vaccine moiety. To fully explore the effectiveness of LPS oligosaccharides as vaccine candidates against N. meningitidis, and to identify the most immunogenic epitopes, it is essential that large amounts of well defined, nontoxic synthetic LPS core structures are available. Despite the great interest in synthetic LPS structures, only a few N. meningitidis LPS-related oligosaccharides containing L-glycero-D-mannoheptose (Hep) and 3-deoxy-D-manno-2-octulosonic acid (Kdo), have been synthesized to date. LPS core tetrasaccharide synthesis is particularly difficult because of two synthetic challenges imposed by the LPS core structure: production of Hep building blocks, and generation of the Hep-Kdo disaccharide.

It is an objective of the present invention to provide a route for a total chemical synthesis of the saccharides 1^{#}, 35^{#}, 36^{#}, and 37^{#} which are derived from the α-GlcNAc-(1→2)-α-Hep-(1→3)-α-Hep-(1→5)-α-Kdo core tetrasaccharide of the lipopolysaccharide from Neisseria meningitidis. Furthermore, it is an objective of the present invention to provide a vaccine for immunization against diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep, especially for providing a vaccine for immunization against meningitis septicaemia, pneumonia and nasopharyngitis caused by Neisseria meningitidis.

The objective of the present invention is solved by the teachings of the independent claims. Further advantageous features, aspects and details of the invention are evident from the dependent claims, the description, the figures, and the examples of the present application.

### DESCRIPTION OF THE INVENTION

The present invention describes the chemical preparation of the synthetic saccharides 1^{#}, 35^{#}, 36^{#} and 37^{#} as well as their precursors 28^{#}, 31^{#}, 39^{#} and 32^{#}.

The present invention describes in particular the first total synthesis of the saccharide derivatives 1^{#}, 35^{#}, 36^{#} and 37^{#} of N. meningitidis LPS. To this end, an effective and reliable de novo synthetic route was also developed that gives ready access to versatile, differentially-protected heptose building blocks. The convergent [2±2] approach used to produce the trisaccharide 37^{#} and the tetrasaccharide 1^{#} also relied on the construction of a sterically demanding 1→5 linked Hep-Kdo disaccharide, a challenge that was met effectively by TMSOTf-promoted glycosylation with heptose trichloroacetimidate. This synthetic strategy may serve more generally as a means to access LPS core structures consisting of heptose and Kdo. As such, this method shall find wide application in the synthesis of larger and more complex LPS structures of Gram-negative bacteria.

The present invention also involves conjugation of these saccharides 1^{#}, 35^{#}, 36^{#} and 37^{#} to a carrier protein such as CRM₁₉₇ and immunization experiments in BalB/C mice in presence of Freund's adjuvant and alum. Significantly higher antibody titers specific to substructures of the LPS core saccharide of N. meningitidis were observed in mice immunized with synthetic saccharides 1 and 37 conjugated to CRM₁₉₇ in comparison to unconjugated tetrasaccharide 1 and unconjugated trisaccharide 37. Immunization with the conjugates of saccharides 1 and 37 resulted in robust boosting responses. Specificity of sera and monoclonal antibodies against synthetic carbohydrate structures from different bacterial LPS was studied by microarray analysis revealing a specific response for the tetrasaccharide 1. Flow cytometry and confocal fluorescence microscopy studies showed that antibodies are able to recognize meningococcal strains across all serogroups. The potential of these antibodies to promote opsonophagocytosis was evaluated by flow cytometry assay using fluorescence-labeled bacteria and serum-dependent enhancement in phagocytosis was demonstrated. Results of these studies show that the synthetic saccharides of general formula (I) and especially the saccharides 1^{#}, 35^{#}, 36^{#} and 37^{#} are highly useful vaccine candidates against Neisseria meningitidis serogroup B.

The present invention relates to the chemical synthesis of synthetic saccharides of general formula (I)

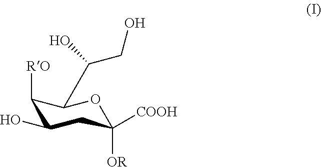

wherein

R represents —Y—NH₂;

Y represents a linker;
R′ represents H
or

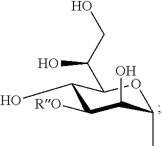

R″ represents H
or

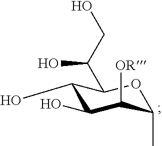

R″ represents H
or

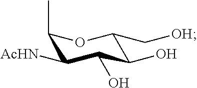

comprising or consisting of the steps:

A1) Reacting compound 26* with a compound HO—Y—NP¹⁴P¹⁵,

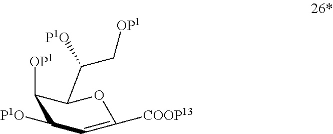

wherein P¹, P¹³, P¹⁴ and P¹⁵ are protecting groups and Y is defined as above, yielding a compound of the formula 28^{#}

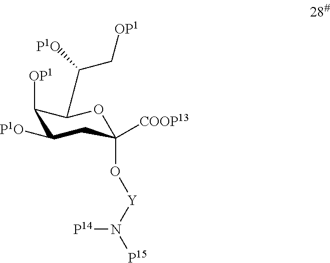

A2) Performing a deprotection reaction with compound 28^{#} yielding a monosaccharide 35^{#},

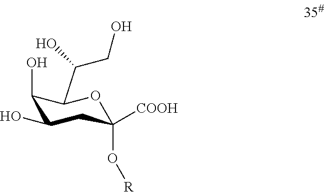

wherein R is defined as above,

or

converting compound 28^{#} to compound 7^{#},

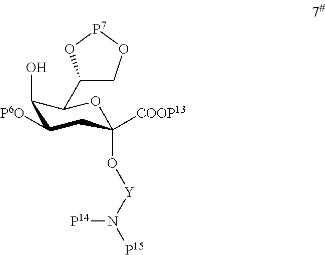

wherein P⁶ and P⁷ are protecting groups, and Y, P¹³, P¹⁴ and P¹⁵ are defined as above;

B1) Reacting compound 6* of the formula

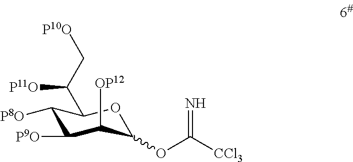

wherein

P⁸-P¹² represent protecting groups with compound 7^{#} in order to obtain a compound 31^{#} of the following chemical formula

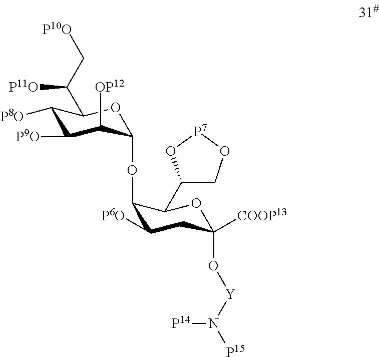

wherein Y and P⁶ to P¹⁵ are defined as above;

B2) Performing deprotection reactions with compound 31^{#} yielding a disaccharide 36^{#},

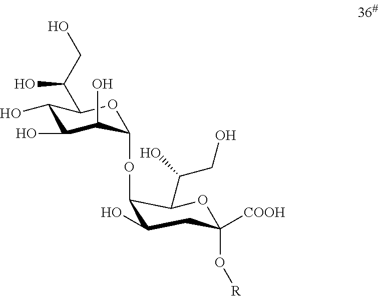

wherein R is defined as above,

or

performing a selective deprotection yielding a compound of the formula 3^{#},

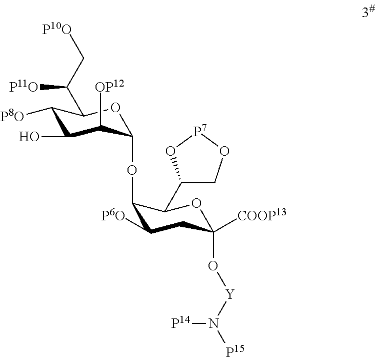

wherein P⁶ to P⁸, P¹⁰ to P¹⁵ and Y are defined as above;

C1) Reacting compound 3^{#} with a compound of the formula 38^{#}

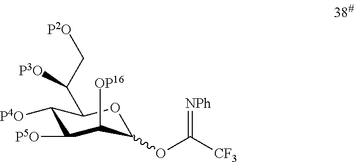

wherein P²-P⁵ and P¹⁶ are protecting groups,

yielding a compound of the formula 39#,

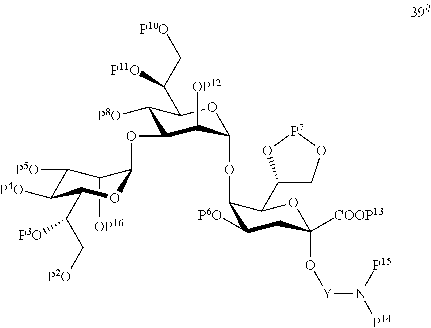

wherein P²-P⁸, P¹⁰-P¹⁵ and Y are defined as above, and

D1) Performing deprotection reactions with compound 39^{#} yielding a trisaccharide 37^{#}

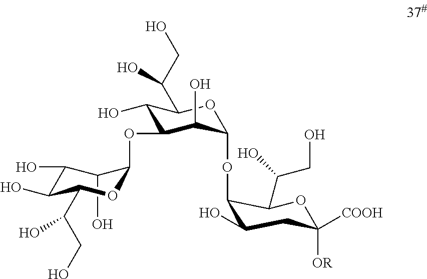

wherein R is defined as above,

or

C2) Reacting the compound 4* of the formula

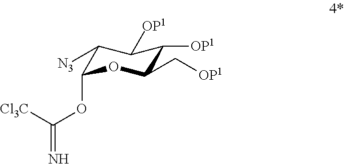

wherein

the groups P¹ represent the same protecting group

with the compound 5* of the following chemical formula

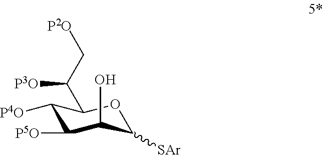

wherein

P²-P⁵ are defined as above and

Ar represents an aromatic ring or aromatic ring system

in order to obtain compound 30* of the following chemical formula

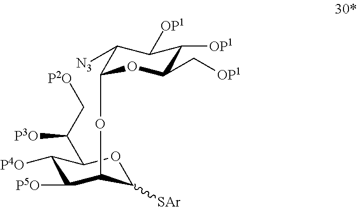

wherein

the group —SAr is converted to the group —O—C(═NPh)-CF₃ in order to obtain compound 2* of the following chemical formula

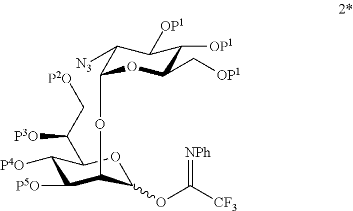

wherein P¹-P⁵ are defined as above,

Reacting compound 3^{#} with a compound 2*,

yielding a compound of the formula 32^{#},

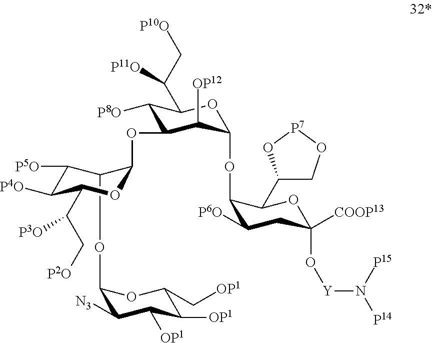

wherein P¹-P⁸, P¹⁰-P¹⁵ and Y are defined as above,

and

D2) Converting the azide group of compound 32^{#} into an acetamide group and performing deprotection reactions yielding a tetrasaccharide 1^{#}

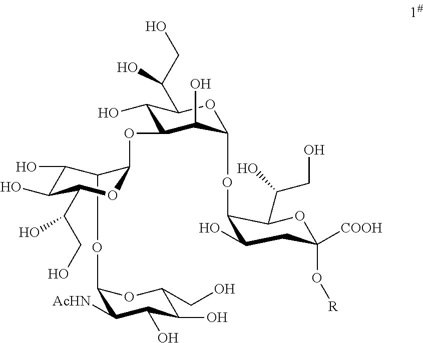

wherein R is defined as above.

As used herein the term “linker” refers a molecular moiety which is capable of connecting two molecular fragments or atoms.

In above formula (I) or in any other formula of the description comprising a linker Y represents any suitable linker. Preferably Y represents a linker containing up to 50 carbon atoms. This carbon atom based linker contains up to 60 carbon atoms and preferably up to 40 carbon atoms and more preferably between 2 and 26 carbon atoms and most preferably between 3 and 20 carbon atoms. Such a linker may further contain between 1 to 30 oxygen atoms, 1 to 10 nitrogen atoms and 1 to 10 sulfur atoms. Further preferred is that this linker Y is linked through the C2 connected oxygen atom of the Kdo moiety with an amino group. Therefore, it is further preferred that the linker Y is capable of presenting an amino group in a suitable distance from the Kdo moiety for binding to an interconnecting molecule and/or a carrier such as a carrier protein or a glass plate.

Thus function of the linker is to covalently connect the sugar moiety to which the residue R is bond directly or through another chemical residue or chemical group to a carrier protein or to a solid carrier such as a glas plate.

An interconnecting molecule according to the present invention refers to a bifunctional molecule wherein one functional group is capable of reacting with the amino group connected to the linker Y and the second functional group is capable of binding to a carrier such as a carrier protein or a glass plate.

The present invention refers to saccharides of the formula (I), wherein the linker is capable of establishing, keeping and/or bridging a special distance between the Kdo moiety and a carrier such as a carrier protein or a glass plate.

Thus, the term “linker” as used herein is a chain of at least one carbon atom and up to 50 carbon atoms, wherein this chain of 1 to 50 carbon atoms may be branched and may further contain 1 to 30 oxygen atoms and/or 1 to 10 nitrogen atoms and/or 1 to 10 sulfur atoms and/or 1 to 3 phenyl moieties and/or 1 to 3 saturated 3-, 4-, 5- or 6-membered carbocyclic rings and/or 1 to 3 saturated 3-, 4-, 5- or 6-membered heterocyclic rings containing one or two heteroatoms selected from O, N and S, wherein this chain, the phenyl moieties and/or the carbocyclic or heterocyclic rings within said chain can be substituted with 1 to 10 substituents selected from the group consisting of:

═O (carbonyl group), —OH, —OCH₃, —OC₂H₅, —OC₃H₇, —O-cyclo-C₃H₅, —OCH(CH₃)₂, —OC(CH₃)₃, —OC₄H₉, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —COCH₃, —COC₂H₅, —COC₃H₇, —CO-cyclo-C₃H₅, —COCH(CH₃)₂, —COC(CH₃)₃, —COOH, —COOCH₃, —COOC₂H₅, —COOC₃H₇, —OOC-cyclo-C₃H₅, —COOCH(CH₃)₂, —COOC(CH₃)₃, —OOC—CH₃, —OOC—C₂H₅, —OOC—C₃H₇, —OOC-cyclo-C₃H₅, —OOC—CH(CH₃)₂, —OOC—C(CH₃)₃, —CONH₂, —CONHCH₃, —CONHC₂H₅, —CONHC₃H₇, —CONH-cyclo-C₃H₅, —CONH[CH(CH₃)₂], —CONH[C(CH₃)₃], —CON(CH₃)₂, —CON(C₂H₅)₂, —CON(C₃H₇)₂, —CON(cyclo-C₃H₅)₂, —CON[CH(CH₃)₂]₂, —CON[C(CH₃)₃]₂, —NHCOCH₃, —NHCOC₂H₅, —NHCOC₃H₇, —NHCO-cyclo-C₃H₅, —NHCO—CH(CH₃)₂, —NHCO—C(CH₃)₃, —NHCO—OCH₃, —NHCO—OC₂H₅, —NHCO—OC₃H₇, —NHCO—O-cyclo-C₃H₅, —NHCO—OCH(CH₃)₂, —NHCO—OC(CH₃)₃, —NHCH₃, —NHC₂H₅, —NHC₃H₇, —NH-cyclo-C₃H₅, —NHCH(CH₃)₂, —NHC(CH₃)₃, —N(CH₃)₂, —N(C₂H₅)₂, —N(C₃H₇)₂, —N(cyclo-C₃H₅)₂, —N[CH(CH₃)₂]₂, —N[C(CH₃)₃]₂, —NH—CO—NH₂, —NH—CO—NHCH₃, —NH—CO—NHC₂H₅, —NH—CS—N(C₃H₇)₂, —NH—CO—NHC₃H₇, —NH—CO—N(C₃H₇)₂, —NH—CO—NH[CH(CH₃)₂], —NH—CO—NH[C(CH₃)₃], —NH—CO—N(CH₃)₂₃—NH—CO—N(C₂H₅)₂, —NH—CO—NH-cyclo-C₃H₅, —NH—CO—N(cyclo-C₃H₅)₂, —NH—CO—N[CH(CH₃)₂]₂, —NH—CO—N[C(CH₃)₃]₂, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(OH₃)₂, —C₆H₁₃, —C₇H₁₅, —C₈H₁₇, —C₃H₆—CH(CH₃)₂, —C₂H₄—CH(OH₃)—C₂H₅, —CH(CH₃)—C₄H₉, —CH₂—CH(CH₃)—C₃H₇, —CH(CH₃)—CH₂—CH(OH₃)₂, —CH(CH₃)—CH(OH₃)—C₂H₅, —CH₂—CH(CH₃)—CH(CH₃)₂, —CH₂—C(OH₃)₂—O₂H₅, —C(CH₃)₂—O₃H₇, —C(CH₃)₂—CH(CH₃)₂, —C₂H₄—C(CH₃)₃ and —CH(CH₃)—C(CH₃)₃.

The amino group of the residue R is attached to one carbon atom of this chain which is preferably a terminal carbon atom thus having beside the nitrogen atom of the amino group only one further carbon atom to which this terminal carbon atom is attached. Also one carbon atom of this chain is attached to the oxygen atom of the sugar residue.

Further the present invention refers saccharides of the formula (I), wherein the linker is a molecular fragment capable of connecting saccharides selected from the group comprising or consisting of α-GlcNAc-(1→2)-α-Hep-(1-3)-α-Hep-(1→5)-α-Kdo, α-Hep-(1→3)-α-Hep-(1→5)-α-Kdo, α-Hep-(1→5)-α-Kdo and α-Kdo via the amino group with a carrier, preferably a protein, glass plate or diagnostic tool, optionally by binding to at least one further interconnecting molecule, preferably a succinimide, between amino group and carrier.

More preferably Y represents —Y¹—Y²—Y³—, —Y¹—Y³—, —Y¹—, or —Y¹—Y²—Y⁴—Y⁵—Y³—, wherein

Y¹ and Y⁴ represent independently of each other —(CH₂)ₙ—, —(CH₂)ₘ—, —(CH₂)ₙ—CHR¹—(CH₂)ₘ—, —(CH₂)ₙ—CR¹R²—(CH₂)ₘ—, —(C₂H₄O)ₙ—CH₂—, —(C₂H₄O)ₘ—C₂H₄—, -o-C₆H₄—, -m-C₆H₄—, -p-C₆H₄—,

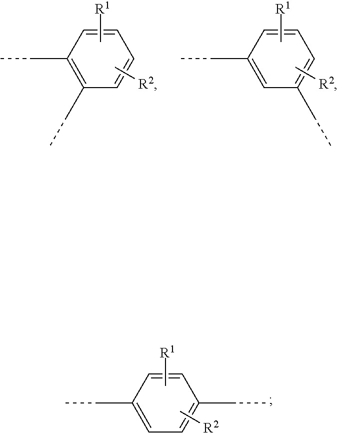

Y² and Y⁵ represent independently of each other —(CH₂)ₚ—, —(CH₂)_{q}—, —CHR³—, —CR³R⁴—, —O—, —S—, —S—S—, —CO—, —COO—, —O—CO—, —NH—CO—, —CO—NH—, —NH—CO—NH—, -o-C₆H₄—, -m-C₆H₄—, -p-C₆H₄—, —NR⁷—, —CH═CH—;

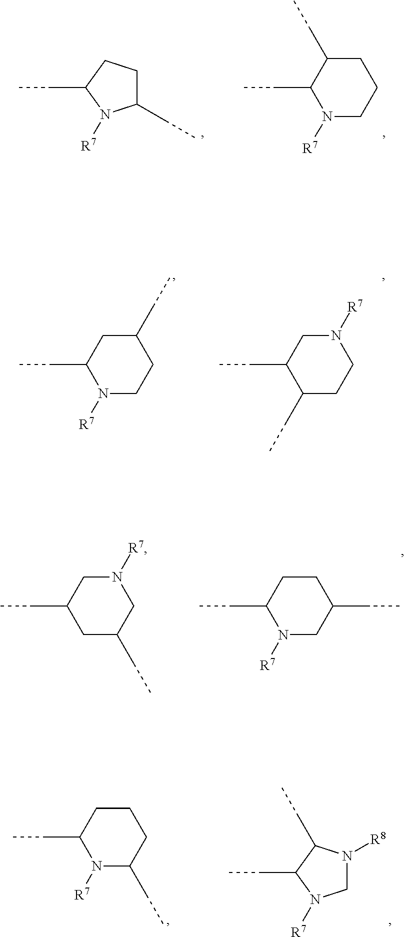

Y³ represents —(CH₂)ᵣ, —(CH₂)ᵣCR⁵R⁶—(CH₂)ₛ—, -o-C₆H₄—, -m-C₆H₄—, -p-C₆H₄—,

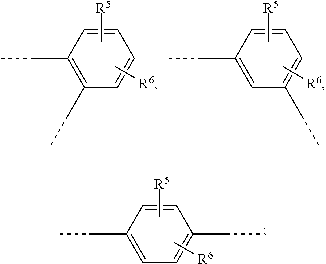

R¹ to R⁶ represent independently of each other —H, —NH₂, —OH, —OCH₃, —OC₂H₅, —OC₃H₇, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —OH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃, —CH₂—C₆H₄—OH,

R⁷ and R⁸ represent independently of each other cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CH═CH₂, —CH₂—CH═CH₂, —C(CH₃)═CH₂, —CH═CH—CH₃, —C₂H₄—CH═CH₂, —C₇H₁₅, —C₈H₁₇, —CH₂—CH═CH—CH₃, —CH═CH—C₂H₅, —CH₂—C(CH₃)═CH₂, —CH(CH₃)—CH═CH, —CH═C(CH₃)₂, —C(CH₃)═CH—CH₃, —CH═CH—CH═CH₂, —C₃H₆—CH═CH₂, —C₂H₄—CH═CH—CH₃, —CH₂—CH═CH—C₂H₅, —CH═CH—C₃H₇, —CH₂—CH═CH—CH═CH₂, —CH═CH—CH═CH—CH₃, —CH₂NH₂, —CH₂OH, —CH₂SH, —CH₂—CH₂NH₂, —CH₂—CH₂SH, —C₆H₄—OCH₃, —C₆H₄—OH, —CH₂—CH₂—OCH₃, —CH₂—CH₂OH, —CH₂—OCH₃, —CH₂—C₆H₄—OCH₃, —CH₂—C₆H₄—OH,

n, m, r and s represent independently of each other an integer from 1 to 20;

p and q represent independently of each other an integer from 0 to 5.

In a more preferred embodiment of the present invention the linker moiety Y represents —Y¹—Y²—Y³—, —Y¹—Y³—, —Y¹—, or —Y¹—Y²—Y⁴—Y⁵—Y³—, wherein

Y¹ and Y⁴ represent independently of each other —(CH₂)ₙ—, —(CH₂)ₘ—, —(CH₂)ₙ—CHR¹—(CH₂)ₘ—, —(CH₂)ₙ—CR¹R²—(CH₂)ₘ—, —(C₂H₄O)ₙ—CH₂—, —(C₂H₄O)ₘ—C₂H₄—;

Y² and Y⁵ represent independently of each other —(CH₂)ₚ—, —(CH₂)_{q}—, —CHR³—, —CR³R⁴—, —O—, —S—S—, —S—;

Y³ represents —(CH₂)ᵣ, —(CH₂)ᵣCR⁵R⁶—(CH₂)ₛ—, -o-C₆H₄—, -m-C₆H₄—, -p-C₆H₄—,

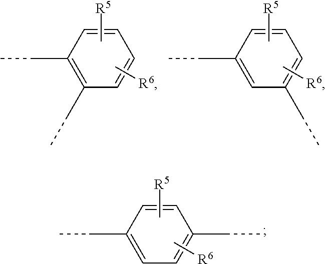

R¹ to R⁶ represent independently of each other —H, —OH, —OCH₃, —OC₂H₅, —OC₃H₇, cyclo-C₃H₅, cyclo-C₄H₇, cyclo-C₅H₉, cyclo-C₆H₁₁, cyclo-C₇H₁₃, cyclo-C₈H₁₅, -Ph, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂NH₂, —CH₂OH, —CH₂SH, —CH₂—CH₂NH₂, R⁷ and R⁸ represent independently of each other —CH₂-Ph, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂,

n, m, r and s represent independently of each other an integer from 1 to 15;

p and q represent independently of each other an integer from 0 to 5.

In an even more preferred embodiment of the present invention the linker moiety Y represents —Y¹—Y²—Y³—, —Y¹—Y³—, or —Y¹— wherein

Y¹ represents —(CH₂)ₙ—, —(CH₂)ₙ—CHR¹—(CH₂)ₘ—, —(C₂H₄O)ₘ—C₂H₄—;

Y² represents —(CH₂)ₚ—, —(CH₂)_{q}—, —CHR³—, —CR³R⁴—,

Y³ represents —(CH₂)ᵣ, —(CH₂)ᵣCR⁵R⁶—(CH₂)ₛ—,

R¹ to R⁶ represent independently of each other —H, —OH, —OCH₃, —OC₂H₅, —OC₃H₇, —CH₂-Ph, —CPh₃, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂NH₂, —CH₂OH, —CH₂SH, —CH₂—CH₂NH₂,

n, m, r and s represent independently of each other an integer from 1 to 10;

p and q represent independently of each other an integer from 0 to 5.

In a yet even more preferred embodiment of the present invention the linker moiety Y represents —Y¹— wherein

Y¹ represents —CH₂—, —C₂H₄—, —C₃H₆—, —C₄H₈—, —C₅H₁₀—, —C₆H₁₂—, —C₇H₁₄—, —C₈H₁₆—.

It is particularly preferred if

Y represents

—C₂H₄—, —C₂₂H₄₄—, —C₄₀H₈₀—, —C₂H₄—O—C₂H₄—, —(C₂H₄O)₁₂—, —CH₂—S—S—CH₂—, —C₂H₄—N(CH₃)—C₂H₄—, —C₂H₄—COO—C₃H₆—, —CH₂—C(CH₃)₂—C₆H₁₂—, —C₂H₄—S—CH₂—, —CH₂—NH—CO—NH—C₂H₄—,

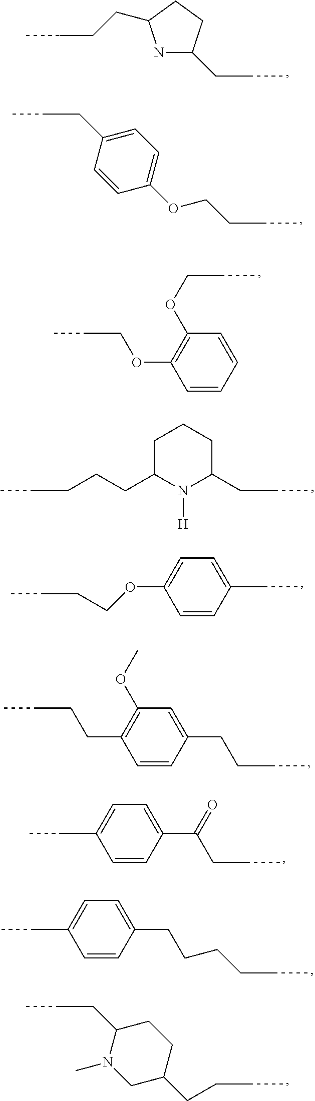

The term “protection groups” as used herein refers to commonly used protection groups in organic synthesis, preferably for amines, hydroxyl groups, thiols, imines, carbonyls or other common functional groups, and particularly preferred for amines and hydroxyl groups.

More specifically, P¹-P¹² and P¹⁶ preferably are suitable protection groups for hydroxyl groups, more preferably different suitable protection groups for hydroxyl groups capable of being removed subsequently one after another by a suitable sequence of deprotection reactions. Therefore protection groups for hydroxyl groups, namely P¹-P¹² and P¹⁶ may be selected from the group consisting of or comprising: acetyl, benzyl, benzoyl, p-methoxybenzyl, p-methoxyphenyl, para-bromobenzyl, p-nitrophenyl, allyl, acetyl, isopropyl, levulinyl, dimethoxytrityl, trityl, 2-naphthylmethyl, pyvaloyl, triisopropylsilyl, tert-butyldimethylsilyl, tert-butyldiphenylsilyl, tert-butylmethoxphenylsilyl, triethylsilyl, trimethylsilyl, 2-trimethylsilylethoxymethyl. More specifically, in a preferred embodiment of the present invention P¹, P¹⁰, P¹² and P¹⁶ may be acetyl, P² and P⁵ may be benzoyl, P³, P⁶ and P¹¹ may be benzyl, P⁴ and P⁸ may be para-bromobenzyl, P⁷ may be isopropyl and P⁹ may be levulinyl.

Protection groups for amines may form carbamates. Therefore protection groups P¹⁴ and P¹⁵ may be selected from the group consisting of or comprising tert-butyloxy carbonyl, 9-fluorenylmethoxy carbonyl, allyloxy carbonyl, 2,2,2-trichloroethyloxy carbonyl, benzyloxy carbonyl; carbonyls such as trifluoro acetyl, trichloro acetyl, acetyl, benzoyl. More specifically, in a preferred embodiment of the present invention P¹⁴ may be benzyloxy carbonyl and P¹⁵ may be benzyl.

Protection groups for carboxylic acids may form esters. Therefore P¹³ may be selected from the group consisting of or comprising methyl, ethyl, allyl, isopropyl, tert-butyl, phenyl, benzyl, p-methoxybenzyl. More specifically, in a preferred embodiment of the present invention P¹³ may be methyl.

The protection strategy is directed to protection groups which can be removed in one final step. Therein, it is preferable that on the one hand several kinds of protection groups can be removed and on the other hand several protection groups of one kind can be removed simultaneously. Such strategy facilitates a subsequent introduction of several different kind of substituents and further one final deprotection step in order to generate the compounds of the general formula (I), (II), (III) and 1^{#}, and in particular of tetrasaccharide 1.

A preferred embodiment of the present invention relates to the synthesis of synthetic saccharides of general formula (II)

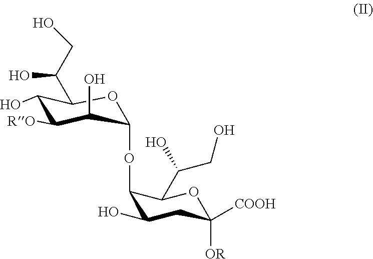

wherein

R represents —Y—NH₂;

Y represents a linker;
R″ represents H or

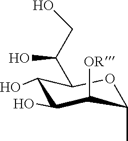

R′″ represents H or

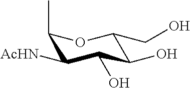

comprising the steps:

A1) Reacting compound 26* with a compound HO—Y—NP¹⁴P¹⁵

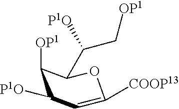

wherein P¹, P¹³, P¹⁴ and P¹⁵ are protecting groups and Y is as defined above, yielding a compound of the formula 28^{#}

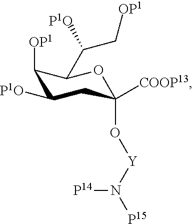

A2) Converting compound 28^{#} to compound 7^{#},

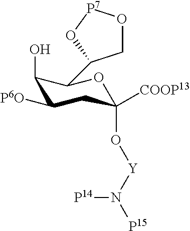

wherein P⁶ and P⁷ are protecting groups, and Y, P¹³, P¹⁴ and P¹⁵ are defined as above;

B1) Reacting compound 6* of the formula

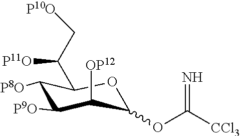

wherein

P⁸-P¹² represent protecting groups

with compound 7^{#} in order to obtain a compound 31^{#} of the following chemical formula

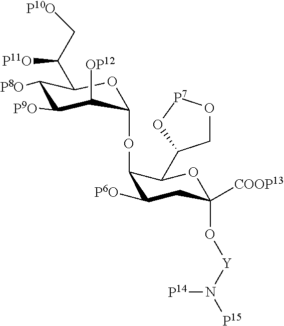

wherein Y and P⁶ to P¹⁵ are defined as above;

B2) Performing deprotection reactions with compound 31^{#} yielding a disaccharide 36^{#},

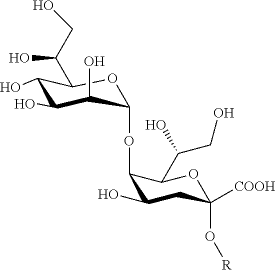

wherein R is defined as above,

or

performing a selective deprotection yielding a compound of the formula 3^{#},

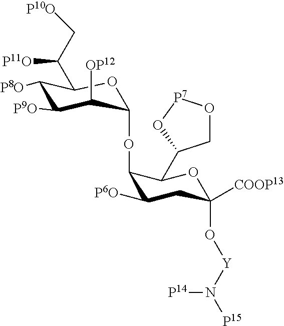

wherein P⁶ to P⁸, P¹⁰ to P¹⁵ and Y are defined as above;

C1) Reacting compound 3^{#} with a compound of the formula 38^{#}

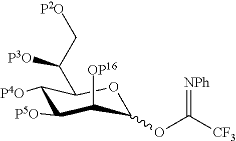

wherein P²-P⁵ and P¹⁶ are protecting groups,

yielding a compound of the formula 39^{#},

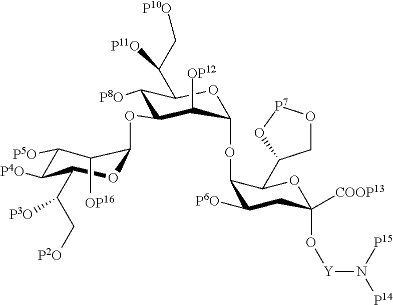

wherein P²-P⁸, P¹⁰-P¹⁵ and Y are defined as above, and

D1) Performing deprotection reactions with compound 39^{#} yielding a trisaccharide 37^{#}

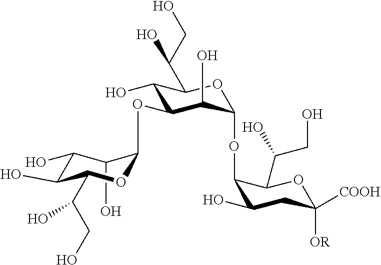

wherein R is defined as above,

or

C2) Reacting the compound 4* of the formula

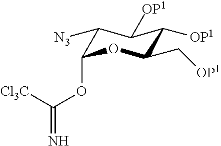

wherein

the groups P¹ represent the same protecting group

with the compound 5* of the following chemical formula

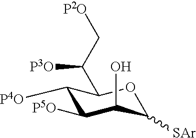

wherein

P²-P⁵ are defined as above and

Ar represents an aromatic ring or aromatic ring system

in order to obtain compound 30* of the following chemical formula

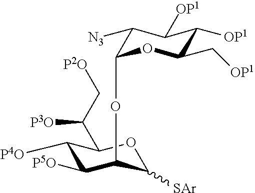

wherein

the group —SAr is converted to the group —O—C(═NPh)-CF₃ in order to obtain compound 2* of the following chemical formula

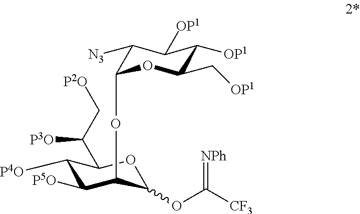

wherein P¹-P⁵ are defined as above,

Reacting compound 3^{#} with a compound 2*,

yielding a compound of the formula 32^{#},

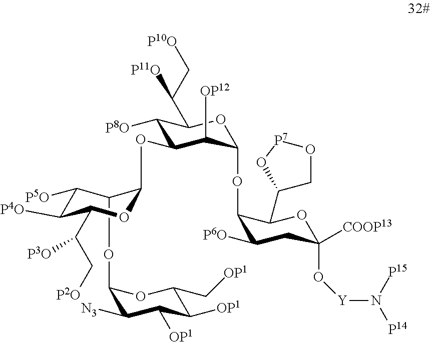

wherein P¹-P⁸, P¹⁰-P¹⁵ and Y are defined as above,

and

D2) Converting the azide group of compound 32^{#} into an acetamide group and performing deprotection reactions yielding a tetrasaccharide 1^{#}

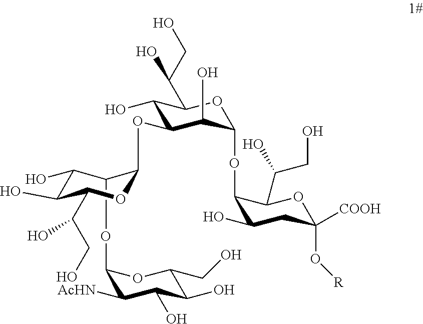

wherein R is defined as above.

Another preferred embodiment of the present invention relates to the synthesis of synthetic saccharides of general formula (III)

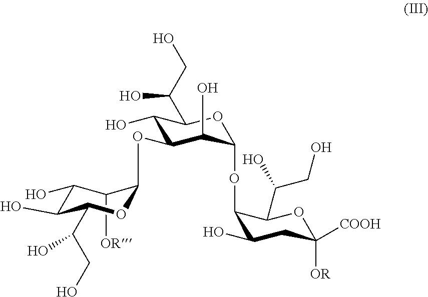

wherein

R represents —Y—NH₂;

Y represents a linker;
R′″ represents H or

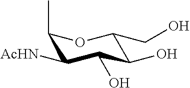

comprising the steps:

A1) Reacting compound 26* with a compound HO—Y—NP¹⁴P¹⁵,

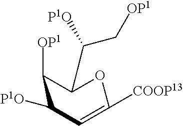

wherein P¹, P¹³, P¹⁴ and P¹⁵ are protecting groups and Y is defined as above, yielding a compound of the formula 28^{#}

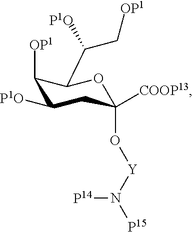

A2) Converting compound 28^{#} to compound 7^{#},

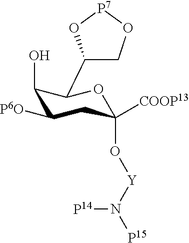

wherein P⁶ and P⁷ are protecting groups, and Y, P¹³, P¹⁴ and P¹⁵ are defined as above;

B1) Reacting compound 6* of the formula

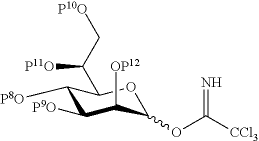

wherein

P⁸-P¹² represent protecting groups

with compound 7^{#} in order to obtain a compound 31^{#} of the following chemical formula

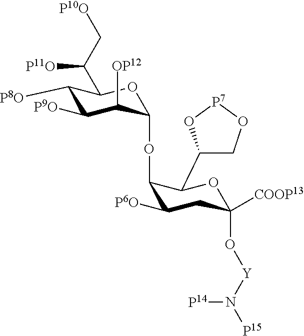

wherein Y and P⁶ to P¹⁵ are defined as above;

B2) Performing a selective deprotection yielding a compound of the formula 3^{#},

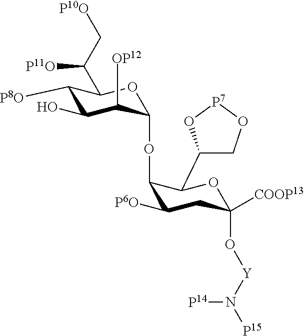

wherein P⁶ to P⁸, P¹⁰ to P¹⁵ and Y are defined as above;

C1) Reacting compound 3^{#} with a compound of the formula 38^{#}

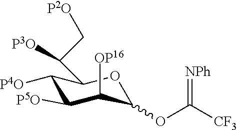

wherein P²-P⁵ and P¹⁶ are protecting groups,

yielding a compound of the formula 39^{#},

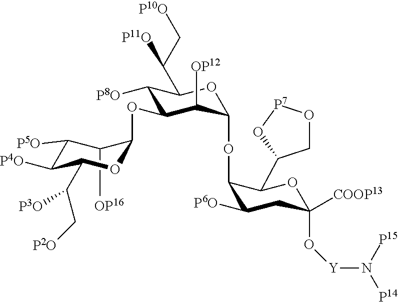

wherein P²-P⁸, P¹⁰-P¹⁵ and Y are defined as above,

and

D1) Performing deprotection reactions with compound 39^{#} yielding a trisaccharide 37^{#}

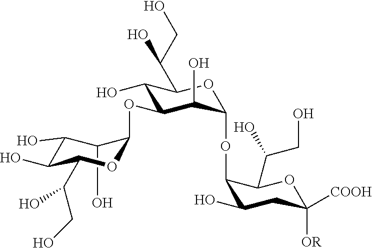

wherein R is defined as above,

or

C2) Reacting the compound 4* of the formula

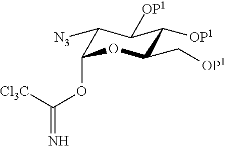

wherein

the groups P¹ represent the same protecting group

with the compound 5* of the following chemical formula

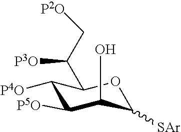

wherein

P²-P⁵ are defined as above and

Ar represents an aromatic ring or aromatic ring system

in order to obtain compound 30* of the following chemical formula

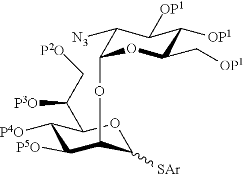

wherein the group —SAr is converted to the group —O—C(═NPh)-CF₃ in order to obtain compound 2* of the following chemical formula

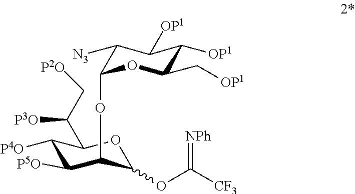

wherein P¹-P⁵ are defined as above,

reacting compound 3^{#} with a compound 2*,

yielding a compound of the formula 32^{#},

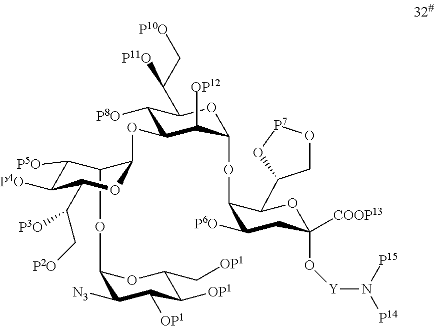

wherein P¹-P⁸, P¹⁰-P¹⁵ and Y are defined as above,

and

D2) Converting the azide group of compound 32^{#} into an acetamide group and performing deprotection reactions yielding a tetrasaccharide 1^{#}

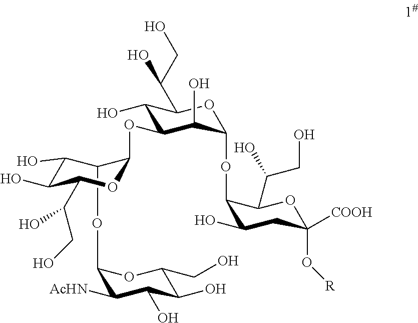

wherein R is defined as above.

Another preferred embodiment of the present invention relates to the synthesis of synthetic tetrasaccharide 1^{#}

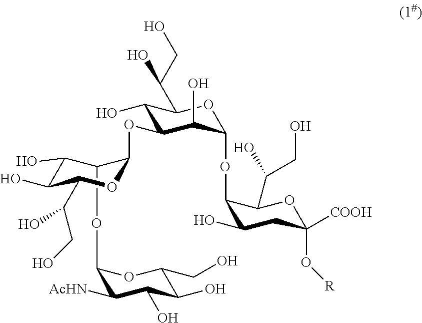

wherein

R represents —Y—NH₂;

Y represents a linker;

comprising the steps:

A1) Reacting compound 26* with a compound HO—Y—NP¹⁴P¹⁵,

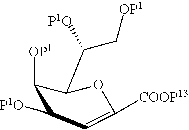

wherein P¹, P¹³, P¹⁴ and P¹⁵ are protecting groups and Y is defined as above,

yielding a compound of the formula 28^{#}

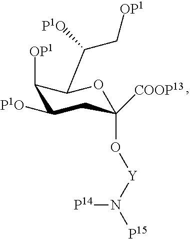

A2) Converting compound 28^{#} to compound 7^{#},

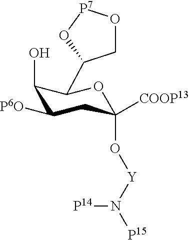

wherein P⁶ and P⁷ are protecting groups, and Y, P¹³, P¹⁴ and P¹⁵ are defined as above;

B1) Reacting compound 6* of the formula

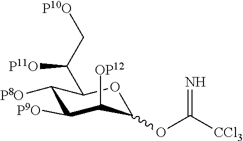

wherein

P⁸-P¹² represent protecting groups

with compound 7^{#} in order to obtain a compound 31^{#} of the following chemical formula

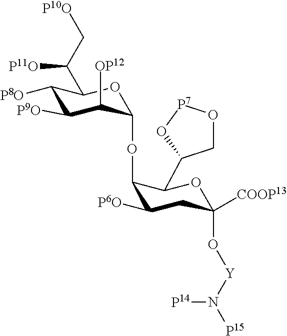

wherein Y and P⁶ to P¹⁵ are defined as above;

B2) Performing a selective deprotection yielding a compound of the formula 3^{#},

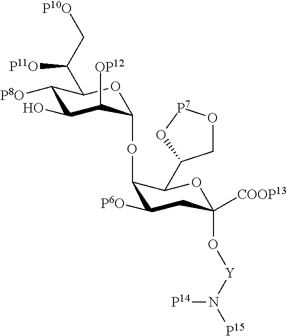

wherein P⁶ to P⁸, P¹⁰ to P¹⁵ and Y are defined as above;

C) Reacting the compound 4* of the formula

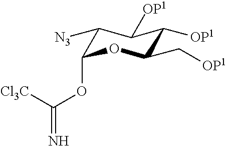

wherein

the groups P¹ represent the same protecting group

with the compound 5* of the following chemical formula

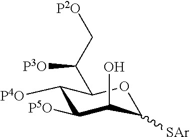

wherein

P²-P⁵ represent protecting groups and

Ar represents an aromatic ring or aromatic ring system

in order to obtain compound 30* of the following chemical formula

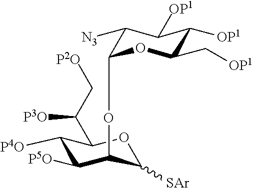

wherein

the group —SAr is converted to the group —O—C(═NPh)-CF₃ in order to obtain compound 2* of the following chemical formula

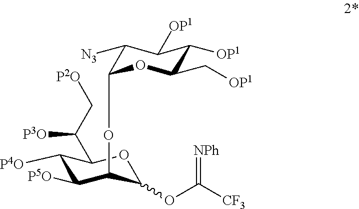

wherein P¹-P⁵ are defined as above,

reacting compound 3^{#} with a compound 2*,

yielding a compound of the formula 32^{#},

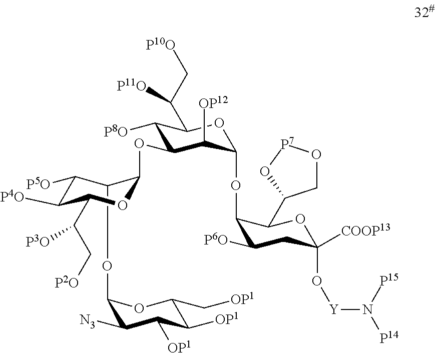

wherein P¹-P⁸, P¹⁰-P¹⁵ and Y are defined as above,

and

D) Converting the azide group of compound 32^{#} into an acetamide group and performing deprotection reactions yielding a tetrasaccharide 1^{#}

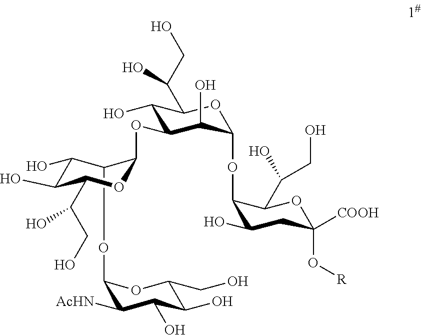

wherein R is defined as above.

A specifically preferred embodiment of the present invention relates to the synthesis of the monosaccharide 35 of the chemical formula

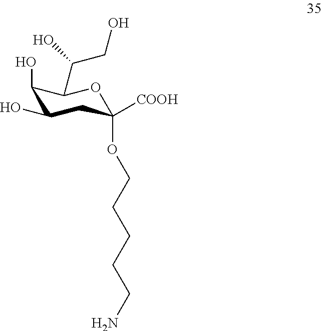

comprising the steps:

A) Reacting compound 26* of the formula

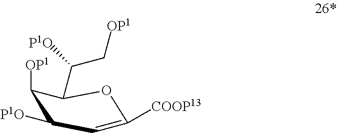

wherein

P¹ and P¹³ represent protecting groups as defined herein

with compound 27* of the formula

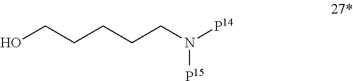

wherein

P¹⁴ and P¹⁵ represent protecting groups

in order to obtain compound 28* of the following formula

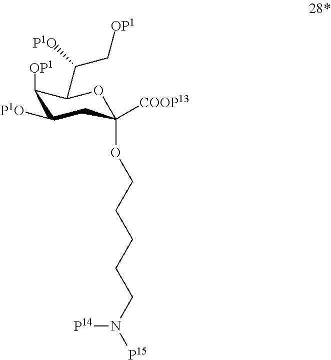

wherein the protecting groups P¹ and P¹³-P¹⁵ are cleaved in order to obtain compound 35.

Another specifically preferred embodiment of the present invention relates to chemical synthesis of the disaccharide 36 of the chemical formula

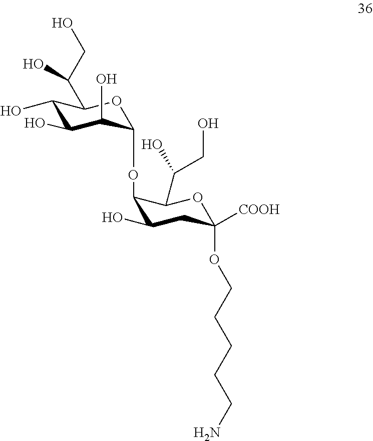

comprising the steps:

A) Reacting compound 6* of the formula

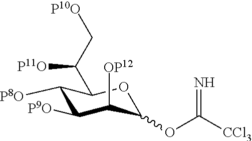

wherein

P⁸-P¹² represent protecting groups

with compound 7* of the formula

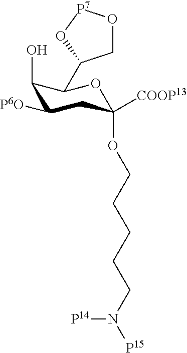

wherein

P⁶, P⁷, P¹³-P¹⁵ represent protecting groups,

in order to obtain compound 31* of the following chemical formula

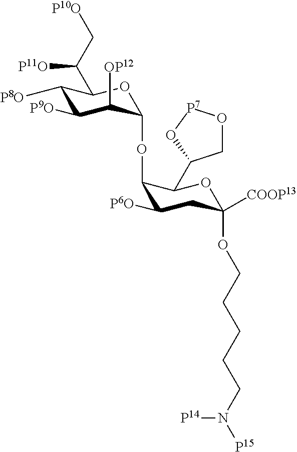

wherein the protecting groups P⁶-P¹⁵ are cleaved in order to obtain compound 36.

Yet another specifically preferred embodiment of the present invention relates to chemical synthesis of the trisaccharide 37 of the chemical formula

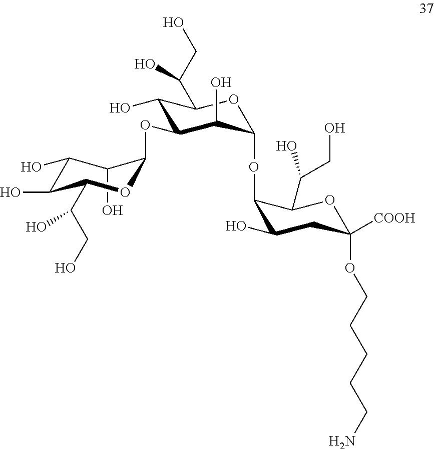

comprising the steps:

A) Reacting compound 6* of the formula

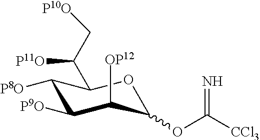

wherein

P⁸-P¹² represent protecting groups

with compound 7* of the formula

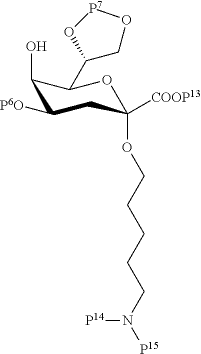

wherein

P⁶, P⁷, P¹³-P¹⁵ represent protecting groups

in order to obtain compound 31* of the following chemical formula

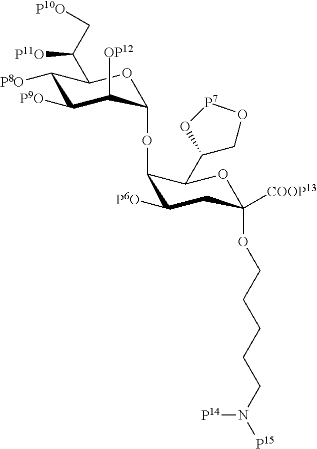

wherein the protecting group P⁹ is selectively cleaved in order to obtain compound 3* of the formula

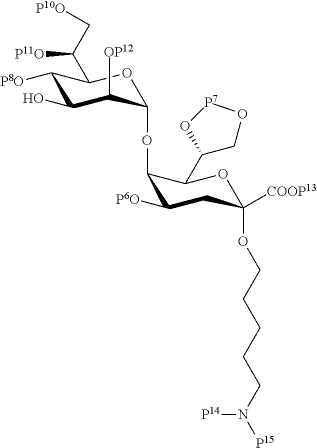

B) Reacting compound 38* of the formula

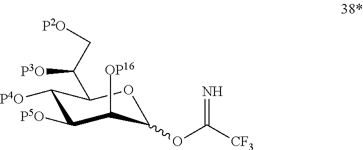

with compound 3* in order to obtain compound 39* of the formula

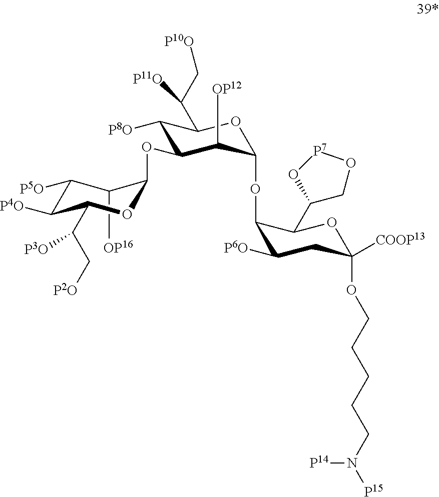

wherein the protecting groups P²-P⁸ and P¹⁰-P¹⁶ are cleaved in order to obtain compound 37.

The linker bearing an amino group on the reducing terminus of the synthetic saccharides of the general formula (I), and in particular of tetrasaccharide 1 can serve as handle for conjugation to a carrier protein. This neoglycoconjugate was used in subsequent immunology studies to demonstrate the efficacy of the saccharides of general formula (I), (II), (III) and especially the saccharids of formula 1^{#}, 35^{#}, 36^{#} and 37^{#} as a vaccine.

Thus the present invention relates in a particularly preferred embodiment to the total chemical synthesis of tetrasaccharide 1.

Scheme 1 provides an overview of the key compounds involved in the chemical synthesis of tetrasaccharide 1.

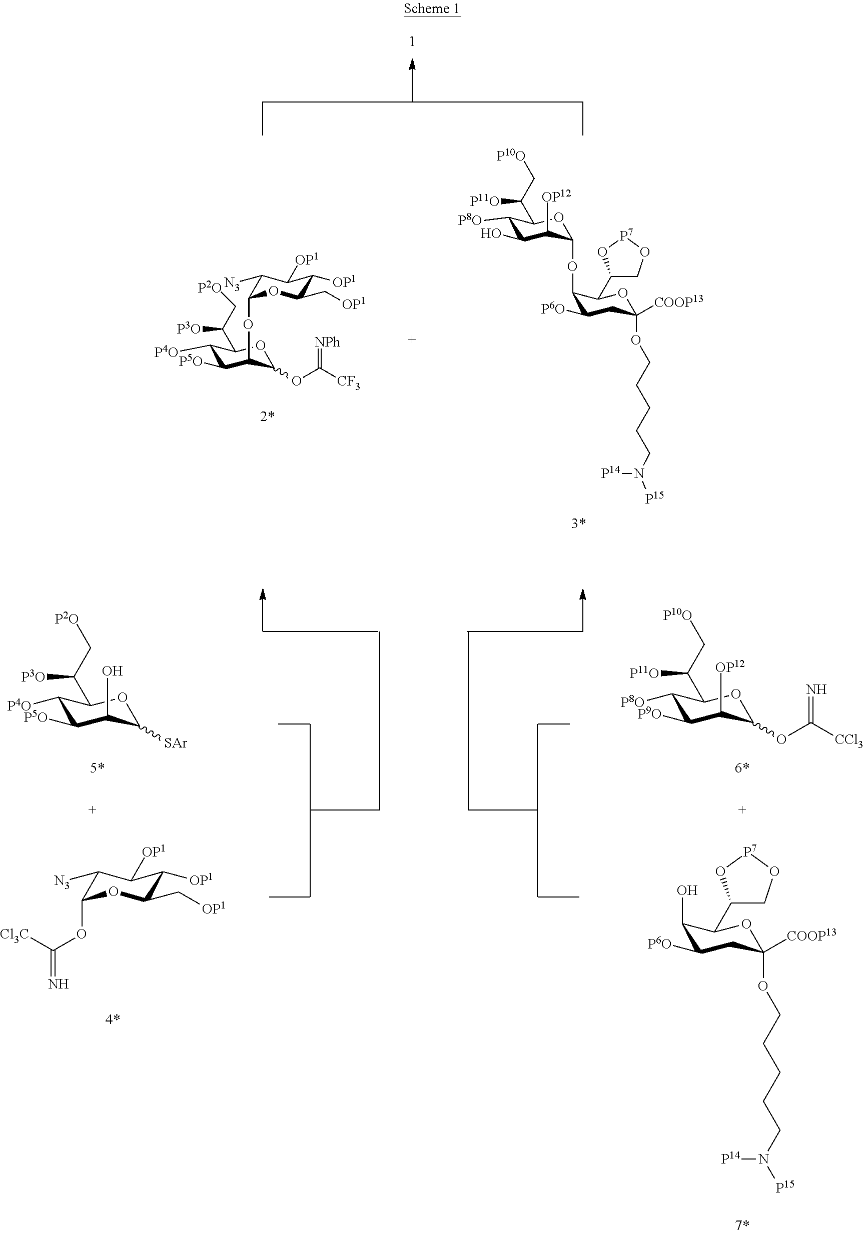

Thus, the present invention relates to the total synthesis of the tetrasaccharide 1 of the chemical formula:

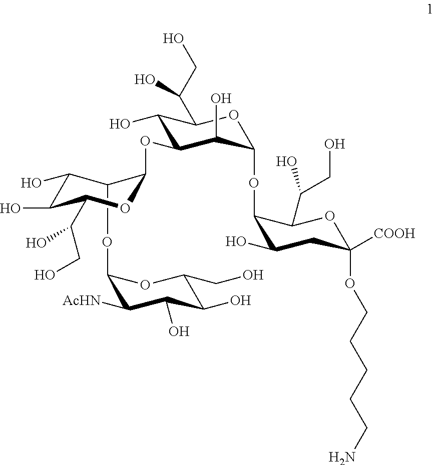

comprising the steps:

A) Reacting the compound 6* of the formula

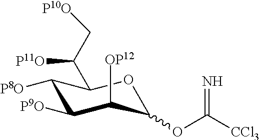

wherein

P⁸-P¹² represent protecting groups

with the compound 7* of the formula

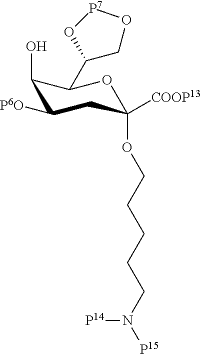

wherein

P⁶, P⁷, P¹³-P¹⁵ represent protecting groups

in order to obtain compound 31* of the following chemical formula

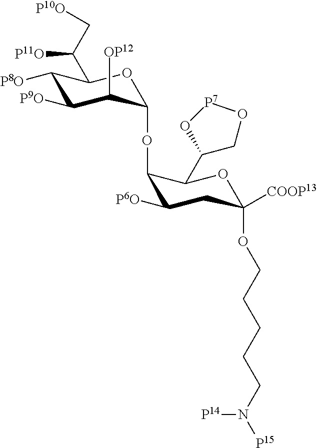

wherein the protecting group P⁹ is selectively cleaved in order to obtain compound 3*.

B) Reacting the compound 4* of the formula

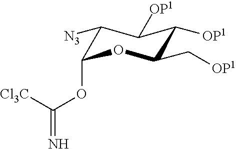

wherein

the groups P¹ represent the same protecting group

with the compound 5* of the following chemical formula

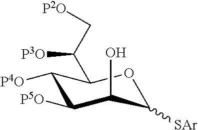

wherein

P²-P⁵ represent protecting groups and

Ar represents an aromatic ring or aromatic ring system

in order to obtain compound 30* of the following chemical formula

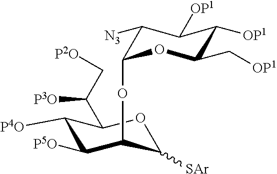

wherein

the group —SAr is converted to the group —O—C(═NPh)-CF₃ in order to obtain compound 2* of the following chemical formula

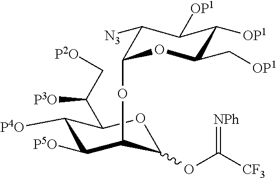

C) Reacting the compound 2* with compound 3* of the formula

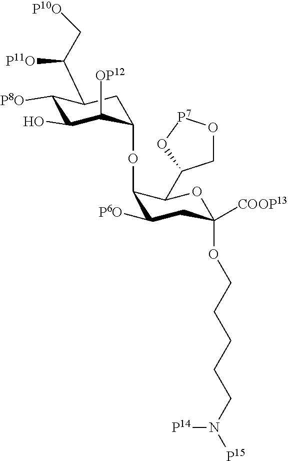

in order to obtain compound 32* of the formula

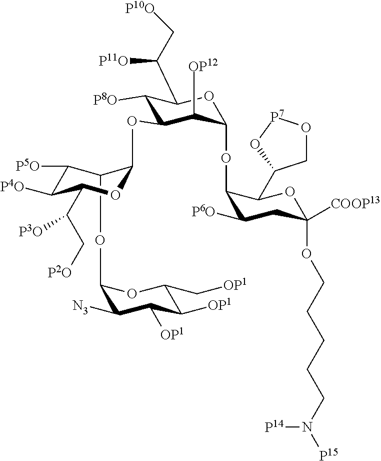

wherein the azide group is converted into an acetamide group and wherein the protecting groups P¹-P⁸ and P¹⁰-P¹⁵ are cleaved in order to obtain compound 1.

Preferably the compound 2* has the following formula

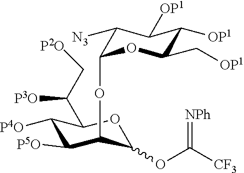

wherein P¹ represents acetyl, P² and P⁵ represent benzoyl, P³ represents benzyl and P⁴ represents para-bromobenzyl.

Preferably the compound 3* has the following formula

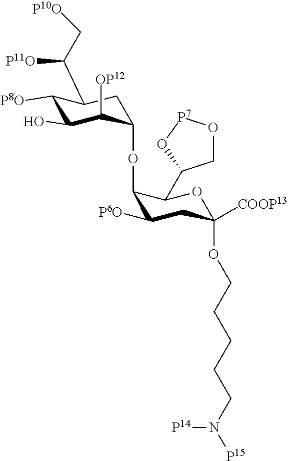

wherein P⁶, P¹¹ and P¹⁴ represent benzyl, P⁷ represents isopropyl, P⁸ represents para-bromobenzyl, P¹⁰ and P¹² represent acetyl and P¹⁵ represents benzyloxy carbonyl.

The glycan moiety of tetrasaccharide 1 according to the present invention is α-GlcNAc-(1→2)-α-Hep-(1→3)-α-Hep-(1→5)-α-Kdo.

Other aspects of the present invention are directed to the saccharides (I), (II), (III), 1^{#}, 35^{#}, 36^{#} and 37^{#} as well as to the specific saccharides 1, 35, 36 and 37 obtainable or obtained by the total chemical synthesis as disclosed herein.

A further aspect of the present invention is related to the saccharides (I), (II), (III), 1^{#}, 35^{#}, 36^{#} and 37^{#} as well as to the tetrasaccharide 1, the trisaccharide 37, the disaccharide 36 and the monosaccharide 35 which are free of any phosphoethanolamine (PEA) which is inevitably present in any extracts or isolates from biological material and especially bacterial isolates. Also the saccharides (I), (II), (III), 1^{#}, 35^{#}, 36^{#}, 37^{#}, 1, 35, 36 and 37 are free of any endotoxin lipid A which is present in the extracts or isolates from biological material and especially bacterial isolates. Therefore, one preferred embodiment of this invention is tetrasaccharide 1, trisaccharide 37, disaccharide 36 and monosaccharide 35 or any other saccharide of one of the general formula (I), (II), (III), 1^{#}, 35^{#}, 36^{#} or 37^{#} which is not contaminated with PEA and/or endotoxin lipid A. Due to these contaminations the oligosaccharides obtained or derived from natural sources were and could so far not used as vaccines. Moreover the tetrasaccharide 1, the trisaccharide 37, the disaccharide 36, the monosaccharide 35 or any other saccharide of one of the general formula (I), (II), (III), 1^{#}, 35^{#}, 36^{#} or 37^{#} according to the present invention is substantially pure, having a purity of ≧95%, preferably ≧96%, more preferably ≧97%, still more preferably ≧98%, and most preferably ≧99%. In addition the chemically synthesized saccharides of the present invention and especially tetrasaccharide 1, trisaccharide 37, disaccharide 36 and monosaccharide 35 do not have any microheterogenicity as the oligosaccharides from biological sources do.

Microheterogenicity as used herein is defined as slight differences in structure between essentially identical molecules. In case of the glycan moiety of the LPS of N. meningitidis it refers to a slightly varied glycosyl linkage and variation in the substitution of hydroxyl groups of the LPS glycan moiety by for example phosphoethanolamine (PEA) or acetyl. The glycans of bacterial LPS frequently show high degrees of microheterogenicity in the outer core, which corresponds to the short saccharides in FIG. 1. An advantage of the synthetic saccharides of the present invention is that they show no microheterogenicity which means that the synthetic saccharides of the present invention have no dissimilarity or variety in their structure, also not in regard to the glycosidic linkage or substituents.

A further aspect of the present invention is related to the saccharides selected from the following group comprising the following compounds:
tetrasaccharide 1: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(5-amino)pentyl-3-deoxy-α-D-manno-oct-2-ulopyranosidonic acid
monosaccharide 35: 2-(5-amino)pentyl-3-deoxy-a-D-manno-oct-2-ulopyranosidonic acid
disaccharide 36: L-glycero-a-D-manno-heptopyranosyl-(1→5)-2-(5-amino) pentyl-3-deoxy-a-D-manno-oct-2-ulopyranosidonic acid
trisaccharide 37: L-glycero-α-D-manno-heptopyranosyl-(1→3)-L-glycero-α-D-manno-heptopyranosyl-(1→5)-2-(5-amino)pentyl-3-deoxy-α-D-manno-oct-2-ulopyranosidonic acid
compound 42: 2-(2-amino)ethyl-3-deoxy-α-D-manno-oct-2-ulopyranosidonic acid
compound 45: 2-(22-amino)docosanyl-3-deoxy-α-D-manno-oct-2-ulopyranosidonic acid
compound 48: 2-2-(2-aminoethoxy)ethyl-3-deoxy-a-D-manno-oct-2-ulpyrano sidonic acid
compound 51: 2-2-(5-aminomethyl)pyrrolidin-2-yl)ethyl-3-deoxy-a-D-man no-oct-2-ulpyranosidonic acid
compound 54: 2-4-(2-aminoethoxy)benzyl-3-deoxy-a-D-manno-oct-2-ulpyranosidonic acid
compound 59: L-glycero-a-D-manno-heptopyranosyl-(1→5)-2-(38-amino-3,6,9,12,15,18,21,24,27,30,33,36-dodecaoxaoctatriacontanyl)-3-deoxy-a-D-manno-oct-2-ulopyranosidonic acid
compound 64: L-glycero-a-D-manno-heptopyranosyl-(1→5)-2-(((aminomethyl)disulfanyl)methyl)-3-deoxy-a-D-manno-oct-2-ulopyranosidonic acid
compound 69: L-glycero-a-D-manno-heptopyranosyl-(1→5)-2-(2-((2-aminoethyl)(methyl)amino)ethyl)-3-deoxy-a-D-manno-oct-2-ulopyranosidonic acid
compound 74: L-glycero-a-D-manno-heptopyranosyl-(1→5)-2-(2-(aminomethoxy)phenoxy)methyl-3-deoxy-a-D-manno-oct-2-ulopyranosidonic acid
compound 79: L-glycero-a-D-manno-heptopyranosyl-(1→5)-2-(3-(6-(aminomethyl)piperidin-2-yl)propanyl)-3-deoxy-a-D-manno-oct-2-ulopyranosidonic acid
compound 86: L-glycero-α-D-manno-heptopyranosyl-(1→3)-L-glycero-α-D-manno-heptopyranosyl-(1→5)-2-(40-amino)tetracontanyl-3-deoxy-α-D-manno-oct-2-ulopyranosidonic acid
compound 93: L-glycero-α-D-manno-heptopyranosyl-(1→3)-L-glycero-α-D-manno-heptopyranosyl-(1→5)-2-(2-(4-aminophenoxy)ethyl-3-deoxy-α-D-manno-oct-2-ulopyranosidonic acid
compound 100: L-glycero-α-D-manno-heptopyranosyl-(1→3)-L-glycero-α-D-manno-heptopyranosyl-(1→5)-2-(2-(4-(2-aminoethyl)-2-methoxyphenyl)ethyl)-3-deoxy-α-D-manno-oct-2-ulopyranosidonic acid
compound 107: L-glycero-α-D-manno-heptopyranosyl-(1→3)-L-glycero-α-D-manno-heptopyranosyl-(1→5)-2-(4-(2-aminoacetyl)phenyl)-3-deoxy-α-D-manno-oct-2-ulopyranosidonic acid
compound 114: L-glycero-α-D-manno-heptopyranosyl-(1→3)-L-glycero-α-D-manno-heptopyranosyl-(1→5)-2-(6-amino-3-oxo)hexyl-3-deoxy-α-D-manno-oct-2-ulopyranosidonic acid
compound 121: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(8-amino-2,2-dimethyl)octyl-3-deoxy-α-D-manno-oct-2-ulopyranosidonic acid
compound 128: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(2-((aminomethyl)thio)ethyl)-3-deoxy-α-D-manno-oct-2-ulopyranosidonic acid
compound 135: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(3-(3-aminopropyl)ureido)methyl-3-deoxy-α-D-manno-oct-2-ulopyranosidonic acid
compound 142: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(5-(2-aminoethyl)-1-methylpiperidin-2-yl)methyl-3-deoxy-α-D-manno-oct-2-ulopyranosidonic acid
compound 149: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(4-(3-aminopropyl)phenyl)-3-deoxy-α-D-manno-oct-2-ulopyranosidonic acid

Preferred embodiments of the present invention are the trisaccharide 37 and the tetrasaccharide 1, especially preferably is the tetrasaccharide 1. This relates also to other aspects and embodiments of the invention, especially to the use as a vaccine and to the use in immunological assays for diagnostics of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

Also this trisaccharide 37 and this tetrasaccharide 1 according to the present invention are preferably not contaminated with PEA and endotoxin lipid A.

VaccineStill another aspect of the present invention is directed to a vaccine containing at least one saccharide of the present invention, preferably the tetrasaccharide 1, the trisaccharide 37, the disaccharide 36 or the monosaccharide 35. These vaccines according to the present invention are preferably without any traces of PEA and/or endotoxin lipid A. The saccharides of the present invention and especially, tetrasaccharide 1, trisaccharide 37, disaccharide 36 and monosaccharide 35 used to prepare the vaccine have preferably a purity of ≧95%, preferably ≧96%, more preferably ≧97%, still more preferably ≧98%, and most preferably ≧99%. The saccharide or the saccharides contained in the vaccine do also not exhibit any microheterogenicity and especially no microheterogenicity in their glycan moiety.

The term “glycan” as used herein refers to an oligosaccharide consisting of monosaccharides having β-glycosidic linkages. The term “glycan moiety” as used herein refers to the portion of a molecule consisting of an oligosaccharide without portion having a chemical structure other than saccharides, like protein or lipid structure. In case of the tetrasaccharide 1 the glycan moiety consists of the tetrasaccharide 1 without the linker, i.e. where the group R is hydrogen, or in other words of the saccharide of formula α-GlcNAc-(1→2)-α-Hep-(1→3)-α-Hep-(1→5)-α-Kdo. This means the glycan moiety of the tetrasaccharide 1 is the structure as shown by FIG. 1 as core tetrasaccharide.

The term “core tetrasaccharide” as used herein refers to the glycan moiety of tetrasaccharide 1. The core tetrasaccharide is a saccharide derived from the core structure of the LPS structure of N.a meningitidis. The position of the core tetrasaccharide in the core structure of the LPS structure of N.a meningitidis shows FIG. 1. FIG. 1 shows also in grey further portions of the LPS molecule of N. meningitidis and their linkage in regard to the core tetrasaccharide. Tetrasaccharide 1 is the core tetrasaccharide with the specific linker as shown by the formula on page 40. Tetrasaccharide 1^{#} refers to the core tetrasaccharide with an unspecified linker. A trisaccharide according to the invention refers to the core tetrasaccharide missing either the monosaccharide α-GlcNAc or the monosaccharide Kdo.

The present invention relates further to a synthetic saccharide of general formula (I)

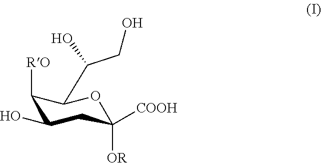

wherein

R represents —Y—NH₂,

Y represents a linker,
R′ represents H or

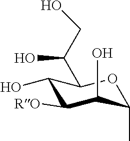

R″ represents H or

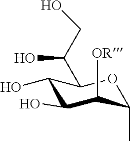

R′″ represents H or

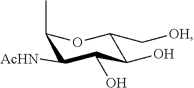

and in a further aspect to a synthetic saccharide of general formula (II)

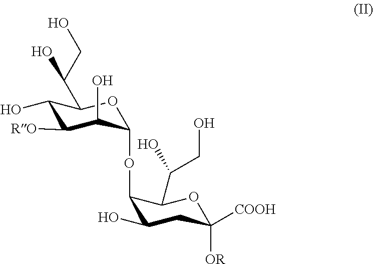

wherein

R represents —Y—NH₂,

Y represents a linker,
R″ represents H or

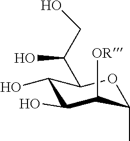

R′″ represents H or

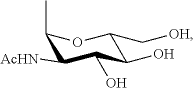

and in a further aspect to a synthetic saccharide of general formula (III)

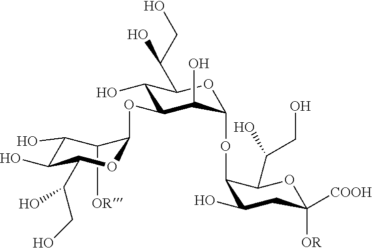

wherein

R represents —Y—NH₂,
Y represents a linker,
R′″ represents H or

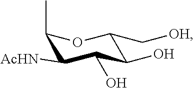

and in a still further aspect to a tetrasaccharide 1^{#}

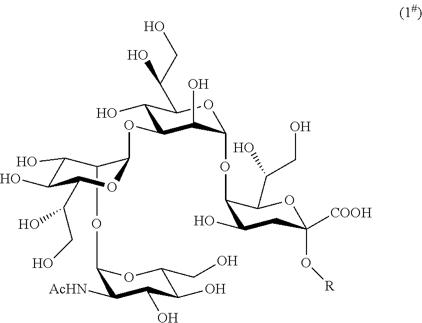

wherein

R represents —Y—NH₂,
Y represents a linker,

for use as a vaccine for immunization against diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. Preferred among said saccharide sequences is α-GlcNAc-(1→2)-α-Hep-(1→3)-α-Hep-(1→5)-α-Kdo. The diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep are preferably selected from the group consisting of meningitis, septicemia, pneumonia and nasopharyngitis. It is further preferred that the bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep are selected from the group consisting of strains of Neisseria meningitidis, wherein the lipo-oligosaccharide (LOS) immunotypes are L1, L2, L3, L4, L5, L6, L7, L8, L9 and/or L11.

The invention relates further to a synthetic saccharide of general formula (I), (II), (III), 1^{#}, 35^{#}, 36^{#} or 37^{#} and preferably to the saccharide 1, 35, 36 and/or 37 for use as a vaccine for immunization against diseases caused by bacteria having a molecule, preferably a LPS, wherein the glycan moiety comprises or contains a saccharide selected from the group comprising or consisting of α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. The diseases caused by bacteria having a molecule, preferably a LPS, wherein the glycan moiety comprises or contains a saccharide selected from the group comprising or consisting of α-GlcNAc-(1→2)-α-Hep-(1→3)-α-Hep-(1→5)-5α-Kdo, α-Hep-(1→3)-α-Hep-(1→5)-α-Kdo and α-GlcNAc-(1→2)-α-Hep-(1→3)-α-Hep are preferably selected from the group consisting of meningitis, septicemia, pneumonia and nasopharyngitis.

The invention relates further to a synthetic saccharide of general formula (I), (II), (III), 1^{#}, 35^{#}, 36^{#} or 37^{#} and preferably to the saccharide 1, 35, 36 and/or 37 for use as a vaccine for immunization against bacterial infections of Neisseria meningitidis. Preferably the infections is caused by N. meningitidis having a membrane compound comprising a saccharide selected from the group comprising or consisting of α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

Bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep as used herein are all bacterial strains, especially all strains of N. meningitidis, wherein one of the saccharides α-GlcNAc-(1→2)-α-Hep-(1→3)-α-Hep-(1→5)-α-Kdo, α-Hep-(1→3)-α-Hep-(1→5)-α-Kdo or α-GlcNAc-(1→2)-α-Hep-(1→3)-α-Hepa are a substructure or a partial sequence of their Lipopolysaccharides (LPS).

The saccharides according to the present invention and pharmaceutical compositions containing at least one saccharide of the present invention and especially the vaccine containing at least one saccharide and preferably the tetrasaccharide 1 are highly useful for eliciting antibodies, including monoclonal antibodies (MAb). Such elicited antibodies are also used for the treatment of diseases or support the treatment of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep such as Neisseria meningitidis. These antibodies could also be used for diagnostic purposes, for instance, to diagnose diseases such as meningitis, septicemia, pneumonia and nasopharyngitis caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

Moreover the inventive saccharide can be used in immunological assays for diagnostics of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. Especially the tetrasaccharide α-GlcNAc-(1→2)-α-Hep-(1→3)-α-Hep-(1→5)-α-Kdo may be used as a marker in immunological assays. Such assays comprise, for instance, microarray and ELISA useful for diagnosis of diseases caused by bacteria containing or comprising at least one of the saccharides α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. Such diseases which can be diagnosed comprise meningitis, septicemia, pneumonia and nasopharyngitis.

The saccharides of the present invention as well as the pharmaceutical compositions containing at least one saccharide and especially the vaccines containing at least one inventive saccharide, especially saccharide 1, 35, 36 and/or 37 are highly useful for the treatment and prophylaxis of diseases such as the group of diseases comprising or consisting of meningitis, septicemia, pneumonia and nasopharyngitis caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. Thus the saccharides of the present invention are preferably used for the preparation of a pharmaceutical composition and especially a vaccine for the treatment and prophylaxis of diseases caused by bacteria such as Neisseria meningitidis containing at least one of the saccharides α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep and more preferably for the preparation of a pharmaceutical composition and especially a vaccine for the treatment and prophylaxis of meningitis, septicemia, pneumonia and nasopharyngitis.

The bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep and preferably the saccharide α-GlcNAc-(1→2)-α-Hep-(1→3)-α-Hep-(1→5)-α-Kdo are preferably selected from the group consisting of all strains of Neisseria meningitidis, wherein the lipooligodaccharide (LOS) immunotypes are L1, L2, L3, L4, L5, L6, L7, L8, L9 and/or L11. The LOS immunotypes L1, L2, L3, L4, L5, L6, L7, L8, L9 and L11 contain the tetrasaccharide α-GlcNAc-(1→2)-α-Hep-(1→3)-α-Hep-(1→5)-α-Kdo. The LOS immunotypes L1-L9, and L11 are subgroups of or associated with serogroups A, B, and C.

A further aspect of the present invention is directed to intermediates of the synthetic saccharides of the general formula (I), (II), (III) and 1^{#}.

Preferred intermediates are the following compounds:

Compound 28^{#} of the following formula

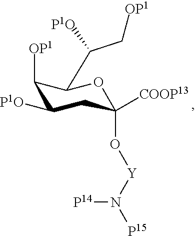

wherein P¹, P¹⁴, P¹³ and P¹⁵ are protecting groups or hydrogen while at least one of the groups P¹, P¹⁴, P¹³ and P¹⁵ is different from hydrogen and wherein P¹ represents preferably hydrogen or acetyl, P¹⁴ represents preferably hydrogen or benzyl, P¹³ represents preferably hydrogen or methyl and P¹⁵ represents preferably hydrogen or benzyloxy carbonyl, and

Compound 31^{#} of the following formula

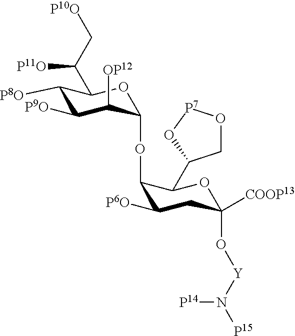

wherein P⁶-P¹⁵ are protecting groups or hydrogen while at least one of the groups P⁶-P¹⁵ is different from hydrogen and wherein P¹⁰ and P¹² represent preferably hydrogen or acetyl, P⁶, P¹¹ and P¹⁴ represent preferably hydrogen or benzyl, P⁷ represents preferably hydrogen or isopropyl, P⁸ represents preferably hydrogen or para-bromobenzyl, P⁹ represents preferably hydrogen or levulinyl, P¹³ represents preferably hydrogen or methyl and P¹⁵ represents preferably hydrogen or benzyloxy carbonyl, and

Compound 39^{#} of the following formula

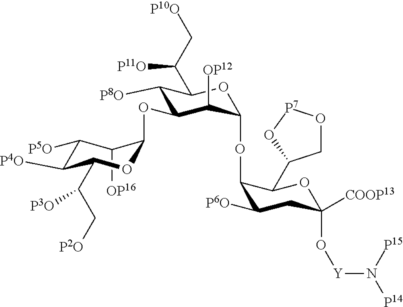

wherein P²-P⁸, P¹⁰-P¹⁵ are protecting groups or hydrogen while at least one of the groups P²-P⁸, P¹⁰-P¹⁵ is different from hydrogen and wherein P¹⁰ and P¹² represent preferably hydrogen or acetyl, P² and P⁵ represent preferably hydrogen or benzoyl, P³, P⁶, P¹¹ and P¹⁴ represent preferably hydrogen or benzyl, P⁷ represents preferably hydrogen or isopropyl, P⁴ and P⁸ represent preferably hydrogen or para-bromobenzyl, P¹³ represents preferably hydrogen or methyl and P¹⁵ represents preferably hydrogen or benzyloxy carbonyl, and

Compound 32^{#} of the following formula

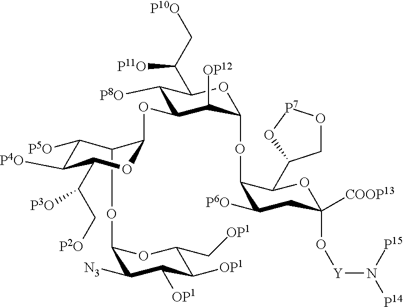

wherein P¹-P⁸, P¹⁰-P¹⁵ are protecting groups or hydrogen while at least one of the groups P¹-P⁸, P¹⁰-P¹⁵ is different from hydrogen and wherein P¹, P¹⁰ and P¹² represent preferably hydrogen or acetyl, P² and P⁵ represent preferably hydrogen or benzoyl, P³, P⁶, P¹¹ and P¹⁴ represent preferably hydrogen or benzyl, P⁷ represents preferably hydrogen or isopropyl, P⁴ and P⁸ represent preferably hydrogen or para-bromobenzyl, P¹³ represents preferably hydrogen or methyl and P¹⁵ represents preferably hydrogen or benzyloxy carbonyl.

In the above disclosed general formula 28^{#}, 31^{#}, 39^{#} and 32^{#} of the preferred intermediates the linker Y represents —Y¹—Y²—Y³—, —Y¹—Y³—, —Y¹—, or —Y¹—Y²—Y⁴—Y⁵—Y³—, with Y¹, Y², Y³, Y⁴ and Y⁵ as defined herein. More preferred the linker Y represents —CH₂—, —C₂H₄—, —C₃H₆—, —C₄H₈—, —C₅H₁₀—, —C₆H₁₂—, —C₇H₁₄—, —C₈H₁₆—, most preferably —C₅H₁₀—.

Particularly preferred intermediates are the following compounds:

Compound 2* of the following formula

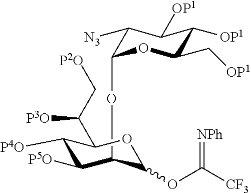

wherein P¹-P⁵ are protecting groups or hydrogen while at least one of the groups P¹-P⁵ is different from hydrogen and wherein P¹ represents preferably hydrogen or acetyl, P² and P⁵ represent preferably hydrogen or benzoyl, P³ represents preferably hydrogen or benzyl and P⁴ represents preferably hydrogen or para-bromobenzyl, and

Compound 3* of the following formula

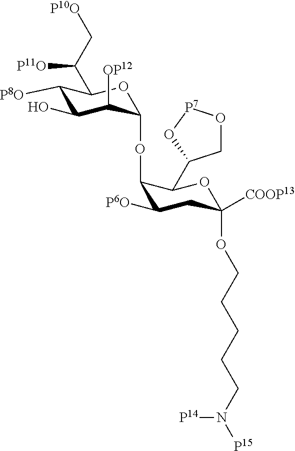

wherein P⁶-P⁸, P¹⁰-P¹⁵ are protecting groups or hydrogen while at least one of the groups P⁶-P⁸, P¹⁰-P¹⁵ is different from hydrogen and wherein P⁶, P¹¹ and P¹⁴ represent preferably hydrogen or benzyl, P⁷ represents preferably hydrogen or isopropyl, P⁸ represents preferably hydrogen or para-bromobenzyl, P¹⁰ and P¹² represent preferably hydrogen or acetyl, P¹⁵ represents preferably hydrogen or benzyloxy carbonyl, and P¹³ represents preferably hydrogen or methyl, and

Compound 39* of the following formula

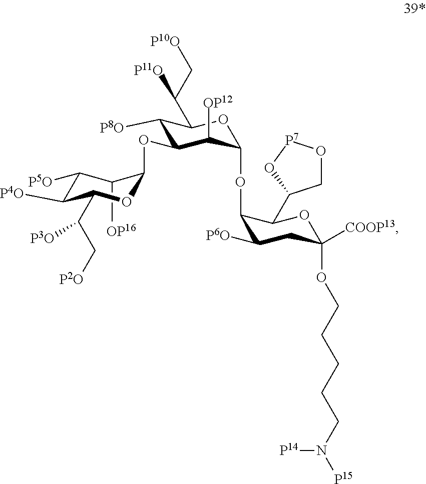

wherein P²-P⁸, P¹⁰-P¹⁵ are protecting groups or hydrogen while at least one of the groups P²-P⁸, P¹⁰-P¹⁵ is different from hydrogen and wherein P² and P⁵ represent preferably hydrogen or benzoyl, P³, P⁶, P¹¹ and P¹⁴ represent preferably hydrogen or benzyl, P⁷ represents preferably hydrogen or isopropyl, P⁴ and P⁸ represents preferably hydrogen or para-bromobenzyl, P¹⁰ and P¹² represent preferably hydrogen or acetyl, P¹³ represents preferably hydrogen or methyl and P¹⁵ represents preferably hydrogen or benzyloxy carbonyl, and

Compound 32* of the following formula

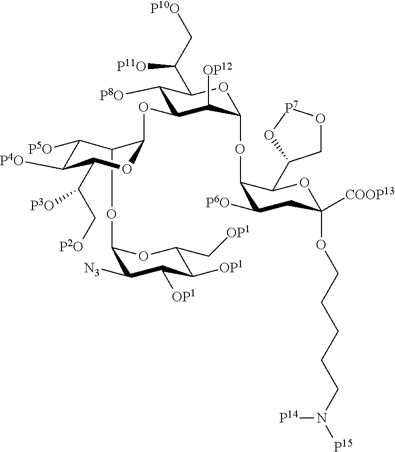

wherein P¹-P⁸, P¹⁰-P¹⁵ are protecting groups or hydrogen while at least one of the groups P¹-P⁸, P¹⁰-P¹⁵ is different from hydrogen and wherein P¹, P¹⁰ and P¹² represent preferably hydrogen or acetyl, P² and P⁵ represent preferably hydrogen or benzoyl, P³, P⁶, P¹¹ and P¹⁴ represent preferably hydrogen or benzyl, P⁷ represents preferably hydrogen or isopropyl, P⁴ and P⁸ represents preferably hydrogen or para-bromobenzyl, P¹³ represents preferably hydrogen or methyl and P¹⁵ represents preferably hydrogen or benzyloxy carbonyl.

The synthetic saccharides of the general formula (I), (II), (III), 1^{#}, 35^{#}, 36^{#} and 37^{#}, and in particular the saccharides 1, 35, 36 and 37 are basic and form salts with various organic and inorganic acids. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphersulfonic acid, china acid, mandelic acid, o-methylmandelic acid, hydrogen-benzenesulfonic acid, picric acid, adipic acid, D-o-tolyltartaric acid, tartronic acid, a-toluic acid, (o,m,p)-toluic acid, naphthylamine sulfonic acid, amino acids such as glycone, alanine, valine, leucine, isoleucine, serine, threonine, phenylalanine, tyrosine, tryptophane, lysine, arginine, histidine, asparaginic acid, glutamic acid, asparagines, glutamine, cysteine, methionine, proline, 4-hydroxyproline, N,N,N-trimethyllysine, 3-methylhistidine, 5-hydroxylysine, O-phosphoserine, γ-carboxyglutamate, ε-N-acetyllysine, (o-N-methylarginine, citrulline and ornithine, and other mineral or carboxylic acids well known to those skilled in the art. The salts are prepared by contacting the free base form with a sufficient amount of the desired acid to produce a salt in the conventional manner.

The free base forms may be regenerated by treating the salt with a suitable dilute aqueous base solution such as dilute aqueous sodium hydroxide, potassium carbonate, ammonia and sodium bicarbonate. The free base forms differ from their corresponding salt forms somewhat in certain physical properties, such as solubility in polar solvents, but the salts are otherwise equivalent to their corresponding free base forms for purposes of this invention.

Thus the inventive syntheses for the synthetic saccharides of the general formula (I) may further comprise step E)

E) preparing a salt of a synthetic saccharides of the general formula (I) or preparing a lyophilisate of a synthetic saccharides of the general formula (I) or of the salt of a synthetic saccharides of the general formula (I).

In a preferred embodiment, the inventive syntheses for the synthetic saccharides of the general formula (II) may further comprise step E)

E) preparing a salt of a synthetic saccharides of the general formula (II) or preparing a lyophilisate of a synthetic saccharides of the general formula (II) or of the salt of a synthetic saccharides of the general formula (II).

In a preferred embodiment, the inventive syntheses for the synthetic saccharides of the general formula (III) may further comprise step E)

E) preparing a salt of a synthetic saccharides of the general formula (III) or preparing a lyophilisate of a synthetic saccharides of the general formula (III) or of the salt of a synthetic saccharides of the general formula (III).

In a preferred embodiment, the inventive syntheses for the synthetic saccharides of the general formula 1^{#} may further comprise step E)

E) preparing a salt of a synthetic saccharides of the general formula 1^{#} or preparing a lyophilisate of a synthetic saccharides of the general formula 1^{#} or of the salt of a synthetic saccharides of the general formula 1^{#}.

In a preferred embodiment, the inventive syntheses for the monosaccharides 35 may further comprise step B)

B) preparing a salt of monosaccharides 35 or preparing a lyophilisate of monosaccharides 35 or of the salt of monosaccharides 35.

In a preferred embodiment, the inventive syntheses for the disaccharides 36 may further comprise step B)

B) preparing a salt of disaccharides 36 or preparing a lyophilisate of disaccharides 36 or of the salt of disaccharides 36.

In a preferred embodiment, the inventive syntheses for the trisaccharides 37 may further comprise step C)

C) preparing a salt of trisaccharides 37 or preparing a lyophilisate of trisaccharides 37 or of the salt of trisaccharides 37.

Also, in the particularly preferred embodiment the inventive synthesis of tetrasaccharide 1 as described above may further comprise step D)

D) preparing a salt of tetrasaccharide 1 or preparing a lyophilisate of tetrasaccharide 1 or of the salt of tetrasaccharide 1.

It is preferred that the reaction of compound 6* and 7*, of compound 6* and 57, of compound 6* and 62, of compound 6* and 67, of compound 6* and 72, of compound 6* and 77, of compound 6* and 82, of compound 6* and 89, of compound 6* and 96, of compound 6* and 103, of compound 6* and 110, of compound 6* and 117, of compound 6* and 124, of compound 6* and 131, of compound 6* and 138 and of compound 6* and 145 is performed in a polar aprotic solvent using trimethylsilyl trifluoromethanesulfonate (TMSOTf). In addition molecular sieve (MS) such as 4 Å molecular sieve can be used. The reaction temperature is between −20° C. and +20° C., preferably the temperature is between −10° C. and +10° C. and more preferably the temperature is between −5° C. and +5° C. and most preferably about 0° C.

The reaction is preferably carried out in a polar aprotic solvent such as acetonitrile, nitromethane, dimethylcarbonate, ethylencarbonate, ether such as tetrahydrofurane (THF) or diethylether, ketones such as acetone, butanone or pentanone, amides such as dimethylformamide (DMF) or dimethylacetamide, halogenated solvents such as chloroform, methylene chloride, carboxylic acid ester such as acetic acid ethyl ester.

It is preferred that the reaction of compound 4* and 5* is performed in an aprotic solvent using trimethylsilyl trifluoromethanesulfonate (TMSOTf). In addition molecular sieve (MS) such as 4 Å molecular sieve can be used. The reaction temperature is between −20° C. and +20° C., preferably the temperature is between −10° C. and +10° C. and more preferably the temperature is between −5° C. and +5° C. and most preferably about 0° C.

The reaction is preferably carried out in a polar aprotic solvent such as acetonitrile, nitromethane, dimethylcarbonate, ethylencarbonate, ether such as tetrahydrofurane (THF) or diethylether, ketones such as acetone, butanone or pentanone, amides such as dimethylformamide (DMF) or dimethylacetamide, halogenated solvents such as chloroform, methylene chloride, carboxylic acid ester such as acetic acid ethyl ester.

Preferably the conversion of compound 30* to compound 2* is performed in two steps. The first step is reacting compound 30* in a polar aprotic solvent and water mixture using N-bromosuccinimide (NBS) and the second step involves reacting the product obtained after the first step with CF₃C(═NPh)Cl and a base in a polar aprotic solvent. Both steps are preferably carried out at room temperature or room temperature ±15° C. For the first step solvent mixtures of a ketone and water are preferred such as acetone/water mixtures. For the second step polar aprotic solvents such as acetonitrile, nitromethane, sulfolane, dimethylcarbonate, ethylencarbonate, dimethylsulfoxide, ether such as tetrahydrofurane (THF) or diethylether, ketones such as acetone, butanone or pentanone, amides such as dimethylformamide (DMF) or dimethylacetamide, halogenated solvents such as chloroform, methylene chloride, carboxylic acid ester such as acetic acid ethyl ester are suitable.

Preferably the conversion of compound 31* to 3*, of compound 83 to 84, of compound 90 to 91, of compound 97 to 98, of compound 104 to 105, of compound III to 112, of compound 118 to 119, of compound 125 to 126, of compound 132 to 133, of compound 139 to 140 and of compound 147 to 146 is performed in a polar aprotic solvent by means of hydrazine or a hydrazinium salt. Suitable polar aprotic solvents are mentioned above. The reaction is preferably conducted at room temperature or room temperature ±15° C. Hydrazinium salts of weak acids are preferred such as hydrazinium acetate or hydrazinium proprionate.

It is also preferred that the reaction of compound 2* and the compound 3*, of compound 2* and the compound 119, of compound 2* and the compound 126, of compound 2* and the compound 133, of compound 2* and the compound 140 and of compound 2* and the compound 146 is performed in a polar aprotic solvent using trimethylsilyl trifluoromethanesulfonate (TMSOTf). In addition molecular sieve (MS) such 4 Å molecular sieve can be used. The reaction temperature is between −20° C. and +20° C., preferably the temperature is between −10° C. and +10° C. and more preferably the temperature is between −5° C. and +5° C. and most preferably about 0° C.

The reaction is preferably carried out in a polar aprotic solvent such as acetonitrile, nitromethane, dimethylcarbonate, ethylencarbonate, ether such as tetrahydrofurane (THF) or diethylether, ketones such as acetone, butanone or pentanone, amides such as dimethylformamide (DMF) or dimethylacetamide, halogenated solvents such as chloroform, methylene chloride, carboxylic acid ester such as acetic acid ethyl ester.

It is also preferred that the reaction of compound 38* and the compound 3*, of compound 38* and the compound 84, of compound 38* and the compound 91, of compound 38* and the compound 98, of compound 38* and the compound 105 and of compound 2* and the compound 112 is performed in a polar aprotic solvent using trimethylsilyl trifluoromethanesulfonate (TMSOTf). In addition molecular sieve (MS) such 4 Å molecular sieve can be used. The reaction temperature is between −20° C. and +20° C., preferably the temperature is between −10° C. and +10° C. and more preferably the temperature is between −5° C. and +5° C. and most preferably about 0° C.

The reaction is preferably carried out in a polar aprotic solvent such as acetonitrile, nitromethane, dimethylcarbonate, ethylencarbonate, ether such as tetrahydrofurane (THF) or diethylether, ketones such as acetone, butanone or pentanone, amides such as dimethylformamide (DMF) or dimethylacetamide, halogenated solvents such as chloroform, methylene chloride, carboxylic acid ester such as acetic acid ethyl ester.

Thus, preferably the reaction of compound 6* and 7*, the reaction of compound 2* and 3* or the reaction of compound 4* and 5* is performed in a polar aprotic solvent using TMSOTf.

Preferably the conversion of compound 32* to compound 33*

of the formula

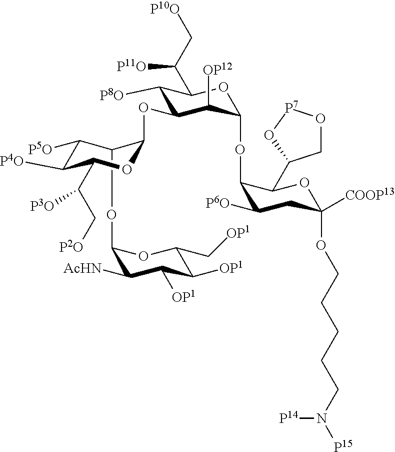

is performed in a polar aprotic solvent to which a base is added or in a polar aprotic basic solvent using thioacetic acid (AcSH). Suitable polar aprotic solvents are mentioned above. Suitable polar aprotic basic solvents are amines such as pyridine. The reaction is preferably conducted at room temperature or room temperature ±15° C.

Preferably the conversion of compound 33* to compound 1, of compound 31 to disaccharide 36, of compound 39 to trisaccharide 37, of compound 58 to disaccharide 59, of compound 36 to disaccharide 64, of compound 68 to disaccharide 69, of compound 73 to disaccharide 74, of compound 78 to disaccharide 79, of compound 85 to trisaccharide 86, of compound 92 to trisaccharide 93, of compound 99 to trisaccharide 100, of compound 106 to trisaccharide 107 and of compound 113 to trisaccharide 114 is performed in three steps, first the acid-labile protecting groups are cleaved in a mixture of an acid in water; second the base-labile protecting groups are cleaved in a polar aprotic solvent/water mixture or a polar protic solvent/water mixture using a base; and third the protecting groups sensitive for hydrogenation are cleaved by means of hydrogen and a catalyst. The first step is preferably carried out in a mixture of a weak acid such as formic acid, acetic acid, propionic acid in water at elevated temperature. The reaction temperature is preferably between 50° C. and 90° C., more preferably between 60° C. and 80° C. and most preferably between 65° C. and 75° C. For the second step suitable protic solvents are alcohols such as methanol, ethanol, propanol, or isopropanol, which are used in combination with water and probably another polar solvent such as acetonitrile, nitromethane, sulfolane, dimethylcarbonate, ethylencarbonate, dimethylsulfoxide, ether such as tetrahydrofurane (THF) or diethylether, amides such as dimethylformamide (DMF) or dimethylacetamide, halogenated solvents such as chloroform, or methylene chloride. The basic cleavage of the protecting groups is preferably performed at room temperature or room temperature ±15° C. The third step involves cleavage of the protecting groups sensitive for hydrogenation by the use of hydrogen gas and a catalyst such as palladium on carbon (Pd/C) and palladium hydroxide on carbon (Pd(OH)₂/C). The hydrogenation is preferably performed at room temperature or room temperature ±15° C. Preferably a solvent mixture of water, a weak acid such as formic acid, acetic acid, or propionic acid and a protic solvent such as alcohols such as methanol, ethanol, propanol, or isopropanol.

Preferably the conversion of compound 120 to tetrasaccharide 121, of compound 127 to tetrasaccharide 128, of compound 134 to tetrasaccharide 135, of compound 141 to tetrasaccharide 142 and of compound 148 to tetrasaccharide 149, is performed in a polar aprotic solvent to which a base is added or in a polar aprotic basic solvent using thioacetic acid (AcSH) and subsequent deprotection in three steps first the acid-labile protecting groups are cleaved in a mixture of an acid in water; second the base-labile protecting groups are cleaved in a polar aprotic solvent/water mixture or a polar protic solvent/water mixture using a base; and third the protecting groups sensitive for hydrogenation are cleaved by means of hydrogen and a catalyst as described abobe.

Preferably the conversion of compound 28* to monosaccharide 35, of compound 41 to monosaccharide 42, of compound 44 to monosaccharide 45, of compound 47 to monosaccharide 48, of compound 50 to monosaccharide 51 and of compound 53 to monosaccharide 54 is performed in two steps, first the base-labile protecting groups are cleaved in a polar aprotic solvent/water mixture or a polar protic solvent/water mixture using a base; and second the protecting groups sensitive for hydrogenation are cleaved by means of hydrogen and a catalyst. For the first step suitable protic solvents are alcohols such as methanol, ethanol, propanol, or isopropanol, which are used in combination with water and probably another polar solvent such as acetonitrile, nitromethane, sulfolane, dimethylcarbonate, ethylencarbonate, dimethylsulfoxide, ether such as tetrahydrofurane (THF) or diethylether, amides such as dimethylformamide (DMF) or dimethylacetamide, halogenated solvents such as chloroform, or methylene chloride. The basic cleavage of the protecting groups is preferably performed at room temperature or room temperature ±15° C. The second step involves cleavage of the protecting groups sensitive for hydrogenation by the use of hydrogen gas and a catalyst such as palladium on carbon (Pd/C) and palladium hydroxide on carbon (Pd(OH)₂/C). The hydrogenation is preferably performed at room temperature or room temperature ±15° C. Preferably a solvent mixture of water, a weak acid such as formic acid, acetic acid, or propionic acid and a protic solvent such as alcohols such as methanol, ethanol, propanol, or isopropanol.

The syntheses of the inventive saccharides as disclosed herein constitute a milestone which finally enables production of sufficient amounts of the desired LPS structure to support research towards understanding and designing a vaccine against N. meningitidis.

According to the present invention the tetrasaccharide 1 as well as any other inventive saccharide disclosed herein is useful for the manufacture of a pharmaceutical composition for use as vaccine for immunization against diseases caused by bacteria containing or comprising the tetrasaccharide α-GlcNAc-(1→2)-α-Hep-(1→3)-α-Hep-(1→5)-α-Kdo or the trisaccharide α-Hep-(1→3)-α-Hep-(1→5)-α-Kdo or the trisaccharide α-GlcNAc-(1→2)-α-Hep-(1→3)-α-Hep, especially for immunization against meningitis, septicaemia, pneumonia and nasopharyngitis caused by Neisseria meningitidis.

Specifically, according to the present invention tetrasaccharide 1 is used as a vaccine for immunization against diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep, especially for immunization against meningitis, septicaemia, pneumonia and nasopharyngitis caused by Neisseria meningitidis.

Example of bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep p is Neisseria meningitidis. All strains of Neisseria meningitidis wherein the LOS immunotypes are L1, L2, L3, L4, L5, L6, L7, L8, L9 and L11 contain such a tetrasaccharide or trisaccharide. The LOS immunotypes L1-L9, and L11 are associated with serogroups A, B, and C.

Examples of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep are meningitis, septicemia, pneumonia and nasopharyngitis.

Scheme 2 provides synthesis of trisaccharide 39* from the donor 38* and disaccharide acceptor 3*

It is preferred that the reaction of compound 38* and 3* is performed in an aprotic solvent using trimethylsilyl trifluoromethanesulfonate (TMSOTf). In addition molecular sieve (MS) such as 4 Å molecular sieve can be used. The reaction temperature is between −20° C. and +20° C., preferably the temperature is between −10° C. and +10° C. and more preferably the temperature is between −5° C. and +5° C. and most preferably about 0° C.

The reaction is preferably carried out in a polar aprotic solvent such as acetonitrile, nitromethane, dimethylcarbonate, ethylencarbonate, ether such as tetrahydrofurane (THF) or diethylether, ketones such as acetone, butanone or pentanone, amides such as dimethylformamide (DMF) or dimethylacetamide, halogenated solvents such as chloroform, methylene chloride, carboxylic acid ester such as acetic acid ethyl ester.

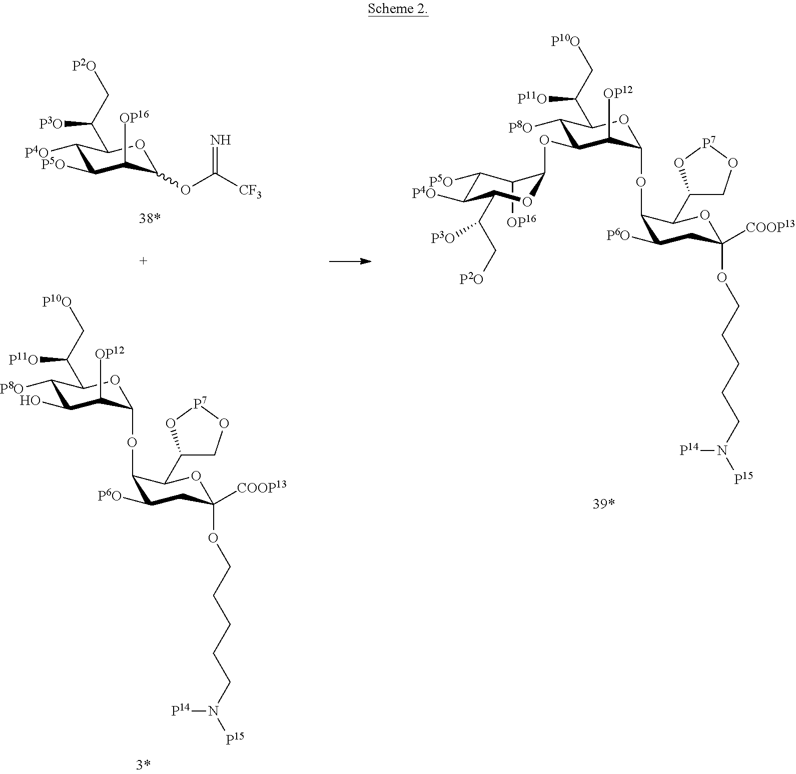

Preferably the conversion of compound 39* to compound 37 is performed in three steps, first the acid-labile protecting groups are cleaved in a mixture of an acid in water; second the base-labile protecting groups are cleaved in a polar aprotic solvent/water mixture or a polar protic solvent/water mixture using a base; and third the protecting groups sensitive for hydrogenation are cleaved by means of hydrogen and a catalyst. The first step is preferably carried out in a mixture of a weak acid such as formic acid, acetic acid, propionic acid in water at elevated temperature. The reaction temperature is preferably between 50° C. and 90° C., more preferably between 60° C. and 80° C. and most preferably between 65° C. and 75° C. For the second step suitable protic solvents are alcohols such as methanol, ethanol, propanol, or isopropanol, which are used in combination with water and probably another polar solvent such as acetonitrile, nitromethane, sulfolane, dimethylcarbonate, ethylencarbonate, dimethylsulfoxide, ether such as tetrahydrofurane (THF) or diethylether, amides such as dimethylformamide (DMF) or dimethylacetamide, halogenated solvents such as chloroform, or methylene chloride. The basic cleavage of the protecting groups is preferably performed at room temperature or room temperature ±15° C. The third step involves cleavage of the protecting groups sensitive for hydrogenation by the use of hydrogen gas and a catalyst such as palladium on carbon (Pd/C) and palladium hydroxide on carbon (PdOH/C). The hydrogenation is preferably performed at room temperature or room temperature ±15° C. Preferably a solvent mixture of water, a weak acid such as formic acid, acetic acid, or propionic acid and a protic solvent such as alcohols such as methanol, ethanol, propanol, or isopropanol.

A synthetic saccharide of general formula (I)

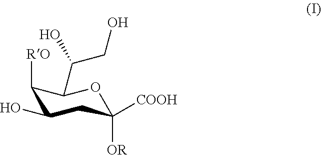

wherein
R represents -L-NH₂
R′ represents H or

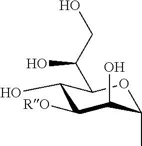

R″ represents H or

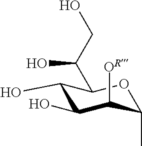

R′″ represents H or

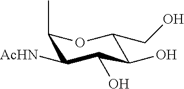

for use as a vaccine for immunization against diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

The synthetic saccharide of any one of the general formula (I), (II), (III), 1^{#}, 35^{#}, 36^{#} and 37^{#} could be used as a vaccine for eliciting antibodies. The synthetic saccharide of any one of the general formula (I), (II), (III), 1^{#}, 35^{#}, 36^{#} and 37^{#} could also be used in immunological assays for diagnostics of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. Preferably the diseases caused by these bacteria are selected from the group comprising or consisting of meningitis, septicemia, pneumonia and nasopharyngitis.

Further was found that extraordinary potent and stable vaccine can be derived when a synthetic saccharide of general formula (I), (II), (III), 1^{#}, 35^{#}, 36^{#} or 37^{#} is bound to a carrier protein. The present invention relates therefore to a synthetic saccharide of any one of the general formula (I), (II), (III), 1^{#}, 35^{#}, 36^{#} and 37^{#} covalently linked to a carrier protein. Particularly preferred is that said carrier protein is a bacterial peptide or a synthetic saccharide derived from a bacterial peptide.

It is particularly preferred that the carrier protein a synthetic saccharide of any one of the general formula (I), (II), (III), 1^{#}, 35^{#}, 36^{#} and 37^{#} is covalently linked to is selected from the group comprising or consisting of: a diphtheria toxoid, a mutated diphtheria toxoid, a modified diphtheria toxoid, a mutated and modified diphtheria toxoid, a tetanus toxoid, a modified tetanus toxoid, a mutated tetanus toxoid, outer membrane protein (OMP), bovine serum albumin (BSA), keyhole limpet hemocyanine (KLH or cholera toxoid (CT). The term “toxoid” as used herein refers to a bacterial toxin (usually an exotoxin) whose toxicity has been inactivated or suppressed either by chemical (formalin) or heat treatment, while other properties, typically immunogenicity, are maintained. A mutated toxoid as used herein is a recombinant bacterial toxin which has been amended to be less toxic or even non-toxic by amending the wild-type amino acid sequence. Such a mutation could be a substitution of one or more amino acids. A modified toxoid as used herein is an activated bacterial toxoid a functional group has been introduced or which has been coupled to a linker for binding of any antigen. It is especially preferred that the synthetic saccharide of general formula (I), (II), (III), 1^{#}, 35^{#}, 36^{#} or 37^{#} and preferably the saccharide 1, 35, 36 or 37 is covalently linked to the non-toxic mutated diphtheria toxin CRM₁₉₇ which is modified by maleimide. CRM₁₉₇ like wild-type diphtheria toxin is a single polypeptide chain of 535 amino acids (58 kD) consisting of two subunits linked by disulfide bridges having a single amino acid substitution of glutamic acid for glycine. It is utilized as a carrier protein in a number of approved conjugate vaccines for diseases such as meningitis and pneumococcal bacterial infections.

In one aspect of the present invention the synthetic saccharide of any one of the formula (I), (II), (III), 1^{#}, 35^{#}, 36^{#} and 37^{#} according to the present invention is attached to a carrier protein. One embodiment of the present invention is a synthetic saccharide of the formula (I), (II), (III), 1^{#}, 35^{#}, 36^{#} or 37^{#} attached to a carrier protein for use as a vaccine for immunization against diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. It is preferred that said diseases are selected from the group consisting of meningitis, septicemia, pneumonia and nasopharyngitis. The bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep are selected from the group consisting of all strains of Neisseria meningitidis, wherein the lipo-oligosaccharide (LOS) immunotypes are L1, L2, L3, L4, L5, L6, L7, L8, L9 and/or L11.

A saccharide-protein conjugate (V) comprises the synthetic saccharide according to any one of the general formula (I), (II), (III), 1^{#}, 35^{#}, 36^{#} and 37^{#} coupled to said protein carrier. A method for conjugation of the synthetic saccharide of the invention to a carrier protein comprises reacting a unique terminal amine of the linker Y with one of the two NHS-activated esters of Di(N-succinimidyl) adipate to form an amide and subsequent coupling of the activated amide moiety to the protein carrier.

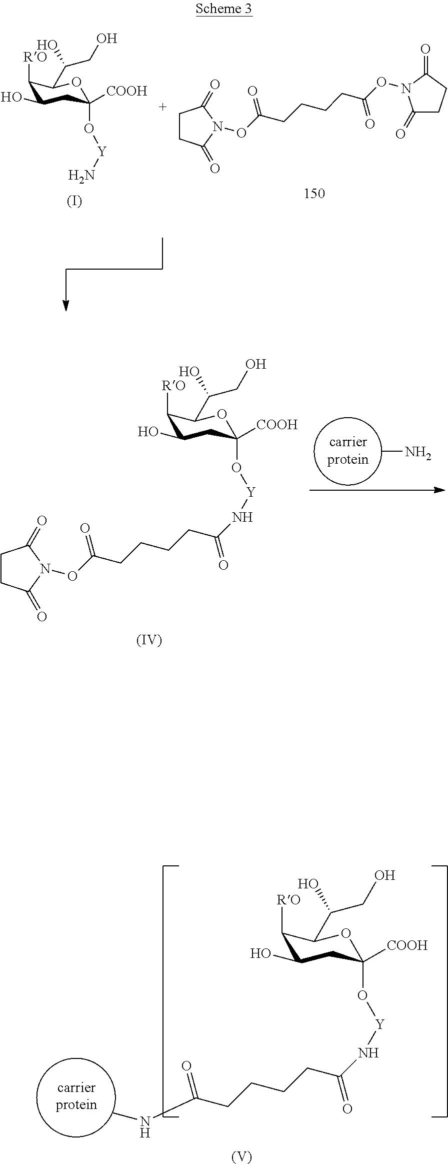

In an embodiment of the present invention a synthetic saccharide of any one of the formula (I), (II), (III), 1^{#}, 35^{#}, 36^{#} and 37^{#} is conjugated to a carrier protein in order to form a synthetic saccharide-protein conjugate (V). The carrier protein is selected from a group of diphtheria toxoid CRM₁₉₇, tetanus toxoid (TT), outer membrane protein (OMP), bovine serum albumin (BSA), keyhole limpet hemocyanine (KLH), diphtheria toxoid (DT) and cholera toxoid (CT).

A method for conjugation of a synthetic saccharide of any one of the general formula (I), (II), (III), 1^{#}, 35^{#}, 36^{#} and 37^{#} to a carrier protein comprises reacting a unique terminal amine of the linker with one of the two NHS-activated esters of Di(N-succinimidyl) adipate 150 to form an amide and subsequent coupling of the activated amide moiety to the protein carrier. A synthetic saccharide-protein conjugate (V) consists of a synthetic saccharide (I) and a carrier protein which connected each other with adipic amide.

In a preferred embodiment of the present invention the synthetic saccharide-protein conjugate (V) comprises a saccharide of any one of the general formula (I), (II), (III), 1^{#}, 35^{#}, 36^{#} and 37^{#}. It is further preferred that the synthetic saccharide-protein conjugate (V) comprises the carrier protein diphtheria toxoid CRM₁₉₇.

In a preferred embodiment of the present invention the synthetic saccharide-protein conjugate (V) comprises a compound selected from a group consisting of saccharides 35, 36, 37 and 1. It is further preferred that the synthetic saccharide-protein conjugate (V) comprises the carrier protein diphtheria toxoid CRM₁₉₇.

In the most preferred embodiment of the present invention the synthetic saccharide-protein conjugate (V) comprises the tetrasaccharide 1 and the carrier protein diphtheria toxoid CRM₁₉₇. In one aspect of the present invention the synthetic saccharide of any one of the formula (I), (II), (III), 1^{#}, 35^{#}, 36^{#} and 37^{#} according to the present invention conjugated to a carrier protein is lyophilized to achieve enhanced stability.

A synthetic saccharide of general formula (I), (II), (III), 1^{#}, 35^{#}, 36^{#} or 37^{#} or a mixture of such saccharides could be immobilized on a microarray surface or any other surface and used for an in vitro method of detecting Neisseria meningitidis. A method of identifying a certain strain of N. meningitidis or an infection caused by N. meningitidis comprises the use of at least one synthetic saccharide of the present invention. Furthermore, the synthetic saccharide of general formula (I), (II), (III), 1^{#}, 35^{#}, 36^{#} or 37^{#} or a mixture of such saccharides can be used as an analytical standard for immunoassays.

The present invention refers also to an antibody against at least one synthetic saccharide of general formula (I), (II), (III), 1^{#}, 35^{#}, 36^{#} or 37^{#} according to the present invention. The term “antibody” as used herein encompasses polyclonal and monoclonal antibody preparations, as well as preparations including hybrid antibodies, F(ab′)₂ fragments, F(ab) molecules, single domain antibodies and functional fragments thereof which exhibit immunological binding properties of the parent antibody molecule.

The antibody according to the invention may be polyclonal or monoclonal. Especially preferred monoclonal antibodies according to the invention are produced by the monoclonal hybridoma 1A5 1G1 or 1B6 4E1. The 1A5 1G1 and 1B6 4E1 hybridomas, producing the 1A5 1G1 and 1B6 4E1 antibodies, respectively, were deposited under the provisions of the Budapest Treaty with the Leibniz Institute DSMZ-German Collection of Microorganisms and Cell Cultures, InhoffenstraRte 7B, 38124 Braunschweig; Germany. The 1A5 1G1 hybridoma was assigned Accession Number ______ and the 1B6 4E1 hybridoma was assigned Accession Number ______. The present invention refers also to monoclonal antibodies against at least one synthetic saccharide of general formula (I), (II), (III), 1^{#}, 35^{#}, 36^{#} or 37^{#}, wherein the hybridoma cells producing this antibodies are deposited with the DSMZ under the name 1A5 1G1 (Accession Number ______) and 1B6 4E1 (Accession Number ______). The present invention refers also to monoclonal antibodies designated 1A5 1G1, produced by the hybridoma cell line 1A5 1G1 and to antibodies designated 1B6 4E1, produced by the hybridoma cell line 1B6 4E1 deposited with the DSMZ.

Further the present invention refers to the use of at least one synthetic saccharide of general formula (I), (II), (III), 1^{#}, 35^{#}, 36^{#} or 37^{#} according to the invention or at least one antibody against at least one synthetic saccharide of the present invention in immunological assays for diagnostics of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. It is preferred if said diseases are selected from the group comprising or consisting of meningitis, septicemia, pneumonia and nasopharyngitis.

Such assays comprise, for instance, microarray and ELISA useful for diagnosis of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep. Therefore another aspect of the present invention refers to the use of a synthetic saccharide of formula (I), (II), (III), 1^{#}, 35^{#}, 36^{#} or 37^{#} and especially of the saccharide 1, 35, 36 or 37 for diagnosis of said diseases. Thus especially preferred embodiments of the present invention relate to pure synthetic saccharides of the present invention for diagnosis of bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

One embodiment of the present invention relates to a kit comprising at least one synthetic saccharide of the present invention immobilized on a carrier by covalent bonding or the synthetic saccharide of the present invention for immobilization on a carrier. The at least one synthetic saccharide of the present invention may be used as a marker in said assays. Another embodiment of the present invention relates to a kit comprising at least one antibody against a synthetic saccharide of general formula (I), (II), (III), 1^{#}, 35^{#}, 36^{#} or 37^{#}.

A kit in molecular biology or in medical diagnostics is a package which includes all necessary ingredients for performing a certain method or singular step. Standard chemicals as present in any standard molecular biology or medical laboratory are normally not included. Nevertheless some of these standard chemicals may be indispensable to carry out the diagnosis or the immobilization properly. It is understood that all ingredients are provided in quantities that allow for a proper execution of the desired reactions for the majority of scientific, diagnostic and industrial applications.

Often, but not always, these ingredients are provided in already prepared solutions ready- or close to ready-for-use. There may be also combinations of different ingredients already added together. A further advantage is that such kits use to be verified. Therefore the operator doesn't have to prove again the viability of the diagnostic method and can save on at least some control experiments. Therefore kits are a very popular tool in laboratories in research, diagnostics and industry.

Such a kit according to the invention shall include at least the following components:
A) at least one synthetic saccharide of any one of general formula (I), (II), (III), 1^{#}, 35^{#}, 36^{#} and 37^{#} immobilized on a carrier by covalent bonding
B) at least one antibody, like detection antibody
C) a standard solution

or a kit according to the invention shall include at least the following components:
A′) at least one antibody against a synthetic saccharide of the present invention
B) at least one further antibody, like detection antibody
C) a standard solution

The following components may also be included in such kits:
D) blocking solution
E) wash solution
F) sample buffer

An antibody in the kit may be a specific antibody which can be used as a capture antibody. But preferably it is at least an enzyme-linked secondary antibody used as detection antibody that binds specifically to antibody's Fc region. For quantitative determinations, the optical density (OD) or fluorescence of the sample is compared to a standard curve, which is typically a serial dilution of a known-concentration solution of the target molecule (a standard solution). A blocking solution may be a solution of a non-reacting protein, such as bovine serum albumin or casein, which is added to block any plastic surface in the well that remains uncoated by the antigen. Washing solutions are used to remove unbound components. A sample buffer may be used to dilute the sample of the patient (blood, serum, urine) so that the concentration of the target molecule is in the range which can normally be detected by the test system used.

If the kit shall be allowed for the immobilization of a synthetic saccharide of general formula (I), (II), (III), 1^{#}, 35^{#}, 36^{#} or 37^{#} on a solid carrier the kit should include at least:
A) At least one synthetic saccharide of any one of general formula (I), (II), (III), 1^{#}, 35^{#}, 36^{#} and 37^{#} or an antibody against at least one synthetic saccharide of general formula (I), (II), (III), 1^{#}, 35^{#}, 36^{#} or 37^{#}
B) A carrier, like a microtiter plate or microarray slide

Thereby the carrier may be modified, for example the carrier may be functionalized with a linker molecule as described above.

The following components may also be included in such kits:
C) blocking solution
D) wash solution
E) reaction buffer

One further aspect of the present invention relates to at least one synthetic saccharide of the present invention or at least one antibody according to the invention for use in the treatment or prophylaxis of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

Preferably said diseases are selected from the group comprising or consisting of meningitis, septicemia, pneumonia and nasopharyngitis.

Pharmaceutical Compositions

One aspect of the present invention relates to pharmaceutical compositions, especially vaccines containing at least one synthetic saccharide of any one of general formula (I), (II), (III), 1^{#}, 35^{#}, 36^{#} and 37^{#}

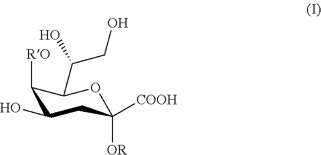

wherein

R represents —Y—NH₂,

Y represents a linker,
R′ represents H or

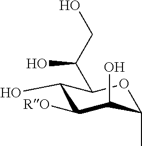

R″ represents H or

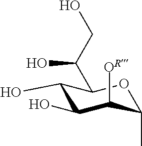

R′″ represents H or

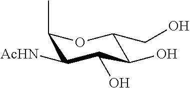

together with at least one pharmaceutically acceptable carrier, cryoprotectant, lyoprotectant, excipient, adjuvant and/or diluent.

Vaccines according to the present invention comprise the at least one saccharide of general formula (I), (II), (III), 1^{#}, 35^{#}, 36^{#} or 37^{#}, especially tetrasaccharide 1, trisaccharide 37, disaccharide 36 or monosaccharide 35 or enantiomers, stereoisomeric forms, mixtures of enantiomers, diastereomers, mixtures of diastereomers, hydrates, tautomers, solvates or racemates or pharmaceutically acceptable salts thereof. Especially the saccharides 1^{#}, 35^{#}, 36^{#}, 37^{#}, 35, 36, 37 and 1 obtained according to the total chemical synthesis disclosed herein are used in these vaccines. The saccharides of general formula (I), (II), (III), 1^{#}, 35^{#}, 36^{#} and 37^{#} as well as the saccharides 1, 35, 36 and 37 obtained by the total chemical synthesis disclosed herein have a purity of at least 95%, more preferably of at least 96%, still more preferably of at least 97%, still more preferably of at least 98%, and most preferably of at least 99% and do not contain any PEA substituents and do not contain endotoxin lipid A and do also not have any microheterogenicity.

The expression tautomer is defined as an organic compound that is interconvertible by a chemical reaction called tautomerization. Tautomerization can be catalyzed preferably by bases or acids or other suitable compounds. The vaccine may be prepared in the form of a suspension or may be lyophilized.

The suspension form may be stored frozen. In the lyophilized form, it is preferable to add one or more stabilizers. Vaccination can be performed at any age. The vaccine many be administered subcutaneously, by spray, by injection, orally, intraocularly, intratracheally or nasally. The amount of vaccine of the invention to be administered a human or animal and the regime of administration can be determined in accordance with standard techniques well known to those of ordinary skill in the pharmaceutical and veterinary arts taking into consideration such factors as the particular antigen, the adjuvant (if present), the age, sex, weight, species and condition of the particular animal or patient, and the route of administration.

In the present invention, the amount of saccharide-protein carrier to provide an efficacious dose for vaccination against N. meningitidis can be from between 0.02 μg to about 10 μg per kg body weight. The saccharide of any one of the formula (I), (II), (III), 1^{#}, 35^{#}, 36^{#} and 37^{#} according to the present invention optionally conjugated to a carrier protein can be administered as a single dose or in a series (i.e., with a “booster” or “boosters”). For example, a child could receive a single dose early in life, then be administered a booster dose up to ten years later, as is currently recommended for other vaccines to prevent childhood diseases.

Intravenous and parenteral administration is preferred. Such compositions may be in admixture with a suitable carrier, diluent, or excipient such as sterile water, physiological saline, glucose or the like. The compositions or the saccharides of any one of the formula (I), (II), (III), 1^{#}, 35^{#}, 36^{#} and 37^{#} according to the present invention optionally conjugated to a carrier protein can also be lyophilized.

Lyophilized compounds of the invention will ultimately be reconstituted with a liquid component to give material suitable for administration to a patient. The reconstitution will typically take place at the point of use. Thus a compound of the invention and an oil-in-water emulsion adjuvant or a buffer solution of an adjuvant may be kept separately in a packaged or distributed vaccine kit, ready for final formulation at the time of use. In a kit containing two containers, one will include liquid for reconstitution and the second container includes lyophilized material. For stability reasons, the lyophilized component of the invention may include a stabilizer such as lactose, sucrose and/or mannitol, as well as mixtures thereof. Using a sucrose/mannitol mixture can speed up the drying process. A lyophilized component may also include sodium chloride. Soluble components in the lyophilized material will be retained in the composition after reconstitution, and so final liquid vaccines may thus contain lactose and/or sucrose.

Formulation of the vaccines of the present invention can be accomplished using methods known by the art. Obviously, the choice of suitable carriers and other additives will depend on the exact route of administration and the nature of the particular dosage form.

The vaccine compositions of the present invention may contain one or more adjuvants. As defined herein, an “adjuvant” is a substance that serves to enhance the immunogenicity of a saccharide of any one of the formula (I), (II), (III), 1^{#}, 35^{#}, 36^{#} and 37^{#} according to the present invention optionally conjugated to a carrier protein. An immune adjuvant may enhance an immune response to an antigen that is weakly immunogenic when administered alone, e.g., inducing no or weak antibody titers or cell-mediated immune response. Further an adjuvant may increase antibody titers to the antigen, and/or lowers the dose of the antigen effective to achieve an immune response in the individual. Thus, adjuvants are often given to boost the immune response and are well known to the skilled person. Suitable adjuvants to enhance effectiveness include, by way of example and not limitation, aluminum adjuvants (e.g., aluminum salts such as aluminum hydroxide, aluminum phosphate, aluminum sulfate or combinations thereof), Freund's Adjuvant (Complete or Incomplete), BAY R1005 (N-(2-Deoxy-2-L-leucylamino-β-D-glucopyranosyl)-N-octadecyldodecanoylamide hydroacetate), DC-chol (dimethylaminoethane)-carbamoyl cholestreol, PCPP (poly[di(carboxylatophenoxy) phosphazene]), monophoshoryl lipid A, CpG oligonucleotides, saccharideS-21 (saccharideuillaja saponaria saponin immunologic adjuvant), cholera toxin and formyl methionyl peptide.

Alternative adjuvants include oil-in-water emulsion formulations for example MF59 as described in PCT Publ. No. WO 90/14837, SAF-1 (Syntex Adjuvant Formulation threonyl-MDP (0.05-1%) in an emulsion vehicle [5% squalane, 2.5% Pluronic® L121, 0.2% Polysorbate 80 and phosphate buffered saline (pH 7.4)], and one or more bacterial cell wall components from the group consisting of monophosphorylipid A (MPL), trehalose dimycolate (TDM), and cell wall skeleton (CWS), saponin adjuvants, such as Stimulon™ (Cambridge Bioscience, Worcester, Mass.) may be used or particles generated thereof. Various oil-in-water emulsion adjuvants are known, and they typically include at least one oil and at least one surfactant, with the oil(s) and surfactant(s) being biodegradable (metabolisable) and biocompatible. Squalane, the saturated analog to squalene, is preferred oil. Other preferred oils are the tocopherols. Mixtures of oils can be used. Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens); copolymers of ethylene oxide (EO), propylene oxide (PO), and/or butylene oxide (BO); octoxynols, which can vary in the number of repeating ethoxy(oxy-1,2-ethanediyl) groups, such as octoxynol-9 (Triton X-100, or toctylphenoxypolyethoxyethanol); phospholipids such as phosphatidylcholine (lecithin); polyoxyethylene fatty ethers derived from lauryl, cetyl, stearyl and oleyl alcohols (known as Brij surfactants). Preferred surfactants for including in the emulsion are Tween 80 (polyoxyethylene sorbitan monooleate), Span 85 (sorbitan trioleate), lecithin and Triton X-100.

Further adjuvants may be cytokines, such as interleukins (e.g., IL-1, IL-2, IL-4, IL-5, IL-6, IL-7, IL-12, etc.), interferons (e.g., gamma interferon), macrophage colony stimulating factor (M-CSF), tumor necrosis factor (TNF).

The compositions can contain auxiliary substances such as wetting or emulsifying agents, pH buffering agents, gelling or viscosity enhancing additives, preservatives, flavoring agents, colors, and the like, depending upon the route of administration and the preparation desired. Compositions of the invention may include an antimicrobial, particularly when packaged in multiple dose format. Antimicrobials such as thiomersal and 2-phenoxyethanol are commonly found in vaccines, but it is preferred to use either a mercury-free preservative or no preservative at all.

A composition may include a temperature protective agent. Examples include glycerin, propylene glycol, and/or polyethylene glycol (PEG).

Compositions of the invention are conveniently provided as liquid preparations, e.g., isotonic aqueous solutions, suspensions, emulsions or viscous compositions that may be buffered to a selected pH. Pharmaceutically acceptable carriers for liquid formulations may be aqueous or nonaqueous solutions, suspensions, emulsions or oils. Examples of nonaqueous solvents are propylene glycol, polyethylene glycol, and injectable organic esters such as ethyl oleate. Aqueous carriers include water, alcoholic solutions, emulsions or suspensions, including saline and buffered media. The pH of a composition after reconstitution is preferably between 6 and 8, and more preferably between 6.5 and 7.5 (e.g. about 7). Compositions of the invention may be maintained by the use of a buffer e.g. a Tris buffer, acetate, glutamate, lactate, maleate, tartrate, phosphate, citrate, carbonate, glycinate, histidine, glycine, succinate and triethanolamine buffer. Thus compositions of the invention will preferably include a buffer. The isotonic agent may be an ionic isotonic agent such as a salt or a non-ionic isotonic agent such as a carbohydrate. Examples of ionic isotonic agents include but are not limited to NaCl, CaCl₂, KCl and MgCl₂. Examples of non-ionic isotonic agents include but are not limited to sorbitol and glycerol.

In a preferred embodiment of the invention, the vaccine composition is formulated as a sterile liquid, pyrogenfree, phosphate-buffered physiological saline, with or without a preservative.

A pharmaceutically acceptable preservative can be employed to increase the shelf life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

The composition of the invention can be formulated as single dose vials, multidose vials or as pre-filled syringes.

Another aspect of the present invention relates to pharmaceutical formulations and pharmaceutical compositions containing at least one compound of the general formula (I) optionally conjugated to a carrier protein or the vaccine as an active ingredient, together with at least one pharmaceutically acceptable carrier, excipient, solvent and/or diluents.

Further preferred, the pharmaceutical composition is formulated in the form of a lyophilisate or liquid buffer solution.

The vaccine or pharmaceutical composition can also be administered in form of its pharmaceutically active salt optionally using substantially nontoxic pharmaceutically acceptable carrier, excipients, adjuvants or diluents. The vaccine or pharmaceutical composition of the present invention is prepared in a conventional solid or liquid carrier or diluents and a conventional pharmaceutically-made adjuvant at suitable dosage level in a known way. The preferred preparations and formulations are in administrable form which is suitable for oral application. These administrable forms, for example, include pills, tablets, film tablets, coated tablets, capsules, powders and deposits. Other than oral administratable forms are also possible. The inventive vaccine or pharmaceutical composition may be administered by any appropriate means, including but not limited to inhalation, injection (intravenous, intraperitoneal, intramuscular, subcutaneous) by absorption through epithelial or mucocutaneous linings (oral mucosa, rectal and vaginal epithelial linings, nasopharyngial mucosa, intestinal mucosa); orally, rectally, transdermally, topically, intradermally, intragastrically, intracutaneously, intravaginally, intravasally, intranasally, intrabuccally, percutaneously, sublingually, or any other means available within the pharmaceutical arts.

The vaccine or pharmaceutical composition of the present invention, containing at least a synthetic saccharide of general formula (I), (II), (III), 1^{#}, 35^{#}, 36^{#} or 37^{#}, preferably the saccharide 1, 35, 36 and/or 37 or a derivative or pharmaceutically acceptable salt thereof as an active ingredient will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active ingredient may be combined with any oral nontoxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent of the tetrasaccharide.

Suitable binders include starch, gelatin, natural sugars, corn sweeteners, natural and synthetic gums such as acacia, sodium alginate, carboxymethyl-cellulose, polyethylene glycol and waxes. Among the lubricants that may be mentioned for use in these dosage forms, boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the vaccine or pharmaceutical composition of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidifies.

Also included are solid form preparations which are intended to be converted, shortly before use, to liquid form preparations for either oral or parenteral administration. Such liquid forms include solutions, suspensions and emulsions.

The inventive vaccine or pharmaceutical composition containing the saccharide of general formula (I), (II), (III), 1^{#}, 35^{#}, 36^{#} or 37^{#}, preferably the saccharide 1, 35, 36 and/or 37 may also be deliverable transdermally. The transdermal compositions may take the form of creams, lotions, aerosols and/or emulsions and can be included in a transdermal patch of the matrix or reservoir type as are conventional in the art for this purpose.

The term capsule refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet means compressed or molded solid dosage form containing the active ingredients with suitable diluents. The tablet can be prepared by compression of mixtures or granulations obtained by wet granulation, dry granulation or by compaction well known to a person skilled in the art.

Oral gels refer to the active ingredients dispersed or solubilized in a hydrophilic semi-solid matrix.

Powders for constitution refer to powder blends containing the active ingredients and suitable diluents which can be suspended in water or juices.

Suitable diluents are substances that usually make up the major portion of the composition or dosage form. Suitable diluents include sugars such as lactose, sucrose, mannitol and sorbitol, starches derived from wheat, corn rice and potato, and celluloses such as microcrystalline cellulose. The amount of diluents in the composition can range from about 5 to about 95% by weight of the total composition, preferably from about 25 to about 75%, more preferably from about 30 to about 60% by weight, and most preferably from about 40 to 50% by weight.

The term disintegrants refers to materials added to the composition to help it break apart (disintegrate) and release the medicaments. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses and cross-linked microcrystalline celluloses such as sodium croscarmellose, alginates such as alginic acid and sodium alginate, clays such as bentonites, and effervescent mixtures. The amount of disintegrant in the composition can range from about 1 to about 40% by weight of the composition, preferably 2 to about 30% by weight of the composition, more preferably from about 3 to 20% by weight of the composition, and most preferably from about 5 to about 10% by weight.

Binders characterize substances that bind or “glue” powders together and make them cohesive by forming granules, thus serving as the “adhesive” in the formulation. Binders add cohesive strength already available in the diluents or bulking agent. Suitable binders include sugars such as sucrose, starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropyl-methylcellulose; polyvinylpyrrolidone; and inorganics such as magnesium aluminum silicate. The amount of binder in the composition can range from about 1 to 30% by weight of the composition, preferably from about 2 to about 20% by weight of the composition, more preferably from about 3 to about 10% by weight, even more preferably from about 3 to about 6% by weight.

Lubricant refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.05 to about 15% by weight of the composition, preferably 0.2 to about 5% by weight of the composition, more preferably from about 0.3 to about 3%, and most preferably from about 0.3 to about 1.5% by weight of the composition.

Glidents are materials that prevent caking and improve the flow characteristics of granulations, so that flow is smooth and uniform. Suitable glidents include silicon dioxide and talc. The amount of glident in the composition can range from about 0.01 to 10% by weight of the composition, preferably 0.1% to about 7% by weight of the total composition, more preferably from about 0.2 to 5% by weight, and most preferably from about 0.5 to about 2% by weight.

Coloring agents are excipients that provide coloration to the composition or the dosage form. Such excipients can include food grade dyes and food grade dyes adsorbed onto a suitable adsorbent such as clay or aluminum oxide. The amount of the coloring agent can vary from about 0.01 to 10% by weight of the composition, preferably from about 0.05 to 6% by weight, more preferably from about 0.1 to about 4% by weight of the composition, and most preferably from about 0.1 to about 1%.

Techniques for the formulation and administration of the vaccine of the present invention may be found in “Remington's Pharmaceutical Sciences” Mack Publishing Co., Easton Pa. A suitable vaccine composition comprising at least one saccharide of the general formula (I), (II), (III), 1^{#}, 35^{#}, 36^{#} or 37^{#}, preferably the saccharide 1, 35, 36 and/or 37 and/or pharmaceutically acceptable salts thereof may be a solution of such saccharide(s) in a suitable liquid pharmaceutical carrier or any other formulation such as tablets, pills, film tablets, coated tablets, dragees, capsules, powders and deposits, gels, syrups, slurries, suspensions, emulsions, and the like.

A therapeutically effective dosage of the saccharide of general formula (I), (II), (III), 1^{#}, 35^{#}, 36^{#} or 37^{#}, preferably of the saccharide 1, 35, 36 and/or 37 refers to that amount of the compound that results in an at least a partial immunization against a disease. Toxicity and therapeutic efficacy of such compounds can be determined by standard pharmaceutical, pharmacological, and toxicological procedures in cell cultures or experimental animals. The dose ratio between toxic and therapeutic effect is the therapeutic index. The actual amount of the composition administered will be dependent on the subject being treated, on the subject's weight, the severity of the affliction, the manner of administration and the judgement of the prescribing physician.

The synthetic saccharide of general formula (I), (II), (III), 1^{#}, 35^{#}, 36^{#} or 37^{#}, preferably the saccharide 1, 35, 36 and/or 37 or the antibody thereof can be used for preparing a pharmaceutical composition especially vaccines for the treatment or prevention of a disease caused by the pathogen Neisseria meningitidis. The synthetic saccharide of the present invention or the antibody thereof can be used for the treatment or prevention of a disease caused by the pathogen Neisseria meningitidis. A method of inducing immune response against Neisseria meningitidis in a subject comprises administering of the synthetic saccharide of the present invention or a mixture thereof. A method of treating or preventing Neisseria meningitidis infection in a subject comprises administering of at least one synthetic saccharide of the present invention or a mixture thereof or the composition or vaccine thereof.

In the following schemes occurring abbreviations mean Ac (acetyl), AIBN (2-2′-azoisobutyronitrile), BCAC (bicinchoninic acid), Bn (benzyl), Bu (butyl), Bz (benzoyl), Cbz(benzyl carbamate), CSA(camphorsulfonic acid), DBU(1,8-diazabicyclo[5.4.0]undec-7-ene), DIPC(N,N′-diisopropylcarbodiimide), DMAP(4-dimethylaminopyridine), DMF (dimethylformamide), DMSO(dimethyl sulfoxide), DMTST(dimethyl(methylthio)sulfoniumtriflate), Et (ethyl), EtOAc(ethyl acetate), Gal (D-galactopyranose), GlcNAc (2-N-acetyl-2-deoxy-D-glucopyranose), Hep (L-gylcero-D-mannoheptose), Kdo (3-deoxy-D-manno-2-octulosonic acid), Lev (levulinyl), Man(D-mannopyranose), Me (methyl), MeOH (methanol), MS (molecular sieve), NBS(N-bromosuccinimide), NCS(N-chlorosuccinimide), NIS(N-iodosuccinimide), PBB (para-bromobenzyl), PBS (phosphate buffered saline), PEA(phosphoethanol amine), PEG (polyethyleneglycol), Ph (phenyl), Py/Pyr (pyridine), TBAB(tetra-n-butylammonium bromide), TBDPS(tert-butyldiphenylsilyl), TBS (tert-butyldimethylsilyl), tBu (tert-butyl), TEA (triethylamine), Tf (trifluoromethanesulfonyl), THF (tetrahydrofurane), TMS (trimethylsilyl), TMSOTf (trimethylsilyl trifluoromethane sulfonate), TsOH (para-toluene sulfonic acid)

### Total Synthesis of Tetrasaccharide 1

Synthesis of tetrasaccharide 1

Traditionally, the synthesis of heptose building blocks relied on the one carbon elongation of mannose building blocks at C-6. Elongation of the carbon skeleton employed toxic heavy metals such as mercury and osmium and often produced inseparable mixtures of diastereomers. Our newly developed de novo synthetic route for heptose synthesis is straightforward and simplifies the production of versatile, differentially-protected heptose building blocks (Scheme 4). First, reduction of known ketone 10 with L-selectride gave a regioisomer in 82% yield due to 1,3 migration of the TBS group. Protection of newly formed hydroxyl group in 10 with para-bromobenzyl bromide and sodium hydride in dimethylformamide gave silyl ether 11 in 87% yield. The silyl ether in 11 was replaced by a benzyl ether to afford bisacetal 12 in 91% yield over two steps. Under the influence of CSA, the ketal of 12 was removed selectively in a yield of 84%. The newly formed primary hydroxyl group was protected as a TBDPS ether, and the secondary hydroxyl was masked as a TBS ether, to afford dimethylacetal 13 (73% over three steps). Hydrolysis of 13 with catalytic para-toluenesulfonic acid in acetone proceeded smoothly to yield aldehyde 8. Mukaiyama-type aldol reaction between aldehyde 8 and silyl enolether 9 using MgBr₂-OEt₂ as a chelating activator afforded the seven carbon backbone 14 with an excellent 2,3-anti-3,4-syn-selectivity in a satisfactory 65% yield. Treatment of 14 with trifluoroacetic acid gave lactone 15 as the major product and lactone 16 as the minor product.

Lactone 15 was then reduced with lithium tri-tert-butoxylithiumaluminium hydride into diols 17 and 18 in a straightforward manner. Partial migration of the silyl group from C-2 to C-3 was observed and both regioisomers were easily separated by flash chromatography.

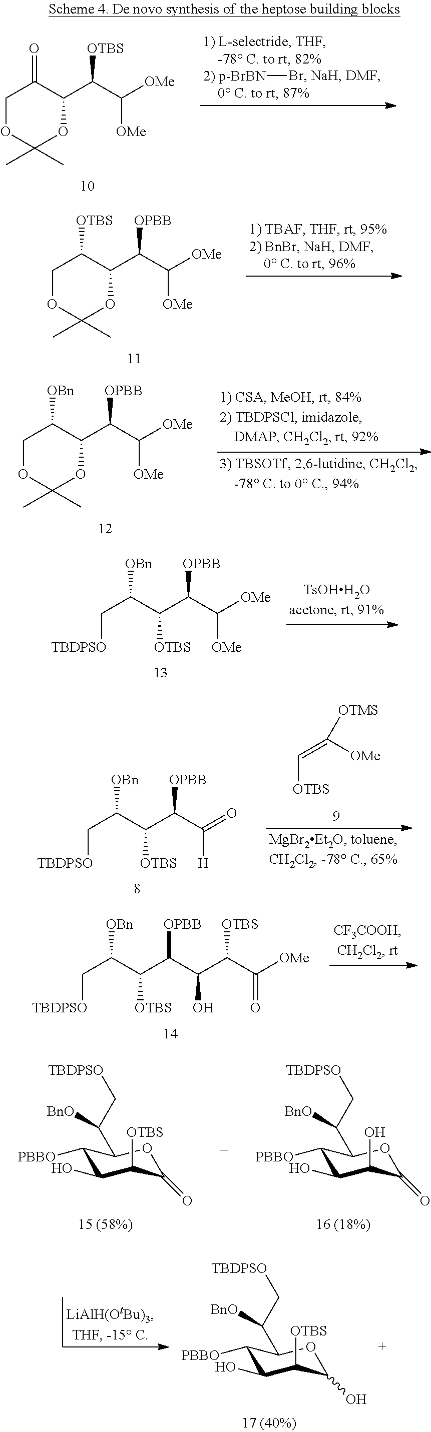

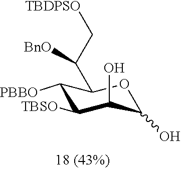

Levulinoylation of both hydroxyl groups present in diol 17 provided diketone 19 in 76% yield (Scheme 5). The silyl groups in 19 were removed with HF in pyridine and subsequent diacetylation gave ester 20 in a yield of 82% over two steps. Ester 20 was then converted into heptose trichloroacetimidate 6 upon selective anomeric delevulinoylation and trichloroacetimidate formation (77% over two steps) or heptose thioglycoside 21 under the agency of boron trifluoride etherate (66%). Diol 18 was diacetylated to diester 22 in an excellent 89% yield. The silyl groups in 22 were replaced by the benzoyl groups analogous to the 19-* 20 conversion, and the resulting anomeric ester was then replaced with 5-tert-butyl-2-methylbenzenethiol to give heptose thioglycoside 23 in 60% yield over three steps. Under the influence of in situ hydrogen chloride, the O-acetyl group in 23 was removed to afford heptose building block 5 in 76% yield.

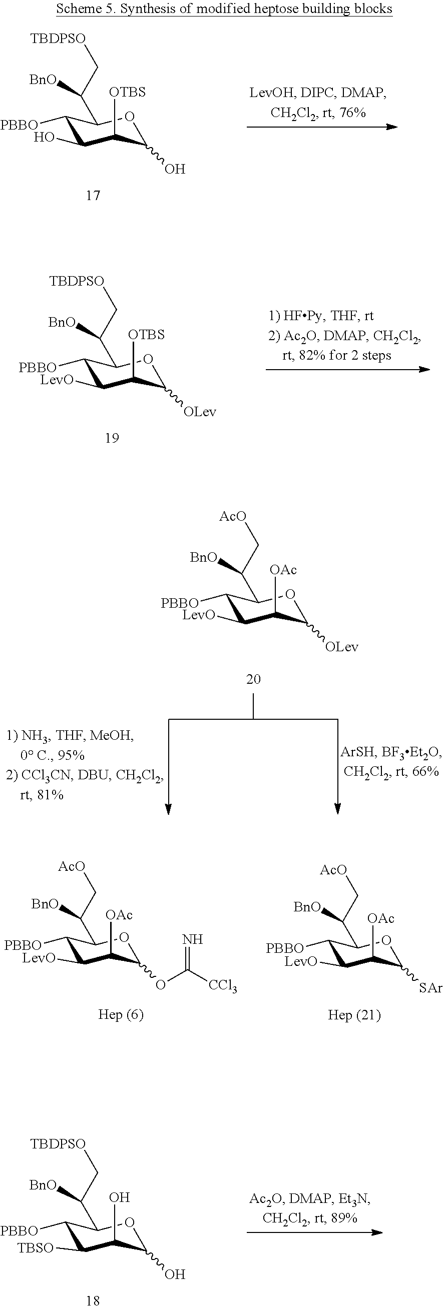

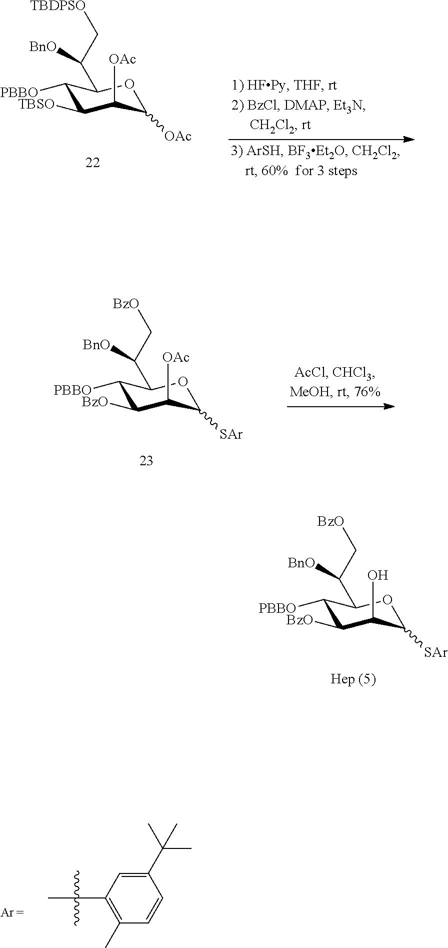

Synthesis of Kdo Building Block with an α-Linked Spacer.

Three different Kdo building blocks 24, 25, 26 and the linker 27 were accessed using modified published methods. In nature, the LPS Kdo moiety of Gram-negative bacteria exists as the a-anomer. Upon activation of the synthetic Kdo building blocks, an elimination reaction to produce the α,β-unsaturated ester frequently occurred due to the presence of the 3-deoxy moiety and the electron-withdrawing C-1 carboxylic function. Therefore stereoselective construction of α-linked Kdo building blocks was dependent on fine tuning of the conditions in the glycosylation reactions. Thus, to find the optimal means to introduce an α-linked spacer into the Kdo moiety, glycosylations of several classes of Kdo building blocks with the linker 27 were briefly examined (Table 1). Linker 27 glycosylation with either glycosyl fluoride 24 or thioglycoside 25 resulted in couplings that produced either β-glycoside or complex mixtures regardless of promoters, solvents and conditions used (entries 1-4, Table 1). Although NIS/TfOH-promoted activation of 2,3-glycal 26 often led to complex mixtures, the coupling of 2,3-glycal 26 with the linker 27 under the catalysis of in situ phenylselenyl triflate proceeded smoothly, and the resulting 3-phenylselenium group was reduced with tributyltin hydride to afford α-linked Kdo glycoside 28 in an overall yield of 63% over 2 steps (entries 5 and 6, Table 1).

Removal of the acetyl group in 28 with sodium methoxide, and subsequent regioselective placement of isopropylidene group at the 7,8-vicinal diol using 2-methoxypropene, proceeded smoothly to give 29 (84% over two steps) (Scheme 6). Finally, regioselective benzylation with dibutyltin oxide activation gave the α-Kdo building block 7 containing a free 5-hydroxyl group in 74% yield.

TABLE 1
Glycosylation of various Kdo building blocks with the linker 27

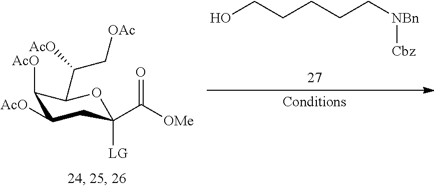

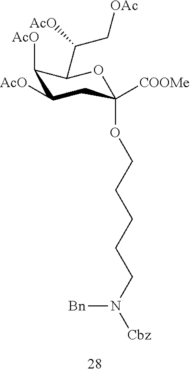

| Entry | Kdo | Conditions | Results |
|---|---|---|---|
| 1 | 24 | BF₃OEt₂, Et₃N, CH₂Cl₂, 0° C. | Complex |
| | | | mixture |
| 2 | 25 | NCS, TfOH, CH₂Cl₂, CH₃CN, −65° C. | >80% (β) |
| 3 | 25 | NCS, TfOH, toluene, −65° C. | No reaction |
| 4 | 25 | NIS, TfOH, CH₂Cl₂, rt | Complex |
| | | | mixture |
| 5 | 26 | (a) NIS, TfOH, CH₂Cl₂, rt | Complex |
| | | (b) Bu₃SnH, AIBN, toluene, 110° C. | mixture |
| 6 | 26 | (a) PhSeCl, AgOTf, TMSOTf, | 63% (α) |
| | | CH₂Cl₂, rt | |
| | | (b) Bu₃SnH, AIBN, toluene, 110° C. | |

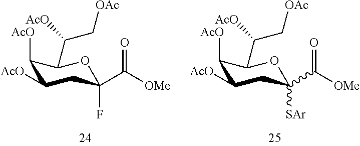

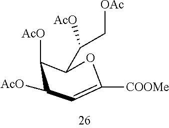

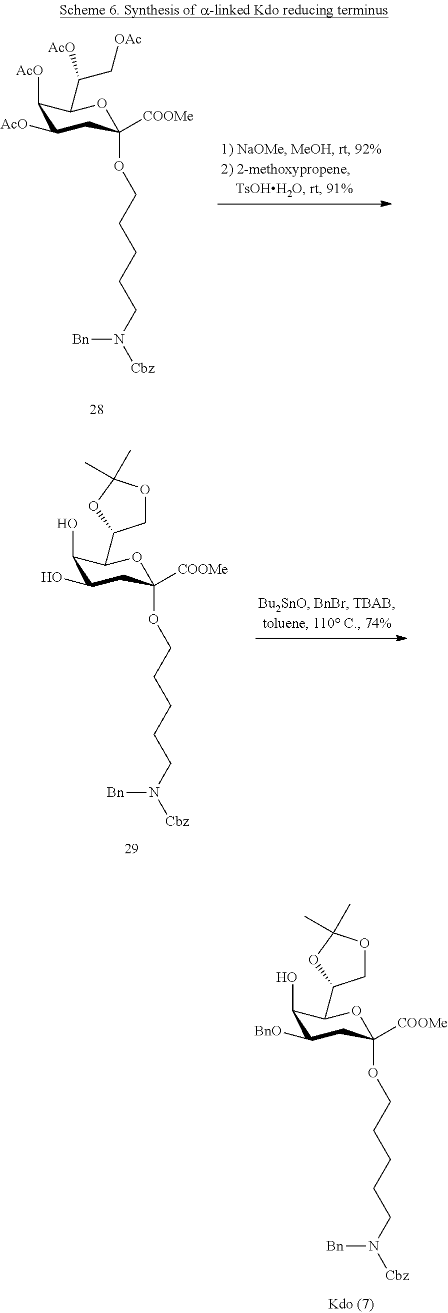

Synthesis of Disaccharide Fragments of N. meningitidis LPS.

With the building blocks in hand, efforts were directed at the synthesis of the disaccharide portions, GlcN-Hep and Hep-Kdo of the LPS core saccharide. Using a catalytic amount of TMSOTf as promoter, coupling of the 2-azidoglucopyranosyl trichloroacetimidate 4 with heptose nucleophile 5 in Et₂O provided the desired α-linked disaccharide 30 in 68% yield, with no detectable production of the β-anomer (Scheme 7). Disaccharide 30 was subjected to selective removal of the anomeric thiol ether with NBS, and subsequent N-phenyl trifluoroacetimidate formation, to produce the disaccharide building block 2 over two steps in a yield of 66%.

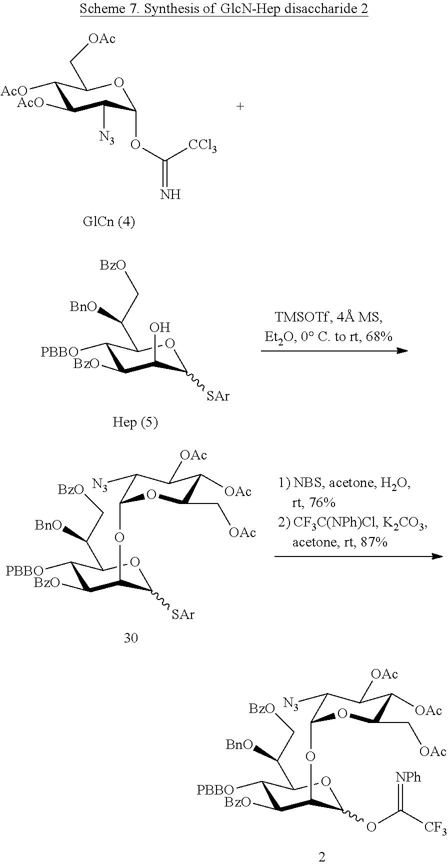

Identification of effective glycosylation conditions for the construction of the 1→5 linked Hep-Kdo disaccharide is extremely challenging due to the mismatch and low stereoselectivity in the coupling of the heptose donors with the inert OH-5 position of Kdo building blocks. NIS-mediated glycosylation of heptose thioglycoside 21 with the unreactive Kdo building block 7 or its 5-O-stannyl ether led to a hydrolyzed derivative of 21, and no desired disaccharide product was detected (entries 1 and 2, Table 2). Surprisingly, heptose trichloroacetimidate 6, activated by catalytic TMSOTf, proved to be superior for glycosylation with Kdo building block 7. The reaction proceeded efficiently affording the desired α-linked Hep-Kdo disaccharide 31 in a yield of 88% (entry 3, Table 2). It was confirmed that disaccharide 31 had the desired configuration by measuring the coupling constant between heptose C-1 and H-1 (¹J_{C-H}=173.0 Hz). Finally, selective cleavage of the levulinoyl ester of disaccharide 31 with hydrazine acetate afforded disaccharide building block 3 in 73% yield.

TABLE 2
Synthesis of disaccharide 3

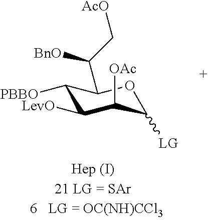

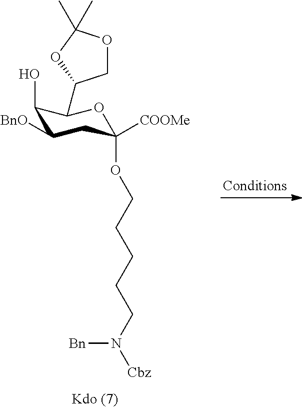

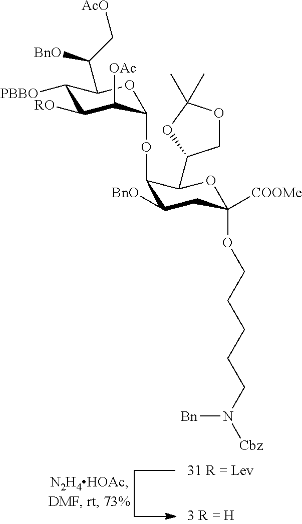

| Enty | Hep | Kdo | Conditions | Results |
|---|---|---|---|---|
| 1 | 21 | 7 | NIS, TMSOTf, 4Å MS, CH₂Cl₂, | Hydrolyzed |
| | | | Et₂O, 0° C. to rt | |
| 2 | 21 | Bu₃SnO- | NIS, TMSOTf, 4Å MS, CH₂Cl₂, | Hydrolyzed |
| | | Kdo | toluene, 0° C. to rt | |
| 3 | 6 | 7 | TMSOTf, 4Å MS, CH₂Cl₂, 0° C. | 88% |

Synthesis of Tetrasaccharide 1.

With the three disaccharide building blocks 30, 2 and 3 in hand, assembly of the tetrasaccharide skeleton of 1 could proceed. Union of thioglycoside 30 and disaccharide 3 gave either complex mixtures or failed regardless of the method of activation (entries 1 and 2, Table 3). Gratifyingly, when disaccharide N-phenyl trifluoroacetimidate 2 was used, coupling with disaccharide 3 under the catalysis of TMSOTf in CH₂Cl₂/Et₂O provided the desired α-linked tetrasaccharide 32 in a satisfactory 72% yield (entry 3, Table 3). The configuration of the newly formed glycosidic linkage was confirmed based on the coupling constants between the C-1 and H-1 of the two corresponding heptoses of the target tetrasaccharide 1 (¹J_{C-H}=169.7 Hz and 172.1 Hz, respectively).

TABLE 3
Assembly of tetrasaccharide skeleton 32

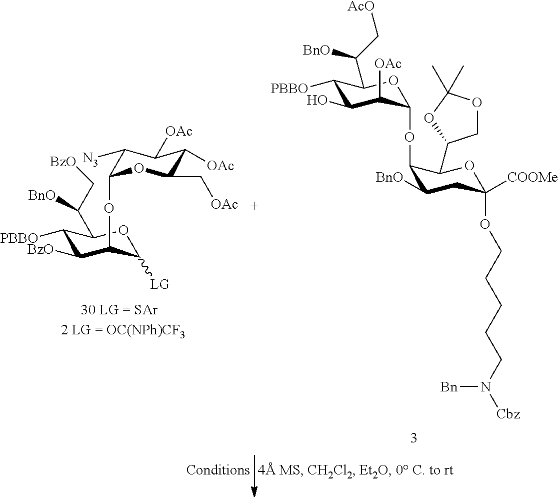

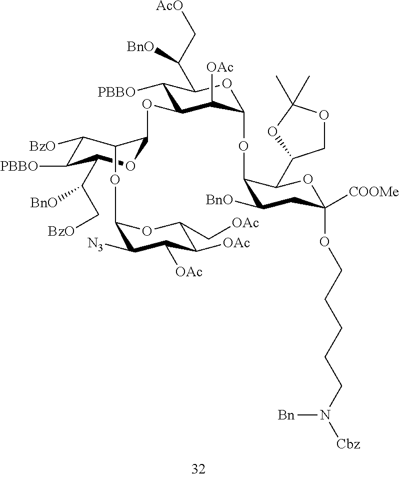

| Entry | GlcN-Hep | Hep-Kdo | Conditions | Results |
|---|---|---|---|---|
| 1 | 30 | 3 | NIS, TMSOTf | Complex mixture |
| 2 | 30 | 3 | DMTST | No reaction |
| 3 | 2 | 3 | TMSOTf | 72% |

The final stage of the synthesis involved the conversion of the azide present in 32 to acetamide 33 (Scheme 8). A one-pot reduction-acetylation sequence using AcSH and pyridine ensured the stability of moderately acid-labile isopropylidene group and a clean reaction. Acetamide 33 was isolated in 91% yield from azide 32. Global deprotection of acetamide 33 followed a straightforward three-step procedure.

Acidic cleavage of the isopropylidene group, saponification of all of the esters, and hydrogenolysis of the benzyl, p-bromobenzyl and benzyloxycarbonyl groups, produced the target tetrasaccharide 1 in a yield of 82% over three steps after final purification on Sephadex LH-20 column.

The structure of the tetrasaccharide 1 was unambiguously confirmed by ¹H, ¹³C and 2D NMR as well as ESI-HRMS. The core tetrasaccharide of N. meningitidis LPS, equipped with an amine linker at the reducing end, was thereby obtained by total synthesis.

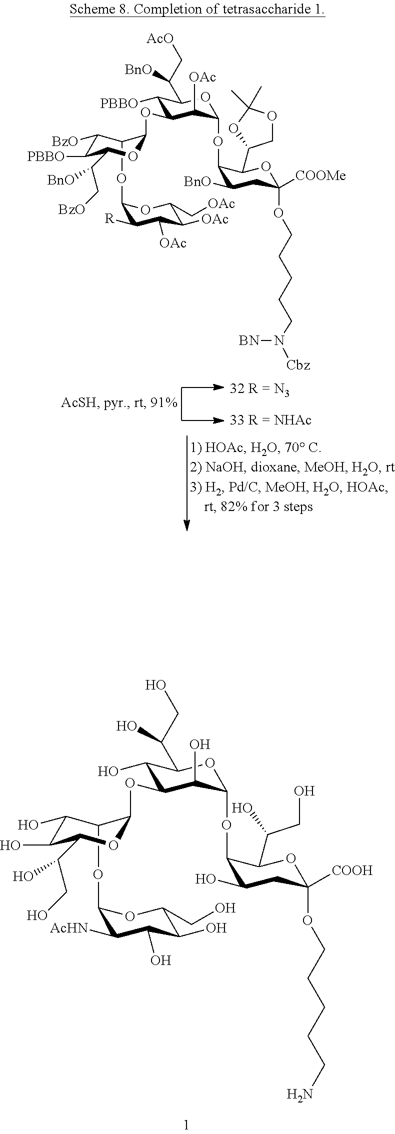

### Syntheses of Sequences 34-37.

To further evaluate the immune response, cross reactivity studies of the deletion sequences of the core tetrasaccharide α-GlcNAc-(1→2)-α-Hep-(1→3)-α-Hep-(1→5)-α-Kdo need to be done through microarray screening. Thus, a series of the deletion sequences 34-37 containing at least one Hep and/or Kdo unit were readily synthesized by modifying previously synthetic approach for the tetrasaccharide 1 (Scheme 9).

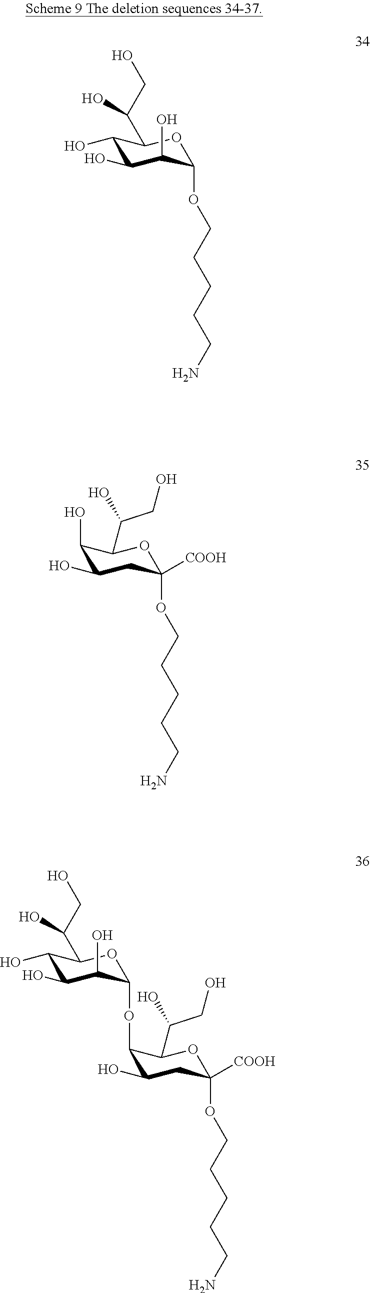

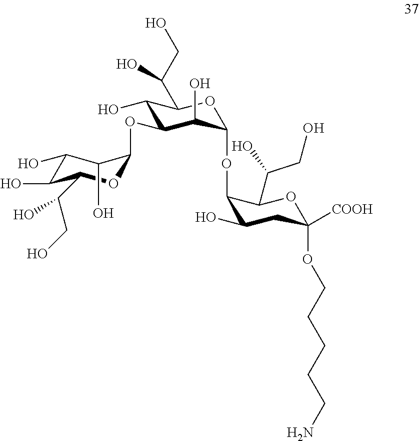

The α-linked Hep monosaccharide was readily prepared by coupling the Hep thioglycoside 23 with the linker 27 in the presence of NIS/TMSOTf in CH₂Cl₂ at 0° C. to room temperature (Scheme 10). The α-linked Kdo monosaccharide 28 and the α-(1→5)-linked Hep-Kdo disaccharide 31 were accessed using previously reported synthetic approaches. Subsequently, the Hep monosaccharide and the Kdo monosaccharides 28 were subjected to basic conditions (NaOMe, MeOH/CH₂Cl₂, rt; or NaOH, dioxane/MeOH/H₂O, rt) to remove all acetyl and benzoyl groups, followed by hydronolysis over Pd/C catalyst under acidic conditions to furnish the Hep 34 and the Kdo 35 in 85% and 86% yields over 2 steps, respectively. Global deprotection of the disaccharide 31 using a three-step procedure, namely, acidic cleavage of the isopropylidene group, saponification of all esters, and hydrogenolysis of the benzyl, p-bromobenzyl and benzyloxycarbonyl groups, afforded the disaccharide 36 in 77% yield over three steps.

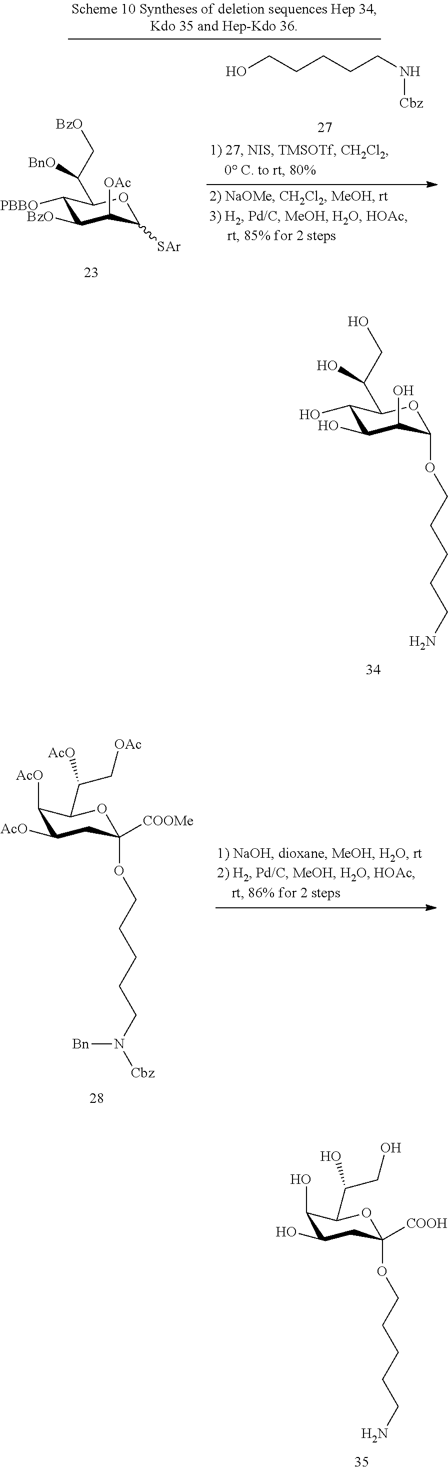

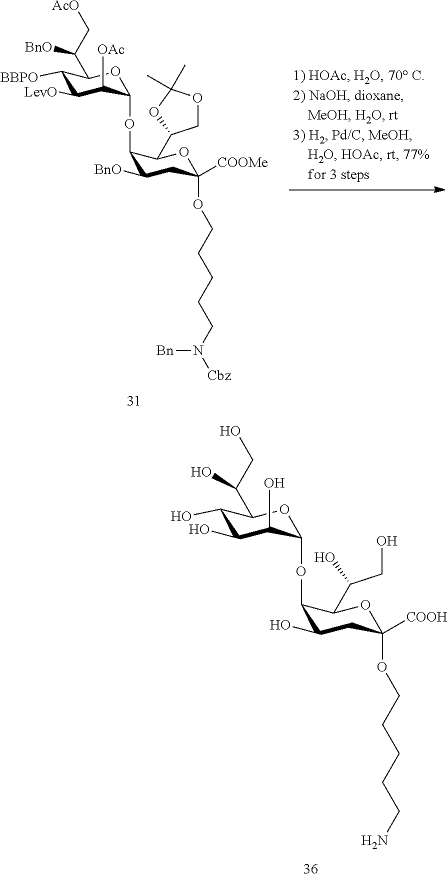

The α-Hep-(1→3)-α-Hep-(1→5)-α-Kdo trisaccharide 37 was assembled by use of a stereocontrolled [1+2]glycosidic coupling as a key step (Scheme 11). Treatment of the thioglycoside 23 with NBS, followed by formation of N-phenyl trifluoroacetimidate, produced the stable Hep building block 38 (74% yield over 2 steps). Coupling of N-phenyl trifluoroacetimidate 38 with the disaccharide 3 under the catalysis of TMSOTf in CH₂Cl₂ provided the desired α-linked trisaccharide 39 in 73% yield. Steps similar to those used for 31→36 were then employed for the conversion of 39 into the deletion sequence Hep-Hep-Kdo trisaccharide 37 in 75% yield over 3 steps.

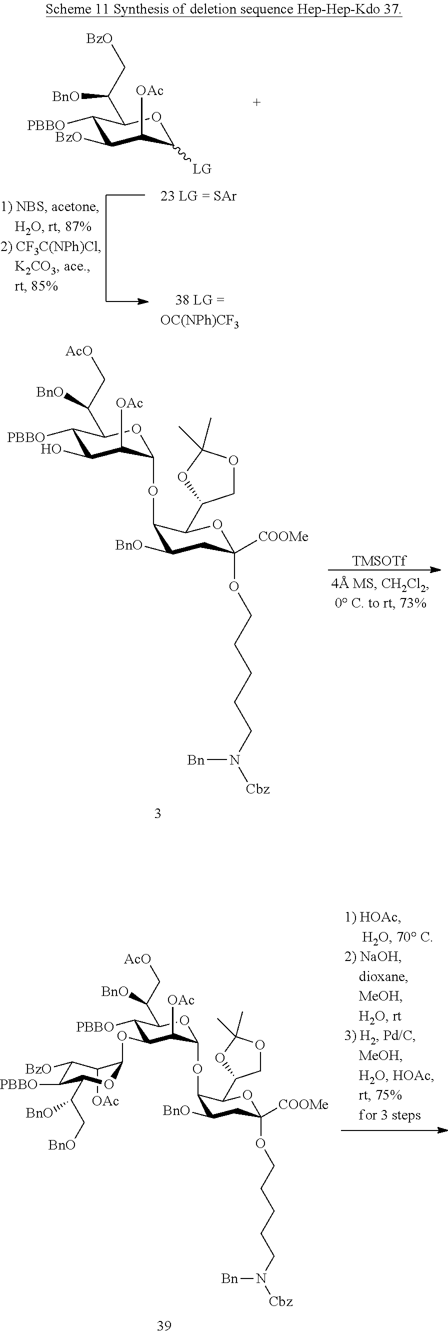

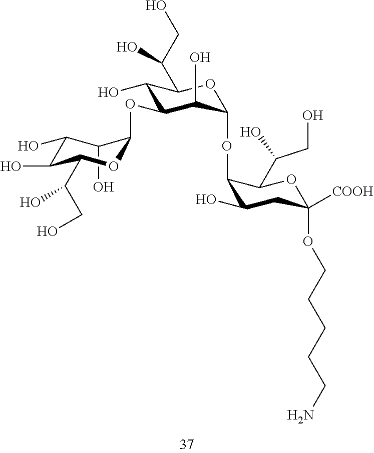

The inventors were able to synthesize tetrasaccharide 1, having the glycan moiety α-GlcNAc-(1→2)-α-Hep-(1→3)-α-Hep-(1→5)-α-Kdo which is a substructure of the inner core saccharide, that is conserved in all meningococcal LPS. Further conjugates in which the tetrasaccharide 1 disaccharide 36 and trisaccharide 37 were conjugated to carrier protein CRM₁₉₇ were produced and their immunogenicity in experimental mouse models could be demonstrated. The minimal saccharide sequence required to elicit robust IgG responses against the LPS has also been identified. The invention refers also to the development of monoclonal antibodies against the tetrasaccharide 1 and their binding efficiency to natural LPS structures on inactivated bacteria. Significantly higher anti-LPS-core saccharide specific antibody titers could be observed in mice immunized with the tetrasaccharide 1 conjugated to a carrier protein in comparison to unconjugated tetrasaccharide 1. Immunization with the conjugate of tetrasaccharide 1 and the carrier protein resulted in robust boosting responses. Specificity of sera and monoclonal antibodies against synthetic carbohydrate structures from different bacterial LPS was studied by microarray analysis revealing a specific response for the meningococcal tetrasaccharide. Flow cytometry analysis and confocal fluorescence microscopy studies showed that the antibodies are able to recognize meningococcal strains across all serogroups. The potential of these antibodies to promote opsonophagocytosis was evaluated by flow cytometry assay using fluorescence-labeled bacteria and serum-dependent enhancement in phagocytosis was demonstrated. Results of these studies showed that the synthetic tetrasaccharide 1 and the tetrasaccharide 37 are a potential vaccine candidate against N. meningitidis B infections.

### DESCRIPTION OF FIGURES

FIG. 1: Scheme showing the LPS-core structure of N. meningitidis, the core tetrasaccharide α-GlcNAc-(1→2)-α-Hep-(1→3)-α-Hep-(1→5)-α-Kdo is shown in black and the other proportions of the structure are grey. The short saccharides are variable, especially between the different serogroups and LOS immunotypes. Further isolated LPS structures of N. meningitidis, shows microheterogenity especially in the position of phosphoethaniolamine, which may be attached to Hep₂ in 3 and/or 6 position.

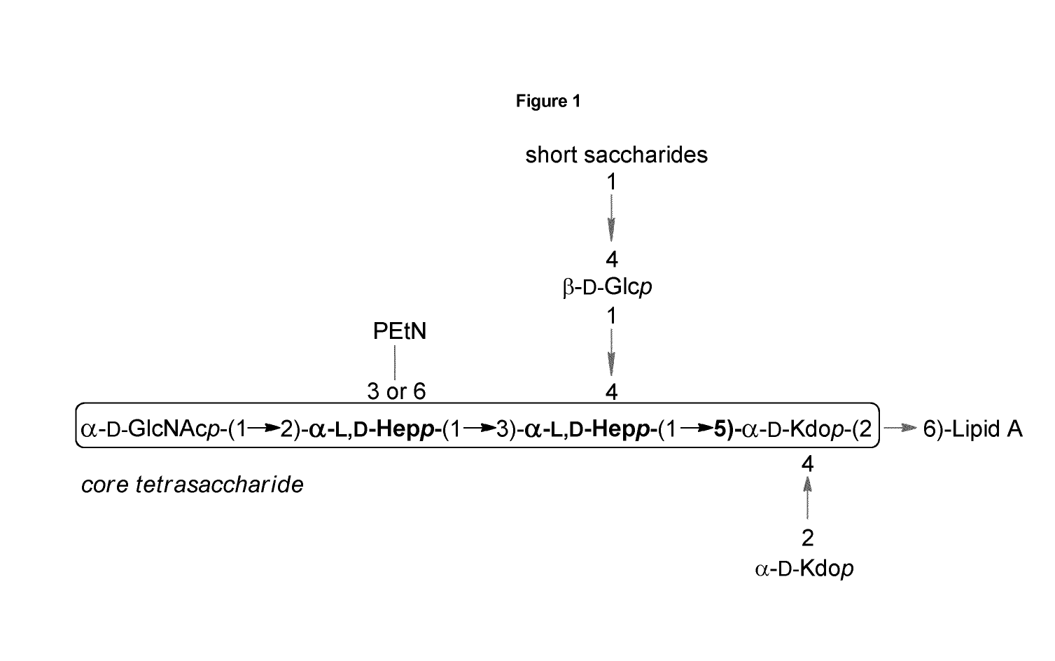

FIG. 2: Scheme showing conjugation of tetrasaccharide 1 to the carrier protein CRM₁₉₇ via a spacer molecule, disuccinimidyl adipate

FIG. 3: SDS-PAGE of tetrasaccharide 1-CRM₁₉₇ conjugate and CRM₁₉₇

FIG. 4: Determination of molecular mass of CRM₁₉₇ and tetrasaccharide1-CRM₁₉₇ conjugate by MALDI-TOF MS

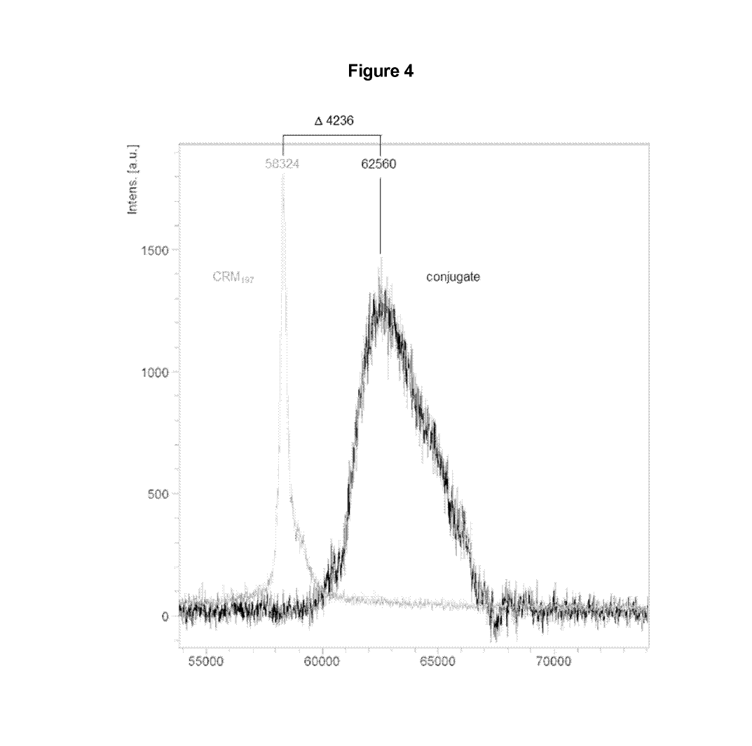

FIG. 5: Glycan microarray pattern of tetrasaccharide 1 (KdoHep₂GlcNAc), CRM₁₉₇, BSA-spacer-GlcNAc and Man₃Gal

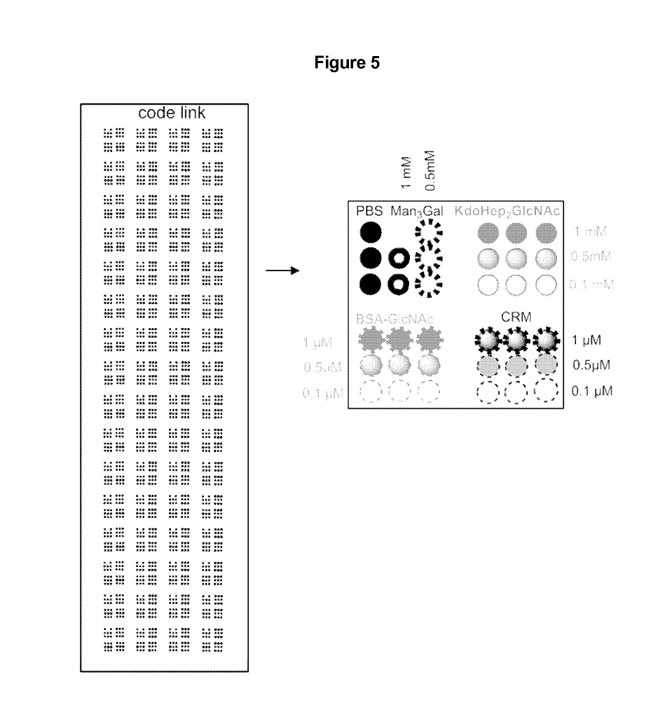

FIG. 6: Glycan array analysis of initial IgG and IgM antibody responses in tetrasaccharide 1 (unconjugated) and tetrasaccharide1-CRM conjugate (conjugated) immunized mice

FIG. 7: Initial IgG antibody responses of tetrasaccharide 1 (tetra LPS-core) and tetrasaccharide1-CRM₁₉₇ conjugate (tetra-CRM) immunized mice in 1:80 dilution considering reactivity against tetrasaccharide1-CRM₁₉₇ (tetra LPS-core) and CRM₁₉₇.

FIG. 8: IgG antibody responses of tetrasaccharide 1 (tetra LPS core) and tetrasaccharide1-CRM conjugate (tetra-CRM) immunized mice in 1:100 dilution considering reactivity against tetrasaccharide1-CRM₁₉₇ (tetra LPS-core) and CRM₁₉₇ after 2. Boost (week 8).

FIG. 9: Glycan microarray pattern consisting of IgM, IgG, tetrasaccharide 1 (KdoHep₂GlcNAc), CRM₁₉₇ and BSA-spacer-GlcNAc

FIG. 10: Glycan array of dose-dependent immune response generated against 1, 2, and 3 μg of tetrasaccharide 1-CRM conjugate in presence of alum and 3 μg of tetrasaccharide 1-CRM₁₉₇ conjugate with FA at week 2 showing IgG and IgM response

FIG. 11: Initial immune response generated against 1, 2, and 3 μg of tetrasaccharide 1-CRM conjugate in presence of alum and 3 μg of tetrasaccharide 1-CRM₁₉₇ conjugate with FA in a 1:100 dilution considering reactivity against tetrasaccharide 1 (tetra LPS-core) and CRM₁₉₇.

FIG. 12: Isotyping of IgG response against tetrasaccharide 1 (tetra LPS core) and tetrasaccharide 1-CRM conjugate (tetra-CRM) by glycan array analysis considering reactivity against tetrasaccharide 1 (tetra LPS core) and CRM

FIG. 13: Deletion sequence glycan microarray pattern consisting of tetrasaccharide 1, mono-Hep 34, mono-Kdo 35, disaccharide 36, trisaccharide 37, chlamydial LPS-core tri-Kdo (KKK) and tri-Heptose (HHH) LPS-core from Yersinia pestis

FIG. 14: IgG responses of post-immune sera and monoclonal antibodies 1A5 1G1 and 1B6 4E1 against deletion sequence glycan array (tetrasaccharide 1, mono-Hep 34, mono-Kdo 35, disaccharide 36, trisaccharide 37, chlamydial LPS-core tri-Kdo (KKK) and tri-Heptose (HHH) LPS-core from Yersinia pestis)

FIG. 15: Glycan array analysis of single mouse sera raised against tetrasaccharide 1-CRM₁₉₇ in 1:800 dilution, considering reactivity against tetrasaccharide 1 (tetra LPS-core), CRM and spacer whereby mouse 12 elicited highest antibody response against tetrasaccharide 1 (grey bar). Shown are the average (ave) values.

FIG. 16: Isotyping of mAb 1A5 1G1 and 1B6 4E1 (kdo=mono-Kdo 35, di=disaccharide 36, tri=trisaccharide 37, tetra=tetrasaccharide 1) by deletion sequence glycan array. STDEV means standard deviation.

FIG. 17: Confocal laser scanning microscopy of N. meningitidis B; with 1:100 post-ad pre-immune sera dilutions

FIG. 18: General FACS analysis of inactivated N. meningitidis

FIG. 19: Reactivity of pre-immune and post-immune sera dilutions against N. meningitidis Y analysed by FACS

FIG. 20: Labeling of 8 meninogococcal serogroups with 1:100 dilution of pre-immune and post-immune sera analysed by FACS

FIG. 21: FACS analysis of monoclonal Antibody 1A5 1G1 binding against inactivated N. meningitidis B

FIG. 22: FACS analysis of different Gram-negative bacteria labeled with 10 ng/μl of the monoclonal Antibody 1A5 1G1

FIG. 23: FACS analysis of macrophages treated with bacteria preincubated with 1:100 diluted post-immune sera or PBS

FIG. 24: Percentage of meningococci B opsonized by macrophages after treatment with 1:100 diluted post-immune sera, monoclonal Antibody 1A5 1G1 supernatant, PBS or media.

### EXAMPLES

### 1. General Information for Chemical Synthesis

Commercial reagents were used without further purification except where noted. Solvents were dried and redistilled prior to use in the usual way. All reactions were performed in oven-dried glassware under an inert atmosphere unless noted otherwise. Analytical thin layer chromatography (TLC) was performed on silica gel 60 F254 glass plates precoated with a 0.25 mm thickness of silica gel. The TLC plates were visualized with UV light and by staining with Hanessian solution (ceric sulfate and ammonium molybdate in aqueous sulfuric acid) or sulfuric acid-ethanol solution. Column chromatography was performed on Fluka silica gel 60 (230-400 mesh). Optical rotations (OR) were measured with a Schmidt & Haensch UniPol L1000 polarimeter at a concentration (c) expressed in g/100 mL. ¹H and ¹³C NMR spectra were measured with a Varian 400-MR or Varian 600 spectrometer with Me₄Si as the internal standard. NMR chemical shifts (δ) were recorded in ppm and coupling constants (J) were reported in Hz. High-resolution mass spectra (HRMS) were recorded with an Agilent 6210 ESI-TOF mass spectrometer at the Freie Universitit Berlin, Mass Spectrometry Core Facility.

### 2. Experimental Details and Characterization Data of New Compounds

### 2.1. Synthesis of 2,7-di-O-acetyl-3-O-levulinoyl-4-O-para-bromobenzyl-6-O-benzyl-L-glycero-D-manno-heptopyranosyl levulinoate 20

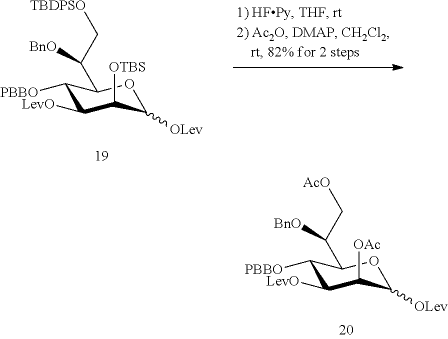

Compound 19 (500 mg, 0.49 mmol) was dissolved in THF (2 mL) at room temperature, followed by addition of 70% HF-pyridine (0.4 mL). After stirring for 2 days, the reaction mixture was carefully quenched with sat. aq. NaHCO₃ and the resulting solution was diluted with EtOAc. The organic layer was washed with brine, dried over Na₂SO₄, filtered and concentrated in vacuo to give the corresponding diol as a colorless syrup. To a solution of the above diol and DMAP (12 mg, 0.1 mmol) in CH₂Cl₂ (20 mL), was added Ac₂O (1 mL). After being stirred at room temperature for overnight, the mixture was washed with saturated aqueous NaHCO₃ and brine. The organic layer was dried over Na₂SO₄, filtered, and concentrated in vacuo. The residue was purified by silica gel column chromatography (cyclohexane/EtOAc: 1/1) to give 20 (300 mg, 82% for 2 steps) as a pale yellow syrup: [α]²⁰_{D}=+53.3 (c 0.3, CHCl₃); ¹H NMR (400 MHz, CDCl₃) δ 7.42-7.27 (m, 7H), 7.03 (m, 2H), 6.06 (d, J=2.4 Hz, 1H), 5.31 (dd, J=3.6, 9.2 Hz, 1H), 5.21 (dd, J=2.4, 3.6 Hz, 1H), 4.80 (d-like, J=12.0 Hz, 1H), 4.50 (t, J=11.6 Hz, 2H), 4.44 (dd, J=6.0, 11.6 Hz, 1H), 4.21 (dd, J=6.0, 11.6 Hz, 1H), 4.14 (d-like, J=11.6 Hz, 1H), 4.03 (t, J=9.6 Hz, 1H), 3.98 (m, 1H), 3.89 (dd, J=1.6, 9.6 Hz, 1H), 2.77-2.57 (m, 6H), 2.42 (m, 2H), 2.17 (s, 3H), 2.14 (s, 3H), 2.13 (s, 3H), 2.04 (s, 3H); ¹³C NMR (100 MHz, CDCl₃) δ 206.1, 205.9, 171.8, 170.6, 170.2, 169.8, 137.9, 137.0, 131.5, 129.0, 128.5, 128.0, 127.9, 121.5, 90.9, 73.5, 73.4, 73.3, 73.1, 72.4, 72.2, 72.1, 68.5, 62.8, 37.7, 29.7, 29.6, 27.8, 27.7, 20.9, 20.8; HRMS (ESI) m/z calcd for C₃₅H₄₁BrO₁₃Na [M+Na]⁺ 773.1608. found 773.1645.

### 2.2. Synthesis of 2,7-di-O-acetyl-3-O-levulinoyl-4-O-para-bromobenzyl-6-β-benzyl-L-glycero-D-manno-heptopyranosyl trichloroacetimidate 6

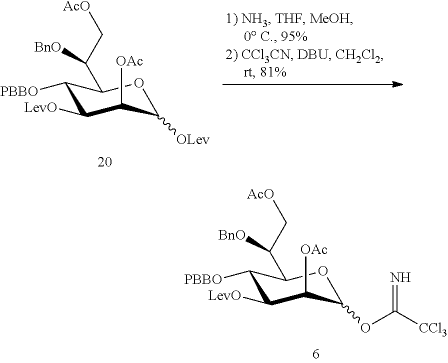

To a solution of compound 20 (170 mg, 0.227 mmol) in THF and methanol (7:3, 10 mL) at 0° C., was bubbled through gaseous ammonium at a modest rate. After stirring for 30 min at 0° C., the solution was evaporated in vacuo to give a residue, which was purified by silica gel column chromatography (CH₂Cl₂/MeOH: 20/1) to afford the corresponding hemiacetal (140 mg, 95%) as a colorless syrup. To a solution of the above hemiacetal (140 mg, 0.215 mmol) in CH₂Cl₂ (5 mL) was added CCl₃CN (107 μL, 1.07 mmol) and DBU (7 μL, 0.046 mmol). After being stirred at room temperature for 2 h, TLC revealed almost complete conversion of the starting material. The solution was concentrated in vacuo to a residue, which was purified by silica gel column chromatography (cyclohexane/EtOAc: 2/1) to give 6 (138 mg, 81%) as a colorless syrup: ¹H NMR (400 MHz, CDCl₃) δ 8.72 (s, 1H), 7.44-7.30 (m, 7H), 7.06 (d, J=8.4 Hz, 1H), 6.28 (d, J=2.0 Hz, 1H), 5.44 (dd, J=2.0, 3.2 Hz, 1H), 5.40 (dd, J=3.2, 9.2 Hz, 1H), 4.82 (d-like, J=12.0 Hz, 1H), 4.56 (d-like, J=11.6 Hz, 1H), 4.47 (m, 2H), 4.17 (m, 3H), 4.02 (m, 2H), 2.68 (m, 2H), 2.45 (m, 2H), 2.18 (s, 3H), 2.16 (s, 3H), 2.01 (s, 3H); LRMS (ESI) m/z calcd for C₃₂H₃₅BrCl₃NO₁₁Na [M+Na]⁺ 816.0. found 815.9.

### 2.3. Synthesis of 5-tert-butyl-2-methylphenyl 2,7-di-O-acetyl-3-O-levulinoyl-4-O-para-bromobenzyl-6-O-benzyl-1-thio-L-glycero-D-manno-heptopyranoside 21

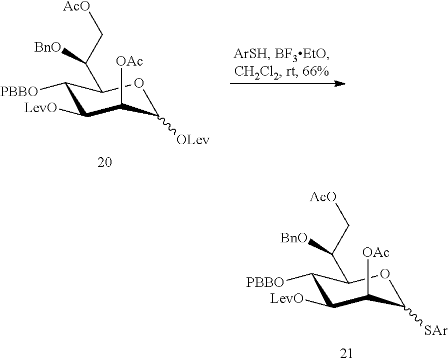

To a solution of compound 20 (50 mg, 0.067 mmol) in CH₂Cl₂ (2.5 mL), was added 5-tert-butyl-2-methylbenzenethiol (61 μL, 0.33 mmol) and BF₃OEt₂ (18 μL, 0.14 mmol). After being stirred at room temperature for overnight, the mixture was quenched with Et₃N and concentrated in vacuo. The residue was purified by silica gel column chromatography (cyclohexane/EtOAc: 3/1) to give 21 (36 mg, 66%) as a colorless syrup: [α]²⁰_{D}=+98.8 (c 1.0, CHCl₃); ¹H NMR (400 MHz, CDCl₃) δ 7.46-7.29 (m, 8H), 7.18 (dd, J=2.0, 8.0 Hz, 1H), 7.07 (m, 3H), 5.54 (d, J=2.0 Hz, 1H), 5.51 (dd, J=2.0, 3.2 Hz, 1H), 5.37 (dd, J=3.2, 9.2 Hz, 1H), 4.79 (d-like, J=11.6 Hz, 1H), 4.54 (d-like, J=12.0 Hz, 1H), 4.50 (d-like, J=12.0 Hz, 1H), 4.44 (dd, J=6.0, 11.2 Hz, 1H), 4.27 (dd, J=1.2, 9.6 Hz, 1H), 4.18 (d-like, J=12.0 Hz, 1H), 4.09 (t, J=9.6 Hz, 1H), 4.06 (m, 1H), 3.99 (m, 1H), 2.69 (m, 2H), 2.44 (m, 2H), 2.36 (s, 3H), 2.17 (s, 3H), 2.16 (s, 3H), 1.91 (s, 3H), 1.29 (s, 9H); ¹³C NMR (100 MHz, CDCl₃) δ 206.1, 171.8, 170.4, 170.1, 150.0, 137.8, 137.1, 135.8, 132.1, 131.5, 130.1, 128.9, 128.5, 128.1, 128.0, 127.8, 124.9, 121.5, 85.3, 73.5, 73.4, 73.0, 72.9, 72.4, 72.3, 71.6, 62.6, 37.7, 34.5, 31.3, 29.8, 27.8, 26.9, 21.0, 20.7, 20.2; HRMS (ESI) m/z calcd for C₄₁H₄₉BrSO₁₀Na [M+Na]⁺ 837.2107. found 837.2085.

### 2.4. Synthesis of 5-tert-butyl-2-methylphenyl 2-O-acetyl-3,7-di-O-benzoyl-4-O-para-bromobenzyl-6-O-benzyl-1-thio-L-glycero-D-manno-heptopyranoside 23

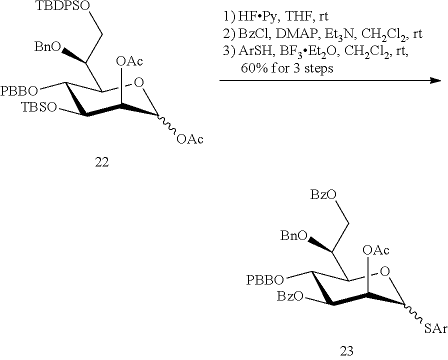

Compound 22 (930 mg, 1.03 mmol) was dissolved in THF (4 mL) at room temperature, followed by addition of 70% HF-pyridine (0.8 mL). After stirring for 2 days, the reaction mixture was carefully quenched with sat. aq. NaHCO₃ and the resulting solution was diluted with EtOAc. The organic layer was washed with brine, dried over Na₂SO₄, filtered and concentrated in vacuo to give the corresponding diol as a colorless syrup. To a solution of the above diol and DMAP (200 mg, 1.64 mmol) in CH₂Cl₂ (20 mL), was added Et₃N (2.0 mL) and benzoyl chloride (1.0 mL). After being stirred at room temperature for overnight, the mixture was concentrated in vacuo to give a residue, which was purified by silica gel column chromatography (cyclohexane/EtOAc: 5/1 to 3/1) to give the corresponding ester (709 mg) as a white solid. To a solution of the above ester (709 mg, 0.93 mmol) and freshly activated 4 Å MS in CH₂Cl₂ (20 mL), was added 5-tert-butyl-2-methylbenzenethiol (0.98 mL, 5.31 mmol) and BF₃OEt₂ (0.73 mL, 5.77 mmol). After being stirred at room temperature for overnight, the mixture was quenched with Et₃N and concentrated in vacuo. The residue was purified by silica gel column chromatography (cyclohexane/EtOAc: 10/1 to 8/1) to provide 23 (531 mg, α/β=4.0, 60% for 3 steps) as a white foam: [α]²⁰_{D} α-anomer: +64.0 (c 0.3, CHCl₃); β-anomer: +52.3 (c 1.5, CHCl₃); ¹H NMR (400 MHz, CDCl₃) α-anomer: δ 7.88-7.81 (m, 4H), 7.51-7.15 (m, 14H), 7.06 (dd, J=2.0, 8.0 Hz, 1H), 6.96-6.81 (m, 3H), 5.59 (m, 3H), 4.82 (d-like, J=11.6 Hz, 1H), 4.70 (dd, J=5.6, 10.8 Hz, 1H), 4.51 (d-like, J=12.0 Hz, 1H), 4.42 (m, 2H), 4.18 (m, 4H), 2.26 (s, 3H), 2.09 (s, 3H), 1.20 (s, 9H); β-anomer: δ 8.07-7.92 (m, 4H), 7.61-7.36 (m, 14H), 7.14 (dd, J=2.0, 8.0 Hz, 1H), 7.04 (m, 3H), 5.78 (dd, J=2.0, 3.2 Hz, 1H), 5.63 (d, J=2.0 Hz, 1H), 5.51 (dd, J=3.2, 9.6 Hz, 1H), 4.92 (d-like, J=11.6 Hz, 1H), 4.80 (dd, J=6.0, 10.8 Hz, 1H), 4.52 (m, 2H), 4.47 (dd, J=1.6, 9.6 Hz, 1H), 4.32 (m, 3H), 4.28 (m, 1H), 2.38 (s, 3H), 1.91 (s, 3H), 1.25 (s, 9H); ¹³C NMR (100 MHz, CDCl₃) α-anomer: δ 169.8, 165.9, 165.2, 149.9, 137.8, 136.7, 135.5, 133.4, 133.0, 132.2, 131.3, 130.1, 129.6, 129.5, 129.4, 129.3, 128.8, 128.5, 128.4, 128.3, 128.1, 128.0, 127.3, 124.7, 121.5, 85.2, 73.6, 73.5, 73.4, 72.8, 72.6, 72.3, 72.1, 62.3, 34.5, 31.3, 21.0, 20.1; β-anomer: δ 169.8, 166.0, 165.4, 149.9, 138.0, 137.0, 136.1, 133.4, 133.1, 132.1, 131.5, 130.1, 129.9, 129.6, 129.5, 128.7, 128.5, 128.4, 128.3, 127.8, 127.7, 125.0, 121.5, 85.5, 74.0, 73.6, 73.3, 72.6, 72.5, 72.4, 72.3, 62.6, 34.5, 31.3, 20.8, 20.3; HRMS (ESI) m/z calcd for C₄₈H₄₉BrSO₉Na [M+Na]⁺ 905.2158. found 905.2164.

### 2.5. Synthesis of 5-tert-butyl-2-methylphenyl 3,7-di-O-benzoyl-4-O-para-bromobenzyl-6-O-benzyl-1-thio-L-glycero-D-manno-heptopyranoside 5

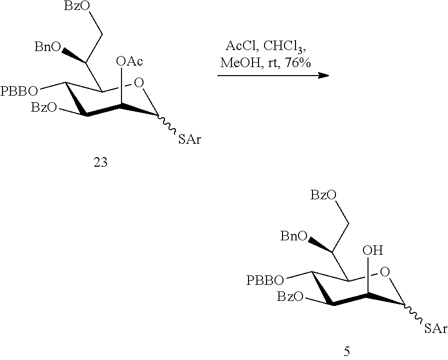

To a solution of thioglycoside 23 (220 mg, 0.249 mmol) in MeOH/CHC₃ (5/2, v/v, 12.3 mL), was added acetyl chloride (0.53 mL). After being stirred at room temperature for 1d, the mixture was diluted with CH₂Cl₂, washed with saturated aqueous NaHCO₃, and brine. The organic layer was dried over Na₂SO₄, filtered, and concentrated in vacuo. The residue was purified by silica gel column chromatography (hexane/EtOAc: 6/1) to afford 5 (160 mg, 76%) as a white solid: [α]²⁰_{D}=+79.1 (c 1.0, CHCl₃); ¹H NMR (400 MHz, CDCl₃) δ 8.02-7.83 (m, 4H), 7.58-7.21 (m, 14H), 7.10 (dd, J=2.0, 8.0 Hz, 1H), 7.01-6.87 (m, 3H), 5.67 (d, J=2.0 Hz, 1H), 5.56 (dd, J=3.2, 9.2 Hz, 1H), 4.85 (d-like, J=12.0 Hz, 1H), 4.76 (dd, J=6.0, 11.2 Hz, 1H), 4.54 (d-like, J=11.6 Hz, 1H), 4.46 (m, 3H), 4.28 (m, 3H), 4.19 (m, 1H), 2.30 (s, 3H), 1.27 (s, 9H); ¹³C NMR (100 MHz, CDCl₃) δ 165.9, 165.5, 149.9, 137.7, 136.8, 134.9, 133.5, 133.0, 132.7, 131.3, 130.0, 129.7, 129.6, 129.5, 129.0, 128.5, 128.2, 128.1, 128.0, 126.4, 124.3, 121.5, 86.9, 73.7, 73.5, 73.1, 72.6, 72.2, 71.4, 62.4, 34.5, 31.3, 20.1; HRMS (ESI) m/z calcd for C₄₆H₄₇BrSO₈Na [M+Na]⁺ 863.2052. found 863.2017.

### 2.6. Synthesis of methyl (N-benzyl-benzyloxycarbonyl-5-aminopentyl 4,5,7,8-tetra-O-acetyl-3-deoxy-α-D-manno-oct-2-ulopyranosid)onate 28

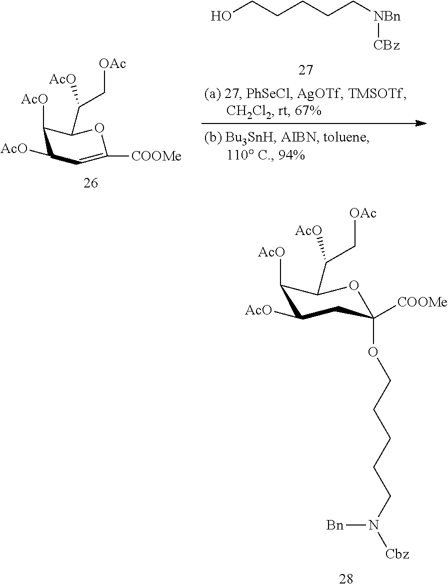

To a stirred solution of phenylselenyl chloride (2.73 g, 14.3 mmol) in CH₂Cl₂ (50 mL) was added AgOTf (2.55 g, 9.92 mmol) and TMSOTf (0.16 mL, 0.86 mmol). After stirring at room temperature for 30 min, a solution of glycal 26 (2.85 g, 7.08 mmol) and linker 27 (3.25 g, 9.92 mmol) in CH₂Cl₂ (70 mL) was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (cyclohexane/EtOAc: 3/1) to give a white solid (4.2 g, 67%). To a solution of the above solid (4.2 g, 4.75 mmol) in toluene (140 mL), was added tri-n-butyltin hydride (3.8 mL, 14.24 mmol) and AIBN (779 mg, 4.75 mmol). After being refluxed for 1.5 h, the mixture was concentrated in vacuo and purified by silica gel column chromatography (cyclohexane/EtOAc: 5/2 to 2/1) to afford 28 (3.26 g, 94%) as a colorless syrup: [α]²⁰_{D}=+43.5 (c 0.4, CHCl₃); ¹H NMR (400 MHz, CDCl₃) δ 7.29-7.11 (m, 10H), 5.29 (br s, 1H), 5.24 (m, 1H), 5.13 (m, 3H), 4.51 (d-like, J=12.0 Hz, 1H), 4.43 (br s, 2H), 4.05 (dd, J=3.6, 12.4 Hz, 1H), 3.98 (m, 1H), 3.71 (s, 3H), 3.37 (m, 1H), 3.16 (m, 3H), 2.09 (dd, J=4.8, 13.2 Hz, 1H), 2.00 (s, 3H), 1.95 (m, 1H), 1.91 (s, 6H), 1.90 (s, 3H), 1.47 (m, 4H), 1.22 (m, 2H); ¹³C NMR (100 MHz, CDCl₃) δ 170.4, 170.3, 169.9, 169.6, 167.8, 137.8, 128.5, 128.4, 127.9, 127.8, 127.3, 127.2, 98.8, 68.1, 67.6, 67.1, 66.4, 64.3, 63.8, 62.0, 52.6, 32.1, 29.7, 29.2, 23.4, 20.8, 20.7, 20.6; HRMS (ESI) m/z calcd for C₃₇H₄₇NO₁₄Na [M+Na]⁺ 752.2894. found 752.2921.

### 2.7. Synthesis of methyl (N-benzyl-benzyloxycarbonyl-5-aminopentyl 4-O-benzyl-7,8-O-isopropylidene-3-deoxy-α-D-manno-oct-2-ulopyranosid)onate 7

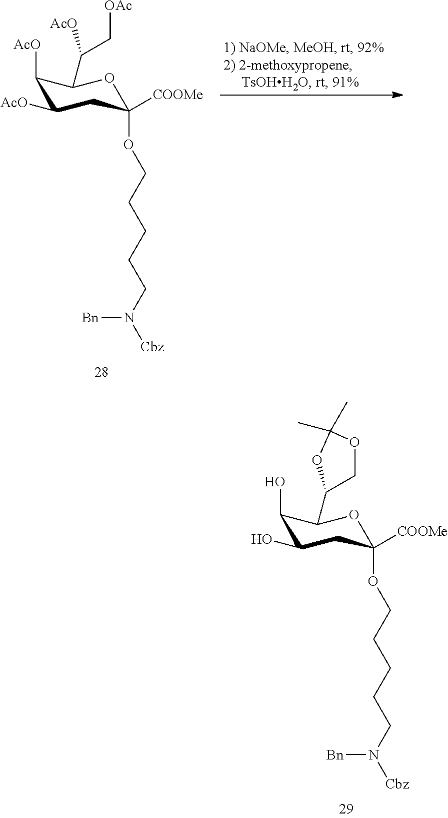

To a stirred solution of compound 28 (1 g, 1.37 mmol) in MeOH (25 mL) was added NaOMe (74 mg, 1.37 mmol). The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol (710 mg, 92%) as a white solid. To a stirred solution of the above alcohol (500 mg, 0.89 mmol) in DMF (9 mL), was added 2-methoxypropene (153 μL, 1.60 mmol) and p-toluenesulfonic acid monohydrate (40 mg, 0.21 mmol). The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded 29 (490 mg, 91%) as a colorless syrup: [α]²⁰_{D}=+30.3 (c 0.5, CHCl₃); ¹H NMR (400 MHz, CDCl₃) δ 7.29-7.10 (m, 10H), 5.09 (d-like, J=12.0 Hz, 2H), 4.42 (br s, 2H), 4.32 (m, 1H), 4.07 (dd, J=6.0, 8.4 Hz, 1H), 3.97 (m, 1H), 3.88 (m, 2H), 3.67 (s, 3H), 3.42 (m, 1H), 3.31 (m, 1H), 3.15 (m, 3H), 2.05 (dd, J=4.8, 12.8 Hz, 1H), 1.78 (t, J=12.0 Hz, 1H), 1.44 (m, 4H), 1.32 (s, 3H), 1.29 (s, 3H), 1.18 (m, 2H); ¹³C NMR (100 MHz, CDCl₃) δ 168.7, 137.8, 128.5, 128.4, 127.9, 127.8, 127.3, 127.2, 109.4, 99.0, 73.6, 72.8, 67.2, 67.1, 66.7, 65.7, 63.6, 52.5, 35.0, 29.7, 29.2, 26.9, 25.3; HRMS (ESI) m/z calcd for C₃₂H₄₃NO₁₀Na [M+Na]⁺ 624.2785. found 624.2736.

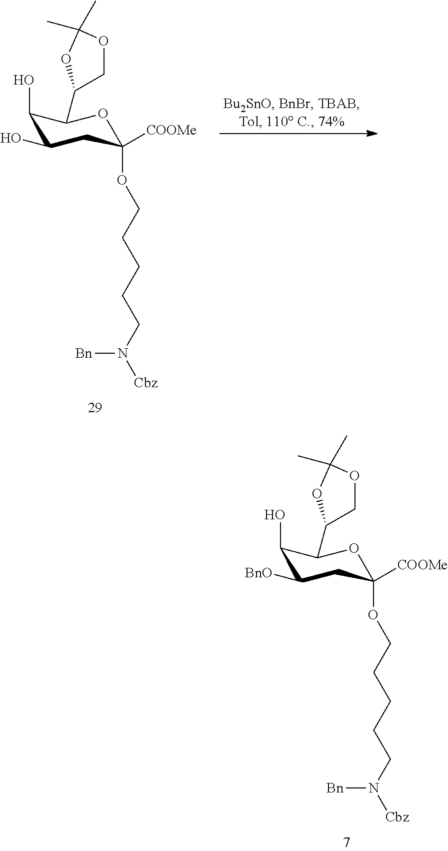

A mixture of compound 29 (490 mg, 0.81 mmol), dibutyltin oxide (304 mg, 1.22 mmol) and 4 Å MS (500 mg) in toluene (15 mL) was heated at 110° C. for 2 h. After cooling to room temperature, benzyl bromide (0.17 mL, 1.47 mmol) and tetrabutylammonium bromide (158 mg, 0.49 mmol) were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (cyclohexane/EtOAc: 4/1) to give a syrup, which was dissolved in MeOH (8 mL) and treated with NaOMe (24 mg, 0.44 mmol). The mixture was stirred at room temperature for 3 h, and then neutralized with Amberlite IR120 H⁺ resin. Filtration, concentration in vacuo, and purification by silica gel column chromatography (cyclohexane/EtOAc: 3/1) provided 7 (418 mg, 74%) as a colorless syrup: [α]²⁰_{D}=+21.7 (c 0.8, CHCl₃); ¹H NMR (400 MHz, CDCl₃) δ 7.31-7.12 (m, 15H), 5.12 (d-like, J=13.2 Hz, 2H), 4.53 (dd, J=11.6, 16.4 Hz, 2H), 4.41 (m, 3H), 4.09 (m, 2H), 3.92 (dd, J=4.8, 8.8 Hz, 1H), 3.85 (m, 1H), 3.69 (s, 3H), 3.42 (m, 1H), 3.34 (m, 1H), 3.20 (m, 3H), 2.16 (dd, J=4.8, 12.8 Hz, 1H), 1.93 (t, J=12.8 Hz, 1H), 1.48 (m, 4H), 1.35 (s, 3H), 1.32 (s, 3H), 1.20 (m, 2H); ¹³C NMR (100 MHz, CDCl₃) δ 168.6, 137.9, 137.7, 128.5, 128.4, 127.9, 127.8, 127.7, 127.6, 127.3, 127.1, 109.2, 98.9, 73.5, 72.9, 72.7, 70.3, 67.1, 67.0, 64.1, 63.5, 52.5, 32.2, 29.2, 26.9, 25.3, 23.4; HRMS (ESI) m/z calcd for C₃₉H₄₉NO₁₀Na [M+Na]⁺ 714.3254. found 714.3221.

### 2.8. Synthesis of 5-tert-butyl-2-methylphenyl (3,4,6-tri-O-acetyl-2-azido-2-deoxy-α-D-glucopyranosyl)-(1→2)-3,7-di-O-benzoyl-4-O-para-bromobenzyl-6-O-benzyl-1-thio-L-glycero-D-manno-heptopyranoside 30

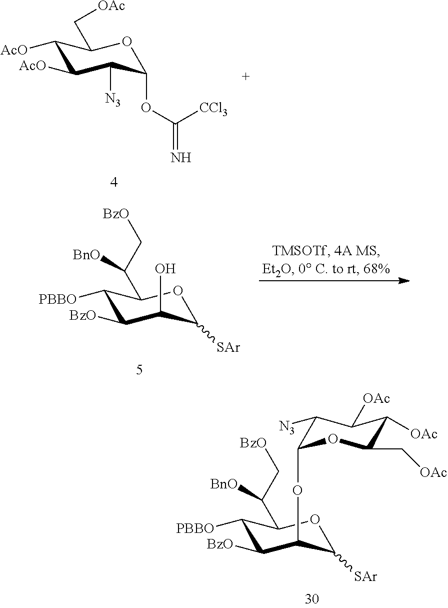

To a stirred mixture of the donor 4 (70 mg, 0.147 mmol), acceptor 5 (28 mg, 0.033 mmol), and freshly activated 4 Å MS in dry Et₂O (3 mL) at 0° C., was added dropwise TMSOTf in CH₂Cl₂ (0.05 M, 0.34 mL) under nitrogen. After 0.5 h, the temperature was allowed to warm up naturally to room temperature and the stirring continued for overnight. The mixture was then filtered and concentrated in vacuo. The residue was purified silica gel column chromatography (hexane/EtOAc: 7/1 to 6/1) provided 30 (26 mg, 68%) as a white solid: [α]²⁰_{D}=+78.61 (c 0.8, CHCl₃); ¹H NMR (400 MHz, CDCl₃) δ 8.07-7.91 (m, 4H), 7.58-7.21 (m, 14H), 7.16 (dd, J=2.0, 8.0 Hz, 1H), 7.08-6.85 (m, 3H), 5.83 (d, J=2.0 Hz, 1H), 5.66 (dd, J=9.2, 10.4 Hz, 1H), 5.58 (m, 1H), 5.07 (dd, J=9.2, 10.4 Hz, 1H), 5.05 (d, J=3.2 Hz, 1H), 4.85 (d-like, J=12.0 Hz, 1H), 4.77 (dd, J=5.6, 10.8 Hz, 1H), 4.58 (m, 2H), 4.55 (m, 1H), 4.42 (m, 3H), 4.32 (m, 2H), 4.17 (t, J=6.8 Hz, 1H), 3.98 (m, 2H), 3.41 (dd, J=3.6, 10.4 Hz, 1H), 2.36 (s, 3H), 2.13 (s, 3H), 1.95 (s, 3H), 1.85 (s, 3H), 1.28 (s, 9H); ¹³C NMR (100 MHz, CDCl₃) δ 170.5, 169.8, 169.7, 166.0, 165.9, 150.0, 137.9, 136.9, 135.7, 133.4, 133.0, 132.6, 131.2, 130.2, 129.7, 129.6, 129.5, 129.3, 129.0, 128.7, 128.5, 128.3, 128.1, 127.9, 124.9, 121.3, 99.1, 85.9, 73.3, 73.1, 72.6, 72.2, 71.7, 70.7, 70.5, 68.6, 68.4, 68.2, 62.6, 61.6, 61.2, 34.5, 31.3, 20.7, 20.5, 20.3, 20.2; HRMS (ESI) m/z calcd for C₅₈H₆₂BrSN₃O₁₅Na [M+Na]⁺ 1176.2962. found 1176.2992.

### 2.9. Synthesis of N-Phenyl Trifluoroacetimidate (3,4,6-tri-O-acetyl-2-azido-2-deoxy-α-D-glucopyranosyl)-(1→2)-3,7-di-O-benzoyl-4-O-para-bromobenzyl-6-O-benzyl-L-glycero-D-manno-heptopyranoside 2

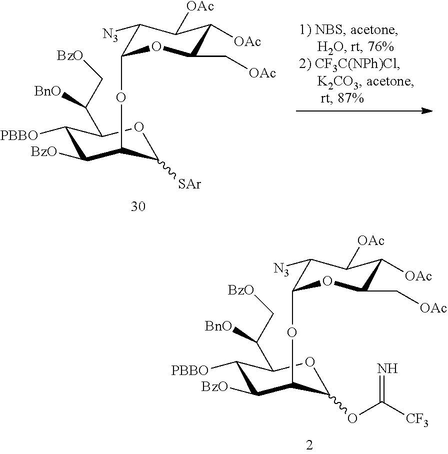

To a solution of compound 30 (83 mg, 0.072 mmol) in acetone/H₂O (10/1, v/v, 2.2 mL), was added NBS (38 mg, 0.22 mmol). After being stirred at room temperature for 1 h, the mixture was diluted with EtOAc, washed with saturated aqueous NaHCO₃ and brine. The organic layer was dried over Na₂SO₄, filtered, and concentrated in vacuo. The residue was purified by silica gel column chromatography (hexane/EtOAc: 2/1) to afford the corresponding hemiacetal (54 mg, 76%) as a colorless syrup. To a solution of the above hemiacetal (69 mg, 0.070 mmol) and K₂CO₃ (27 mg, 0.195 mmol) in acetone (1.5 mL), was added 2,2,2-trifluoro-N-phenylacetimidoyl chloride (100 mg, 0.482 mmol). After being stirred at room temperature for 30 min, the solution was filtered and concentrated in vacuo to a residue, which was purified by silica gel column chromatography (hexane/EtOAc: 3/1) to afford 2 (70 mg, 87%) as a colorless syrup: [α]²⁰_{D}=+47.32 (c 0.8, CHCl₃); ¹H NMR (400 MHz, CDCl₃) δ 8.06-8.01 (m, 4H), 7.61-7.09 (m, 18H), 6.86-6.81 (m, 4H), 5.62 (dd, J=9.2, 10.4 Hz, 1H), 5.59 (dd, J=2.8, 9.2 Hz, 1H), 5.05 (dd, J=9.6, 10.4 Hz, 1H), 4.85 (d-like, J=12.0 Hz, 1H), 4.77 (dd, J=5.2, 10.8 Hz, 1H), 4.60 (d-like, J=12.4 Hz, 1H), 4.49-4.40 (m, 5H), 4.27 (dd, J=4.0, 12.4 Hz, 1H), 4.15 (t, J=6.4 Hz, 1H), 4.08 (d-like, J=9.6 Hz, 1H), 3.99 (d-like, J=12.4 Hz, 1H), 3.40 (dd, J=4.0, 10.8 Hz, 1H), 2.12 (s, 3H), 2.01 (s, 3H), 1.95 (s, 3H); ¹³C NMR (100 MHz, CDCl₃) δ 170.5, 169.8, 169.7, 166.2, 165.9, 143.2, 137.8, 136.7, 133.6, 133.3, 131.4, 129.8, 129.7, 129.6, 129.3, 129.2, 128.9, 128.8, 128.6, 128.5, 128.2, 128.1, 124.6, 121.6, 119.4, 99.3, 77.3, 73.6, 73.5, 72.7, 72.5, 72.2, 70.4, 68.5, 68.2, 62.3, 61.5, 61.1, 20.8, 20.7, 20.6; HRMS (ESI) m/z calcd for C₅₅H₅₂BrF₃N₄O₁₆Na [M+Na]⁺ 1185.2400. found 1185.2423.

### 2.10. Synthesis of methyl [N-benzyl-benzyloxycarbonyl-5-aminopentyl (2,7-di-O-acetyl-3-O-levulinoyl-4-O-para-bromobenzyl-6-O-benzyl-L-glycero-α-D-manno-heptopyranosyl)-(1→5)-4-O-benzyl-7,8-O-isopropylidene-3-deoxy-α-D-manno-oct-2-ulopyranosid]onate 31

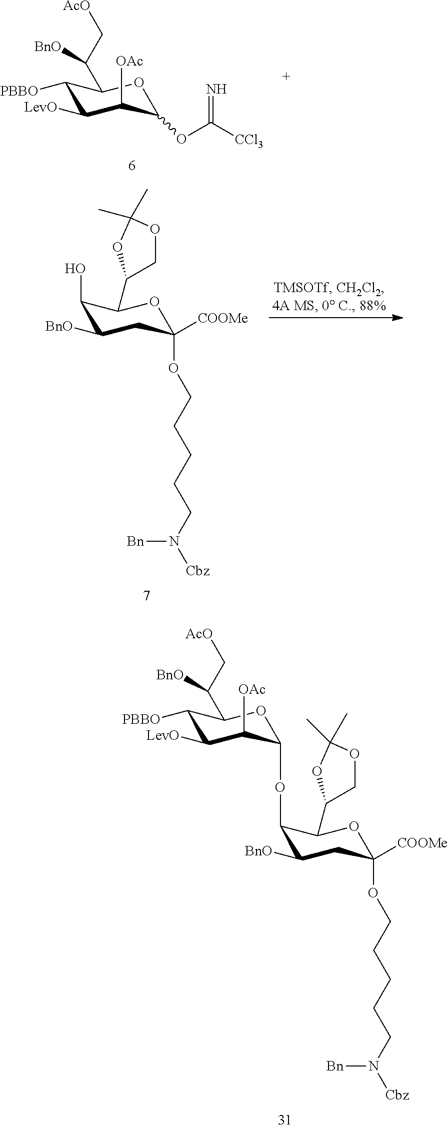

To a stirred mixture of the donor 6 (100 mg, 0.126 mmol), acceptor 7 (65 mg, 0.094 mmol), and freshly activated 4 Å MS in dry CH₂Cl₂ (5.5 mL) at 0° C., was added dropwise TMSOTf in CH₂Cl₂ (0.05 M, 138 μL) under nitrogen. After being stirred at 0° C. for 1 h, the mixture was quenched with Et₃N, filtered and concentrated in vacuo. The residue was purified by silica gel column chromatography (cyclohexane/EtOAc: 2/1 to 3/2) to afford 31 (110 mg, 88%) as a colorless syrup: [α]²⁰_{D}=+35.1 (c 1.0, CHCl₃); ¹H NMR (400 MHz, CDCl₃) δ 7.45-7.05 (m, 24H), 5.40 (dd, J=3.2, 9.6 Hz, 1H), 5.33 (dd, J=2.0, 3.2 Hz, 1H), 5.19 (d, J=1.6 Hz, 1H), 5.16 (m, 2H), 4.76 (d-like, J=12.0 Hz, 1H), 4.64 (d-like, J=11.6 Hz, 1H), 4.50 (m, 3H), 4.42 (m, 2H), 4.33 (m, 2H), 4.19 (m, 3H), 4.09 (br s, 1H), 4.01 (t, J=9.6 Hz, 1H), 3.92 (dd, J=2.8, 12.4 Hz, 1H), 3.86 (m, 1H), 3.77 (m, 1H), 3.76 (s, 3H), 3.65 (m, 1H), 3.25 (m, 5H), 2.67 (m, 2H), 2.43 (m, 2H), 2.30 (dd, J=3.6, 12.4 Hz, 1H), 2.15 (s, 3H), 2.11 (s, 3H), 2.00 (t, J=12.0 Hz, 1H), 1.95 (s, 3H), 1.49 (m, 4H), 1.25 (s, 3H), 1.24 (s, 3H), 1.23 (m, 2H); ¹³C NMR (100 MHz, CDCl₃) δ 206.2, 171.5, 170.4, 170.0, 168.4, 138.5, 137.7, 137.5, 131.3, 128.9, 128.5, 128.4, 128.3, 128.2, 128.1, 127.9, 127.8, 127.7, 127.6, 127.5, 127.2, 121.3, 109.6, 98.8, 97.4, 77.2, 74.5, 74.4, 73.2, 73.0, 72.6, 72.3, 72.2, 72.1, 71.9, 71.7, 70.2, 70.0, 67.9, 67.1, 66.3, 63.5, 52.4, 37.8, 31.8, 29.8, 29.7, 29.3, 27.9, 26.8, 24.7, 23.4, 22.7, 21.0, 20.9; HRMS (ESI) m/z calcd for C₆₉H₈₂BrNO₂₀Na [M+Na]⁺ 1346.4511. found 1346.4506.

### 2.11. Synthesis of methyl [N-benzyl-benzyloxycarbonyl-5-aminopentyl (2,7-di-O-acetyl-4-O-para-bromobenzyl-6-O-benzyl-L-glycero-α-D-manno-heptopyranosyl)-(1→5)-4-O-benzyl-7,8-O-isopropylidene-3-deoxy-α-D-manno-oct-2-ulopyranosid]onate 3

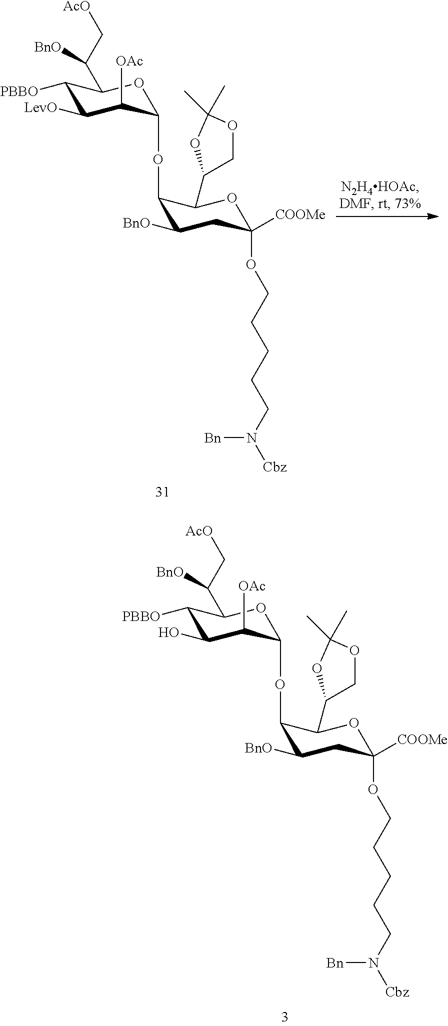

To a solution of 31 (105 mg, 0.079 mmol) in DMF (3 mL) at room temperature, was added hydrazine acetate (30 mg, 0.324 mmol). After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 3 (71 mg, 73%) as a colorless syrup: [α]²⁰_{D}=+28.6 (c 0.8, CHCl₃); ¹H NMR (400 MHz, CDCl₃) δ 7.47-7.10 (m, 24H), 5.26 (d, J=1.2 Hz, 1H), 5.20 (m, 3H), 4.76 (d-like, J=12.0 Hz, 2H), 4.65 (d-like, J=11.6 Hz, 1H), 4.50-4.43 (m, 4H), 4.38-4.20 (m, 6H), 4.11 (m, 2H), 3.95 (dd, J=2.4, 12.0 Hz, 1H), 3.83 (m, 2H), 3.76 (s, 3H), 3.68 (m, 1H), 3.35 (m, 2H), 3.25 (m, 3H), 2.32 (dd, J=4.0, 12.8 Hz, 1H), 2.13 (s, 3H), 1.96 (s, 3H), 1.94 (t, J=12.0 Hz, 1H), 1.51 (m, 4H), 1.27 (s, 3H), 1.26 (s, 3H), 1.25 (m, 2H); ¹³C NMR (100 MHz, CDCl₃) δ 171.1, 170.6, 170.5, 168.5, 138.5, 137.9, 137.8, 137.7, 131.4, 131.2, 129.0, 128.6, 128.5, 128.4, 128.2, 128.0, 127.9, 127.8, 127.7, 127.6, 127.5, 127.3, 127.1, 121.3, 109.6, 98.8, 97.0, 77.2, 75.7, 74.8, 74.5, 73.4, 72.7, 72.6, 72.2, 72.1, 71.6, 70.6, 70.2, 67.9, 67.1, 66.8, 63.6, 60.4, 52.5, 31.9, 29.2, 26.9, 24.8, 23.4, 21.1, 21.0; HRMS (ESI) m/z calcd for C₆₄H₇₆BrNO₁₈Na [M+Na]⁺1250.4123. found 1250.4144.

### 2.12. Synthesis of methyl [N-benzyl-benzyloxycarbonyl-5-aminopentyl (3,4,6-tri-O-acetyl-2-azido-2-deoxy-α-D-glucopyranosyl)-(1→2)-(3,7-di-O-benzoyl-4-O-para-bromobenzyl-6-O-benzyl-L-glycero-α-D-manno-heptopyranosyl)-(1→3)-(2,7-di-O-acetyl-4-O-para-bromobenzyl-6-O-benzyl-L-glycero-α-D-manno-heptopyranosyl)-(1→5)-4-O-benzyl-7,8-O-isopropylidene-3-deoxy-α-D-manno-oct-2-ulopyranosid]onate 32

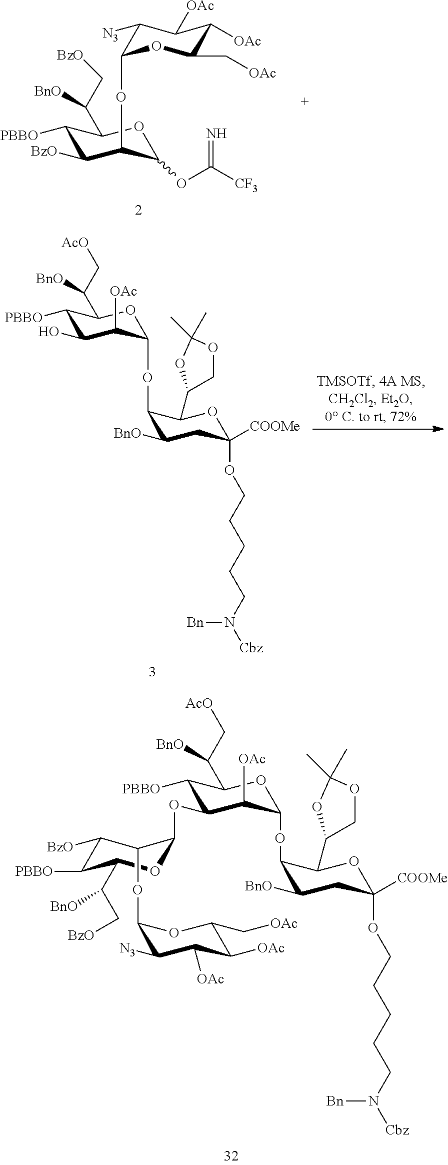

To a stirred mixture of the disaccharide donor 2 (49 mg, 42 μmol), disaccharide acceptor 3 (38 mg, 31 μmol), and freshly activated 4 Å MS in dry diethyl ether and dichloromethane (1/1, v/v, 3.6 mL) at 0° C., was added TMSOTf in CH₂Cl₂ (0.05 M, 90 μL) under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (hexane/EtOAc/TEA: 5/2/0.07) to afford 32 (49 mg, 72%) as a white solid: [α]²⁰_{D}=+19.3 (c 1.0, CHCl₃); ¹H NMR (600 MHz, Pyridine-d₅) δ 8.49-8.33 (m, 4H), 7.85 (m, 2H), 7.66-7.31 (m, 33H), 7.16 (d, J=7.8 Hz, 2H), 6.96 (d, J=7.8 Hz, 2H), 6.11 (dd, J=2.4, 9.0 Hz, 1H), 6.04 (m, 1H), 6.02 (br s, 1H), 5.84 (br s, 1H), 5.50 (t, J=9.6 Hz, 1H), 5.45-5.37 (m, 3H), 5.20 (m, 2H), 5.02 (m, 4H), 4.93-4.83 (m, 7H), 4.77 (d-like, J=11.4 Hz, 2H), 4.73-4.62 (m, 6H), 4.56 (m, 3H), 4.48 (m, 1H), 4.43 (m, 2H), 4.28 (d-like, J=11.4 Hz, 1H), 4.18 (m, 2H), 4.07-4.01 (m, 3H), 3.91 (m, 1H), 3.89 (s, 3H), 3.76 (m, 1H), 3.42 (m, 3H), 3.29 (m, 1H), 2.62 (d-like, J=9.6 Hz, 1H), 2.32 (t, J=12.0 Hz, 1H), 2.27 (s, 3H), 2.16 (s, 3H), 2.15 (s, 3H), 2.01 (s, 3H), 1.99 (s, 3H), 1.55 (m, 4H), 1.41 (s, 3H), 1.35 (s, 3H), 1.26 (m, 2H); ¹³C NMR (150 MHz, Pyridine-d₅) δ 171.1, 171.0, 170.9, 170.7, 170.4, 169.1, 167.2, 166.7, 150.7, 140.2, 139.4, 139.3, 139.0, 138.9, 138.5, 138.3, 136.3, 134.1, 134.0, 132.3, 131.8, 131.2, 130.9, 130.8, 130.5, 130.3, 129.7, 129.6, 129.5, 129.4, 129.3, 129.2, 129.1, 128.7, 128.6, 128.2, 128.0, 124.3, 123.6, 122.1, 121.7, 110.4, 101.1, 99.8, 98.1, 76.7, 76.1, 75.3, 75.0, 74.5, 73.7, 73.5, 73.2, 73.1, 72.5, 72.3, 71.6, 70.9, 69.5, 69.4, 68.5, 67.7, 67.6, 67.3, 64.4, 62.7, 62.3, 54.7, 53.0, 51.2, 50.8, 47.9, 47.0, 30.5, 30.0, 27.4, 25.6, 24.1, 23.4, 21.5, 21.3, 21.1, 21.0, 20.9; HRMS (ESI) m/z calcd for C₁₁₁H₁₂₂Br₂N₄O₃₃Na [M+Na]⁺ 2222.6275. found 2222.6349.

### 2.13. Synthesis of methyl [N-benzyl-benzyloxycarbonyl-5-aminopentyl (3,4,6-tri-O-acetyl-2-N-acetyl-2-deoxy-α-D-glucopyranosyl)-(1→2)-(3,7-di-O-benzoyl-4-O-para-bromobenzyl-6-O-benzyl-L-glycero-α-o-manno-heptopyranosyl)-(1→3)-(2,7-di-O-acetyl-4-O-para-bromobenzyl-6-O-benzyl-L-glycero-α-D-manno-heptopyranosyl)-(1→5)-4-O-benzyl-7,8-O-isopropylidene-3-deoxy-α-D-manno-oct-2-ulopyranosid]onate 33

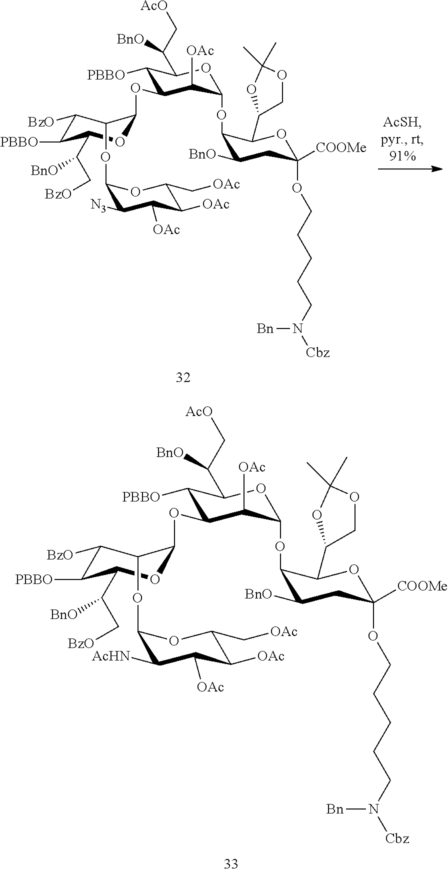

To a solution of compound 32 (37 mg, 0.017 mmol) in dry pyridine (0.3 mL), was added thioacetic acid (0.3 mL, 4.18 mmol). After being stirred at room temperature for 24 h, the solution was coevaporated with toluene to give a residue, which was purified by silica gel column chromatography (hexane/EtOAc/TEA: 3/2/0.05 to 1/1/0.02) to give 33 (34 mg, 91%) as a pale yellow solid: [α]²⁰_{D}=+26.1 (c 0.8, CHCl₃); ¹H NMR (400 MHz, Pyridine-d₅) δ 8.51-8.19 (m, 4H), 7.79-7.18 (m, 37H), 6.99 (d, J=8.4 Hz, 2H), 6.12 (m, 2H), 6.04 (br s, 1H), 5.88 (m, 1H), 5.83 (br s, 1H), 5.56 (t, J=10.0 Hz, 1H), 5.39 (m, 4H), 5.24 (d-like, J=12.0 Hz, 1H), 5.05-4.89 (m, 9H), 4.86-4.68 (m, 8H), 4.64-4.54 (m, 6H), 4.39 (m, 2H), 4.17 (m, 2H), 4.07 (m, 2H), 4.00 (m, 2H), 3.93 (s, 3H), 3.75 (d-like, J=8.8 Hz, 1H), 3.46 (m, 3H), 3.29 (m, 1H), 2.63 (m, 1H), 2.28 (s, 3H), 2.25 (m, 1H), 2.23 (s, 3H), 2.02 (s, 3H), 1.98 (s, 3H), 1.97 (s, 6H), 1.55 (m, 4H), 1.41 (s, 3H), 1.34 (s, 3H), 1.25 (m, 2H); ¹³C NMR (100 MHz, Pyridine-d) δ 171.6, 171.1, 171.0, 170.4, 170.3, 169.0, 167.3, 166.2, 140.2, 139.5, 139.4, 138.9, 138.6, 138.3, 134.4, 134.0, 132.4, 131.9, 131.2, 130.8, 130.5, 130.4, 130.2, 129.7, 129.6, 129.5, 129.4, 129.3, 129.2, 128.7, 128.6, 128.3, 128.1, 128.0, 122.2, 110.4, 100.6, 99.8, 98.2, 76.0, 75.3, 74.8, 73.7, 73.5, 73.1, 72.5, 72.0, 70.9, 69.7, 68.5, 67.7, 67.2, 64.4, 62.9, 60.7, 53.5, 53.0, 51.2, 50.8, 48.0, 47.1, 30.5, 30.0, 27.4, 25.7, 24.1, 22.8, 21.6, 21.4, 21.3, 21.2, 21.1, 20.9; HRMS (ESI) m/z calcd for C₁₁₃H₁₂₆Br₂N₂O₃₄Na [M+Na]⁺ 2238.6476. found 2238.6545.

### 2.14. Synthesis of 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(5-amino)pentyl-3-deoxy-α-D-manno-oct-2-ulopyranosidonic acid 1

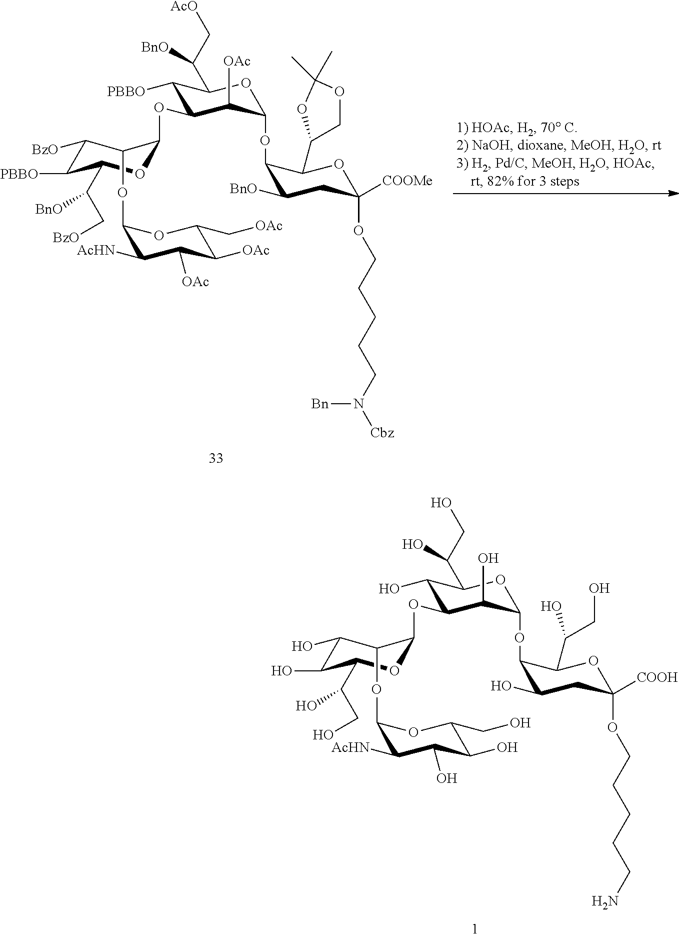

A solution of compound 33 (35 mg, 0.016 mmol) in acetic acid/water (8/1, v/v, 1.80 mL) was stirred at 70° C. for overnight. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v, 1.25 mL). After being stirred at room temperature for overnight, the reaction mixture was diluted with methanol and neutralized with Amberlite IR120 H⁺ resin. After filtration, the filtrate was concentrated in vacuo to give the corresponding tetrasaccharide as a white solid. A mixture of the above tetrasaccharide and Pd/C (70 mg, 10%) in methanol, water and acetic acid (50/25/1, v/v/v, 3.04 mL) was stirred under an atmosphere of H₂ at room temperature for 24 h. Filtration, concentration in vacuo and elution through Sephadex LH-20 column (H₂O) provided 1 (12 mg, 82% for 3 steps) as a white solid: [α]²⁰_{D}=+64.2 (c 0.3, H₂O); ¹H NMR (600 MHz, D₂O) δ 5.42 (br s, 1H), 5.12 (d, J=3.6 Hz, 1H), 5.09 (d, J=1.2 Hz, 1H), 4.16 (m, 2H), 4.09 (m, 2H), 4.06-4.01 (m, 4H), 3.98 (m, 3H), 3.95 (dd, J=3.0, 12.0 Hz, 1H), 3.91 (dd, J=3.6, 10.8 Hz, 1H), 3.85-3.76 (m, 6H), 3.73-3.63 (m, 6H), 3.49 (t, J=9.6 Hz, 1H), 3.44 (m, 1H), 3.30 (m, 1H), 3.01 (t, J=7.8 Hz, 2H), 2.10 (dd, J=4.8, 12.6 Hz, 1H), 2.07 (s, 3H), 1.84 (t, J=12.6 Hz, 1H), 1.69 (m, 2H), 1.62 (m, 2H), 1.44 (m, 2H); ¹³C NMR (150 MHz, D₂O) δ 175.0, 174.4, 101.3, 100.3, 99.7, 99.2, 78.6, 76.7, 75.0, 72.2, 71.9, 71.6, 71.3, 70.6, 70.4, 70.1, 69.9, 69.2, 68.7, 68.5, 66.3, 65.9, 65.6, 63.3, 63.2, 62.9, 60.4, 53.9, 39.3, 34.8, 28.1, 26.4, 22.4, 21.9; HRMS (ESI) m/z calcd for C₃₅H₆₁N₂O₂₅ [M−H]⁻ 909.3563. found 909.3629.

### 2.15. Synthesis of 5-aminopentyl-L-glycero-a-D-manno-heptopyranoside 34 (reference example)

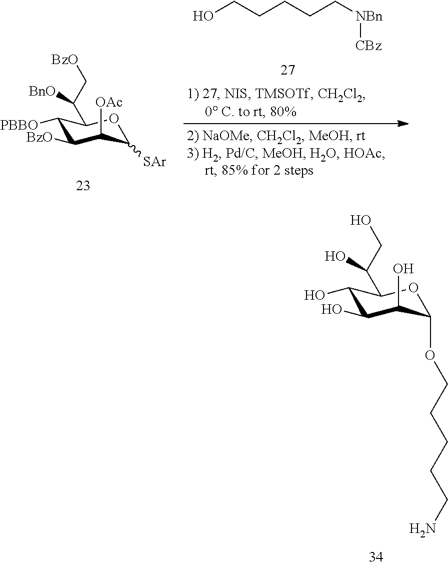

To a stirred mixture of the donor 23 (150 mg, 0.17 mmol), acceptor 27 (105 mg, 0.32 mmol), and freshly activated 4 Å MS in dry CH₂Cl₂ (10 mL) at 0° C., was added NIS (77 mg, 0.34 mmol) and TMSOTf in CH₂Cl₂ (0.05 M, 0.68 mL) under nitrogen. After 0.5 h, the temperature was allowed to warm up naturally to room temperature and the stirring continued for 2 h. The mixture was diluted with CH₂Cl₂, and filtered. The filtrate was washed with 10% aq. Na₂S₂O₃, saturated NaHCO₃, and brine, respectively. The organic layer was dried over Na₂SO₄, filtered and concentrated in vacuo. The residue was purified by silica gel column chromatography (hexane/EtOAc: 5/1) to give α-linked heptose glycoside (140 mg, 80%) as a white foam: HRMS (ESI) m/z calcd for C₅₇H₅₈BrNO₁₂Na [M+Na]⁺ 1050.3040. found 1050.2997. To a solution of the above heptose glycoside (130 mg, 0.126 mmol) in CH₂Cl₂/MeOH (2/5, v/v, 7 mL), was added sodium methoxide (26 mg, 0.481 mmol). After being stirred at room temperature for overnight, the mixture was neutralized with Amberlite IR120 H⁺ resin. After filtration, the filtrate was concentrated in vacuo to give a residue, which was purified by silica gel column chromatography (CH₂Cl₂/MeOH: 20/1) to give the corresponding triol as a white solid. A mixture of the above triol and Pd/C (150 mg, 10%) in methanol, water and acetic acid (83/25/1, v/v/v, 6.56 mL) was stirred under an atmosphere of H₂ at room temperature for overnight. Filtration, concentration in vacuo and elution through Sephadex LH-20 column (MeOH) provided 34 (33 mg, 89% over 2 steps) as a white solid: [α]²⁰_{D}=+ 51.7 (c 1.0, MeOH); ¹H NMR (400 MHz, CD₃OD) δ 4.80 (s, 1H, H-1), 4.01 (t, J=6.4 Hz, 1H), 3.87 (t, J=9.6 Hz, 1H), 3.81 (m, 1H), 3.78-3.60 (m, 5H), 3.46 (m, 1H, OCH₂), 2.98 (t, J=7.6 Hz, 2H, NCH₂), 1.71 (m, 4H, CCH₂C), 1.53 (m, 2H, CCH₂C); ¹³C NMR (100 MHz, CD₃OD) δ 101.6 (C-1, J_{C,H}=170.4 Hz), 72.8, 72.5, 72.2, 70.6, 68.1, 67.8, 64.3, 40.8 (NCH₂), 30.0, 28.4, 24.3; HRMS (ESI) m/z calcd for C₁₂H₂₆NO₇ [M+H]⁺ 296.1709. found 296.1702.

### 2.16. Synthesis of 2-(5-amino)pentyl-3-deoxy-a-D-manno-oct-2-ulopyranosidonic acid 35

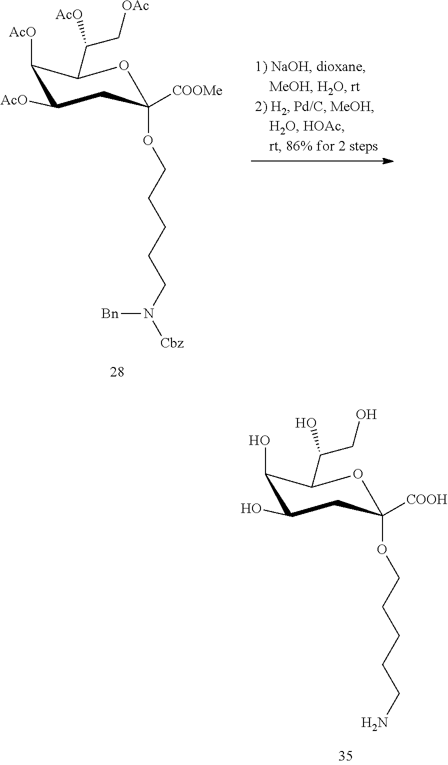

Compound 28 (230 mg, 0.315 mmol) was dissolved in a mixture of dioxane, methanol and 1 M aq NaOH (3/1/1, v/v/v, 12.5 mL). After being stirred at room temperature for overnight, the reaction mixture was diluted with methanol and neutralized with Amberlite IR120 H⁺ resin. After filtration, the filtrate was concentrated in vacuo to give the corresponding monosaccharide as a white solid. A mixture of the above monosaccharide and Pd/C (500 mg, 10%) in methanol, water and acetic acid (50/25/1, v/v/v, 9.12 mL) was stirred under an atmosphere of H₂ at room temperature for 20 h. Filtration, concentration in vacuo and elution through Sephadex LH-20 column (H₂O) provided 35 (87 mg, 86% over 2 steps) as a white solid: [α]²⁰_{D}=+58.0 (c 0.6, H₂O); ¹H NMR (400 MHz, D₂O) δ 4.08 (m, 1H, H-4), 4.01 (m, 1H), 3.94 (m, 2H), 3.64 (dd, J=6.4, 11.6 Hz, 1H), 3.57 (d-like, J=9.2 Hz, 1H), 3.41 (m, 1H, OCH₂), 3.29 (m, 1H, OCH₂), 3.00 (t, J=7.6 Hz, 2H, NCH₂), 2.04 (dd, J=4.8, 12.8 Hz, 1H, H-3e), 1.76 (t, J=12.8 Hz, 1H, H-3a), 1.70-1.58 (m, 4H, CCH₂C), 1.43 (m, 2H, CCH₂C); ¹³C NMR (100 MHz, D₂O) δ 175.4 (C(O)), 99.9 (C-2), 71.2, 69.2, 66.2, 66.0, 62.9, 62.8 (OCH₂), 39.2 (NCH₂), 34.1 (C-3), 28.1, 26.4, 22.3; HRMS (ESI) m/z calcd for C₁₃H₂₄NO₈ [M−H]⁻ 322.1502. found 322.1518.

### 2.17. Synthesis of L-glycero-a-D-manno-heptopyranosyl-(1→5)-2-(5-amino) pentyl-3-deoxy-a-D-manno-oct-2-ulopyranosidonic acid 36

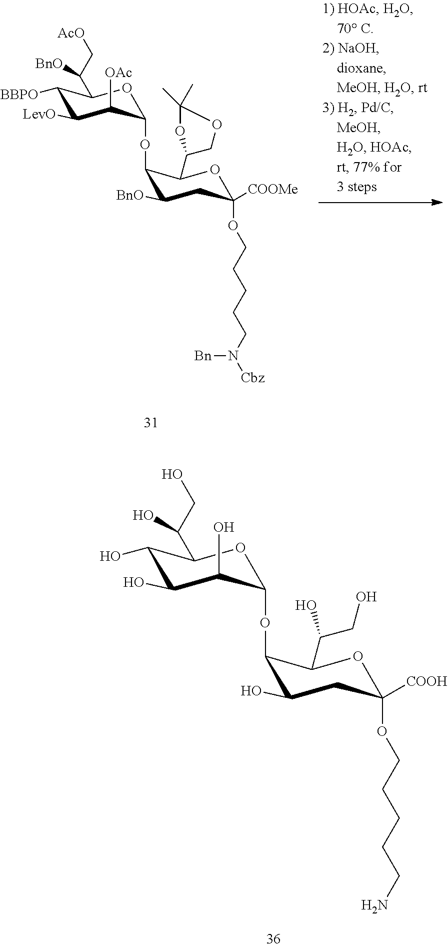

A solution of compound 31 (34 mg, 0.026 mmol) in acetic acid/water (8/1, v/v, 1.80 mL) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v, 1.25 mL). After being stirred at room temperature for overnight, the reaction mixture was diluted with methanol and neutralized with Amberlite IR120 H⁺ resin. After filtration, the filtrate was concentrated in vacuo to give the corresponding disaccharide as a white solid. A mixture of the above tetrasaccharide and Pd/C (70 mg, 10%) in methanol, water and acetic acid (50/25/1, v/v/v, 3.04 mL) was stirred under an atmosphere of H₂ at room temperature for overnight. Filtration, concentration in vacuo and elution through Sephadex LH-20 column (H₂O) provided 36 (10 mg, 77% over 3 steps) as a white solid: [α]²⁰_{D}=+70.6 (c 0.5, H₂O); ¹H NMR (400 MHz, D₂O) δ 5.09 (d, J=1.6 Hz, 1H, H-1), 4.15 (m, 1H, H-4′), 4.10 (m, 2H), 4.03 (m, 1H), 3.94 (m, 3H), 3.88 (m, 1H), 3.85 (m, 1H), 3.70 (m, 3H), 3.62 (m, 1H), 3.42 (m, 1H, OCH₂), 3.29 (m, 1H, OCH₂), 3.01 (t, J=7.6 Hz, 2H, NCH₂), 2.06 (dd, J=4.8, 13.6 Hz, 1H, H-3′e), 1.83 (t, J=12.8 Hz, 1H, H-3′a), 1.70 (m, 2H, CCH₂C), 1.61 (m, 2H, CCH₂C), 1.45 (m, 2H, CCH₂C); ¹³C NMR (100 MHz, D₂O) δ 175.2 (C(O)), 101.5 (C-1), 99.8 (C-2′), 75.2, 71.9, 71.2, 70.3, 70.2, 69.0, 68.8, 66.1, 65.6, 63.1, 63.0, 62.9 (OCH₂), 39.3 (NCH₂), 34.8 (C-3′), 28.1, 26.4, 22.3; HRMS (ESI) m/z calcd for C₂₀H₃₇NO₁₄Na [M+Na]⁺ 538.2112. found 538.2129.

### 2.18. Synthesis of N-Phenyl Trifluoroacetimidate 2-O-acetyl-3,7-di-O-benzoyl-4-O-para-bromobenzyl-6-O-benzyl-L-glycero-D-manno-heptopyranoside 38

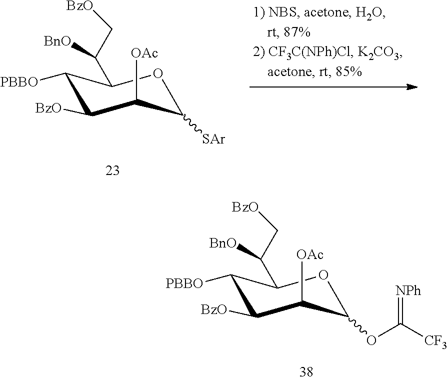

To a solution of compound 23 (100 mg, 0.113 mmol) in acetone/H₂O (10/1, v/v, 3.3 mL), was added NBS (96 mg, 0.54 mmol). After being stirred at room temperature for 2 h, the mixture was diluted with EtOAc, washed with saturated aqueous NaHCO₃ and brine. The organic layer was dried over Na₂SO₄, filtered, and concentrated in vacuo. The residue was purified by silica gel column chromatography (hexane/EtOAc: 5/2) to afford the corresponding hemiacetal (71 mg, 87%) as a colorless syrup. To a solution of the above hemiacetal (71 mg, 0.099 mmol) and K₂CO₃ (38 mg, 0.28 mmol) in acetone (3 mL), was added 2,2,2-trifluoro-N-phenylacetimidoyl chloride (123 mg, 0.59 mmol). After being stirred at room temperature for 30 min, the solution was filtered and concentrated in vacuo to a residue, which was purified by silica gel column chromatography (hexane/EtOAc: 4/1) to afford 38 (75 mg, 85%) as a white solid: [α]²⁰_{D}=+30.2 (c 0.7, CHCl₃); ¹H NMR (400 MHz, CDCl₃) δ 7.96-7.85 (m, 4H, Ar), 7.53-7.16 (m, 15H, Ar), 7.01 (m, 1H, Ar), 6.84-6.73 (m, 4H, Ar), 6.28 (br s, 1H), 5.62 (m, 1H), 5.53 (br s, 1H), 4.84 (d-like, J=12.0 Hz, 1H, OCH₂Ar), 4.72 (dd, J=5.6, 11.2 Hz, 1H), 4.52-4.42 (m, 3H), 4.21 (m, 2H), 4.14 (t-like, J=6.8 Hz, 1H), 4.07 (d-like, J=10.0 Hz, 1H), 2.08 (s, 3H, C(O)CH₃); ¹³C NMR (100 MHz, CDCl₃) δ 169.5, 166.2, 165.2, 143.1, 137.8, 136.4, 133.5, 133.3, 131.4, 129.6, 129.5, 129.3, 129.1, 128.8, 128.6, 128.5, 128.4, 128.3, 128.0, 127.9, 124.5, 121.7, 119.2, 93.8 (C-1), 73.6, 73.5, 73.0, 72.8, 72.5, 71.9, 68.7, 62.2, 20.7; HRMS (ESI) m/z calcd for C₄₅H₃₉BrF₃NO₁₀Na [M+Na]⁺ 912.1607. found 912.1602.

### 2.19. Synthesis of methyl [N-benzyl-benzyloxycarbonyl-5-aminopentyl (2-O-acetyl-3,7-di-O-benzoyl-4-O-para-bromobenzyl-6-O-benzyl-L-glycero-α-D-manno-heptopyranosyl)(1→3)-(2,7-di-O-acetyl-4-O-para-bromobenzyl-6-O-benzyl-L-glycero-α-D-manno-heptopyranosyl)-(1→5)-4-O-benzyl-7,8-O-isopropylidene-3-deoxy-α-D-manno-oct-2-ulopyranosid]onate 39

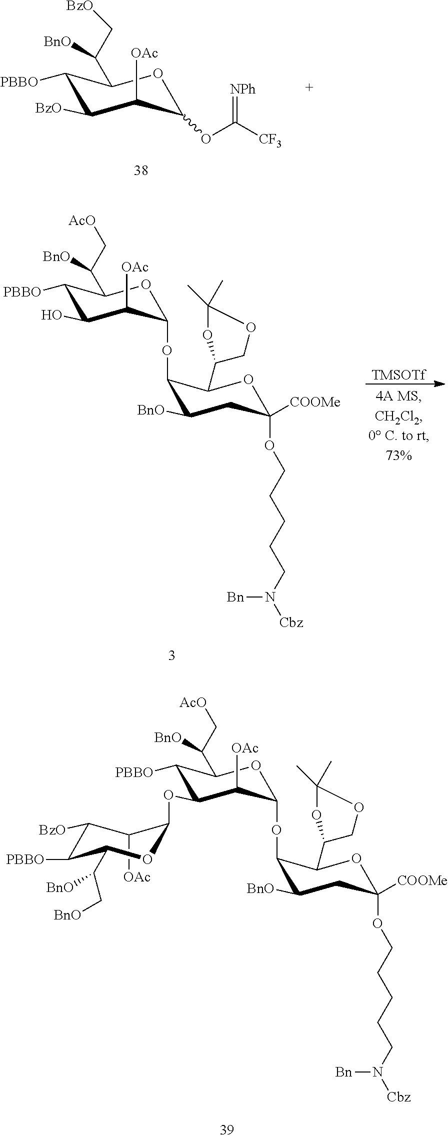

To a stirred mixture of the donor 38 (46 mg, 51.6 μmol), disaccharide acceptor 3 (42 mg, 34.2 μmol), and freshly activated 4 Å MS in dry dichloromethane (4.5 mL) at 0° C., was added TMSOTf in CH₂Cl₂ (0.05 M, 110 μL) under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (toluene/acetone: 20/1) to afford 39 (48 mg, 73%) as a white foam: [α]²⁰_{D}=+8.9 (c 0.4, CHCl₃); ¹H NMR (400 MHz, Pyridine-d₅) δ 8.48-8.15 (m, 6H, Ar), 7.77-7.05 (m, 37H, Ar), 6.14 (dd, J=3.2, 8.8 Hz, 1H), 6.03 (br s, 1H), 5.95 (t, J=2.8 Hz, 1H), 5.85 (s, 1H, H-1), 5.72 (d, J=2.0 Hz, 1H, H-1′), 5.44 (m, 3H), 5.30 (d-like, J=12.0 Hz, 1H), 5.11-4.62 (m, 18H), 4.55 (m, 3H), 4.38 (d-like, J=11.2 Hz, 1H), 4.15 (m, 2H), 4.04 (m, 2H), 3.84 (s, 3H, C(O)OCH₃), 3.77 (m, 1H), 3.46-3.29 (m, 4H), 2.63 (m, 1H, H-3″e), 2.35 (t, J=12.0 Hz, 1H, H-3″a), 2.14 (s, 3H, C(O)CH₃), 2.00 (s, 3H, C(O)CH₃), 1.99 (s, 3H, C(O)CH₃), 1.56 (m, 4H, CCH₂C), 1.42 (s, 3H, C(CH₃)₂), 1.35 (s, 3H, C(CH₃)₂), 1.30 (m, 2H, CCH₂C); ¹³C NMR (100 MHz, Pyridine-d₅) δ 171.1 (C(O)), 171.0 (C(O)), 170.3 (C(O)), 169.1 (C(O)), 167.1 (C(O)), 166.3 (C(O)), 140.2, 139.7, 139.4, 139.0, 138.7, 138.3, 134.2, 134.0, 132.2, 132.1, 132.0, 131.9, 131.2, 130.8, 130.6, 130.5, 130.4, 130.3, 130.1, 130.0, 129.9, 129.6, 129.5, 129.4, 129.3, 129.2, 129.1, 128.7, 128.6, 128.5, 128.4, 128.3, 128.2, 128.1, 122.1, 121.9, 110.4 (C(CH₃)₂), 100.6 (C-1′, J_{C,H}=176.4 Hz), 99.9 (C-2″), 98.1 (C-1, J_{C,H}=176.0 Hz), 77.1, 76.5, 76.2, 76.0, 75.3, 74.7, 74.6, 73.6, 73.5, 73.4, 73.3, 73.1, 72.9, 72.8, 72.3, 71.2, 71.0, 68.6, 67.7, 67.3, 64.6, 64.4, 52.9 (C(O)OCH₃), 32.9 (C-3″), 30.0, 27.4, 25.6, 24.1, 21.4 (C(O)CH₃), 21.2 (C(O)CH₃), 20.9 (C(O)CH₃); HRMS (ESI) m/z calcd for C₁₀₁H₁₀₉Br₂NO₂₇Na [M+Na]⁺ 1948.5451. found 1948.5485.

### 2.20. Synthesis of L-glycero-α-D-manno-heptopyranosyl-(1→3)-L-glycero-α-D-manno-heptopyranosyl-(1→5)-2-(5-amino)pentyl-3-deoxy-α-D-manno-oct-2-ulopyranosidonic acid 37

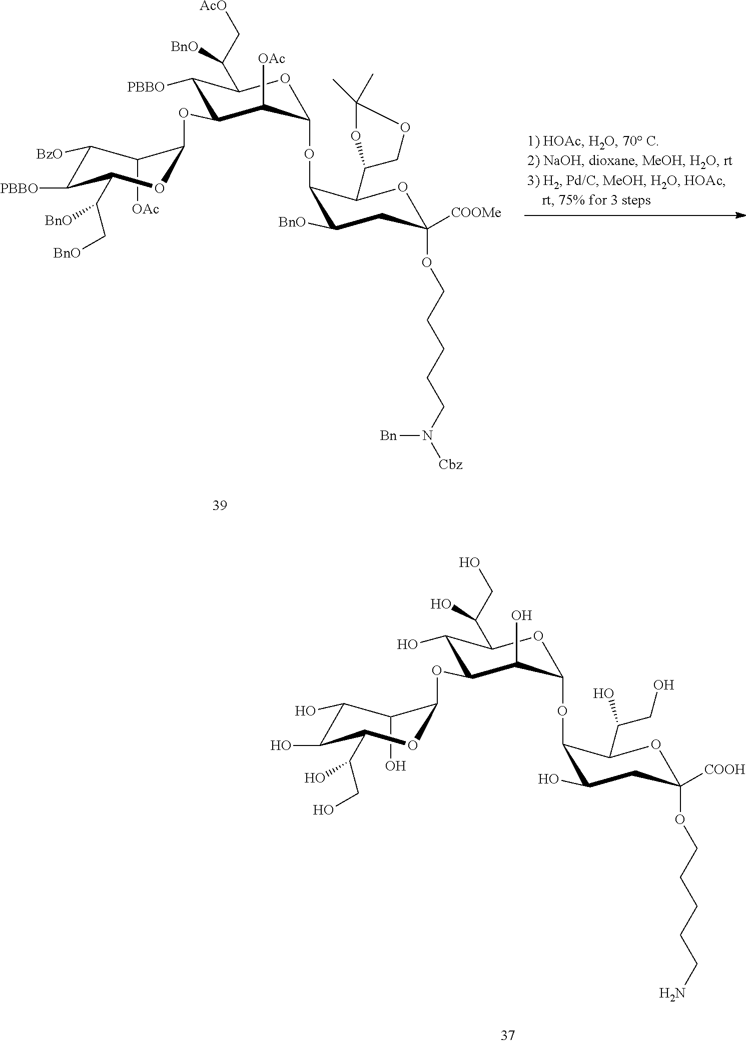

A solution of compound 39 (44 mg, 0.023 mmol) in acetic acid/water (8/1, v/v, 2.25 mL) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v, 1.66 mL). After being stirred at room temperature for overnight, the reaction mixture was diluted with methanol and neutralized with Amberlite IR120 H⁺ resin. After filtration, the filtrate was concentrated in vacuo to give the corresponding trisaccharide as a white solid. A mixture of the above trisaccharide and Pd/C (100 mg, 10%) in methanol, water and acetic acid (88/24/1, v/v/v, 5.65 mL) was stirred under an atmosphere of H₂ at room temperature for 24 h. Filtration, concentration in vacuo and elution through Sephadex LH-20 column (H₂O) provided 37 (12 mg, 75% for 3 steps) as a white solid: [α]²⁰_{D}=+92.2 (c 0.6, H₂O); ¹H NMR (400 MHz, D₂O) δ 5.22 (s, 1H, H-1), 5.09 (s, 1H, H-1′), 4.16 (m, 2H, H-4″), 4.10-3.90 (m, 9H), 3.71-3.65 (m, 9H), 3.46 (m, 1H, OCH₂), 3.30 (m, 1H, OCH₂), 3.01 (t, J=7.6 Hz, 2H, NCH₂), 2.10 (dd, J=4.4, 12.8 Hz, 1H, H-3″e), 1.85 (t, J=12.8 Hz, 1H, H-3″a), 1.70 (m, 2H, CCH₂C), 1.63 (m, 2H, CCH₂C), 1.43 (m, 2H, CCH₂C); ¹³C NMR (100 MHz, D₂O) δ 175.0 (C(O)), 101.7 (C-1), 101.3 (C-1′), 99.7 (C-2″), 76.5, 75.0, 71.9, 71.6, 71.3, 70.5, 70.0, 69.1, 68.7, 68.4, 66.0, 65.9, 65.6, 63.2, 63.0, 62.9 (OCH₂), 39.3 (NCH₂), 34.8 (C-3″), 28.1, 26.4, 22.3; HRMS (ESI) m/z calcd for C₂₇H₄₉NO₂₀Na [M+Na]⁺ 730.2746. found 730.2742.

### 2.21. Synthesis of methyl (N-benzyl-benzyloxycarbonyl-2-aminoethyl 4,5,7,8-tetra-O-acetyl-3-deoxy-α-D-manno-oct-2-ulopyranosid)onate 41

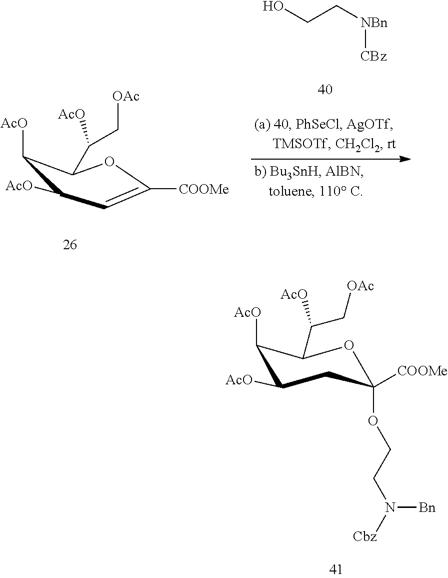

To a stirred solution of phenylselenyl chloride (2.73 g, 14.3 mmol) in CH₂Cl₂ (50 mL) was added AgOTf (2.55 g, 9.92 mmol) and TMSOTf (0.16 mL, 0.86 mmol). After stirring at room temperature for 30 min, a solution of glycal 26 (2.85 g, 7.08 mmol) and linker 40 (2.83 g, 9.92 mmol) in CH₂Cl₂ (70 mL) was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene (140 mL), was added tri-n-butyltin hydride (3.8 mL, 14.24 mmol) and AIBN (779 mg, 4.75 mmol). After being refluxed for 1.5 h, the mixture was concentrated in vacuo and purified by silica gel column chromatography (hexane/EtOAc: 5/2) to afford 41 as a colorless syrup. HRMS (ESI) m/z calcd for C₃₄H₄₁NO₁₄Na [M+Na]⁺ 710.2425.

### 2.22. Synthesis of 2-(2-amino)ethyl-3-deoxy-a-D-manno-oct-2-ulopyranosidonic acid 42

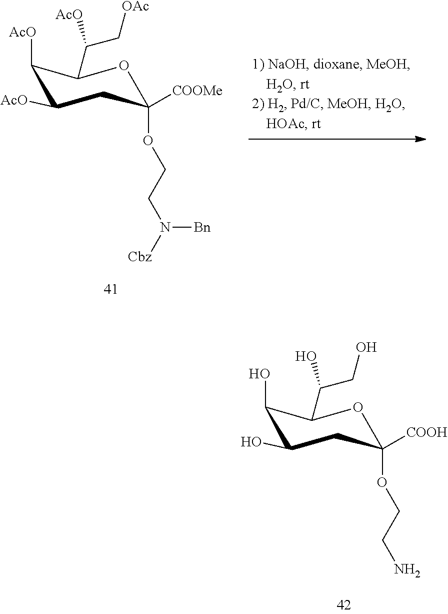

Compound 41 (216 mg, 0.315 mmol) was dissolved in a mixture of dioxane, methanol and 1 M aq NaOH (3/1/1, v/v/v, 12.5 mL). After being stirred at room temperature for overnight, the reaction mixture was diluted with methanol and neutralized with Amberlite IR120 H⁺ resin. After filtration, the filtrate was concentrated in vacuo to give the corresponding monosaccharide as a white solid. A mixture of the above monosaccharide and Pd/C (500 mg, 10%) in methanol, water and acetic acid (50/25/1, v/v/v, 9.12 mL) was stirred under an atmosphere of H₂ at room temperature for 20 h. Filtration, concentration in vacuo and elution through Sephadex LH-20 column (H₂O) provided 42 as a white solid: HRMS (ESI) m/z calcd for C₁₀H₁₈NO₈ [M−H]⁻ 280.1032.

### 2.23. Synthesis of methyl (N-benzyl-benzyloxycarbonyl-22-aminodocosanyl 4,5,7,8-tetra-O-acetyl-3-deoxy-α-D-manno-oct-2-ulopyranosid)onate 44

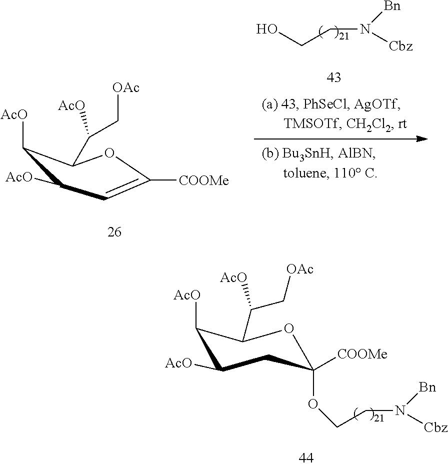

To a stirred solution of phenylselenyl chloride (2.73 g, 14.3 mmol) in CH₂Cl₂ (50 mL) was added AgOTf (2.55 g, 9.92 mmol) and TMSOTf (0.16 mL, 0.86 mmol). After stirring at room temperature for 30 min, a solution of glycal 26 (2.85 g, 7.08 mmol) and linker 43 (5.60 g, 9.92 mmol) in CH₂Cl₂ (70 mL) was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene (140 mL), was added tri-n-butyltin hydride (3.8 mL, 14.24 mmol) and AIBN (779 mg, 4.75 mmol). After being refluxed for 1.5 h, the mixture was concentrated in vacuo and purified by silica gel column chromatography (hexane/EtOAc: 5/2) to afford 44 as a colorless syrup. HRMS (ESI) m/z calcd for C₅₄H₈₁NO₁₄Na [M+Na]⁺ 990.5555.

### 2.24. Synthesis of 2-(22-amino)docosanyl-3-deoxy-a-D-manno-oct-2-ulopyranosidonic acid 45

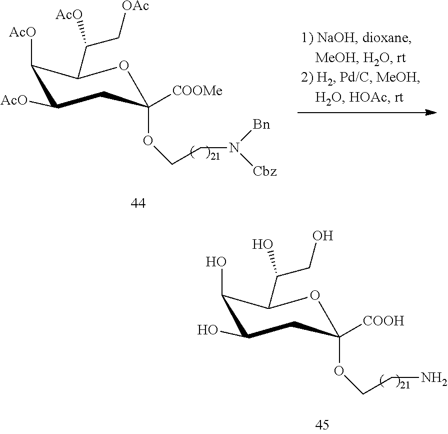

Compound 44 (305 mg, 0.315 mmol) was dissolved in a mixture of dioxane, methanol and 1 M aq NaOH (3/1/1, v/v/v, 12.5 mL). After being stirred at room temperature for overnight, the reaction mixture was diluted with methanol and neutralized with Amberlite IR120 H⁺ resin. After filtration, the filtrate was concentrated in vacuo to give the corresponding monosaccharide as a white solid. A mixture of the above monosaccharide and Pd/C (500 mg, 10%) in methanol, water and acetic acid (50/25/1, v/v/v, 9.12 mL) was stirred under an atmosphere of H₂ at room temperature for 20 h. Filtration, concentration in vacuo and elution through Sephadex LH-20 column (H₂O) provided 45 as a white solid: HRMS (ESI) m/z calcd for C₃₀H₅₈NO₈ [M−H]⁻ 560.4162.

### 2.25. Synthesis of methyl [2-(N-benzyl-benzyloxycarbonyl-2-aminoethoxy)ethyl 4,5,7,8-tetra-O-acetyl-3-deoxy-α-D-manno-oct-2-ulopyranosid]onate 47

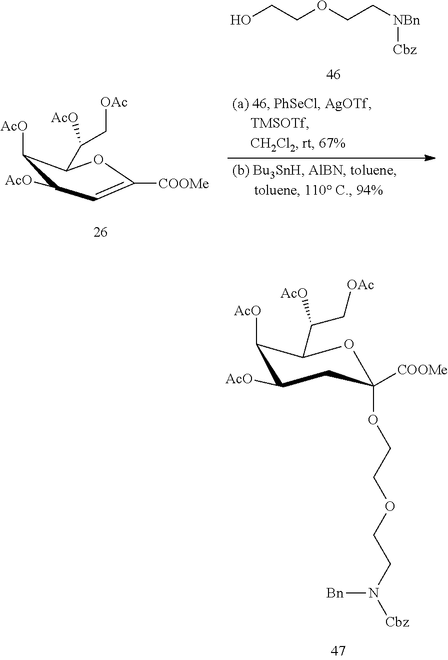

To a stirred solution of phenylselenyl chloride (2.73 g, 14.3 mmol) in CH₂Cl₂ (50 mL) was added AgOTf (2.55 g, 9.92 mmol) and TMSOTf (0.16 mL, 0.86 mmol). After stirring at room temperature for 30 min, a solution of glycal 26 (2.85 g, 7.08 mmol) and linker 46 (3.26 g, 9.92 mmol) in CH₂Cl₂ (70 mL) was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene (140 mL), was added tri-n-butyltin hydride (3.8 mL, 14.24 mmol) and AIBN (779 mg, 4.75 mmol). After being refluxed for 1.5 h, the mixture was concentrated in vacuo and purified by silica gel column chromatography (hexane/EtOAc: 5/2) to afford 47 as a colorless syrup. HRMS (ESI) m/z calcd for C₃₆H₄₅NO₁₅Na [M+Na]⁺ 754.2687.

### 2.26. Synthesis of 2-2-(2-aminoethoxy)ethyl-3-deoxy-a-D-manno-oct-2-ulpyrano sidonic acid 48

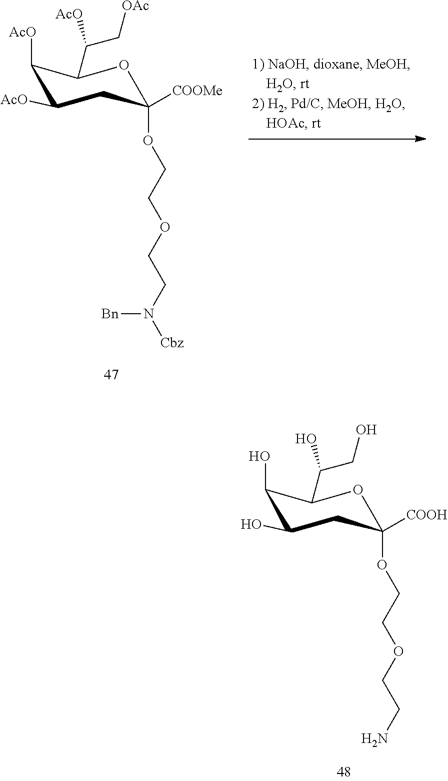

Compound 47 (230 mg, 0.315 mmol) was dissolved in a mixture of dioxane, methanol and 1 M aq NaOH (3/1/1, v/v/v, 12.5 mL). After being stirred at room temperature for overnight, the reaction mixture was diluted with methanol and neutralized with Amberlite IR120 H⁺ resin. After filtration, the filtrate was concentrated in vacuo to give the corresponding monosaccharide as a white solid. A mixture of the above monosaccharide and Pd/C (500 mg, 10%) in methanol, water and acetic acid (50/25/1, v/v/v, 9.12 mL) was stirred under an atmosphere of H₂ at room temperature for 20 h. Filtration, concentration in vacuo and elution through Sephadex LH-20 column (H₂O) provided 48 as a white solid: HRMS (ESI) m/z calcd for C₁₂H₂₂NO₉ [M−H]⁻ 324.1295.

### 2.27. Synthesis of methyl [2-(5-N-benzyl-benzyloxycarbonyl-aminomethyl)pyrrolidin-2-yl)ethyl 4,5,7,8-tetra-O-acetyl-3-deoxy-a-D-manno-oct-2-ulopyra nosid]onate 50

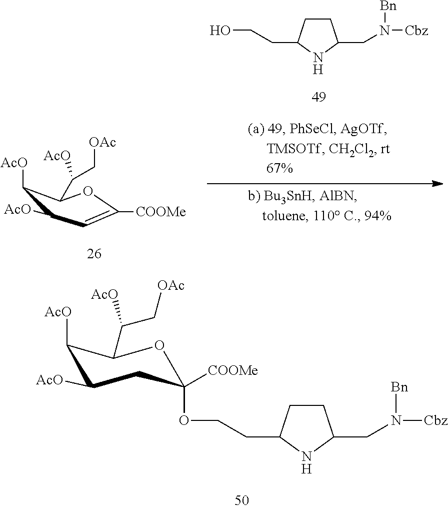

To a stirred solution of phenylselenyl chloride (2.73 g, 14.3 mmol) in CH₂Cl₂ (50 mL) was added AgOTf (2.55 g, 9.92 mmol) and TMSOTf (0.16 mL, 0.86 mmol). After stirring at room temperature for 30 min, a solution of glycal 26 (2.85 g, 7.08 mmol) and linker 49 (3.65 g, 9.92 mmol) in CH₂Cl₂ (70 mL) was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene (140 mL), was added tri-n-butyltin hydride (3.8 mL, 14.24 mmol) and AIBN (779 mg, 4.75 mmol). After being refluxed for 1.5 h, the mixture was concentrated in vacuo and purified by silica gel column chromatography (hexane/EtOAc: 5/2) to afford 50 as a colorless syrup. HRMS (ESI) m/z calcd for C₃₉H₅₀N₂O₁₄Na [M+Na]⁺ 793.3160.

### 2.28. Synthesis of 2-2-(5-aminomethyl)pyrrolidin-2-yl)ethyl-3-deoxy-a-D-man no-oct-2-ulpyranosidonic acid 51

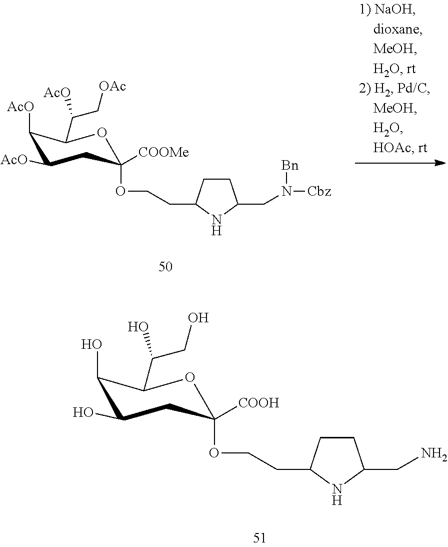

Compound 50 (243 mg, 0.315 mmol) was dissolved in a mixture of dioxane, methanol and 1 M aq NaOH (3/1/1, v/v/v, 12.5 mL). After being stirred at room temperature for overnight, the reaction mixture was diluted with methanol and neutralized with Amberlite IR120 H⁺ resin. After filtration, the filtrate was concentrated in vacuo to give the corresponding monosaccharide as a white solid. A mixture of the above monosaccharide and Pd/C (500 mg, 10%) in methanol, water and acetic acid (50/25/1, v/v/v, 9.12 mL) was stirred under an atmosphere of H₂ at room temperature for 20 h. Filtration, concentration in vacuo and elution through Sephadex LH-20 column (H₂O) provided 51 as a white solid: HRMS (ESI) m/z calcd for C₁₅H₂₇N₂O₈ [M−H]⁻ 363.1767.

### 2.29. Synthesis of methyl [4-(N-benzyl-benzyloxycarbonyl-2-aminoethoxy)benzyl 4,5,7,8-tetra-O-acetyl-3-deoxy-α-D-manno-oct-2-ulo pyranosid]onate 53

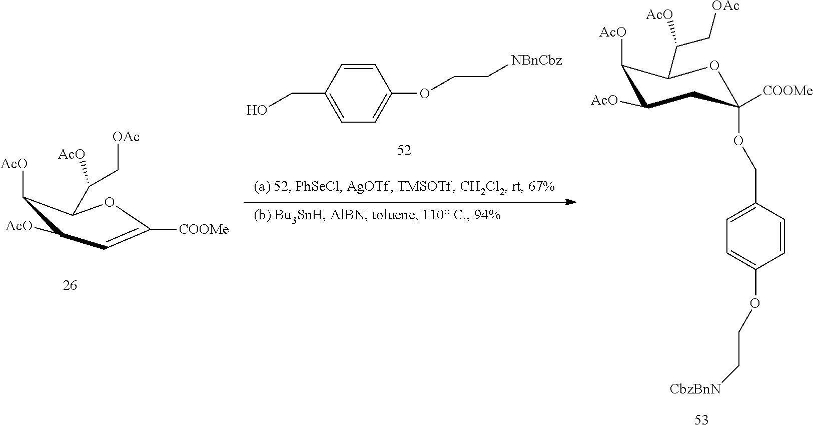

To a stirred solution of phenylselenyl chloride (2.73 g, 14.3 mmol) in CH₂Cl₂ (50 mL) was added AgOTf (2.55 g, 9.92 mmol) and TMSOTf (0.16 mL, 0.86 mmol). After stirring at room temperature for 30 min, a solution of glycal 26 (2.85 g, 7.08 mmol) and linker 52 (3.88 g, 9.92 mmol) in CH₂Cl₂ (70 mL) was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene (140 mL), was added tri-n-butyltin hydride (3.8 mL, 14.24 mmol) and AIBN (779 mg, 4.75 mmol). After being refluxed for 1.5 h, the mixture was concentrated in vacuo and purified by silica gel column chromatography (hexane/EtOAc: 5/2) to afford 53 as a colorless syrup. HRMS (ESI) m/z calcd for C₄₁H₄₇NO₁₅Na [M+Na]⁺ 816.2843.

### 2.30. Synthesis of 2-4-(2-aminoethoxy)benzyl-3-deoxy-a-D-manno-oct-2-ulpyranosidonic acid 54

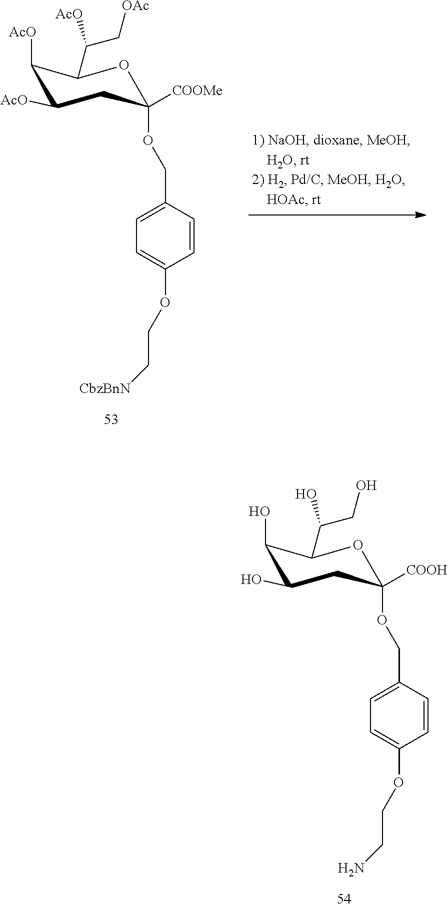

Compound 53 (250 mg, 0.315 mmol) was dissolved in a mixture of dioxane, methanol and 1 M aq NaOH (3/1/1, v/v/v, 12.5 mL). After being stirred at room temperature for overnight, the reaction mixture was diluted with methanol and neutralized with Amberlite IR120 H⁺ resin. After filtration, the filtrate was concentrated in vacuo to give the corresponding monosaccharide as a white solid. A mixture of the above monosaccharide and Pd/C (500 mg, 10%) in methanol, water and acetic acid (50/25/1, v/v/v, 9.12 mL) was stirred under an atmosphere of H₂ at room temperature for 20 h. Filtration, concentration in vacuo and elution through Sephadex LH-20 column (H₂O) provided 54 as a white solid: HRMS (ESI) m/z calcd for C₁₇H₂₄NO₉ [M−H]⁻ 386.1451.

### 2.31. Synthesis of L-glycero-a-D-manno-heptopyranosyl-(1→5)-2-(38-amino-3,6,9,12,15,18,21,24,27,30,33,36-dodecaoxaoctatriacontanyl)-3-deoxy-a-D-manno-oct-2-ulopyranosidonic acid 59

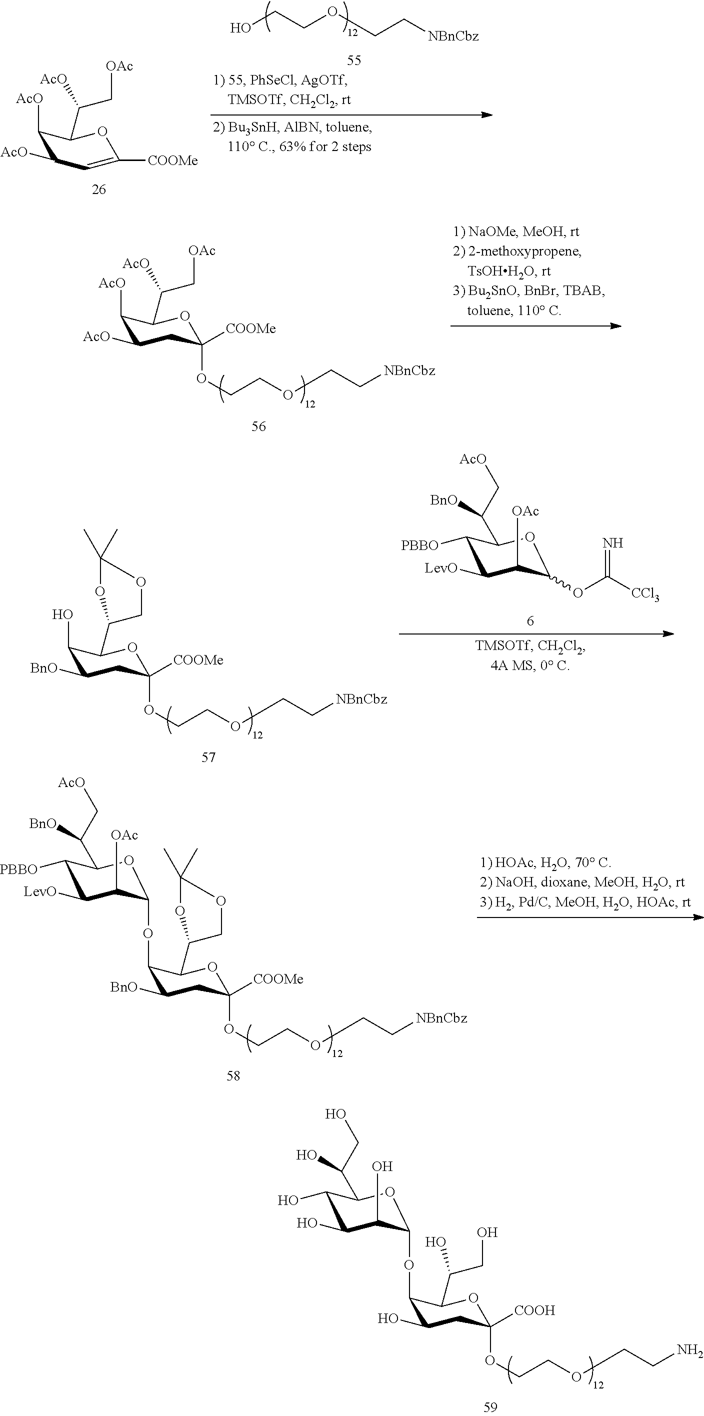

To a stirred solution of phenylselenyl chloride in CH₂Cl₂ was added AgOTf and TMSOTf. After stirring at room temperature for 30 min, a solution of glycal 26 and linker 55 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 56 as a colorless syrup.

To a stirred solution of compound 56 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 57 as a colorless syrup.

To a stirred mixture of the donor 6, acceptor 57, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 58 as a colorless syrup.

A solution of compound 58 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding disaccharide as a white solid. A mixture of the above disaccharide and Pd/C in methanol, water and acetic acid (50/25/1, v/v/v) was stirred under an atmosphere of H₂ at room temperature for overnight. Filtration, concentration in vacuo and elution through Sephadex LH-20 column (H₂O) provided 59 as a white solid. HRMS (ESI) m/z calcd for C₄₁H₇₉NO₂₆Na [M+Na]⁺ 1024.4788.

### 2.32. Synthesis of L-glycero-a-D-manno-heptopyranosyl-(1→5)-2-(((aminomethyl)disulfanyl)methyl)-3-deoxy-a-D-manno-oct-2-ulopyranosidonic acid 64

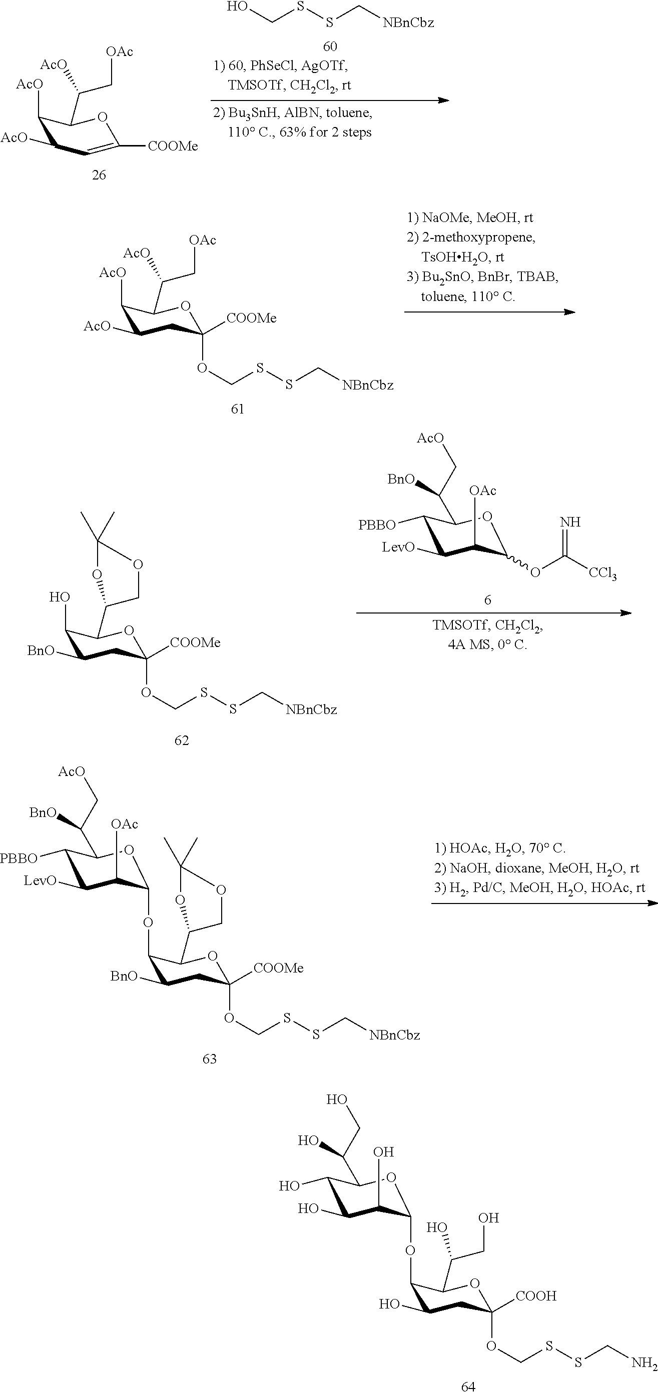

To a stirred solution of phenylselenyl chloride in CH₂Cl₂ was added AgOTf and TMSOTf. After stirring at room temperature for 30 min, a solution of glycal 26 and linker 60 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 61 as a colorless syrup.

To a stirred solution of compound 61 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 62 as a colorless syrup.

To a stirred mixture of the donor 6, acceptor 62, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 63 as a colorless syrup.

A solution of compound 63 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding disaccharide as a white solid. A mixture of the above disaccharide and Pd/C in methanol, water and acetic acid (50/25/1, v/v/v) was stirred under an atmosphere of H₂ at room temperature for overnight. Filtration, concentration in vacuo and elution through Sephadex LH-20 column (H₂O) provided 64 as a white solid. HRMS (ESI) m/z calcd for C₁₇H₃₁NO₁₄S₂Na [M+Na]⁺ 560.1084.

### 2.33. Synthesis of L-glycero-a-D-manno-heptopyranosyl-(1→5)-2-(2-((2-aminoethyl)(methyl)amino)ethyl)-3-deoxy-a-D-manno-oct-2-ulopyranosidonic acid 69

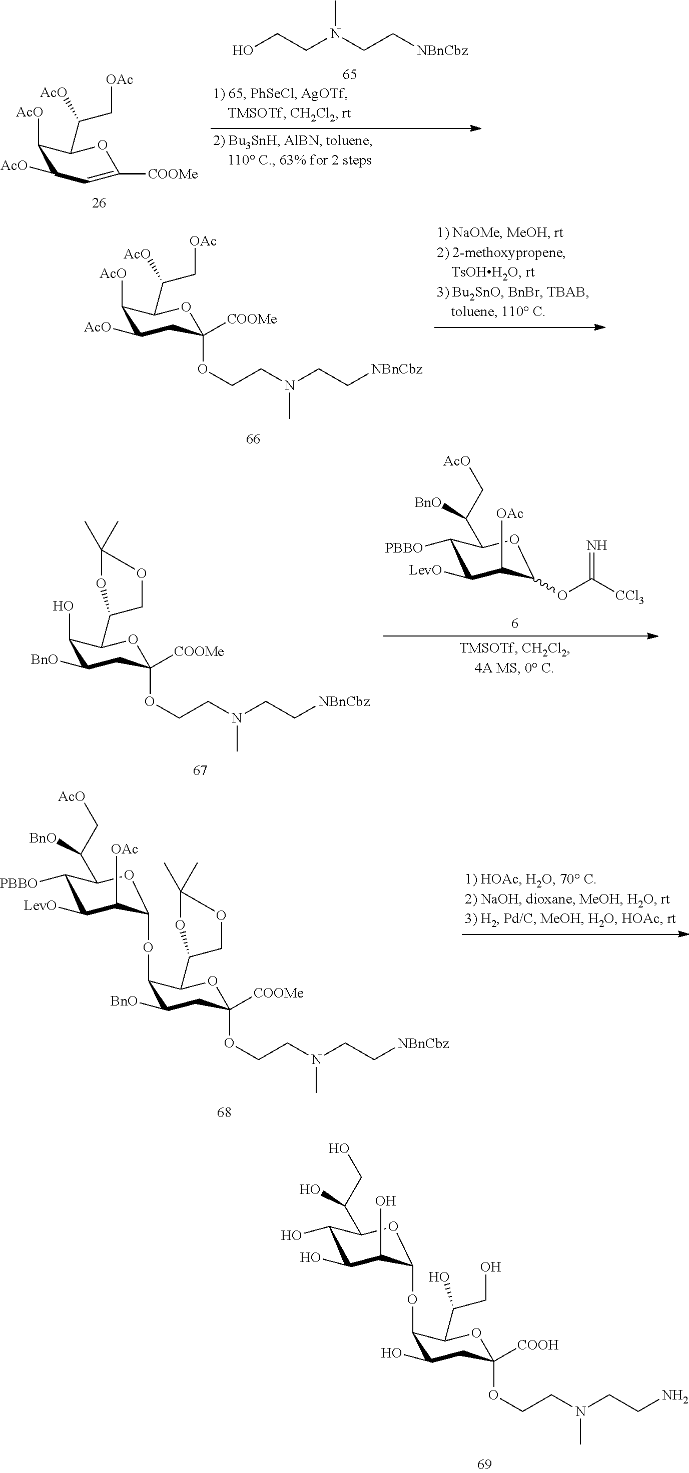

To a stirred solution of phenylselenyl chloride in CH₂Cl₂ was added AgOTf and TMSOTf. After stirring at room temperature for 30 min, a solution of glycal 26 and linker 65 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 66 as a colorless syrup.

To a stirred solution of compound 66 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 67 as a colorless syrup.

To a stirred mixture of the donor 6, acceptor 67, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 68 as a colorless syrup.

A solution of compound 68 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding disaccharide as a white solid. A mixture of the above disaccharide and Pd/C in methanol, water and acetic acid (50/25/1, v/v/v) was stirred under an atmosphere of H₂ at room temperature for overnight. Filtration, concentration in vacuo and elution through Sephadex LH-20 column (H₂O) provided 69 as a white solid. HRMS (ESI) m/z calcd for C₂₀H₃₈N₂O₁₄Na [M+Na]⁺ 553.2221.

### 2.34. Synthesis of L-glycero-a-D-manno-heptopyranosyl-(1→5)-2-(2-(aminomethoxy)phenoxy)methyl-3-deoxy-a-D-manno-oct-2-ulopyranosidonic acid 74

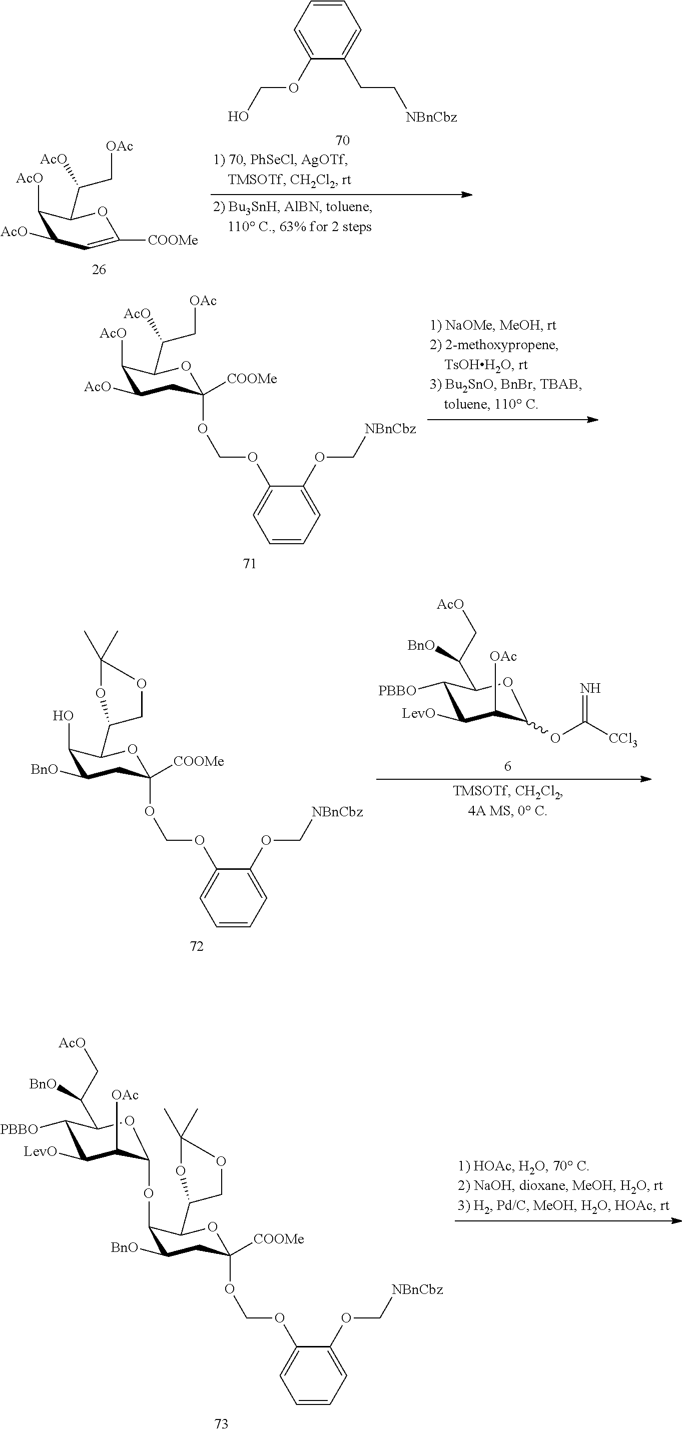

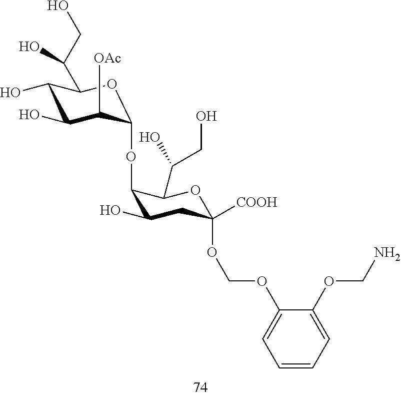

To a stirred solution of phenylselenyl chloride in CH₂Cl₂ was added AgOTf and TMSOTf. After stirring at room temperature for 30 min, a solution of glycal 26 and linker 70 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 71 as a colorless syrup.

To a stirred solution of compound 71 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 72 as a colorless syrup.

To a stirred mixture of the donor 6, acceptor 72, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 73 as a colorless syrup.

A solution of compound 73 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding disaccharide as a white solid. A mixture of the above disaccharide and Pd/C in methanol, water and acetic acid (50/25/1, v/v/v) was stirred under an atmosphere of H₂ at room temperature for overnight. Filtration, concentration in vacuo and elution through Sephadex LH-20 column (H₂O) provided 74 as a white solid. HRMS (ESI) m/z calcd for C₂₃H₃₅NO₁₆Na [M+Na]⁺ 604.1854.

### 2.35. Synthesis of L-glycero-a-D-manno-heptopyranosyl-(1→5)-2-(3-(6-(aminomethyl)piperidin-2-yl)propanyl)-3-deoxy-a-D-manno-oct-2-ulopyranosidonic acid 79

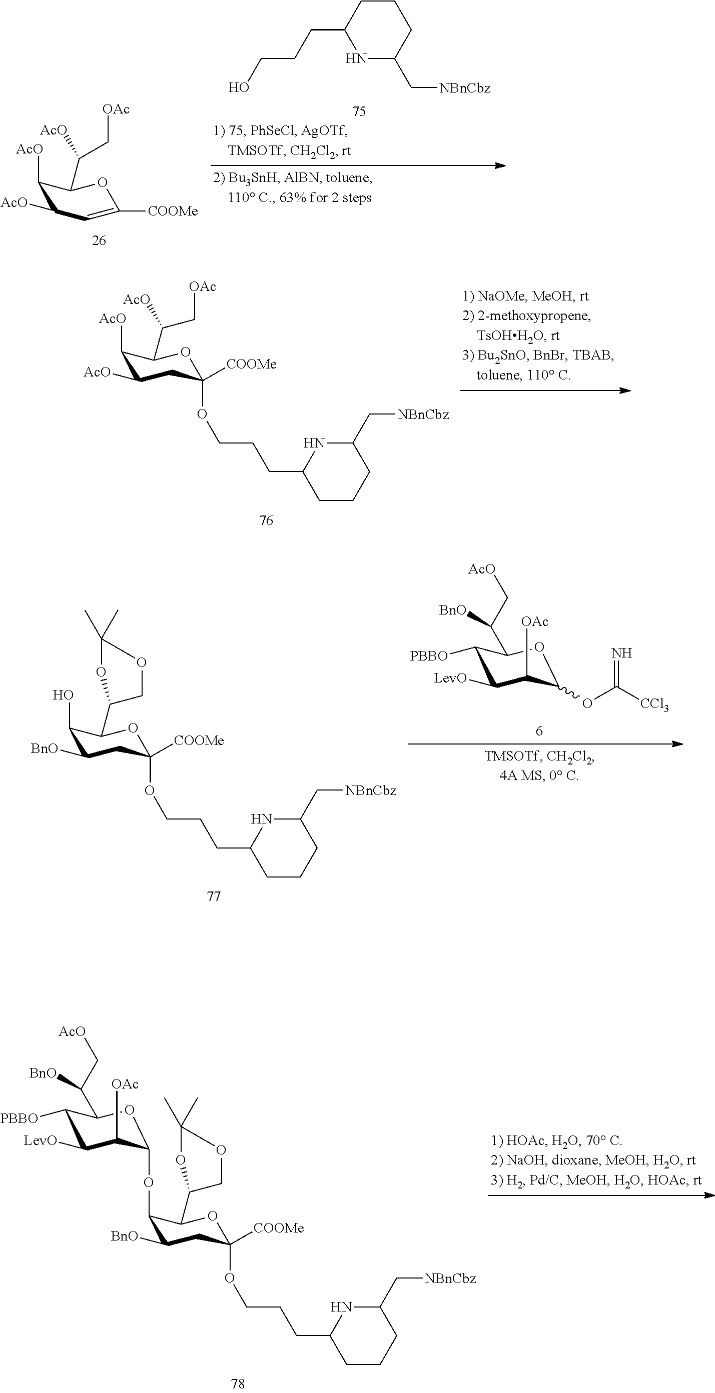

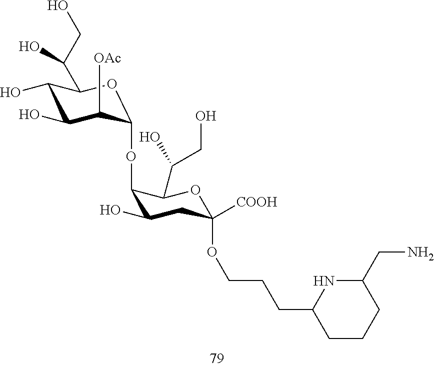

To a stirred solution of phenylselenyl chloride in CH₂Cl₂ was added AgOTf and TMSOTf. After stirring at room temperature for 30 min, a solution of glycal 26 and linker 75 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 76 as a colorless syrup.

To a stirred solution of compound 76 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 77 as a colorless syrup.

To a stirred mixture of the donor 6, acceptor 77, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 78 as a colorless syrup.

A solution of compound 78 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding disaccharide as a white solid. A mixture of the above disaccharide and Pd/C in methanol, water and acetic acid (50/25/1, v/v/v) was stirred under an atmosphere of H₂ at room temperature for overnight. Filtration, concentration in vacuo and elution through Sephadex LH-20 column (H₂O) provided 79 as a white solid. HRMS (ESI) m/z calcd for C₂₄H₄₄N₂O₁₄Na [M+Na]⁺ 607.2690.

### 2.36. Synthesis of L-glycero-α-D-manno-heptopyranosyl-(1→3)-L-glycero-α-D-manno-heptopyranosyl-(1→5)-2-(40-amino)tetracontanyl-3-deoxy-α-D-manno-oct-2-ulopyranosidonic acid 86

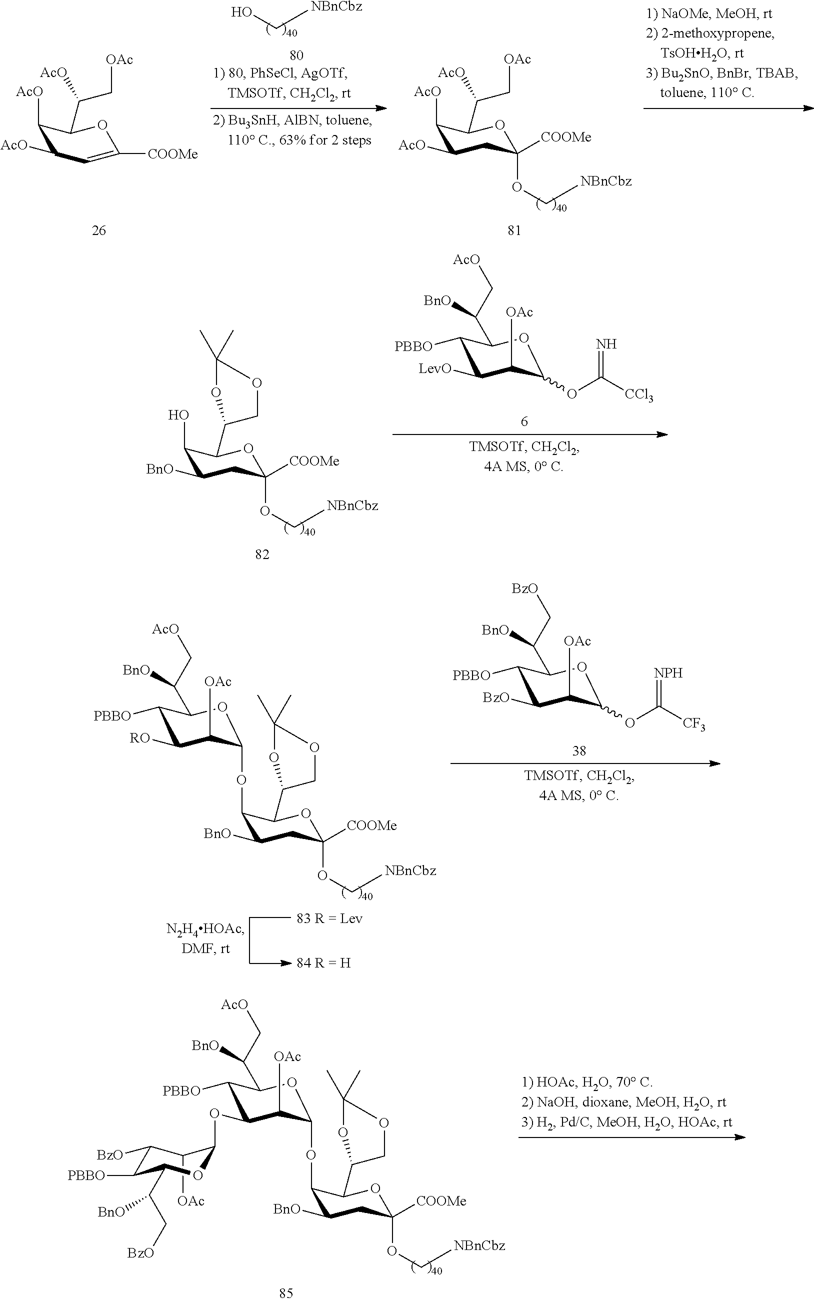

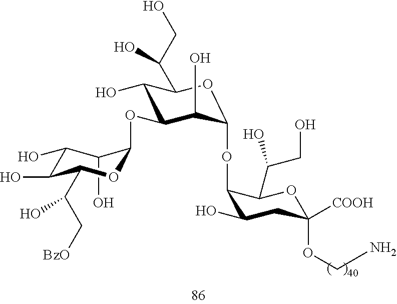

To a stirred solution of phenylselenyl chloride in CH₂Cl₂ was added AgOTf and TMSOTf. After stirring at room temperature for 30 min, a solution of glycal 26 and linker 80 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 81 as a colorless syrup.

To a stirred solution of compound 81 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 82 as a colorless syrup.

To a stirred mixture of the donor 6, acceptor 82, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 83 as a colorless syrup.

To a solution of 83 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 84 as a colorless syrup. To a stirred mixture of the donor 38, disaccharide acceptor 84, and freshly activated 4 Å MS in dry dichloromethane at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (toluene/acetone: 20/1) to afford 85 as a white foam.

A solution of compound 85 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding trisaccharide as a white solid. A mixture of the above trisaccharide and Pd/C in methanol, water and acetic acid (88/24/1, v/v/v) was stirred under an atmosphere of H₂ at room temperature for 24 h. Filtration, concentration in vacuo and elution through Sephadex LH-20 column (H₂O) provided 86 as a white solid. HRMS (ESI) m/z calcd for C₆₂H₁₁₉NO₂₀Na [M+Na]⁺ 1220.8223.

### 2.37. Synthesis of L-glycero-α-D-manno-heptopyranosyl-(1→3)-L-glycero-α-D-manno-heptopyranosyl-(1→5)-2-(2-(4-aminophenoxy)ethyl-3-deoxy-α-D-manno-oct-2-ulopyranosidonic acid 93

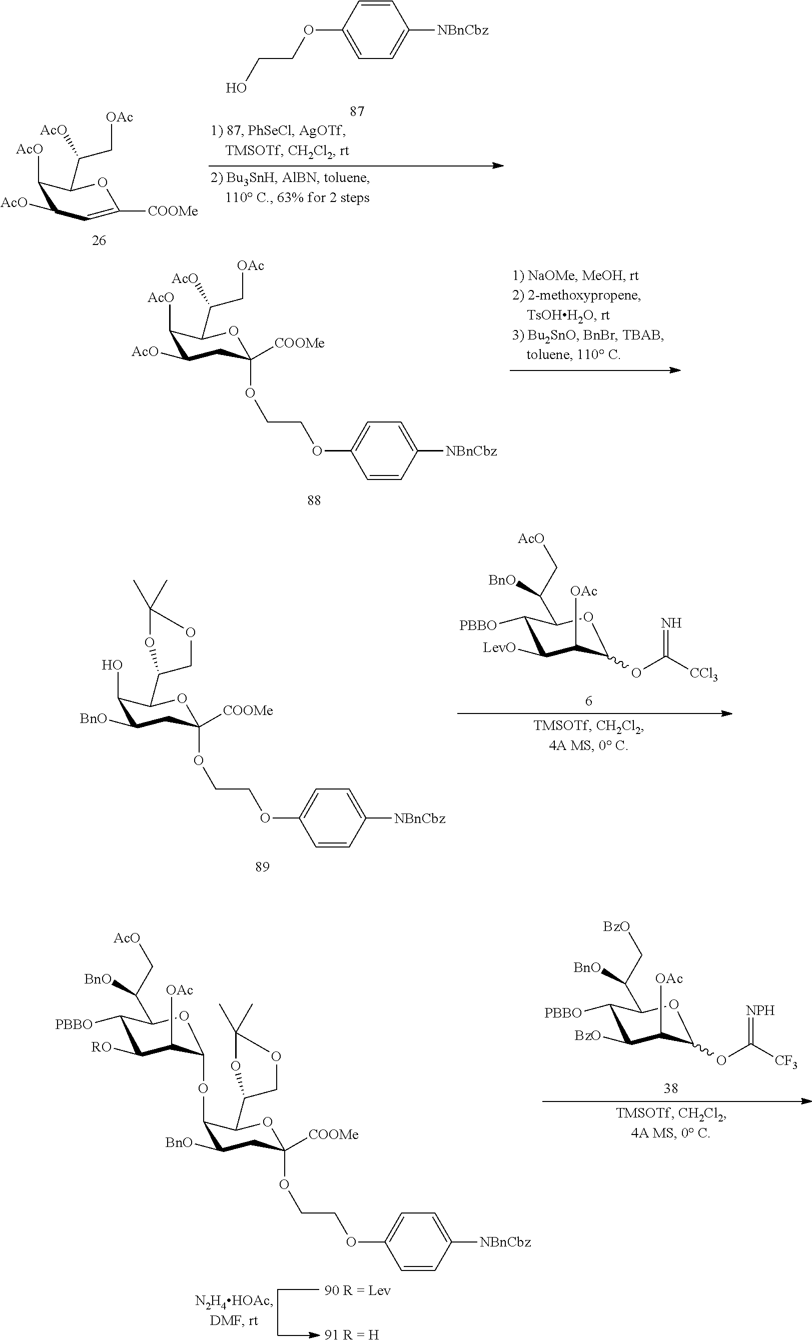

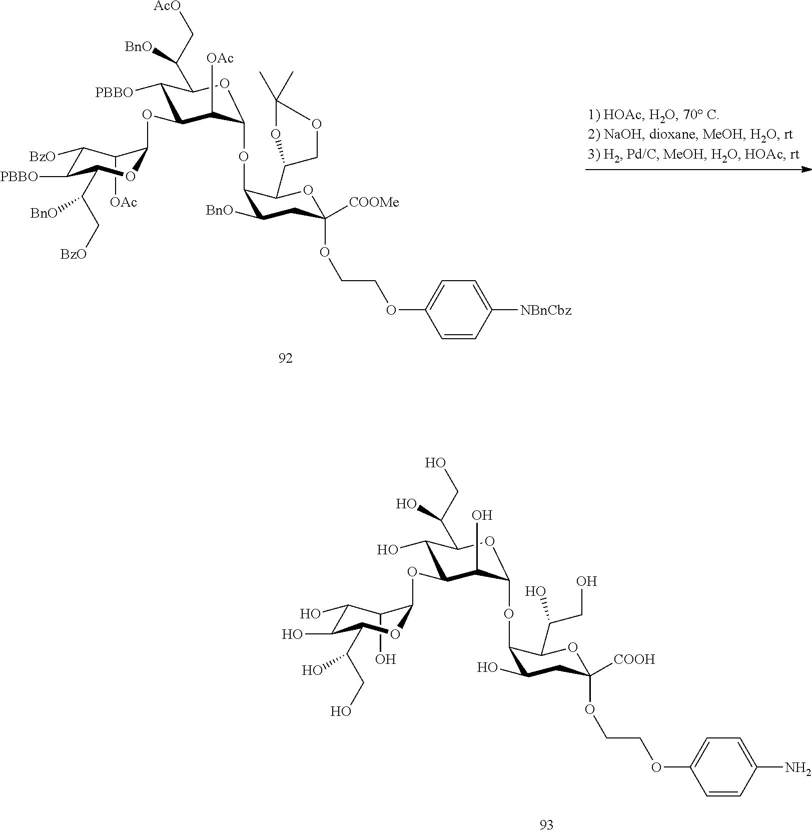

To a stirred solution of phenylselenyl chloride in CH₂Cl₂ was added AgOTf and TMSOTf. After stirring at room temperature for 30 min, a solution of glycal 26 and linker 87 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 88 as a colorless syrup.

To a stirred solution of compound 88 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 89 as a colorless syrup.

To a stirred mixture of the donor 6, acceptor 89, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 90 as a colorless syrup.

To a solution of 90 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 91 as a colorless syrup. To a stirred mixture of the donor 38, disaccharide acceptor 91, and freshly activated 4 Å MS in dry dichloromethane at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (toluene/acetone: 20/1) to afford 92 as a white foam.

A solution of compound 92 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding trisaccharide as a white solid. A mixture of the above trisaccharide and Pd/C in methanol, water and acetic acid (88/24/1, v/v/v) was stirred under an atmosphere of H₂ at room temperature for 24 h. Filtration, concentration in vacuo and elution through Sephadex LH-20 column (H₂O) provided 93 as a white solid. HRMS (ESI) m/z calcd for C₃₀H₄₇NO₂₁Na [M+Na]⁺ 780.2538.

### 2.38. Synthesis of L-glycero-α-D-manno-heptopyranosyl-(1→3)-L-glycero-α-D-manno-heptopyranosyl-(1→5)-2-(2-(4-(2-aminoethyl)-2-methoxyphenyl)ethyl)-3-deoxy-α-D-manno-oct-2-ulopyranosidonic acid 100

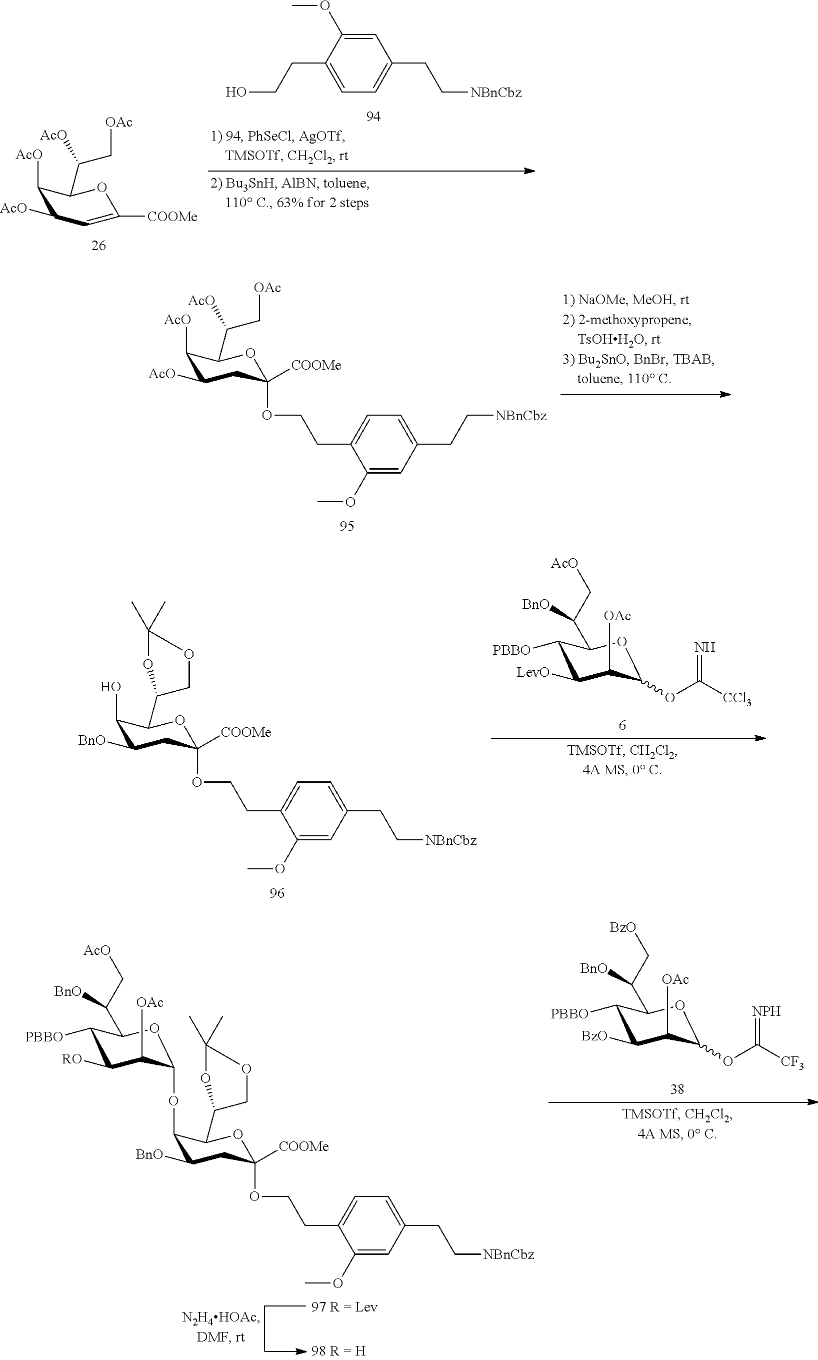

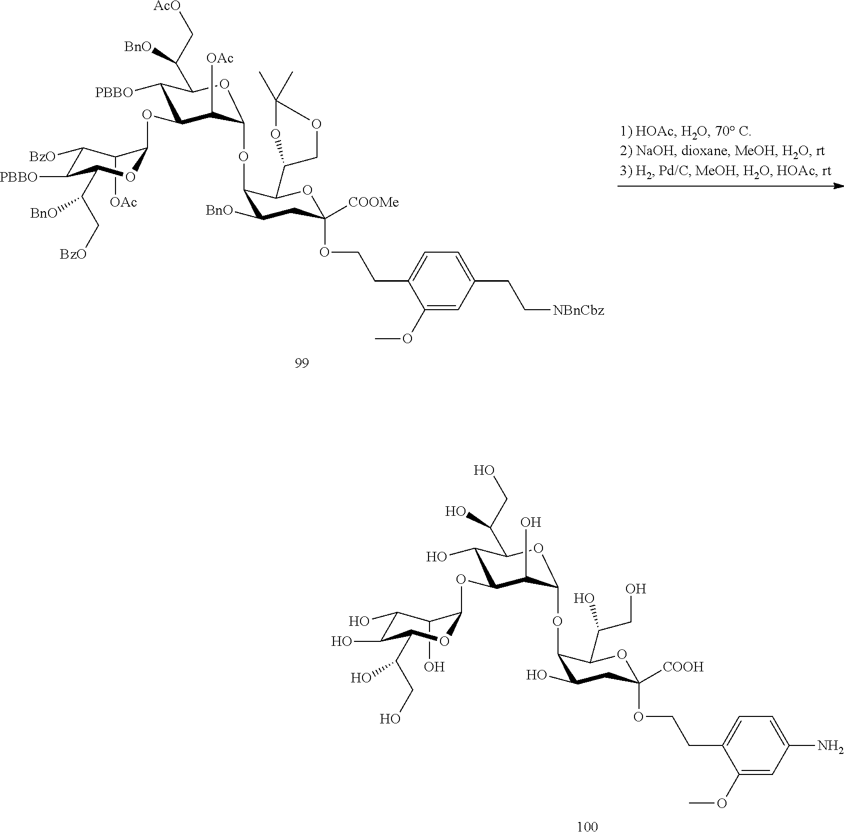

To a stirred solution of phenylselenyl chloride in CH₂Cl₂ was added AgOTf and TMSOTf. After stirring at room temperature for 30 min, a solution of glycal 26 and linker 94 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 95 as a colorless syrup.

To a stirred solution of compound 95 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 96 as a colorless syrup.

To a stirred mixture of the donor 6, acceptor 96, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 97 as a colorless syrup.

To a solution of 97 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 98 as a colorless syrup. To a stirred mixture of the donor 38, disaccharide acceptor 98, and freshly activated 4 Å MS in dry dichloromethane at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (toluene/acetone: 20/1) to afford 99 as a white foam.

A solution of compound 99 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding trisaccharide as a white solid. A mixture of the above trisaccharide and Pd/C in methanol, water and acetic acid (88/24/1, v/v/v) was stirred under an atmosphere of H₂ at room temperature for 24 h. Filtration, concentration in vacuo and elution through Sephadex LH-20 column (H₂O) provided 100 as a white solid. HRMS (ESI) m/z calcd for C₃₃H₅₃NO₂₁Na [M+Na]⁺ 822.3008.

### 2.39. Synthesis of L-glycero-α-D-manno-heptopyranosyl-(1→3)-L-glycero-α-D-manno-heptopyranosyl-(1→5)-2-(4-(2-aminoacetyl)phenyl)-3-deoxy-α-D-manno-oct-2-ulopyranosidonic acid 107

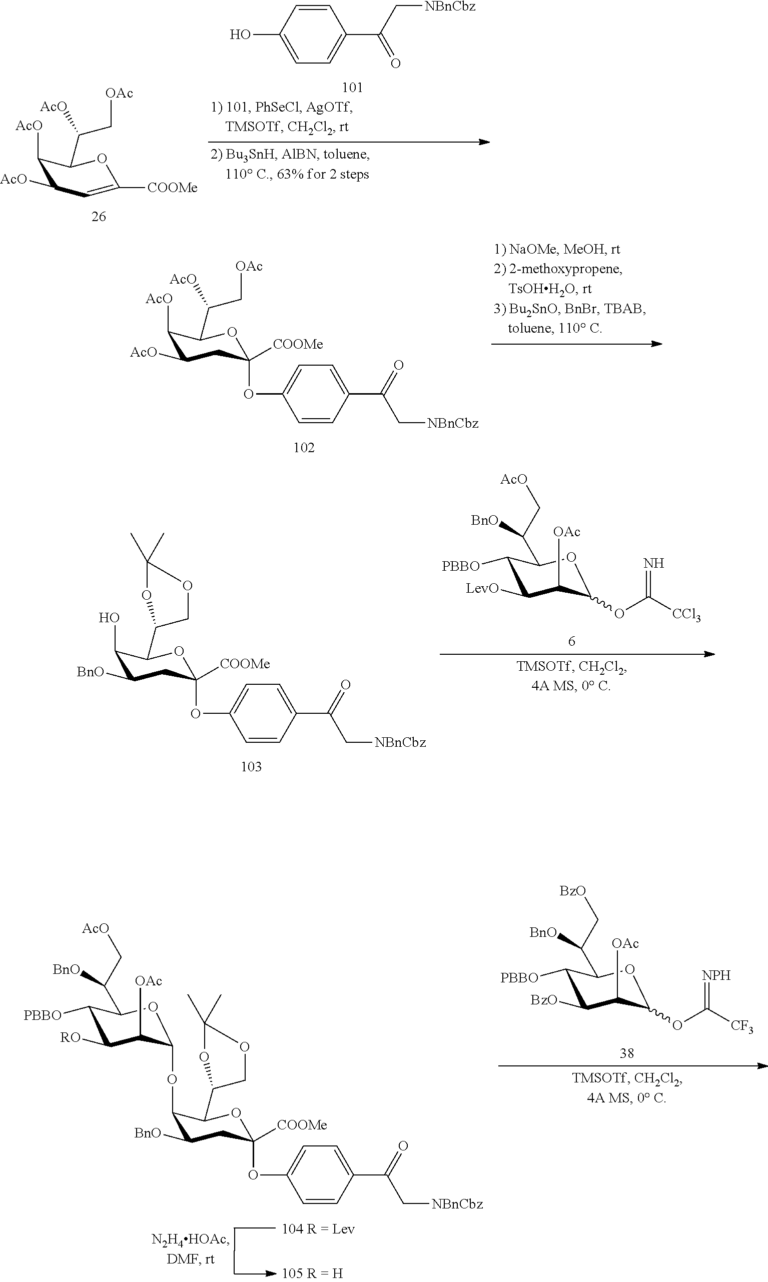

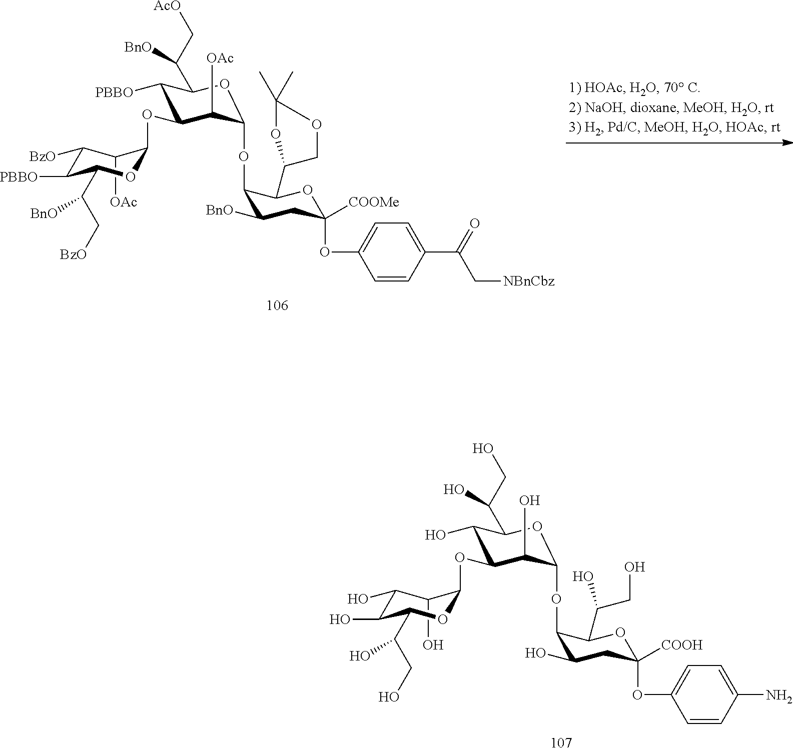

To a stirred solution of phenylselenyl chloride in CH₂Cl₂ was added AgOTf and TMSOTf. After stirring at room temperature for 30 min, a solution of glycal 26 and linker 101 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 102 as a colorless syrup.

To a stirred solution of compound 102 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 103 as a colorless syrup.

To a stirred mixture of the donor 6, acceptor 103, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 104 as a colorless syrup.

To a solution of 104 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 105 as a colorless syrup. To a stirred mixture of the donor 38, disaccharide acceptor 105, and freshly activated 4 Å MS in dry dichloromethane at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (toluene/acetone: 20/1) to afford 106 as a white foam.

A solution of compound 106 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding trisaccharide as a white solid. A mixture of the above trisaccharide and Pd/C in methanol, water and acetic acid (88/24/1, v/v/v) was stirred under an atmosphere of H₂ at room temperature for 24 h. Filtration, concentration in vacuo and elution through Sephadex LH-20 column (H₂O) provided 107 as a white solid. HRMS (ESI) m/z calcd for C₃₀H₄₅NO₂₁Na [M+Na]⁺ 778.2382.

### 2.40. Synthesis of L-glycero-α-D-manno-heptopyranosyl-(1→3)-L-glycero-α-D-manno-heptopyranosyl-(1→5)-2-(6-amino-3-oxo)hexyl-3-deoxy-α-D-manno-oct-2-ulopyranosidonic acid 114

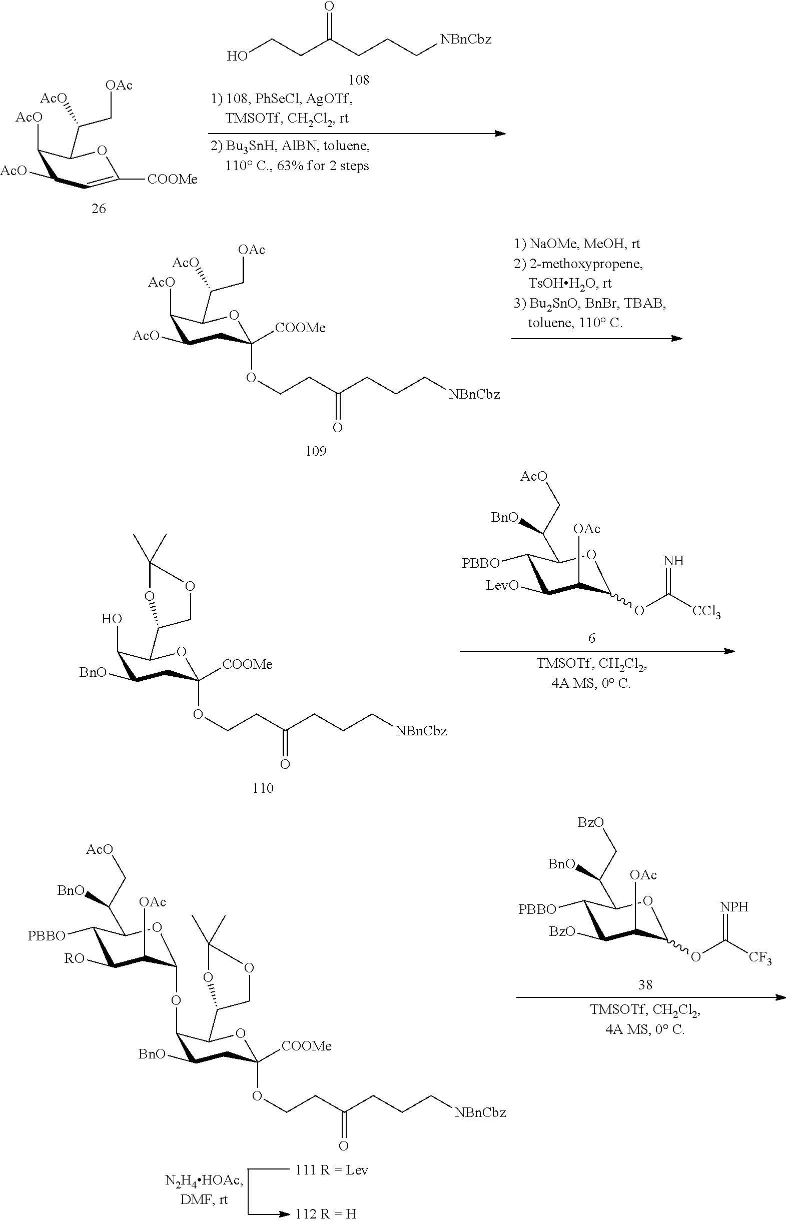

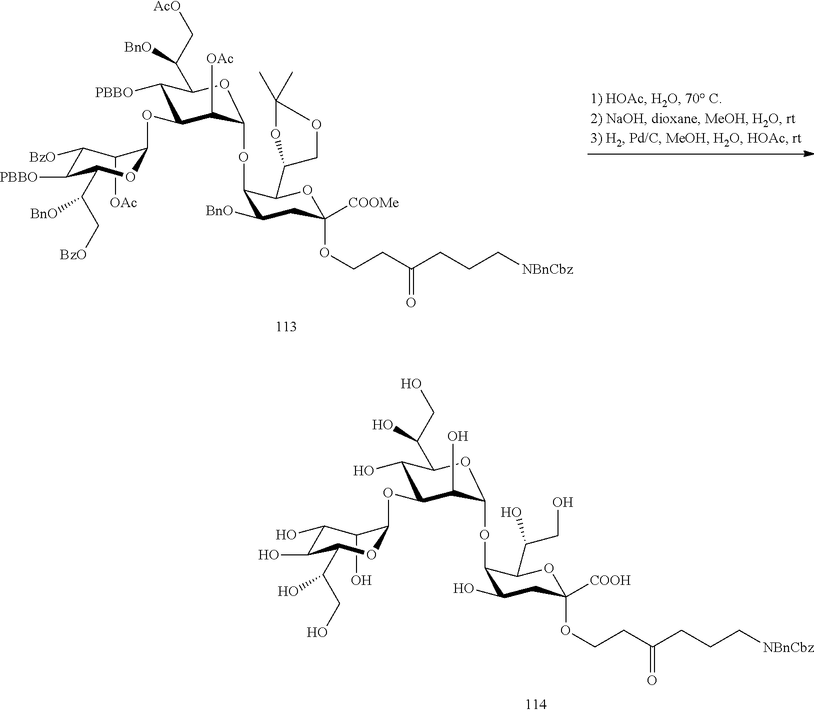

To a stirred solution of phenylselenyl chloride in CH₂Cl₂ was added AgOTf and TMSOTf. After stirring at room temperature for 30 min, a solution of glycal 26 and linker 108 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 109 as a colorless syrup.

To a stirred solution of compound 109 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 110 as a colorless syrup.

To a stirred mixture of the donor 6, acceptor 110, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 111 as a colorless syrup.

To a solution of 111 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 112 as a colorless syrup. To a stirred mixture of the donor 38, disaccharide acceptor 112, and freshly activated 4 Å MS in dry dichloromethane at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (toluene/acetone: 20/1) to afford 113 as a white foam.

A solution of compound 113 in acetic acid/water (8/1, v/v) was stirred at 70° C. for 5 h. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding trisaccharide as a white solid. A mixture of the above trisaccharide and Pd/C in methanol, water and acetic acid (88/24/1, v/v/v) was stirred under an atmosphere of H₂ at room temperature for 24 h. Filtration, concentration in vacuo and elution through Sephadex LH-20 column (H₂O) provided 114 as a white solid. HRMS (ESI) m/z calcd for C₂₈H₄₉NO₂₁Na [M+Na]⁺ 758.2695.

### 2.41. Synthesis of 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(8-amino-2,2-dimethyl)octyl-3-deoxy-α-D-manno-oct-2-ulopyranosidonic acid 121

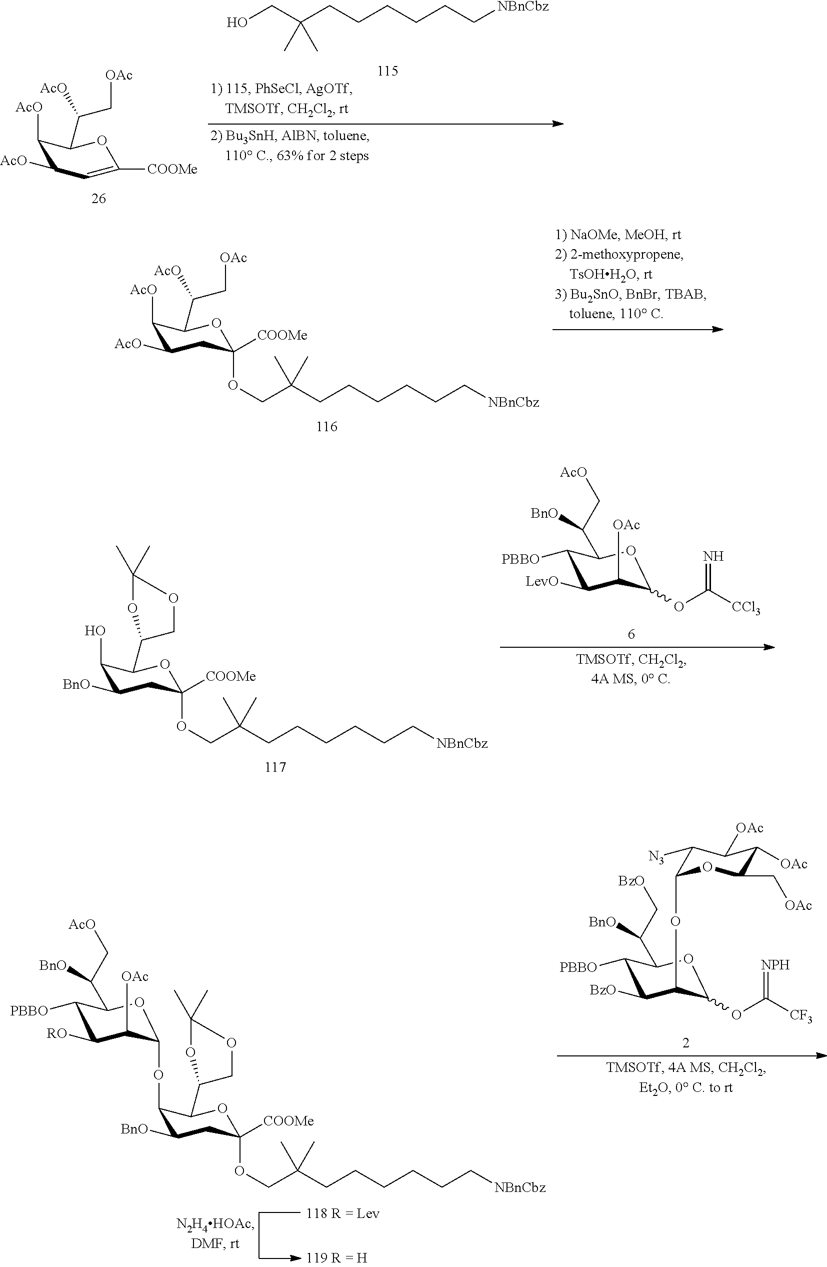

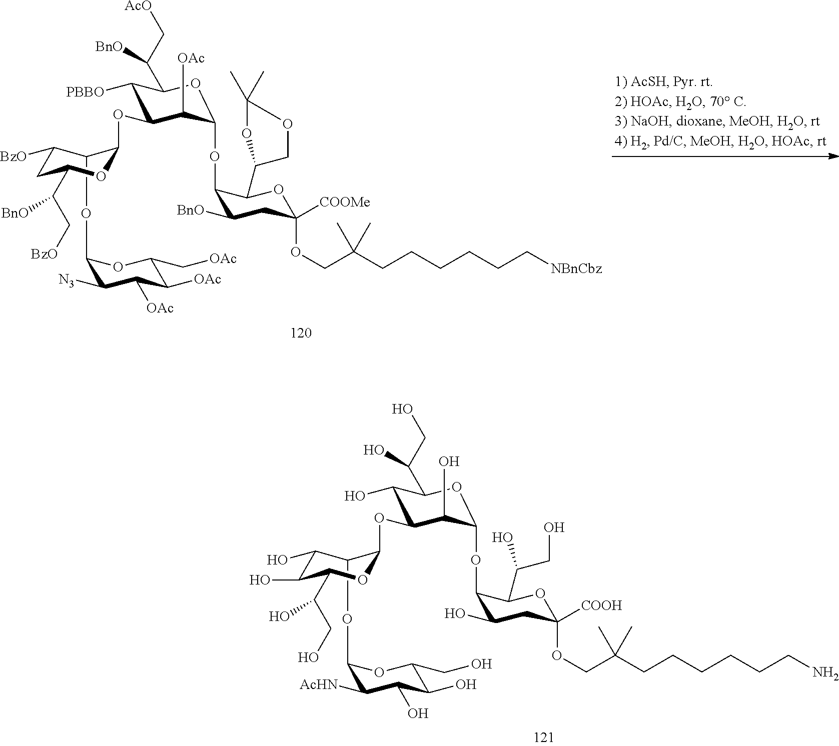

To a stirred solution of phenylselenyl chloride in CH₂Cl₂ was added AgOTf and TMSOTf. After stirring at room temperature for 30 min, a solution of glycal 26 and linker 115 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 116 as a colorless syrup.

To a stirred solution of compound 116 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 117 as a colorless syrup.

To a stirred mixture of the donor 6, acceptor 117, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 118 as a colorless syrup.

To a solution of 118 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 119 as a colorless syrup. To a stirred mixture of the disaccharide donor 2, disaccharide acceptor 119, and freshly activated 4 Å MS in dry diethyl ether and dichloromethane (1/1, v/v) at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (hexane/EtOAc/TEA: 5/2/0.07) to afford 120 as a white solid.

To a solution of compound 120 in dry pyridine, was added thioacetic acid. After being stirred at room temperature for 24 h, the solution was coevaporated with toluene to give a residue, which was purified by silica gel column chromatography (hexane/EtOAc/TEA: 3/2/0.05) to give the corresponding acetamide as a pale yellow solid. A solution of the above acetamide in acetic acid/water (8/1, v/v) was stirred at 70° C. for overnight. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding tetrasaccharide as a white solid. A mixture of the above tetrasaccharide and Pd/C in methanol, water and acetic acid (50/25/1, v/v/v) was stirred under an atmosphere of H₂ at room temperature for 24 h. Filtration, concentration in vacuo and elution through Sephadex LH-20 column (H₂O) provided 121 as a white solid. HRMS (ESI) m/z calcd for C₄₀H₇₁N₂O₂₅ [M−H]⁻ 979.4346.

### 2.42. Synthesis of 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(2-((aminomethyl)thio)ethyl)-3-deoxy-α-D-manno-oct-2-ulopyranosidonic acid

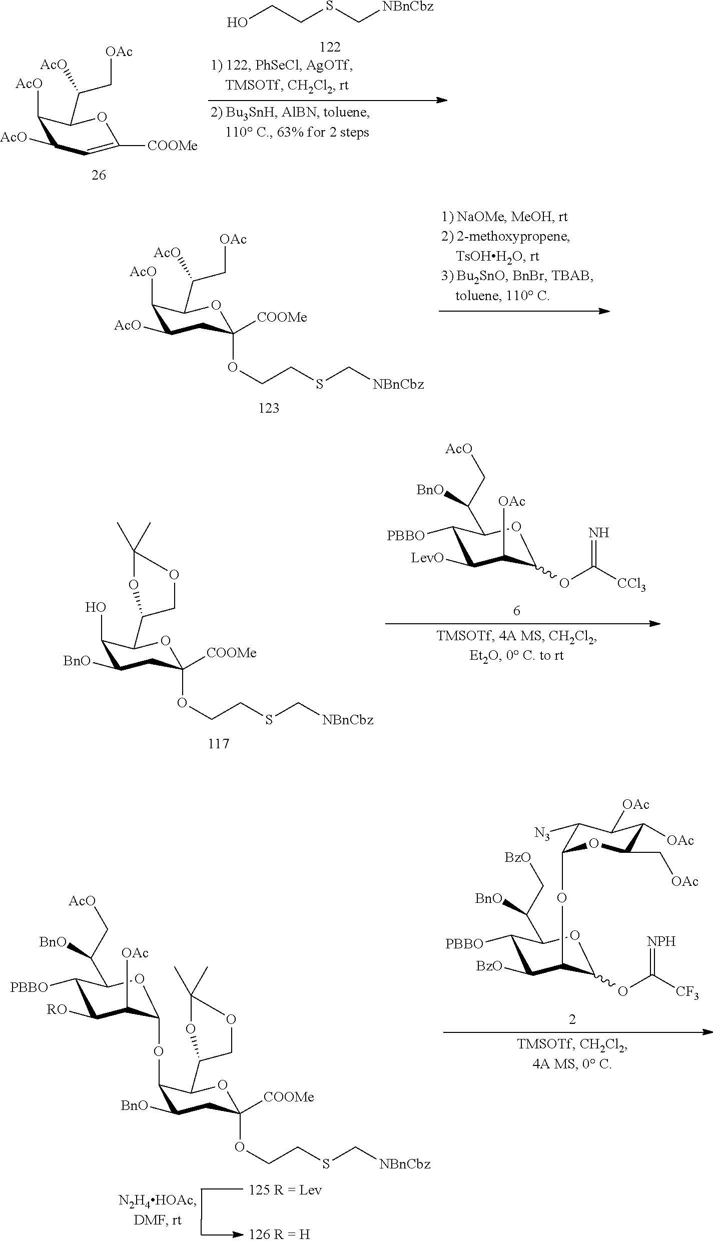

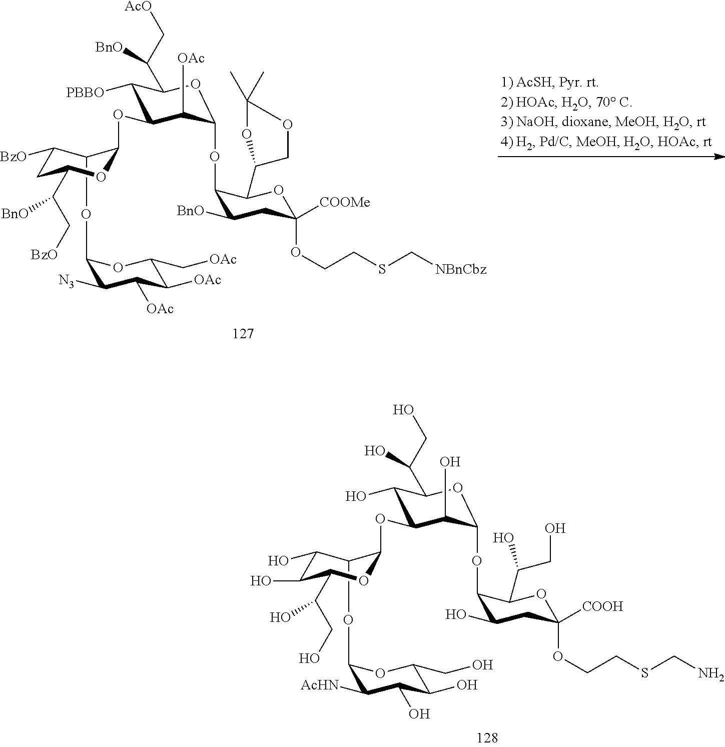

To a stirred solution of phenylselenyl chloride in CH₂Cl₂ was added AgOTf and TMSOTf. After stirring at room temperature for 30 min, a solution of glycal 26 and linker 122 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 123 as a colorless syrup.

To a stirred solution of compound 123 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 124 as a colorless syrup.

To a stirred mixture of the donor 6, acceptor 124, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 125 as a colorless syrup.

To a solution of 125 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 126 as a colorless syrup. To a stirred mixture of the disaccharide donor 2, disaccharide acceptor 126, and freshly activated 4 Å MS in dry diethyl ether and dichloromethane (1/1, v/v) at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (hexane/EtOAc/TEA: 5/2/0.07) to afford 127 as a white solid.

To a solution of compound 127 in dry pyridine, was added thioacetic acid. After being stirred at room temperature for 24 h, the solution was coevaporated with toluene to give a residue, which was purified by silica gel column chromatography (hexane/EtOAc/TEA: 3/2/0.05) to give the corresponding acetamide as a pale yellow solid. A solution of the above acetamide in acetic acid/water (8/1, v/v) was stirred at 70° C. for overnight. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding tetrasaccharide as a white solid. A mixture of the above tetrasaccharide and Pd/C in methanol, water and acetic acid (50/25/1, v/v/v) was stirred under an atmosphere of H₂ at room temperature for 24 h. Filtration, concentration in vacuo and elution through Sephadex LH-20 column (H₂O) provided 128 as a white solid. HRMS (ESI) m/z calcd for C₃₃H₅₇N₂O₂₅S [M−H]⁻ 913.2971.

### 2.43. Synthesis of 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(3-(3-aminopropyl)ureido)methyl-3-deoxy-α-D-manno-oct-2-ulopyranosidonic acid 135

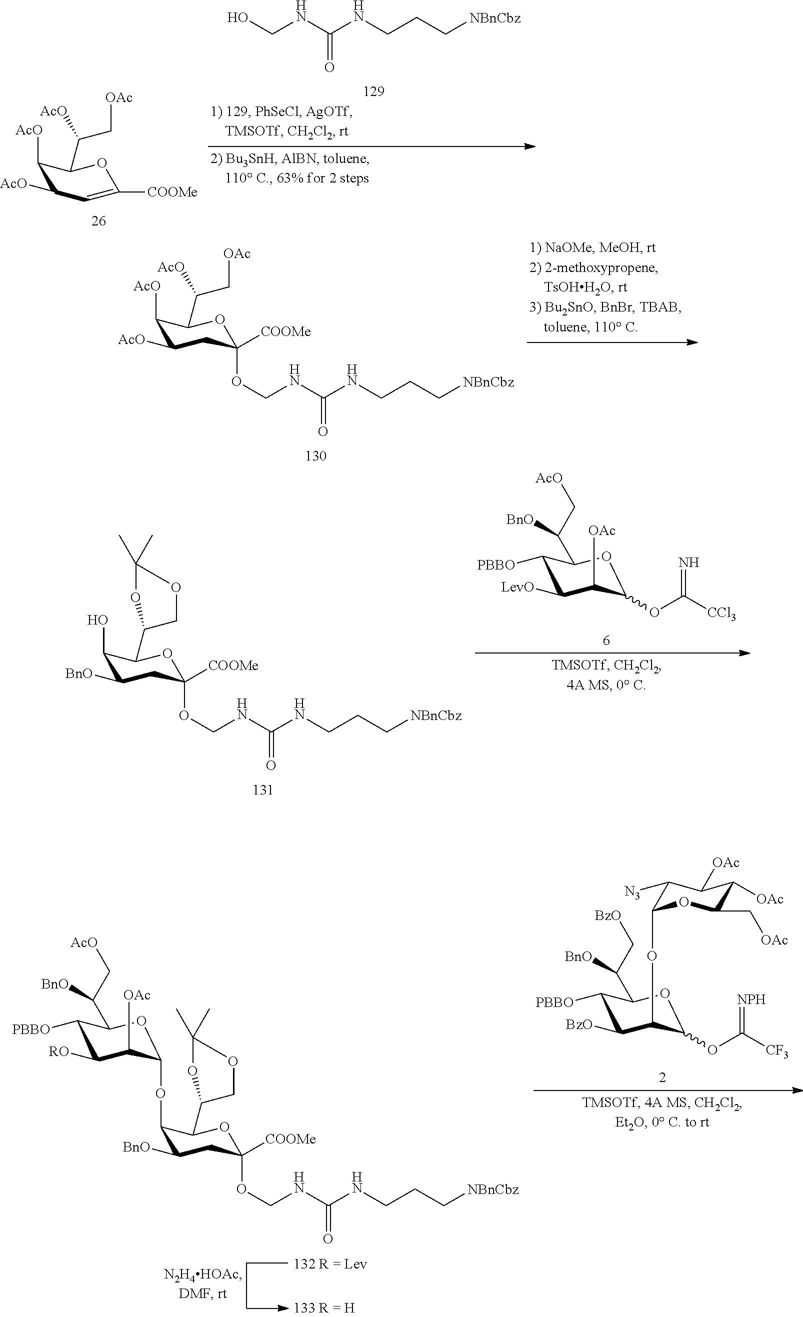

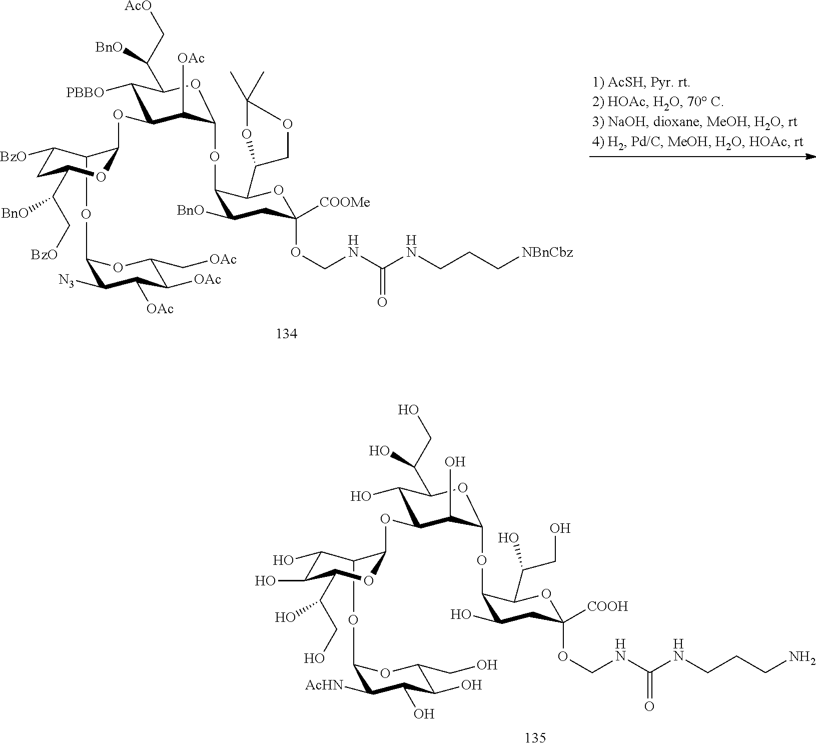

To a stirred solution of phenylselenyl chloride in CH₂Cl₂ was added AgOTf and TMSOTf. After stirring at room temperature for 30 min, a solution of glycal 26 and linker 129 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 130 as a colorless syrup.

To a stirred solution of compound 130 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 131 as a colorless syrup.

To a stirred mixture of the donor 6, acceptor 131, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 132 as a colorless syrup.

To a solution of 132 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 133 as a colorless syrup. To a stirred mixture of the disaccharide donor 2, disaccharide acceptor 133, and freshly activated 4 Å MS in dry diethyl ether and dichloromethane (1/1, v/v) at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (hexane/EtOAc/TEA: 5/2/0.07) to afford 134 as a white solid.

To a solution of compound 134 in dry pyridine, was added thioacetic acid. After being stirred at room temperature for 24 h, the solution was coevaporated with toluene to give a residue, which was purified by silica gel column chromatography (hexane/EtOAc/TEA: 3/2/0.05) to give the corresponding acetamide as a pale yellow solid. A solution of the above acetamide in acetic acid/water (8/1, v/v) was stirred at 70° C. for overnight. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding tetrasaccharide as a white solid. A mixture of the above tetrasaccharide and Pd/C in methanol, water and acetic acid (50/25/1, v/v/v) was stirred under an atmosphere of H₂ at room temperature for 24 h. Filtration, concentration in vacuo and elution through Sephadex LH-20 column (H₂O) provided 135 as a white solid. HRMS (ESI) m/z calcd for C₃₅H₆₁N₄O₂₆ [M−H]⁻ 953.3574.

### 2.44. Synthesis of 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(5-(2-aminoethyl)-1-methylpiperidin-2-yl)methyl-3-deoxy-α-D-manno-oct-2-ulopyranosidonic acid 142

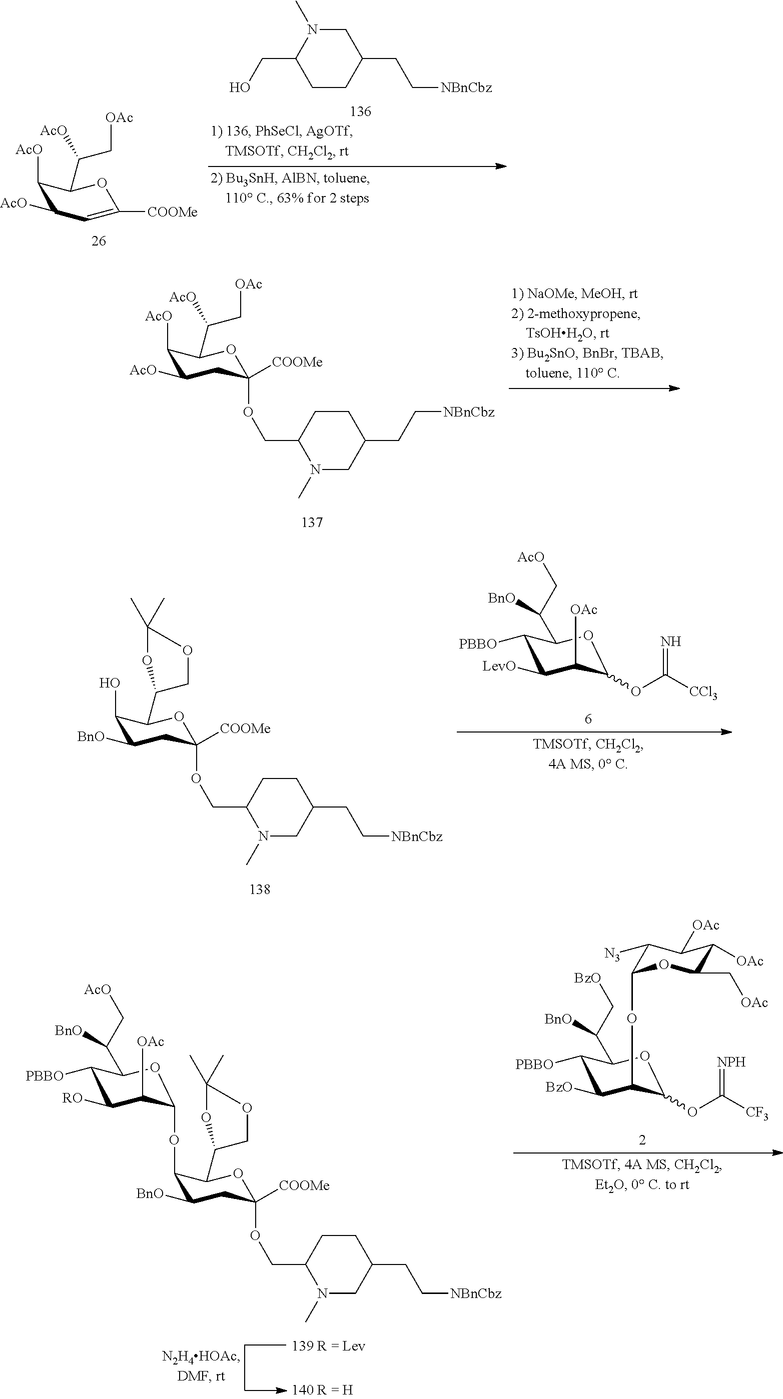

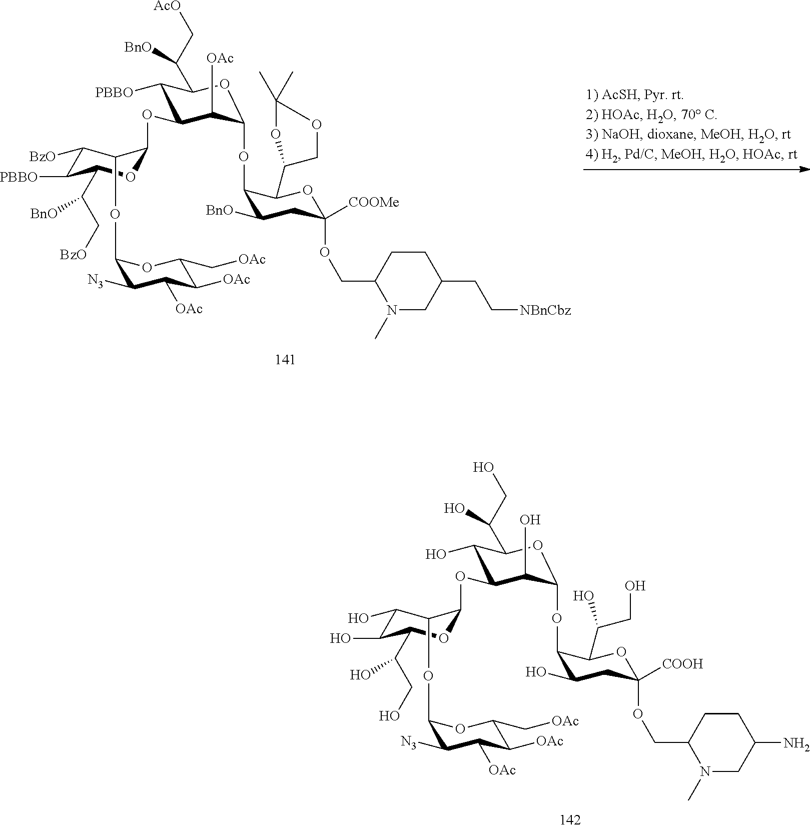

To a stirred solution of phenylselenyl chloride in CH₂Cl₂ was added AgOTf and TMSOTf. After stirring at room temperature for 30 min, a solution of glycal 26 and linker 136 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 137 as a colorless syrup.

To a stirred solution of compound 137 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 138 as a colorless syrup.

To a stirred mixture of the donor 6, acceptor 138, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 139 as a colorless syrup.

To a solution of 139 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 140 as a colorless syrup. To a stirred mixture of the disaccharide donor 2, disaccharide acceptor 140, and freshly activated 4 Å MS in dry diethyl ether and dichloromethane (1/1, v/v) at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (hexane/EtOAc/TEA: 5/2/0.07) to afford 141 as a white solid.

To a solution of compound 141 in dry pyridine, was added thioacetic acid. After being stirred at room temperature for 24 h, the solution was coevaporated with toluene to give a residue, which was purified by silica gel column chromatography (hexane/EtOAc/TEA: 3/2/0.05) to give the corresponding acetamide as a pale yellow solid. A solution of the above acetamide in acetic acid/water (8/1, v/v) was stirred at 70° C. for overnight. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding tetrasaccharide as a white solid. A mixture of the above tetrasaccharide and Pd/C in methanol, water and acetic acid (50/25/1, v/v/v) was stirred under an atmosphere of H₂ at room temperature for 24 h. Filtration, concentration in vacuo and elution through Sephadex LH-20 column (H₂O) provided 142 as a white solid. HRMS (ESI) m/z calcd for C₃₉H₆₈N₃O₂₅ [M−H]⁻ 978.4142.

### 2.45. Synthesis of 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(4-(3-aminopropyl)phenyl)-3-deoxy-α-D-manno-oct-2-ulopyranosidonic acid 149

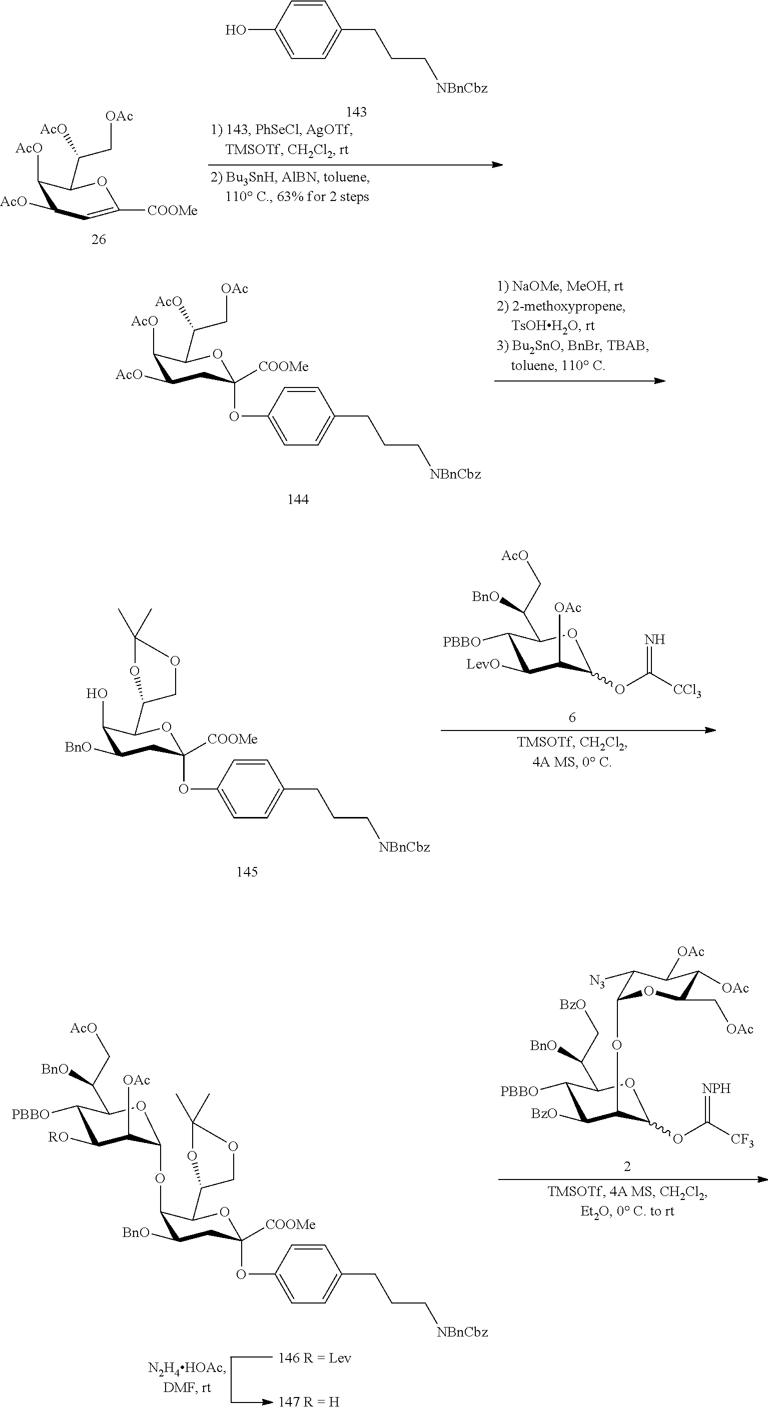

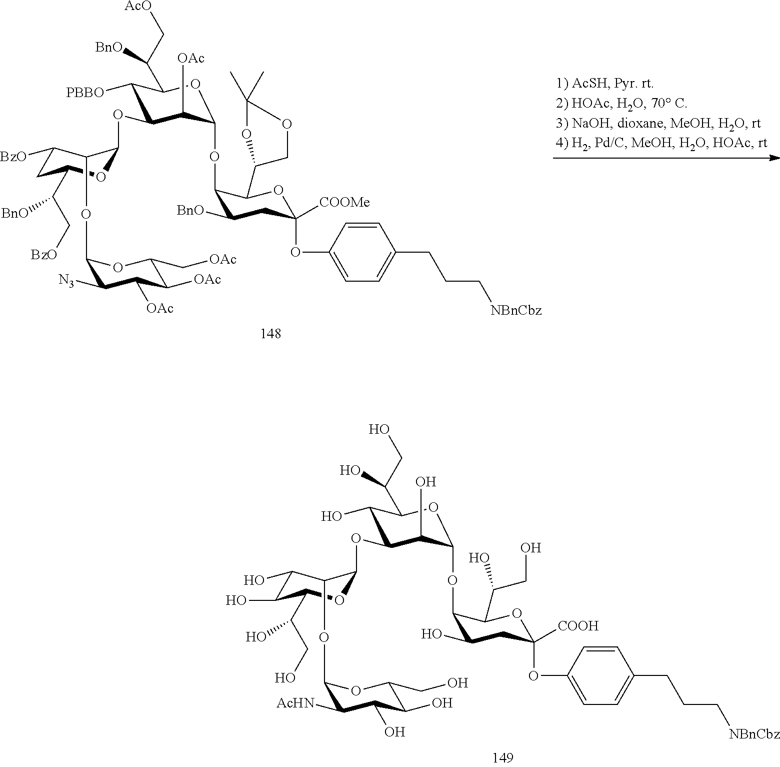

To a stirred solution of phenylselenyl chloride in CH₂Cl₂ was added AgOTf and TMSOTf. After stirring at room temperature for 30 min, a solution of glycal 26 and linker 143 in CH₂Cl₂ was added dropwise. After being stirred at room temperature for 2 h, the mixture was diluted with CH₂Cl₂, washed with saturated aqueous NaHCO₃, and brine. The organic layer was dried over Na₂SO₄, filtered and concentrated in vacuo. The residue was purified by silica gel column chromatography (hexane/EtOAc: 3/1) to give a white solid. To a solution of the above solid in toluene, was added tri-n-butyltin hydride and AIBN. After being refluxed for 1.5 h, the mixture was concentrated in vacuo and purified by silica gel column chromatography (hexane/EtOAc: 5/2) to afford 144 as a colorless syrup.

To a stirred solution of compound 144 in MeOH was added NaOMe. The mixture was stirred at room temperature for 4 h, and then neutralized with Amberlite IR120 H⁺ resin. Filtration, concentration in vacuo, and purification by silica gel column chromatography (CH₂Cl₂/MeOH: 12/1) gave the corresponding alcohol as a white solid. To a stirred solution of the above alcohol in DMF, was added 2-methoxypropene and p-toluenesulfonic acid monohydrate. The mixture was stirred at room temperature for 2 h, and then neutralized with sodium hydrogencarbonate. Filtration, concentration in vacuo, and purification by silica gel column chromatography (CH₂Cl₂/MeOH: 40/1) afforded the corresponding diol as a colorless syrup. A mixture of the above diol, dibutyltin oxide and 4 Å MS in toluene was heated at 110° C. for 2 h. After cooling to room temperature, benzyl bromide and tetrabutylammonium bromide were added, and the mixture was heated at 110° C. for overnight. The cooling mixture was then filtered and the filrate was evaporated. The residue was dissolved in CH₂Cl₂ and washed with water. The organic layer was dried over Na₂SO₄, filtered and concentrated in vacuo to give a residue, which was purified by silica gel column chromatography (hexane/EtOAc: 4/1) to give a syrup, which was dissolved in MeOH and treated with NaOMe. The mixture was stirred at room temperature for 3 h, and then neutralized with Amberlite IR120 H⁺ resin. Filtration, concentration in vacuo, and purification by silica gel column chromatography (hexane/EtOAc: 3/1) provided 145 as a colorless syrup.

To a stirred mixture of the donor 6, acceptor 145, and freshly activated 4 Å MS in dry CH₂Cl₂ at 0° C., was added dropwise TMSOTf in CH₂Cl₂ under nitrogen. After being stirred at 0° C. for 1 h, the mixture was quenched with Et₃N, filtered and concentrated in vacuo. The residue was purified by silica gel column chromatography (hexane/EtOAc: 2/1) to afford 146 as a colorless syrup.

To a solution of 146 in DMF at room temperature, was added hydrazine acetate. After being stirred at room temperature for 40 min, the mixture was diluted with EtOAc, washed with saturated aqueous NaHCO₃, and brine. The organic layer was dried over Na₂SO₄, filtered, and concentrated in vacuo. The residue was purified by silica gel column chromatography (hexane/EtOAc: 1/1) to give 147 as a colorless syrup. To a stirred mixture of the disaccharide donor 2, disaccharide acceptor 147, and freshly activated 4 Å MS in dry diethyl ether and dichloromethane (1/1, v/v) at 0° C., was added TMSOTf in CH₂Cl₂ under nitrogen. The temperature was allowed to warm up naturally to room temperature and the stirring continued for 1 h. The mixture was quenched with Et₃N, and filtered. The filtrate was concentrated in vacuo to give a residue, which was purified by silica gel column chromatography (hexane/EtOAc/TEA: 5/2/0.07) to afford 148 as a white solid.

To a solution of compound 148 in dry pyridine, was added thioacetic acid. After being stirred at room temperature for 24 h, the solution was coevaporated with toluene to give a residue, which was purified by silica gel column chromatography (hexane/EtOAc/TEA: 3/2/0.05) to give the corresponding acetamide as a pale yellow solid. A solution of the above acetamide in acetic acid/water (8/1, v/v) was stirred at 70° C. for overnight. TLC indicated complete conversion of starting material. The mixture was coevaporated with toluene and dried in vacuo to give the corresponding diol as a pale yellow syrup. The above diol was dissolved in a mixture of dioxane, methanol and 1 M aq NaOH (3/1/1, v/v/v). After being stirred at room temperature for overnight, the reaction mixture was diluted with methanol and neutralized with Amberlite IR120 H⁺ resin. After filtration, the filtrate was concentrated in vacuo to give the corresponding tetrasaccharide as a white solid. A mixture of the above tetrasaccharide and Pd/C in methanol, water and acetic acid (50/25/1, v/v/v) was stirred under an atmosphere of H₂ at room temperature for 24 h. Filtration, concentration in vacuo and elution through Sephadex LH-20 column (H₂O) provided 149 as a white solid. HRMS (ESI) m/z calcd for C₃₉H₆₁N₂O₂₅ [M−H]⁻ 957.3563.

### Example 3

### Conjugation of Tetrasaccharide 1 to CRM₁₉₇

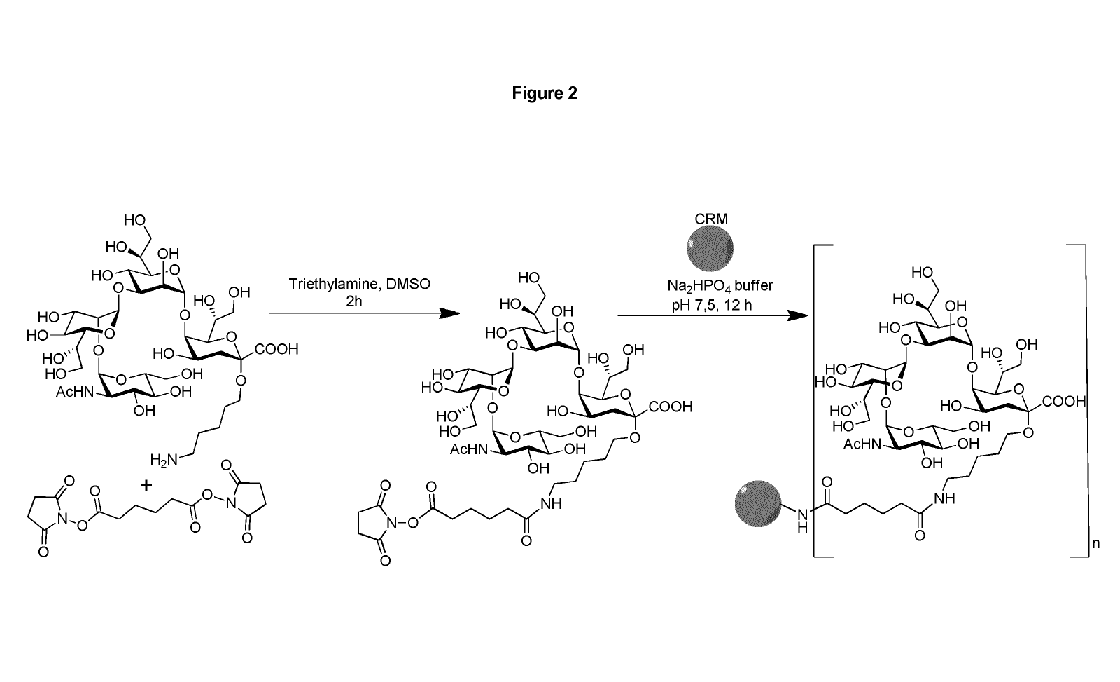

Vaccines based on polysaccharides are characterized by a T-cell independent immune response without inducing an immunological memory. Immunogenicity of polysaccharide vaccines in infants, elderly and immunocompromised patients are weak. Conjugation of carbohydrates to an immunogenic carrier protein creates a T-cell dependent immune response against the carbohydrate. As carrier protein, the nontoxic diphtheria toxoid variant CRM₁₉₇ was used, since it has been approved as a constituent of licensed vaccines. Tetrasaccharide 1 according to the invention was covalently bound to the carrier protein CRM₁₉₇ via a spacer molecule, disuccinimidyl adipate (see FIG. 2). First, the primary amine group of the linker moiety of tetrasaccharide 1 was reacted with one of the ester groups of the spacer molecule disuccinimidyl adipate in water-free DMSO in the presence of triethylamine at room temperature over 2 hours. The tetrasaccharide 1 was added drop-wise into 10-fold molar excess of spacer to avoid dimer formation. After addition of 400 μL 0.1 M Na-phosphate buffer, pH 7.42, unreacted spacer molecules were removed by chloroform extraction. The remaining ester group of the spacer moiety was then reacted with the c-amino groups of lysine residues on the CRM₁₉₇ protein (Pfenex) in 0.1 M Na-phosphate buffer, pH 7.42, at room temperature over 18 hours. For one reaction, 1.8 to 3.5 mg of meningococcal tetrasaccharide 1 and 1 mg of CRM₁₉₇ (solubilized in 1 mL 0.1 M Na-phosphate buffer, pH 7.42) was used. The resulting conjugate was purified by ultrafiltration (10 kDa, Amicon, Millipore) with deionized water. The protein concentration was determined by bicinchoninic acid (BCA) assay (Pierce). Conjugation was checked by SDS-PAGE (Example 4) and the tetrasaccharide 1/CRM₁₉₇ ratio was determined by MALDI-TOF MS (Example 5).

### Example 4

### SDS-PAGE

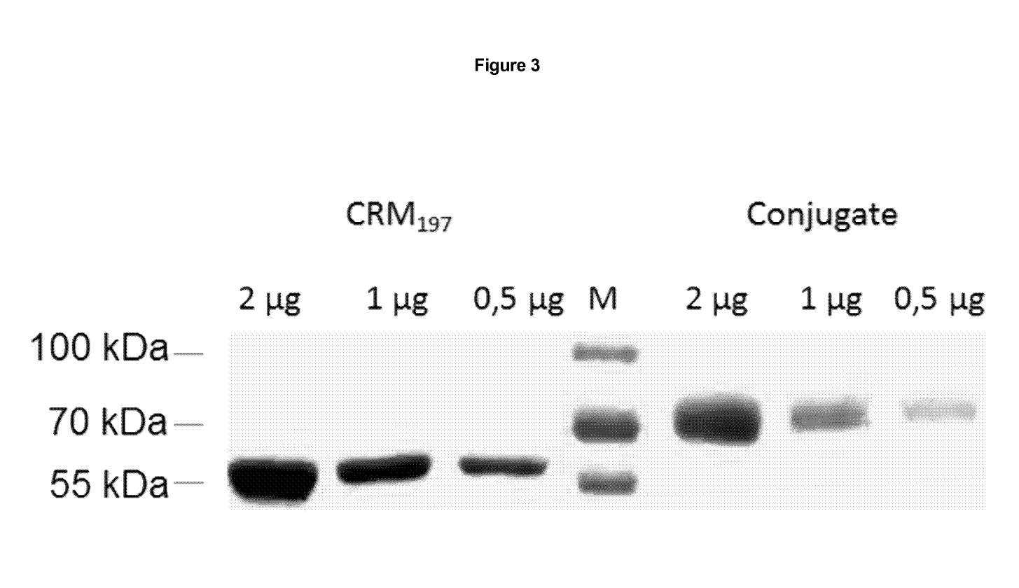

Tetrasaccharide 1-CRM₁₉₇ conjugate of example 3 and unconjugated CRM₁₉₇ were dissolved in Laemmli buffer (0.125 M Tris, 20% (v/v) glycerol, 4% (w/v) SDS, 5% (v/v) beta-mercaptoethanol, bromophenol, pH 6.8) and boiled at 95° C. for 5 minutes. Samples were run in 10% polyacrylamide gels and stained with 0.025% (w/v) Coomassie Brilliant blue R-250 in an aqueous solution containing 40% (v/v) methanol and 7% (v/v) acetic acid. A picture of the resulting gel is shown by FIG. 3.

### Example 5

### MALDI-TOF Mass Spectrometry

Conjugation was confirmed by matrix-assisted laser desorption/ionization-time of flight mass spectrometry (MALDI-TOF MS) using an Autoflex™ Speed instrument (Bruker Daltonics, Bremen, Germany). The mass spectrometer was operated in positive linear mode. Spectra were acquired over an m/z range from 40,000 to 180,000 kDa and data was analyzed with the FlexAnalysis software provided with the instrument. 2′,4′-dihydroxyacetonephenone (DHAP) was used as matrix, samples were spotted using the dried droplet technique. For CRM₁₉₇ an m/z ion of 58.3 kDa was detected, whereas for the conjugate the highest m/z ion was measured at 66.4 kDa resulting in a tetrasaccharide 1/CRM₁₉₇ ratio of 7. Generally an average of 3.9 tetrasaccharide 1 was coupled to CRM₁₉₇. Based on the determined protein concentration of the conjugate by bicinchoninic acid (BCA, Pierce) assay and the average sugar loading the carbohydrate content was calculated to 162±27 μg/mL (mean±SD).

### Example 6

### Immunizations in Presence of Freund's Adjuvants

To evaluate the immunogenicity of the meningococcal tetrasaccharide 1, two groups of six female BALB/c mice, six to eight weeks old, were immunized subcutaneously (s.c.) in presence of Freund's adjuvant (FA). Each mouse of the first group was immunized s.c. with 0.4 μg unconjugated tetrasaccharide 1 antigen in presence of Freund's adjuvant (priming with Freund's Complete Adjuvant, boosting with Freund's Incomplete Adjuvant, both Sigma). Each mouse of the second group received tetrasaccharide 1-CRM₁₉₇ conjugate, whereby the amount of conjugate corresponded to 0.4 μg tetrasaccharide 1 antigen. Two weeks after initial immunizations, first boosting injection was performed with 4 μg tetrasaccharide 1 or the corresponding tetrasaccharide 1-CRM₁₉₇ conjugate.

For all immunizations, antigen was diluted in sterile PBS to a total injection volume of 100 μL per mouse. Blood was collected at the tail vein and erythrocytes were separated from serum by centrifugation. All blood samples from the initial immunization study with FA were diluted with 10 μl sterile PBS before sera separation. Sera were collected in definited time intervals and IgG antibody responses were analyzed by microarray.

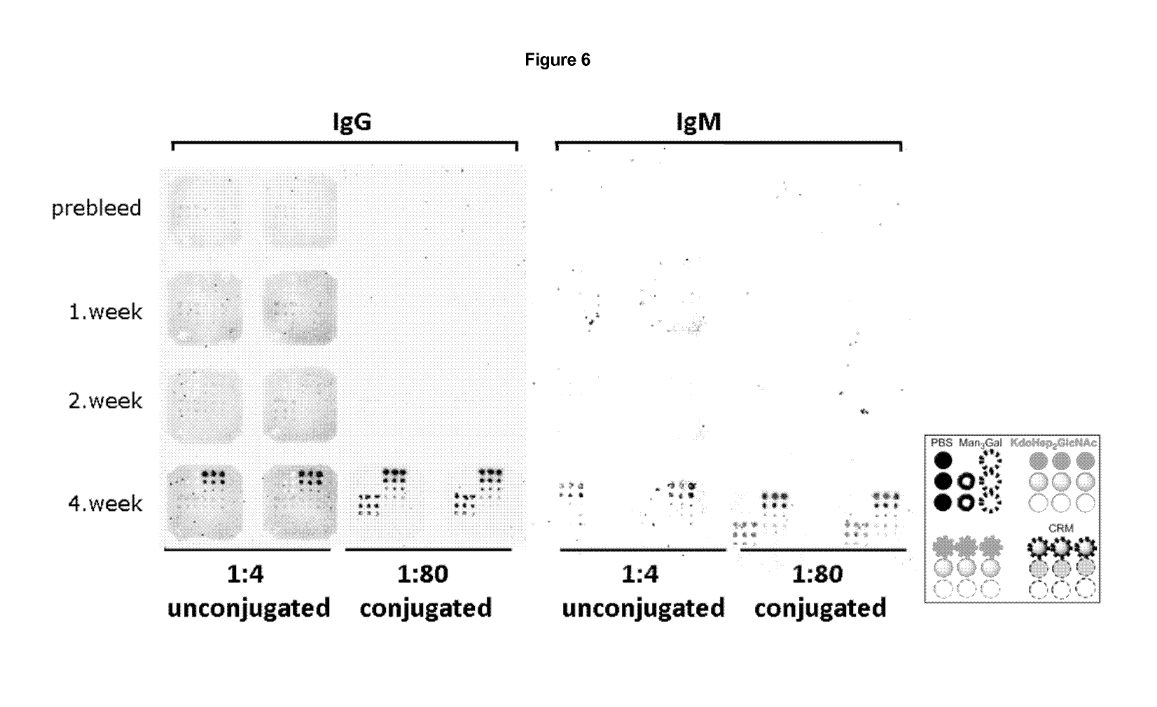

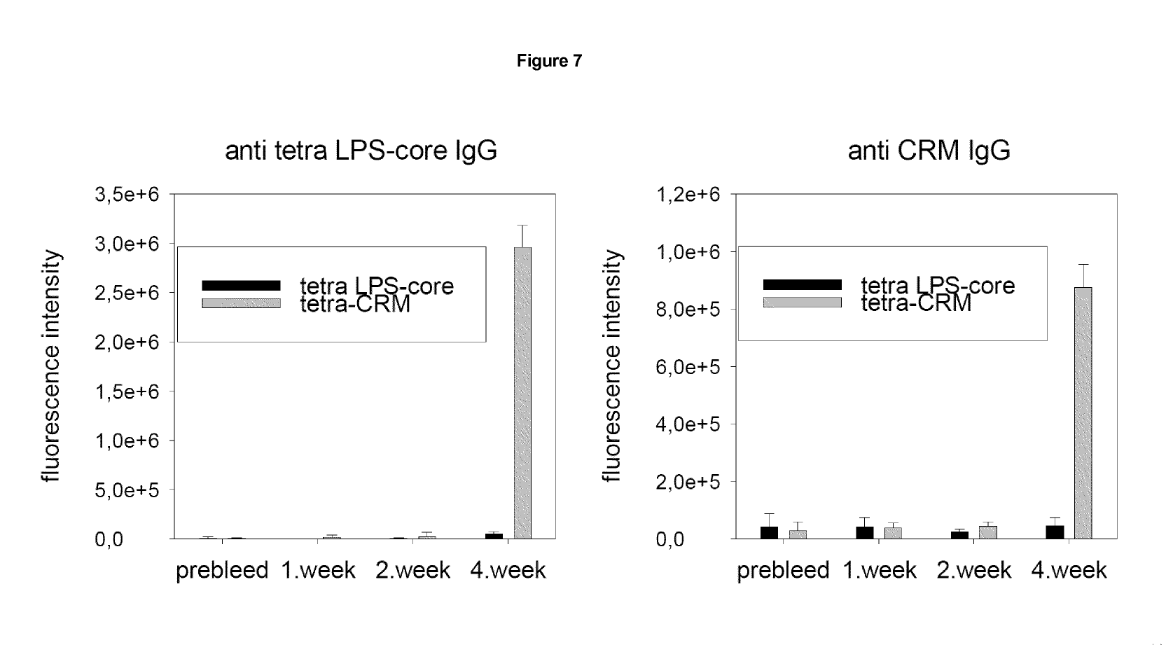

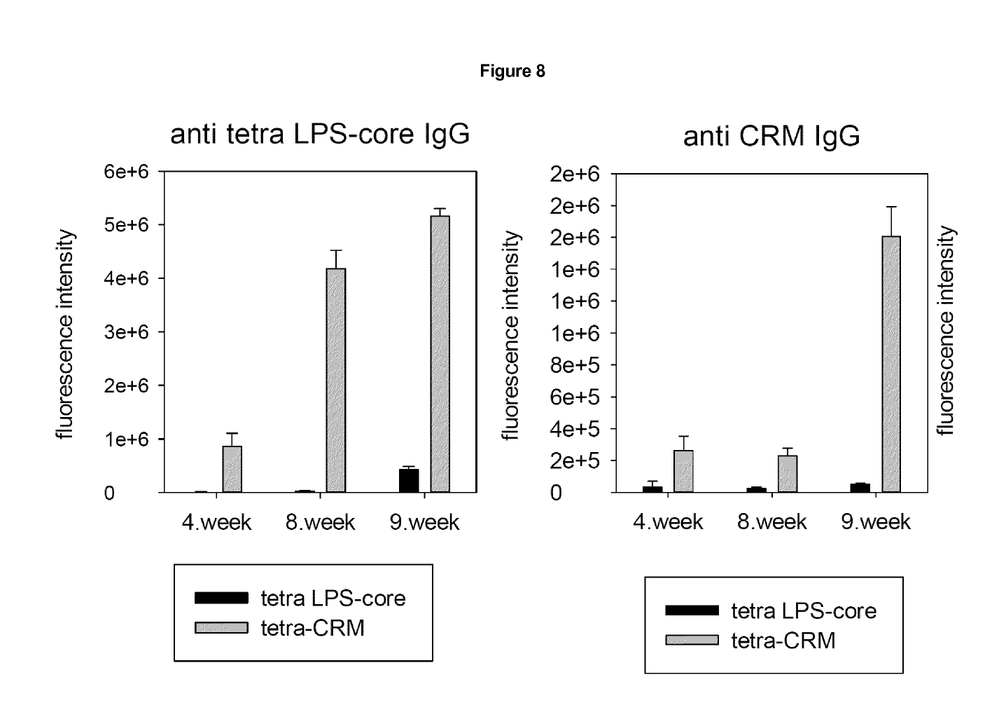

Tetrasaccharide 1-specific IgG and IgM antibody responses were identified in pooled sera in both groups after first boosting with 4 μg tetrasaccharide 1 or the corresponding tetrasaccharide 1-CRM₁₉₇ conjugate (4. week) (see FIG. 6). IgG antibody responses were quantified by determination of the fluorescence intensity values in 1:80 sera dilution. While the unconjugated tetrasaccharide 1 showed relatively weak immunogenicity in presence of FA (FIG. 7, right diagram, black bar), the sera from tetrasaccharide 1-CRM₁₉₇ conjugate immunized mice showed significantly higher anti-tetrasaccharide 1 titers (grey bars). In week 8 mice were boosted a second time with 4 μg antigen, whereby IgG response against tetrasaccharide 1 showed a marginal increase in week 9 (FIG. 8, left diagram, black bar). In contrast, the IgG titer of tetrasaccharide 1-CRM₁₉₇ conjugate is already 4.8 times higher at week 8 compared to week 4 (FIG. 8, left diagram, grey bar). One week later the IgG titer against conjugate was again increased. Second boosting also generated a higher IgG titer against spacer construct.

This study showed that the tetrasaccharide 1-CRM₁₉₇ antigen is immunogenic in the presence of FA and the tetrasaccharide 1-CRM₁₉₇ conjugate elicited robust boosting IgG responses. The optimal dose for inducing the response was observed to be 4 μg whereas in presence of 0.4 μg antigen no immune response was observed.

### Example 7

### Preparation of Microarrays

Tetrasaccharide 1 in three different concentrations (1, 0.5 and 0.1 mM), CRM₁₉₇ (1, 0.5 and 0.1 μM) and bovine serum albumin (BSA)-spacer-GlcNAc conjugate (1, 0.5 and 0.1 μM) were dissolved in sodium phosphate buffer (50 mM, pH 8.5) and printed in triplicate robotically using a piezoelectric spotting device (S11, Scienion, Berlin, Germany) onto the surface of the microarray slides (NHS-activated glass slides; CodeLink). BSA-spacer-GlcNAc was used to assess immunogenicity against the spacer moiety of the conjugate. As negative controls, phosphate-buffer saline (PBS), as well as a unrelated oligosaccharide Man₃Gal at concentrations of 0.5 mM and 1 mM were also included. Microarrays were designed such that high-throughput analysis of 64 samples per array was possible. Slides were incubated in a humid chamber to complete reaction for 24 hours and stored in an anhydrous environment. Prior to the experiment, remaining succinimidyl groups were quenched by incubating slides in 100 mM ethanolamine in sodium phosphate buffer (pH 9, 50 mM) for 1 hour at 50° C. Slides were rinsed three times with deionized water and dried by centrifugation.

### Example 8

### Microarray Binding Assays (Glycan Array)

The quenched array slides were blocked for 1 hour with 1% (w/v) BSA in PBS, then washed three times with PBS and dried by centrifugation. A FlexWell 64 (Grace Bio-Labs, Bend, Oreg., USA) grid was applied to the slides. Resulting 64 wells were used for 64 individual experiments. Slides were incubated with sera dilutions or hybridoma supernatants of example 11 (all dilutions were prepared with PBS) for 1 hour at room temperature in a humid chamber, washed three times with PBS-Tween-20 (0.1% v/v) and dried by centrifugation. Then, slides were incubated with fluorescence-labeled detection antibody diluted in 1% BSA-PBS (w/v) for 1 hour at room temperature in a humid chamber. Slides were washed three times with PBS-Tween-20 (0.1% v/v) and rinsed once with deionized water and dried by centrifugation. Slides were scanned with a GenePix 4300A scanner (Molecular Devices) using the GenePix Pro 7 software. Detection antibodies used were Rabbit Anti-Mouse IgG (whole molecule)-FITC (Sigma), Alexa Fluor 635 Goat Anti-Mouse IgG (H+L) (Life Technologies) and Alexa Fluor 594 Goat Anti-Mouse IgG1 (γ1) (Life Technologies) in 1:400 dilutions, as well as Alexa Fluor 647 Goat Anti-Mouse IgG2a (γ2a), Alexa Fluor 488 Goat Anti-Mouse IgG3 (γ3) (Life Technologies), Alexa Fluor 594 Goat Anti-Mouse IgM (μ chain) (Life Technologies) and Alexa Fluor 594-conjugate-AffiniPure Donkey Anti-Mouse IgM, (μ) (Jackson Immuno Research) in 1:200 dilutions.

### Example 9

### Evaluation of Immunogenicity of Glycoconjugates Formulated Using a Human Approved Adjuvant

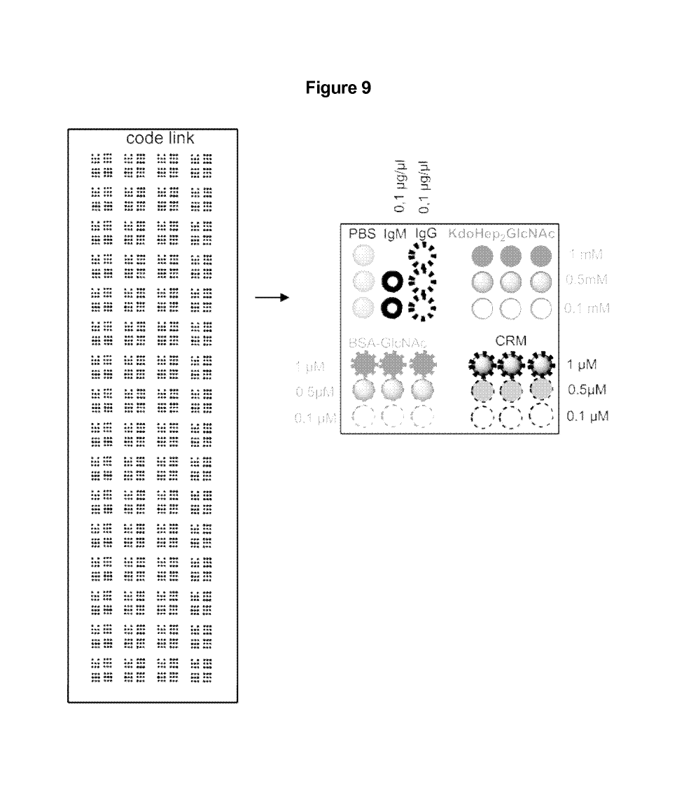

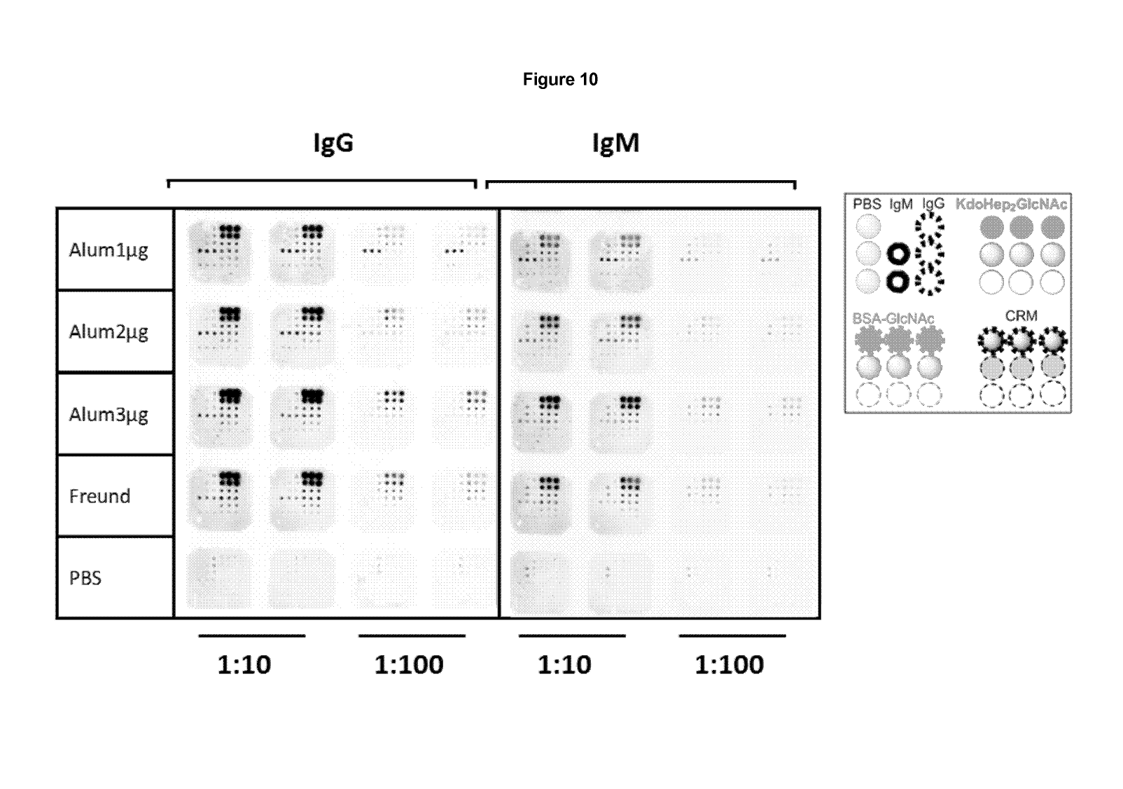

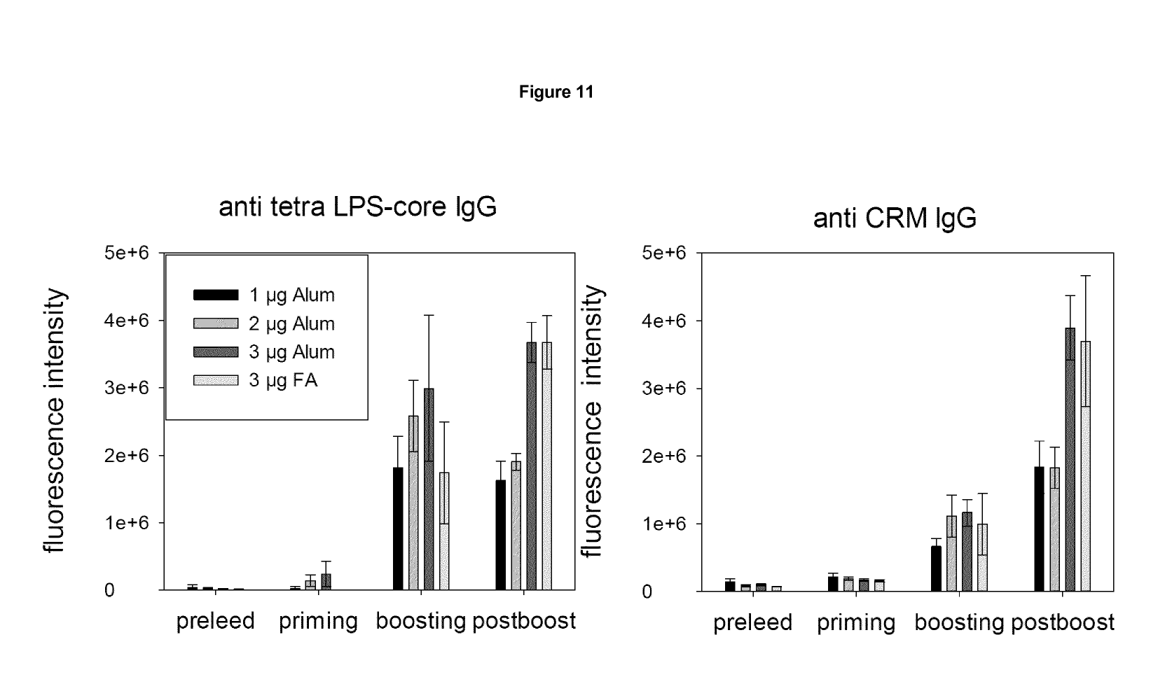

A new immunization study was performed with smaller amounts of conjugate to determine the needful antigen amount, which creates a detectable immune response. Immunogenicity of 3, 2 and 1 μg antigen was checked in a dose-dependent immunization study in presence of alum (Aluminium Hydroxide Gel Adjuvant (Brenntag Biosector, Frederikssund, Denmark), which is a human approved adjuvant. As positive control 3 μg tetrasaccharide 1-CRM₁₉₇ conjugate was immunized in presence of FA, whereas PBS was used for the negative control. Each group contained six female BALB/c mice, six to eight weeks old. Initial immunizations (priming) was followed by a first boosting immunization after two weeks. Sera were collected in one-week intervals. IgG antibody responses were evaluated by microarray. The detailed microarray printing pattern is shown in FIG. 9 and here instead of the Man₃Gal 0.1 μg/μl purified mouse IgG and IgM were printed as control (FIG. 9). IgG antibody responses were quantified by determination of the fluorescence intensity values using the sera of individual mice in 1:100 dilution. Glycan array analysis at the day of first boosting shows that 1, 2 and 3 μg tetrasaccharide 1-CRM conjugate are immunogenic in presence of alum (FIGS. 10 and 11, left diagram), and highest antibody response was observed against 3 μg tetrasaccharide 1-CRM₁₉₇ conjugate in presence of alum, followed by 2 μg and 1 μg tetrasaccharide 1-CRM₁₉₇ conjugate. After first boosting equal IgG titers against 3 μg tetrasaccharide 1-CRM₁₉₇ conjugate in presence of FA and alum were observed. Also IgG response against 1 μg and 2 μg were in the same range. The results indicated that the tetrasaccharide 1-CRM₁₉₇ conjugate are immunogenic when formulated with the human approved adjuvant alum and the minimum effective dose required to elicit anti-tetrasaccharide 1 immune reaction was observed to be 1 μg.

### Example 10

### Characterization of Immune Response in Presence of FA

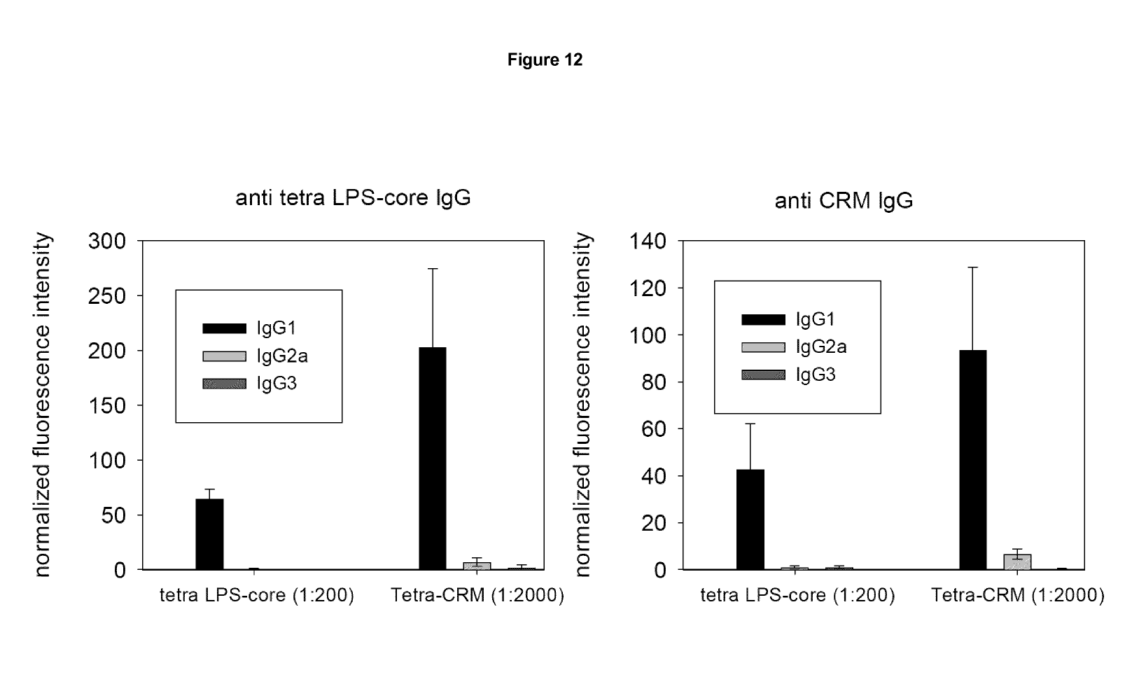

Unconjuagted carbohydrate antigens do not generally induce isotype switching of antibodies. The major aim of anti-carbohydrate vaccines are to induce opsonophagocytic IgGs. IgG isotypes especially IgG1 has higher opsonophagocytic potential as well as better maternal transfer across placenta to the fetus. So to get an insight into the subclasses of IgG antibodies raised against tetrasaccharide 1, microarray analysis with pooled sera using subclass-specific detection antibodies against IgG1, IgG2a and IgG3 was performed. Fluorescence intensity was normalized by calculating the ratio of sample fluorescence A/fluorescence prebleed A. As shown in FIG. 12, left diagram, IgG1 is the major isotype observed in unconjugated tetrasaccharide 1 as well as in tetrasaccharide 1-CRM conjugate immunized mice. The results indicate that the glycoconjugation process improved the immunogenicity of the tetrasaccharide 1 antigen and also promoted isotype switching of antibody responses which is very important in the context of carbohydrate based vaccines.

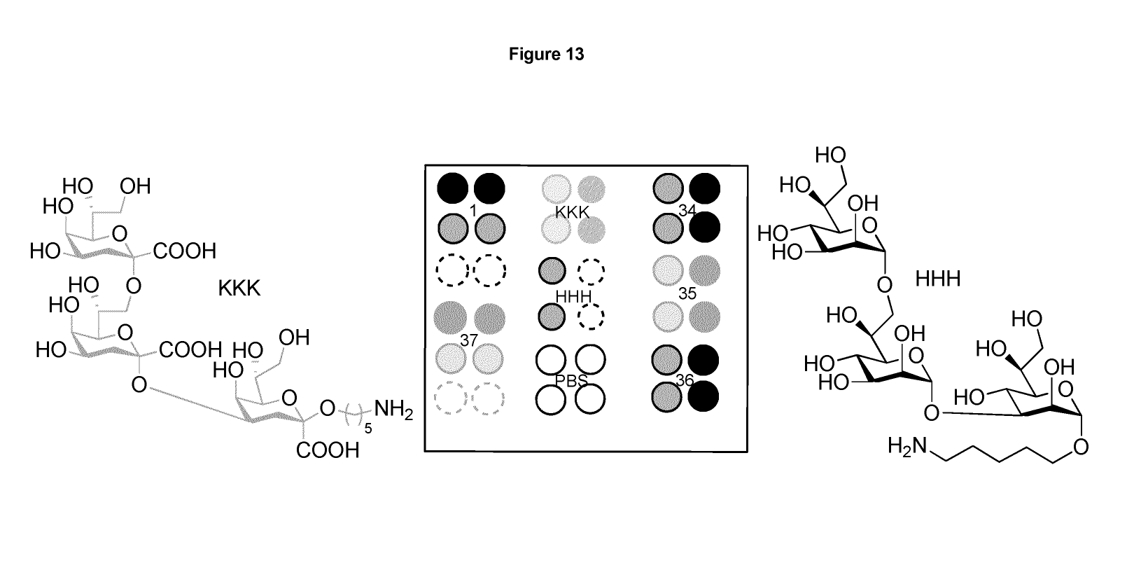

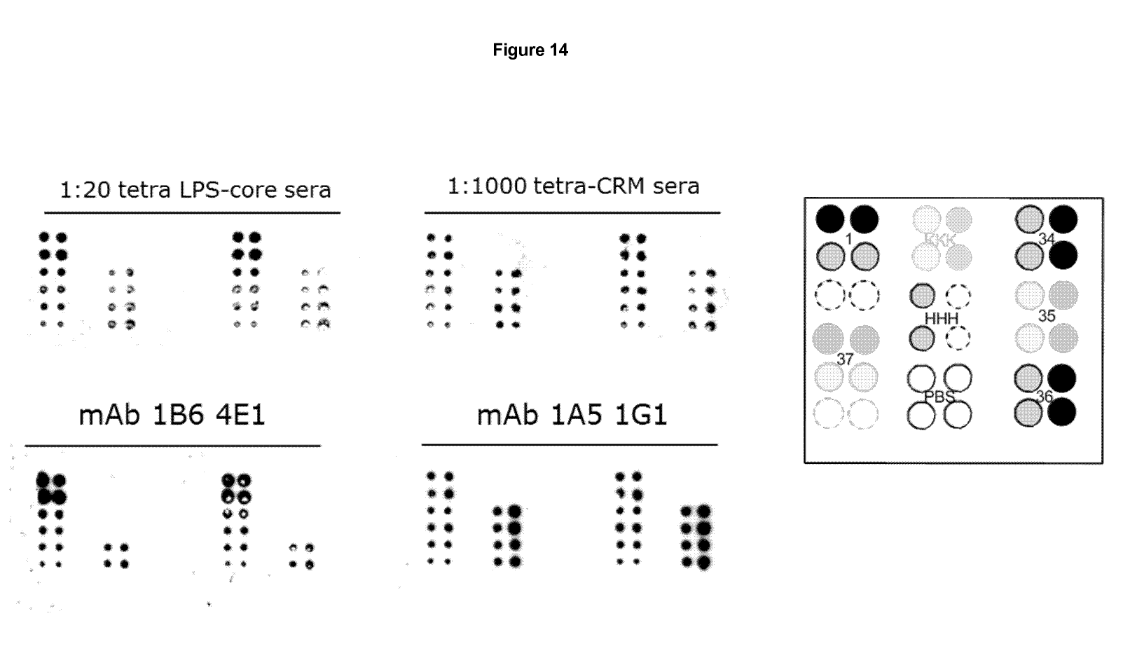

To find out the minimum structure required to bind the antibodies, reactivities of pooled sera against different synthetic saccharides of general formula (I) and related LPS-core structures of other bacteria then N. meningitidis were checked by microarray analysis. The tetrasaccharide 1, the trisaccharide 37, the disaccharide 36, the mono-Kdo 35 as well as the mono-Hep 34 were printed on a glycan array (FIG. 13). The conserved LPS-core structures tri-Hep from Yersinia pestis (HHH) and the chlamydial tri-Kdo (KKK) respectively, were included as related core structures. Both sera, produced against the tetrasaccharide 1 and the tetrasaccharide 1-CRM₁₉₇ conjugate, showed equal reactivity against these substructures (FIG. 14). IgG antibodies against the tetrasaccharide 1, the trisaccharide 37, the disaccharide 36 and the mono-Kdo 35 were observed in pooled sera. No cross reactivity against the LPS-core structures of pestis or chlamydia as well as no reactivity with the mono-Hep 34 was observed. This indicated that the generated antibodies have higher specificities for N. meningitidis in comparison to other related structures.

### Example 11

### Monoclonal Antibodies

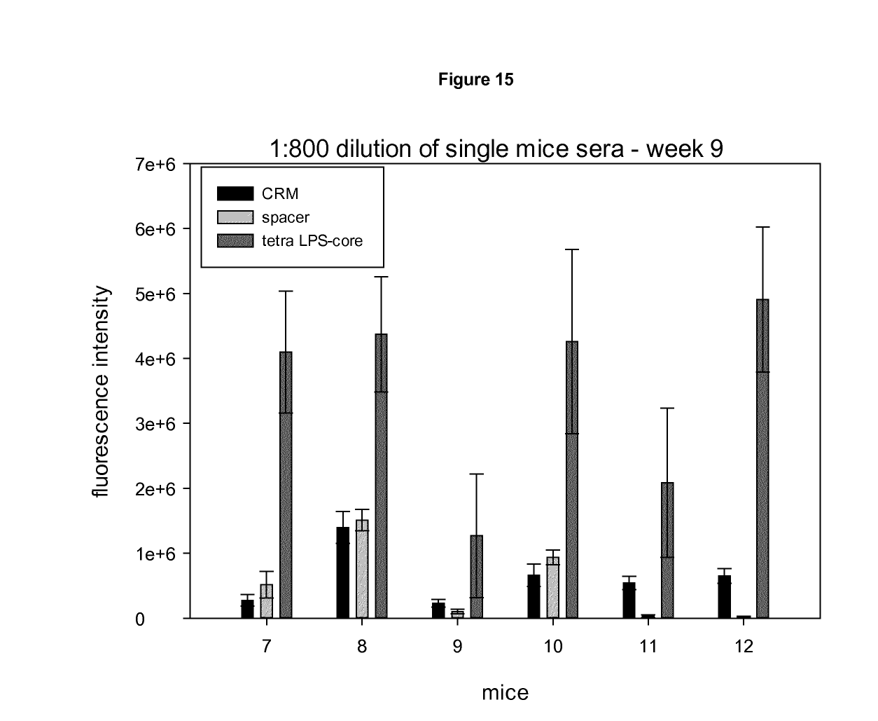

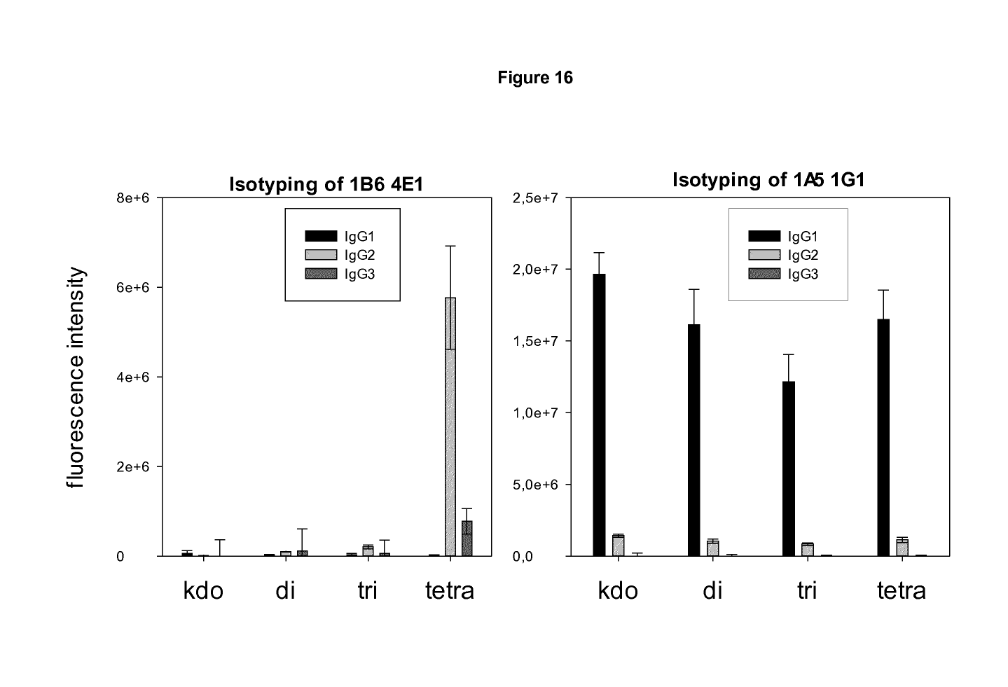

Development of monoclonal antibodies against the tetrasaccharide 1 would be useful for passive immunization as well as detection of N. meningitidis in biological fluids. Monoclonal antibodies (mABs) were generated using the standard method by Koehler and Milstein, 1975. To enable this, mouse 12 (FIG. 15, grey bar), which elicited the highest antibody response against the antigen was selected, splenocytes were isolated and fused with 10⁸ mouse myeloma cells in the presence of 50% PEG 1500 to generate hybridomas. Fused cells were selected with complete growth medium (IMDM supplemented with 10% heat-inactivated fetal calf serum, 2 mM L-glutamine, 24 μM beta-mercaptoethanol, 1 mM sodium-pyruvate, 100 μM hypoxanthine, 16 μM thymidine, non-essential amino acids, 100 U/mL penicillin, 100 μg/mL streptomycin, 50 μg/mL gentamycin, 10% hybridoma cloning supplement (BM Condimed H1, Roche)) with 0.4 μM aminopterin. Cells were maintained at 37° C. at 5% CO₂. Hybridoma cells were subjected to four consecutive subcloning steps by limited dilution. Clones producing antibodies against tetrasaccharide 1 were identified by microarray. Two hybridoma clones producing monoclonal antibodies (mAbs), 1A5 1G1 and 1B6 4E1, were selected for evaluation of the deletion sequence microarray (FIG. 14). Both mAbs bound to the tetrasaccharide 1, the trisaccharide 37 and the disaccharide 36, while the 1A5 1G1 additionally bound to the mono-Kdo 35. Isotyping of hybridoma supernatant-dilutions showed that 1B6 4E1 was isotype IgG2a (FIG. 16, light grey bar) and binds specifically to tetrasaccharide 1. In contrast, mAb 1A5 1G1 was isotype IgG1 and recognized the mono-Kdo 35, disaccharide 36, trisaccharide 37, and tetrasaccharide 1.

### Example 12

### Confocal Laser Scanning Microscopy

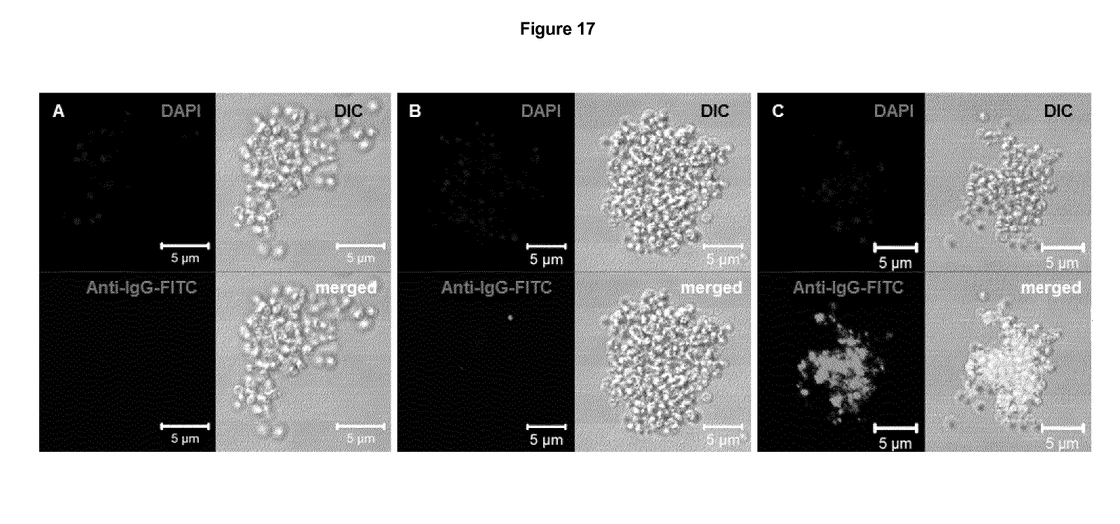

Binding of antibodies against heat-inactivated N. meningitidis serogroups B (DE 8759) was checked by confocal laser scanning microscopy. 10⁸ heat inactivated meningococci were washed three times with PBS, incubated with pooled post-immune (anti tetrasaccharide 1-CRM sera) and pre-immune 1:100 sera dilutions in 1% BSA-PBS for 1 h followed by washing with 1% BSA-PBS. As negative control bacteria were also incubated with PBS. To detect bound primary antibodies bacteria were incubated with Rabbit anti-Mouse IgG (whole molecule)-FITC (fluorescein 5(6)-isothiocyanate) (1:100 in 1% BSA-PBS) and 476 nM DAPI (4′,6-diamidino-2-phenylindole, Life Technologies) was used for nuclear staining. 5-10 μl of washed sample was used for fluorescence microscopy. Imaging of meningococci in presence of pre-immune and post-immune sera was performed with identical settings using Zen 2009 (Zeiss) software and a LSM 700, AXIO Imager.M2 (Zeiss). Green fluorescence of FITC indicated binding of antibodies from sera to the heat inactivated bacteria. The staining with prebleed sera (FIG. 17 B) showed just a weak reactivity, whereas labeling with post-immune sera (FIG. 17 C) gave significantly higher green fluorescence signal which localized well with the nuclear staining and the DIC (differential interference contrast) images. Bacteria incubated with PBS and only secondary Rabbit anti-Mouse IgG-FITC was employed as the negative control. In negative control no green fluorescence was observed and only the blue fluorescence of the nuclear staining was visible (FIG. 17 A). This demonstrated that the sera which was generated against the synthetic tetrasaccharide 1-CRM₁₉₇ conjugate was reactive against heat-inactivated meningococci. Positive labeling with post-immune sera was observed for several representatives of eight meningococcal serogroups.

### Example 13

### FACS Analysis (Fluorescence Activated Cell Sorting)

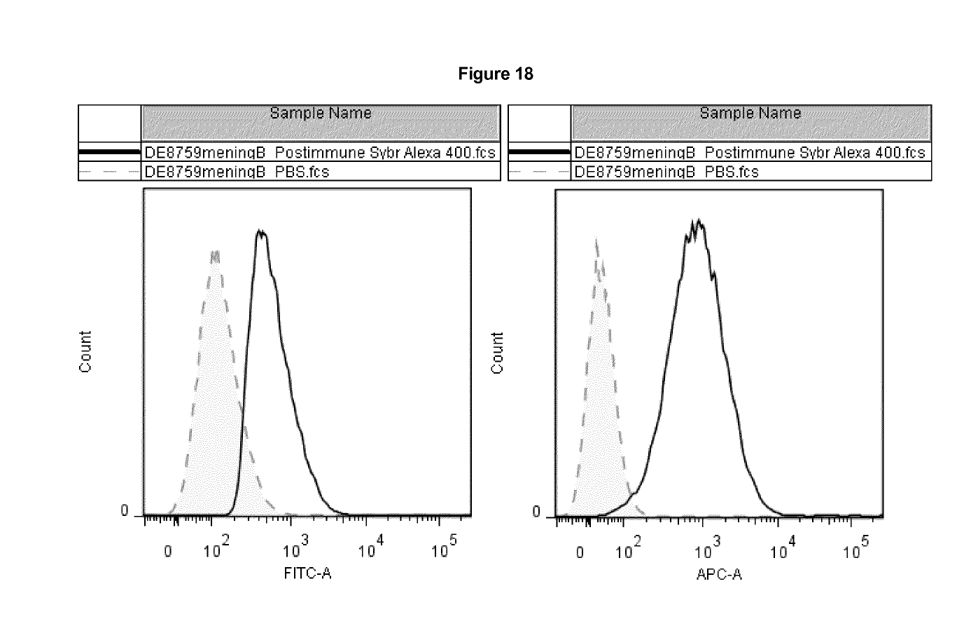

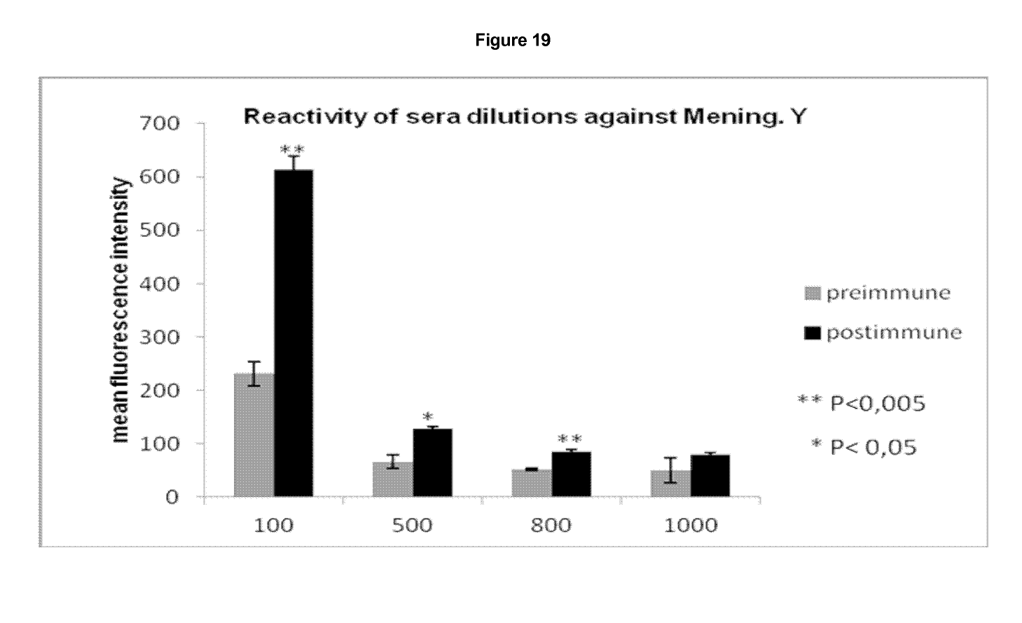

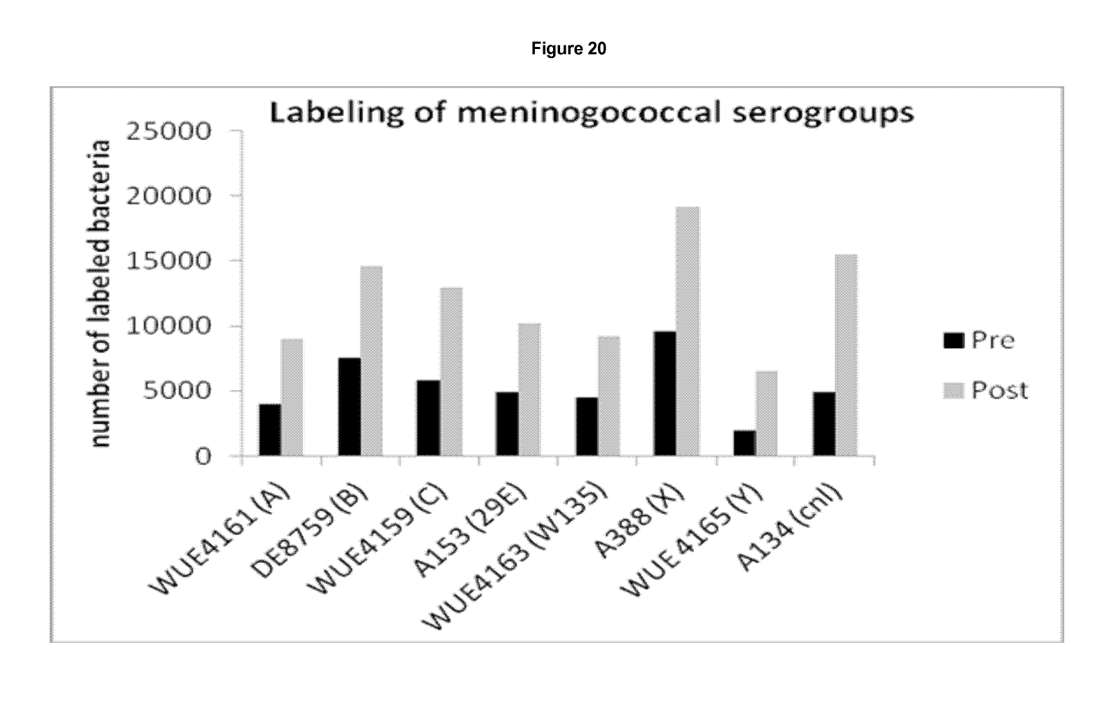

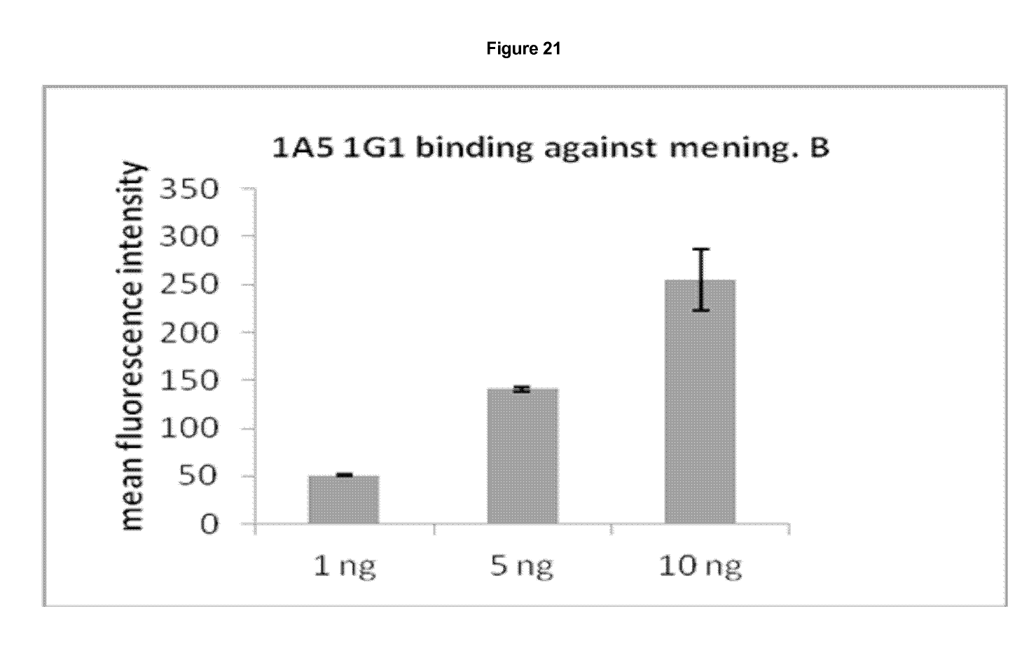

Binding studies against N. meningitidis was evaluated by FACS (fluorescence activated cell sorting). 2×10⁸ inactivated bacteria were washed once with PBS, twice with 1% BSA-PBS and incubated with mAb, pooled post-immune and pre-immune sera dilution in 1% BSA-PBS for 1 h at 4° C. After washing with 1% BSA-PBS primary antibodies on bacteria were detected by Alexa Fluor 635 Goat Anti-Mouse IgG (H+L) secondary antibody (Life Technologies). DNA staining was performed with SYBR® Safe DNA Gel Stain (1:1000, Life Technologies). Washed samples were inactivated with 8% paraformaldehyde, washed and measured by BD FACSCanto™ II, whereby only SYBR® Safe positive (FITC positive) bacteria were analysed for labeling with Alexa Fluor 635 Goat Anti-Mouse IgG (H+L) secondary antibody (APC positive). Percentage of bacteria positive to APC specific fluorescence was considered as bacteria bound to antibody and the results are shown in FIG. 18 at 1:100 postimmune dilution. Dilution-wise analysis of post-immune and pre-immune sera showed a significant binding to N. meningitidis Y (WUE 4165) up to a 1:800 dilution (FIG. 19). Furthermore, one representative of each meningococcal serogroup was stained with 1:100 dilutions of post-immune and pre-immune sera. In FIG. 20 it was visualized that all eight strains were recognized by post-immune sera. The results from FACS analysis corroborated the findings from confocal microscopy. Reactivity of the purified mAb 1A5 1G1 against formalin-inactivated N. meningitidis B was checked dilutions-wise and 10 ng/μl was observed to be the optimal concentration for binding (FIG. 21).

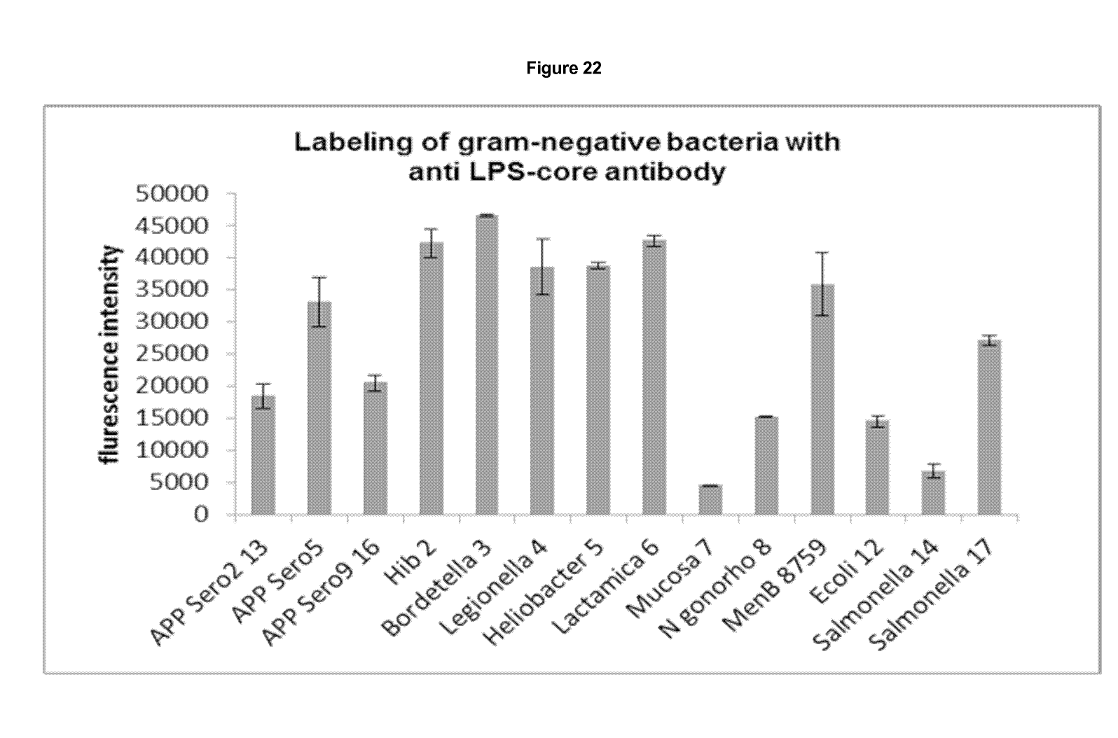

Glycan array studies showed that post-immune sera and mAb 1A5 1G1 are reactive against tetrasaccharide 1 as well as the trisaccharide 37. Because this trisaccharide represents the conserved LPS-core structure of many gram-negative bacteria, reactivity of the mAb 1A5 1G1 (diluted to 10 ng/μl) against several formalin-inactivated gram-negative bacteria were analysed by FACS. As shown in FIG. 22, the mAb 1A5 1G1 recognized all gram-negative bacteria, which were chosen for this analysis except N. mucosa.

### Example 14

### Opsonophagocytosis Assay

To evaluate the opsonophagocytic potential of anti-tetrasacharide 1 sera, an opsonophagocytosis assay (OPA) was performed. FITC labeling of 2×10⁸ meningococci B were performed in 50 mM Na₂CO₃, 100 mM NaCl, pH 7.5 with 500 μg FITC for 30 min at 37° C. Samples were washed five times with washing buffer (0.25% BSA, 2 mM HEPES, PBS). Labeled bacteria were pre-incubated with 1:100 pooled post-immune sera dilution, mAb 1A5 1G1 supernatant and PBS or hybridoma media as negative control at 37° C. Subsequently these bacteria were incubated with 10⁶ confluent macrophages (RAW 264.7 cells) at 4° C. for adhesion of bacteria to the macrophage cell surface followed by washing with PBS to remove the nonspecifically bound bacteria. Then macrophages were incubated at 37° C., 5% CO₂ which promoted uptake of previously opsonized bacteria specifically to the intracellular compartments. Washed macrophages were successively stained with mAb 1A5 1G1 supernatant and Goat Anti-Mouse IgG Alexa Fluor 635 (H+L) secondary antibody (1:500) at 4° C. Macrophages were inactivated with 8% paraformaldehyde, washed with PBS and measured by FACS.

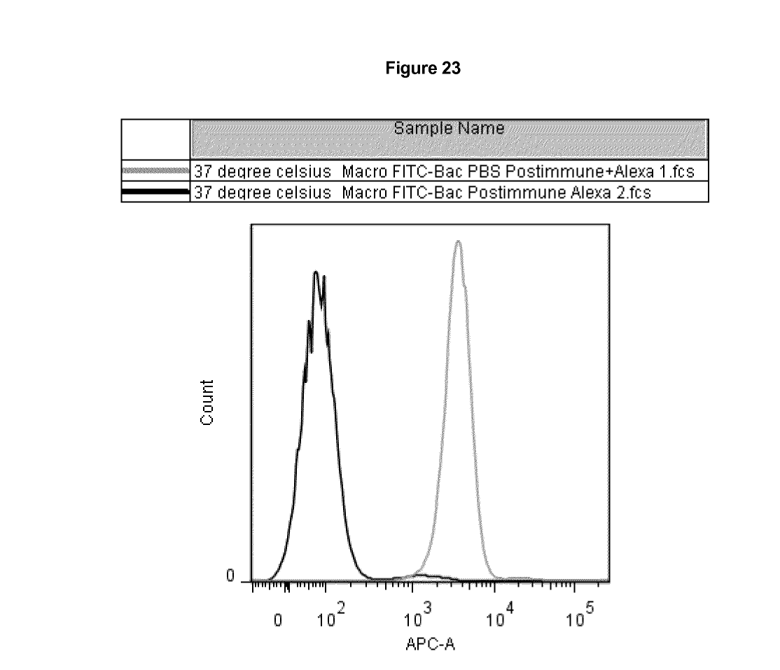

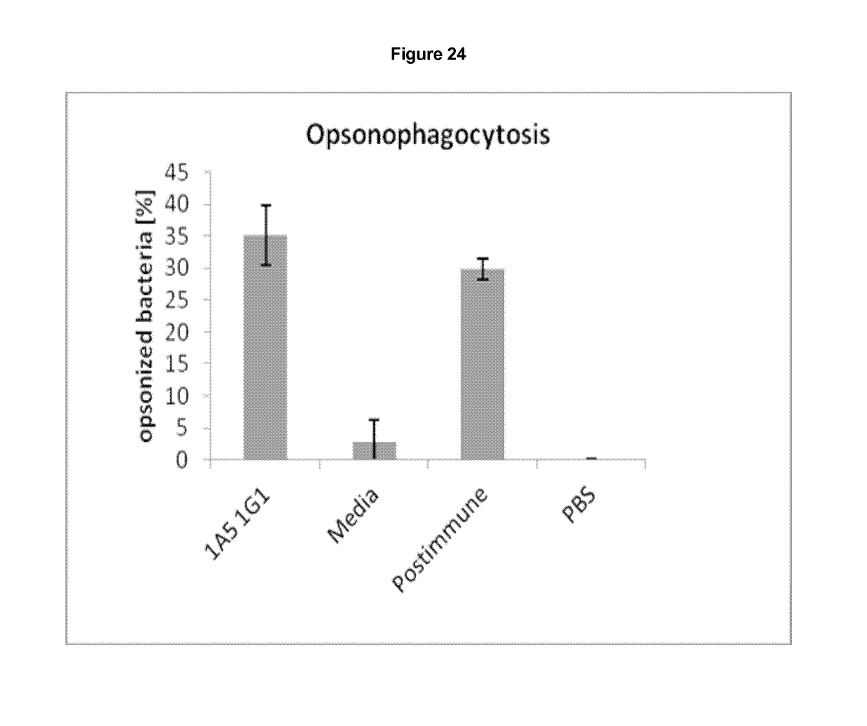

Successful opsonohagocytosis is indicated by FITC positive macrophages in FACS measurement. To detect remaining non-phagocytosed bacteria on the cell surface, samples were stained with mAb 1A5 1G1 supernatant and detected with Goat Anti-Mouse IgG Alexa Fluor 635 (H+L) secondary antibody (APC-FITC double positive). The cells treated with bacteria preincubated with post-immune sera showed higher FITC positive cells and lower APC-FITC double positive cells (black line, FIG. 23) (APC-FITC positive cells is an indication of non-specific binding and FITC positive cells represent fraction of phagocytosed cells). Whereas cells treated with bacteria preincubated with PBS, showed lower FITC positive cells and realtively high APC-FITC double positive cells indicating lower specific uptake and higher non-specific adhesion of bacteria (grey line, FIG. 23). The results indicate that the antibodies present in the post-immune sera are capable of inducing a serum dependent opsonophagocytosis (FIG. 24). This could be beneficial for the immunoprotective effects and supports the vaccine potentail of the conjugated syntheteic saccharides of general formula (I) used in the immunizations.

### Example 15.1

### Formulation of a Meningococcal Vaccine

Materials used in this preparation include tetrasaccharide 1/CRM₁₉₇ conjugates that are prepared in accordance with Example 3 and sterile 100 mM sodium phosphate buffered physiological saline (0.85% sodium chloride). An aliquot of sterile 200-500 mM sodium phosphate buffered physiological saline is added to physiological saline (0.85%) in a stainless steel tank to yield a final vaccine concentration of 10 mM sodium phosphate. An aliquot of the tetrasaccharide 1/CRM₁₉₇ conjugate is added to yield a final concentration of 8 μg/per milliliter of buffer. The conjugate solution is subsequently mixed and filtered for sterilization.

### Example 15.2

### Preparation of Aluminum-Hydroxide Adjuvanted Meningococcal Vaccine

An aliquot of a sterile conjugate of the trisaccharide 37 and diphtheria toxoid conjugates is added to physiological saline to yield a final concentration of 4 μg per milliliter of buffer. An aliquot of sterile aluminum hydroxide in physiological saline (0.85% sodium chloride) is added to the conjugate to achieve a final concentration of 0.44 mg aluminum ion per milliliter vaccine.

### Example 15.3

### Preparation of Meningococcal Vaccine Containing Sucrose as a Lyophilization Stabilizer

In an attempt to achieve the stability goal, the tetrasaccharide 1/CRM₁₉₇ conjugate was lyophilized. The compositions had been lyophilized in the presence of 4.5% mannitol and 1.5% sucrose. The integrity of the individual conjugates appeared to be conserved even after 6 months of post-lyophilization and storage at 4° C. Lyophilization permits the conjugates to be used in combination with an oil-in-water emulsion. Two vaccine formulations of the lyophilized tetrasaccharide 1/CRM₁₉₇ conjugate were prepared. The formulation contains 70 μg of the conjugate, 15 mg PBS, and either 14 mg mannitol or 21 mg mannitol+35 mg sucrose, and may be reconstituted with 700 μl water for injection.

### Example 16

### Further Immunizations

To evaluate the immunogenicity of the meningococcal trisaccharides 37 and monosaccharide 34, female BALB/c mice, six to eight weeks old, were immunized according to example 6.

Each mouse was immunized s.c. with 0.4 μg unconjugated or CRM₁₉₇-conjugated trisaccharide 37 or unconjugated or CRM₁₉₇-conjugated monosaccharide 34 in presence of Freund's adjuvant (the amount of conjugate corresponded to 0.4 μg saccharide). Specific IgG and IgM antibody responses were identified in pooled sera. This study showed that the conjugated trisaccharide 37 is immunogenic in the presence of FA but elicited less boosting IgG responses then using the tetrasaccharide 1/CRM₁₉₇ conjugate. The monosaccharide 34/CRM₁₉₇ conjugate did only elicit a very weak antibody response (see table 2).

| Antigen used | IgG response |
|---|---|
| tetrasaccharide 1 unconjugated | + |
| tetrasaccharide 1/CRM₁₉₇ conjugate | +++ |
| trisaccharide 37 unconjugated | + |
| trisaccharide 37/CRM₁₉₇ conjugate | ++ |
| monosaccharide 34 | − |
| monosaccharide 34/CRM₁₉₇ conjugate | + |
| tetrasaccharide 149 unconjugated | − |
| tetrasaccharide 149/CRM₁₉₇ conjugate | ++ |
| tetrasaccharide 142 unconjugated | + |
| tetrasaccharide 142/CRM₁₉₇ conjugate | ++ |
| tetrasaccharide 135 unconjugated | + |
| tetrasaccharide 135/CRM₁₉₇ conjugate | +++ |
| tetrasaccharide 128 unconjugated | + |
| tetrasaccharide 128/CRM₁₉₇ conjugate | ++ |
| tetrasaccharide 121 unconjugated | − |
| tetrasaccharide 121/CRM₁₉₇ conjugate | ++ |
| trisaccharide 114 unconjugated | − |
| trisaccharide 114/CRM₁₉₇ conjugate | ++ |
| trisaccharide 107 unconjugated | + |
| trisaccharide 107/CRM₁₉₇ conjugate | ++ |
| trisaccharide 100 unconjugated | + |
| trisaccharide 100/CRM₁₉₇ conjugate | ++ |
| trisaccharide 93 unconjugated | + |
| trisaccharide 93/CRM₁₉₇ conjugate | ++ |
| trisaccharide 86 unconjugated | − |
| trisaccharide 86/CRM₁₉₇ conjugate | ++ |
| disaccharide 36 unconjugated | − |
| disaccharide 36/CRM₁₉₇ conjugate | + |
| disaccharide 64 unconjugated | − |
| disaccharide 64/CRM₁₉₇ conjugate | − |
| disaccharide 59 unconjugated | − |
| disaccharide 59/CRM₁₉₇ conjugate | + |
| disaccharide 69 unconjugated | − |
| disaccharide 69/CRM₁₉₇ conjugate | + |
| disaccharide 79 unconjugated | − |
| disaccharide 79/CRM₁₉₇ conjugate | − |
| disaccharide 74 unconjugated | − |
| disaccharide 74/CRM₁₉₇ conjugate | + |
| monosaccharide 48 unconjugated | − |
| monosaccharide 48/CRM₁₉₇ conjugate | − |

1. Synthesis of synthetic saccharides of general formula (I) 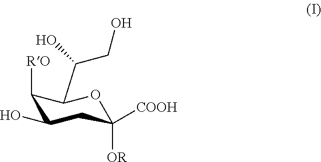
wherein
R represents —Y—NH₂
Y represents a linker
R′ represents
H or 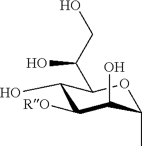
R″ represents
H or 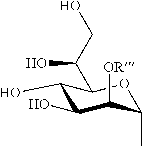
R′″
represents H
or 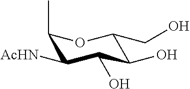
comprising or consisting of the steps:
A1) Reacting compound 26* with a compound HO—Y—NP¹⁴P¹⁵, 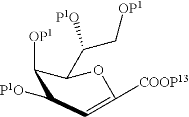
wherein P¹, P¹³, P¹⁴ and P¹⁵ are protecting groups and Y is defined as above, yielding a compound of the formula 28^{#} 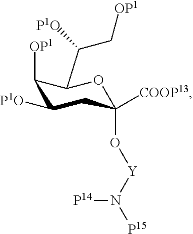
A2) Performing deprotection reactions with compound 28^{#} yielding a monosaccharide 35^{#}, 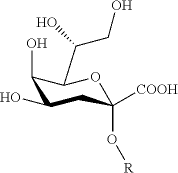
wherein R is defined as above,
or
converting compound 28^{#} to compound 7^{#}, 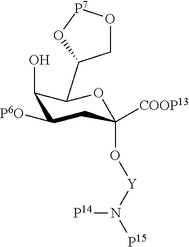
wherein P⁶ and P⁷ are protecting groups, and Y, P¹³, P¹⁴ and P¹⁵ are defined as above;
B1) Reacting compound 6* of the formula 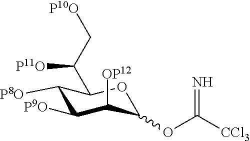
wherein
P⁸-P¹² represent protecting groups
with compound 7^{#} in order to obtain a compound 31^{#} of the following chemical formula 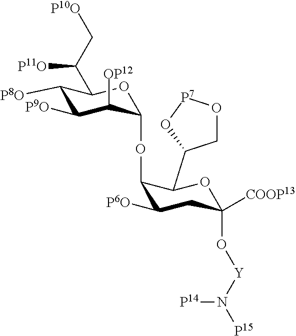
wherein Y and P⁶ to P¹⁵ are defined as above;
B2) Performing deprotection reactions with compound 31^{#} yielding a disaccharide 36^{#}, 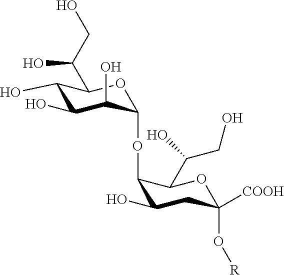
wherein R is defined as above,
or
performing a selective deprotection yielding a compound of the formula 3#, 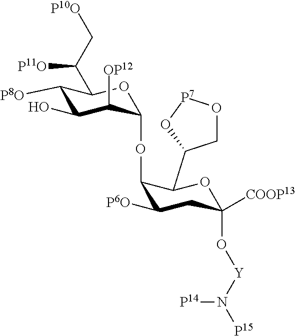
Wherein P⁶ to P⁸, P¹⁰ to P¹⁵ and Y are defined as above;
C1) Reacting compound 3^{#} with a compound of the formula 38^{#} 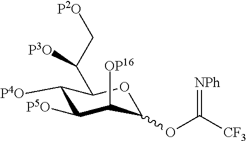
wherein P²-P⁵ and P¹⁶ are protecting groups,
yielding a compound of the formula 39^{#}, 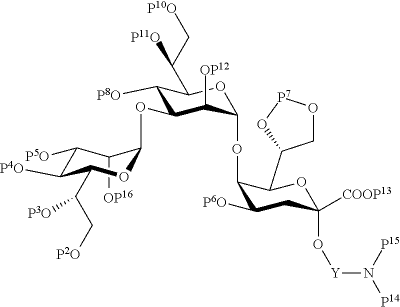
wherein P²-P⁸, P¹⁰-P¹⁶ and Y are defined as above,
and
D1) Performing deprotection reactions with compound 39^{#} yielding a trisaccharide 37^{#} 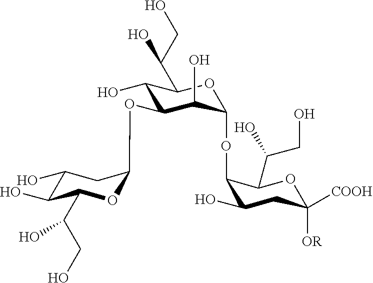
wherein R is defined as above,
or
C2) Reacting the compound 4* of the formula 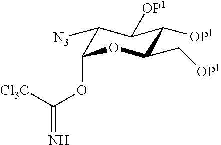
wherein
the groups P¹ represent the same protecting group
with the compound 5* of the following chemical formula 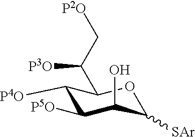
wherein
P²-P⁵ are defined as above
and
Ar represents an aromatic ring or aromatic ring system
in order to obtain compound 30* of the following chemical formula 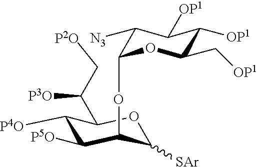
wherein
the group —SAr is converted to the group —O—C(═NPh)-CF₃ in order to obtain compound 2* of the following chemical formula 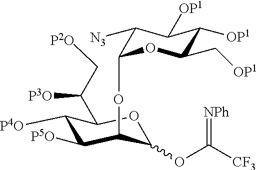
wherein P¹-P⁵ are defined as above,
Reacting compound 3^{#} with a compound 2*,
yielding a compound of the formula 32#, 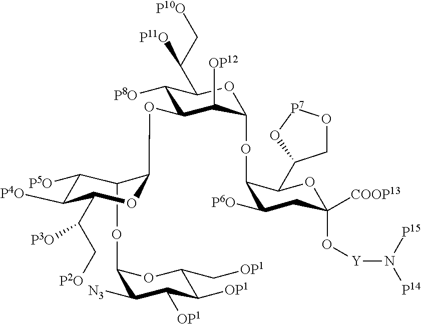
wherein P¹-P⁸, P¹⁰-P¹⁵ and Y are defined as above,
and
D2) Converting the azide group of compound 32^{#} into an acetamide group and performing deprotection reactions yielding a tetrasaccharide 1^{#} 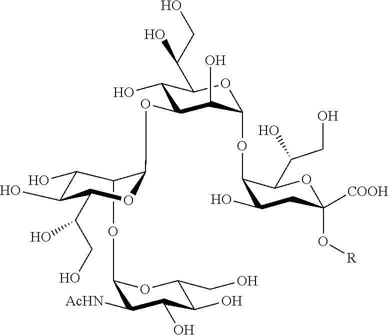
wherein R is defined as above.

2. Synthesis according to claim 1 of the tetrasaccharide 1 of the chemical formula 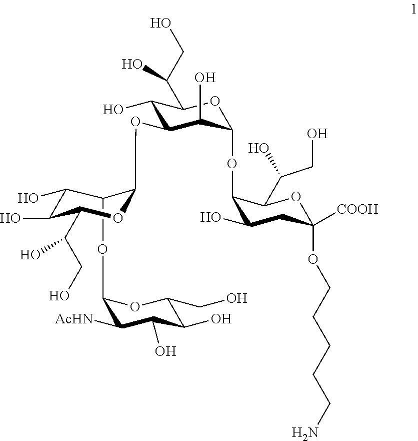
comprising the steps:
A) Reacting compound 6* of the formula 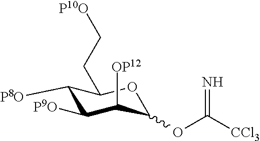
wherein
P⁸-P¹² represent protecting groups
with compound 7* of the formula 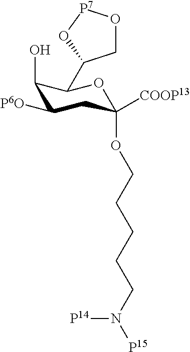
wherein
P⁶, P⁷, P¹³-P¹⁵ represent protecting groups
in order to obtain compound 31* of the following chemical formula 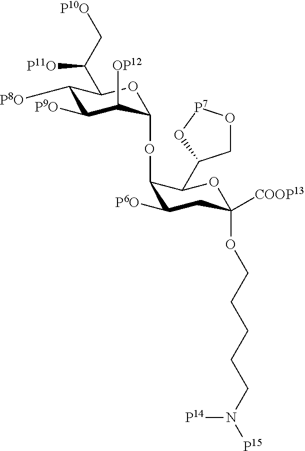
wherein the protecting group P⁹ is selectively cleaved in order to obtain compound 3*.
B) Reacting compound 4* of the formula 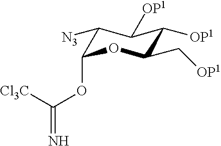
wherein
P¹ represent the same protecting group
with compound 5* of the following chemical formula 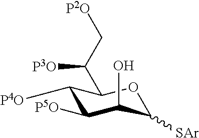
wherein
P²-P⁵ represent protecting groups and
Ar represents an aromatic ring or aromatic ring system
in order to obtain compound 30* of the following chemical formula 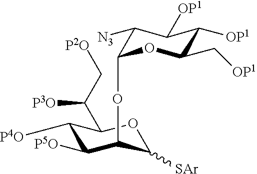
wherein
the group —SAr is converted to the group —O—C(═NPh)-CF₃ in order to obtain compound 2* of the following chemical formula 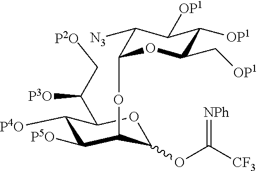
C) Reacting compound 2* with compound 3* of the formula 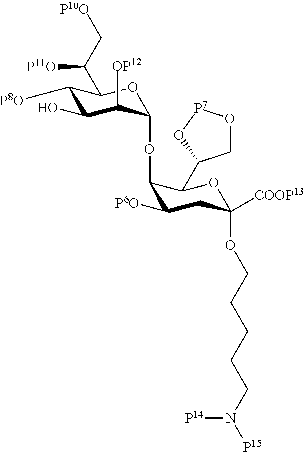
in order to obtain compound 32* of the formula 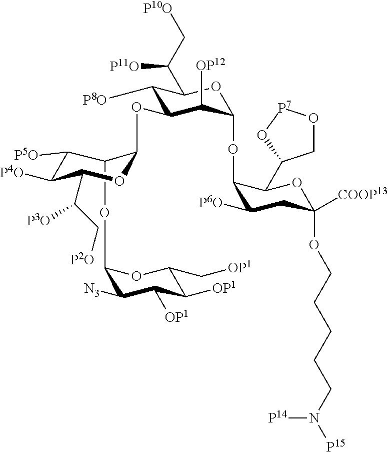
wherein the azide group is converted into an acetamide group and the protecting groups P¹-P⁸ and P¹⁰-P¹⁵ are cleaved in order to obtain tetrasaccharide 1.

3. Synthesis according to claim 1 or 2 comprising step D:
D) preparing a salt of tetrasaccharide 1 or preparing a lyophilisate of tetrasaccharide 1 or of the salt of tetrasaccharide 1.

4. Synthesis according to claim 1 or 2, wherein the reaction of compound 6* and 7*, the reaction of compound 2* and 3* or the reaction of compound 4* and 5* is performed in a polar aprotic solvent using TMSOTf.

5. Synthesis according to claim 1 or 2, wherein the conversion of compound 30* to 2* is performed in two steps, first by reacting compound 30* in a polar aprotic solvent and water mixture using NBS and second by reacting the product obtained after the first step with CF₃C(═NPh)Cl and a base in a polar aprotic solvent.

6. Synthesis according to claim 1 or 2, wherein the conversion of compound 31* to 3* is performed in a polar aprotic solvent by means of hydrazine or a hydrazinium salt.

7. Synthesis according to claim 1 or 2, wherein the conversion of compound 32* to 33* of the formula 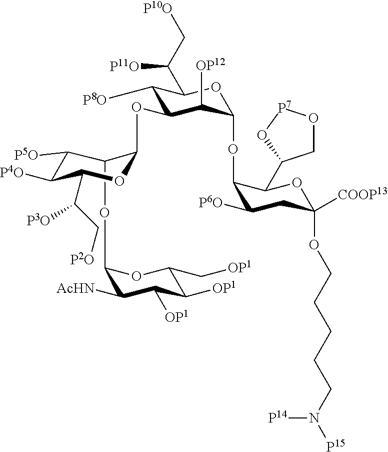
is performed in a polar aprotic solvent to which a base is added or in a polar aprotic basic solvent using thioacetic acid.

8. Synthesis according to claim 1 or 2, wherein the conversion of compound 33* to 1 is performed in three steps, first the acid-labile protecting groups are cleaved in a mixture of an acid in water; second the base-labile protecting groups are cleaved in a polar aprotic solvent or a polar aprotic solvent mixture using a base; and third the protecting groups sensitive for hydrogenation are cleaved by means of hydrogen and a catalyst.

9. The compound 2* of the following formula 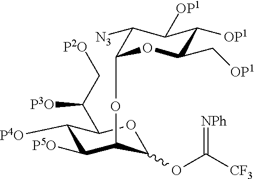
wherein P¹ represents acetyl, P² and P⁵ represent benzoyl, P³ represents benzyl and P⁴ represents para-bromobenzyl.

10. The compound 3* of the following formula 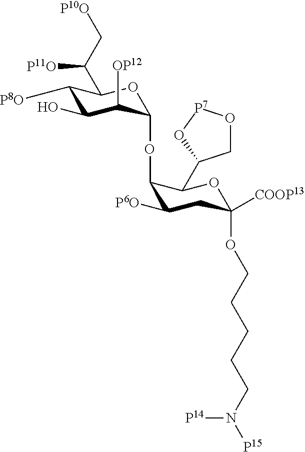
wherein P⁶, P¹¹ and P¹⁴ represent benzyl, P⁷ represents isopropyl, P⁸ represents para-bromobenzyl, P¹⁰ and P¹² represent acetyl and P¹⁵ represents benzyloxy carbonyl and P¹³ represents methyl.

11. Saccharide of the general formula 1^{#} 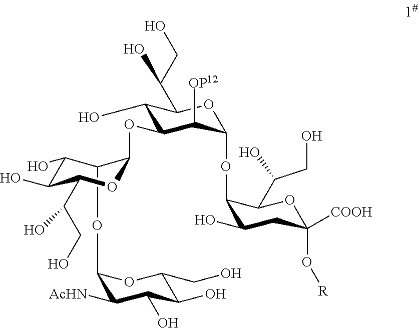
wherein
R represents —Y—NH₂;
Y represents a linker.

12. Saccharide of the general formula 37^{#} 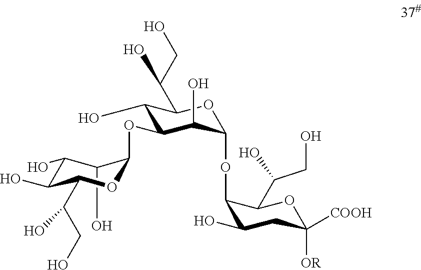
wherein
R represents —Y—NH₂;
Y represents a linker.

13. Saccharide selected from the following group comprising the following compounds:
tetrasaccharide 1: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(5-amino)pentyl-3-deoxy-α-D-manno-oct-2-ulopyranosidonic acid
mono-saccharide 35: 2-(5-amino)pentyl-3-deoxy-a-D-manno-oct-2-ulopyranosidonic acid
disaccharide 36: L-glycero-a-D-manno-heptopyranosyl-(1→5)-2-(5-amino) pentyl-3-deoxy-a-D-manno-oct-2-ulopyranosidonic acid
trisaccharide 37: L-glycero-α-D-manno-heptopyranosyl-(1→3)-L-glycero-α-D-manno-heptopyranosyl-(1→5)-2-(5-amino)pentyl-3-deoxy-α-D-manno-oct-2-ulopyranosidonic acid
compound 42: 2-(2-amino)ethyl-3-deoxy-a-D-manno-oct-2-ulopyranosidonic acid
compound 45: 2-(22-amino)docosanyl-3-deoxy-a-D-manno-oct-2-ulopyranosidonic acid
compound 48: 2-2-(2-aminoethoxy)ethyl-3-deoxy-a-D-manno-oct-2-ulpyrano sidonic acid
compound 51: 2-2-(5-aminomethyl)pyrrolidin-2-yl)ethyl-3-deoxy-a-D-man no-oct-2-ulpyranosidonic acid
compound 54: 2-4-(2-aminoethoxy)benzyl-3-deoxy-a-D-manno-oct-2-ulpyranosidonic acid
compound 59: L-glycero-a-D-manno-heptopyranosyl-(1→5)-2-(38-amino-3,6,9,12,15,18,21,24,27,30,33,36-dodecaoxaoctatriacontanyl)-3-deoxy-a-D-manno-oct-2-ulopyranosidonic acid
compound 64: L-glycero-a-D-manno-heptopyranosyl-(1→5)-2-(((aminomethyl)disulfanyl)methyl)-3-deoxy-a-D-manno-oct-2-ulopyranosidonic acid
compound 69: L-glycero-a-D-manno-heptopyranosyl-(1→5)-2-(2-((2-aminoethyl)(methyl)amino)ethyl)-3-deoxy-a-D-manno-oct-2-ulopyranosidonic acid
compound 74: L-glycero-a-D-manno-heptopyranosyl-(1→5)-2-(2-(aminomethoxy)phenoxy)methyl-3-deoxy-a-D-manno-oct-2-ulopyranosidonic acid
compound 79: L-glycero-a-D-manno-heptopyranosyl-(1→5)-2-(3-(6-(aminomethyl)piperidin-2-yl)propanyl)-3-deoxy-a-D-manno-oct-2-ulopyranosidonic acid
compound 86: L-glycero-α-D-manno-heptopyranosyl-(1→3)-L-glycero-α-D-manno-heptopyranosyl-(1→5)-2-(40-amino)tetracontanyl-3-deoxy-α-D-manno-oct-2-ulopyranosidonic acid
compound 93: L-glycero-α-D-manno-heptopyranosyl-(1→3)-L-glycero-α-D-manno-heptopyranosyl-(1→5)-2-(2-(4-aminophenoxy)ethyl-3-deoxy-α-D-manno-oct-2-ulopyranosidonic acid
compound 100: L-glycero-α-D-manno-heptopyranosyl-(1→3)-L-glycero-α-D-manno-heptopyranosyl-(1→5)-2-(2-(4-(2-aminoethyl)-2-methoxyphenyl)ethyl)-3-deoxy-α-D-manno-oct-2-ulopyranosidonic acid
compound 107: L-glycero-α-D-manno-heptopyranosyl-(1→3)-L-glycero-α-D-manno-heptopyranosyl-(1→5)-2-(4-(2-aminoacetyl)phenyl)-3-deoxy-α-D-manno-oct-2-ulopyranosidonic acid
compound 114: L-glycero-α-D-manno-heptopyranosyl-(1→3)-L-glycero-α-D-manno-heptopyranosyl-(1→5)-2-(6-amino-3-oxo)hexyl-3-deoxy-α-D-manno-oct-2-ulopyranosidonic acid
compound 121: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(8-amino-2,2-dimethyl)octyl-3-deoxy-α-D-manno-oct-2-ulopyranosidonic acid
compound 128: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(2-((aminomethyl)thio)ethyl)-3-deoxy-α-D-manno-oct-2-ulopyranosidonic acid
compound 135: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(3-(3-aminopropyl)ureido)methyl-3-deoxy-α-D-manno-oct-2-ulopyranosidonic acid
compound 142: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(5-(2-aminoethyl)-1-methylpiperidin-2-yl)methyl-3-deoxy-α-D-manno-oct-2-ulopyranosidonic acid
compound 149: 2-N-acetyl-2-deoxy-α-D-glucopyranosyl-(1→2)-L-glycero-α-D-manno-heptopyranosyl-(1→3)-L-glycero-α-D-manno-heptopyranosyl-(1→5)-2-(4-(3-aminopropyl)phenyl)-3-deoxy-α-D-manno-oct-2-ulopyranosidonic acid

14. Saccharide according to claim 11, 12 or 13 which is not contaminated with PEA and/or is not contaminated with endotoxin lipid A.

15. Synthetic saccharide of general formula (I) 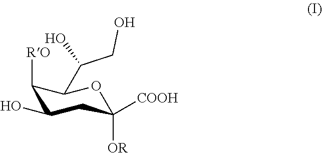
wherein
R represents —Y—NH₂,
Y represents a linker,
R′ represents H or 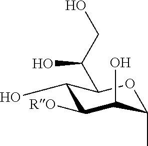
R″ represents H or 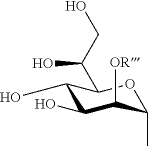
R″ represents H or 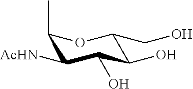
for use as a vaccine for immunization against diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

16. Synthetic saccharide according to claim 15 conjugated to a carrier protein.

17. Synthetic saccharide according to claim 15 or 16, wherein the diseases are selected from the group consisting of meningitis, septicemia, pneumonia and nasopharyngitis.

18. Synthetic saccharide according to claim 15, 16 or 17, wherein the bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep are selected from the group consisting of all strains of Neisseria meningitidis, wherein the lipo-oligosaccharide (LOS) immunotypes are L1, L2, L3, L4, L5, L6, L7, L8, L9 and/or L11.

19. Vaccine composition containing at least one synthetic saccharide of general formula (I) 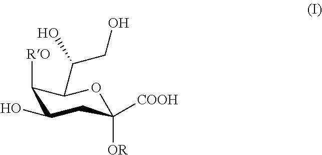
wherein
R represents —Y—NH₂,
Y represents a linker,
R′ represents H or 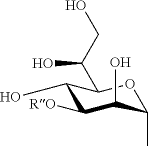
R″ represents H or 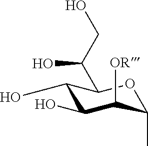
R″ represents H or 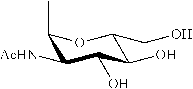
together with at least one pharmaceutically acceptable carrier, cryoprotectant, lyoprotectant, excipient, adjuvant and/or diluent.

20. Antibody against at least one synthetic saccharide of general formula (I) as defined by claim 15.

21. Antibody according to claim 20, wherein the antibody is produced by the monoclonal hybridoma 1A5 1G1 or 1B6 4E1.

22. Use of at least one synthetic saccharide of general formula (I) as defined in claim 14 or at least one antibody of claim 20 or 21 in immunological assays for diagnostics of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

23. Use according to claim 22 wherein the diseases are selected from the group consisting of meningitis, septicemia, pneumonia and nasopharyngitis.

24. At least one synthetic saccharide of general formula (I) as defined by claim 15 or at least one antibody of claim 20 or 21 for use in the treatment or prophylaxis of diseases caused by bacteria containing LPS comprising a saccharide sequence selected from α-GlcNAc-(1→2)-α-Hep-(1→3)-α-Hep-(1→5)-α-Kdo, α-Hep-(1→3)-α-Hep-(1→5)-α-Kdo and α-GlcNAc-(1→2)-α-Hep-(1→3)-α-Hep.

25. At least one synthetic saccharide or at least one antibody according to claim 24 wherein the diseases are selected from the group consisting of meningitis, septicemia, pneumonia and nasopharyngitis.
